(19) United States
(12) Patent Application Publication
(10) Pub. No.: US 2006/0003959 A1
(43) Pub. Date: Jan. 5, 2006

(54) Methods and agents for maintaining muscle mass and for preventing muscle atrophy and biomarkers for monitoring same

(76) Inventors: Steven Burden, Sharon, CT (US); Daniel Littman, New York, NY (US)

Correspondence Address: KLAUBER & JACKSON, 411 HACKENSACK AVENUE, HACKENSACK, NJ, 07601, US

(21) Appl. No.: 11/150,888

(22) Filed: Jun. 10, 2005

Related U.S. Application Data
(60) Provisional application No. 60/578,795, filed on Jun. 10, 2004.

(51) Int. Cl. A61K 48/00

(52) U.S. Cl. 514/44

(57) ABSTRACT

The present invention relates to methods for stimulating muscle growth and/or for inhibiting muscle atrophy in a subject suffering from or predisposed to having a muscle wasting disease by administering an agent that increases the expression of the runx1 gene or gene product in an individual. The invention further provides a biomarker for a muscle wasting disease or condition and methods for screening or diagnosing a subject having, or predisposed to having, a muscle wasting disease by measuring the level of expression of the runx1 gene, or its target genes or gene products in an individual. The invention further relates to the preparation of pharmaceutical compositions using agents that may aid in muscle growth and that may prevent or inhibit muscle atrophy by increasing the expression and/or activity of the runx1 gene and/or gene product.

### CROSS-REFERENCE TO RELATED APPLICATIONS

The present application is a non-provisional application claiming the priority of copending provisional application Ser. No. 60/578,795, filed Jun. 10, 2004, the disclosure of which is incorporated by reference herein in its entirety. Applicants claim the benefits of this application under 35 U.S.C. §119 (e).

### GOVERNMENT RIGHTS CLAUSE

The research leading to the present invention was supported, at least in part, by grant number 5R01 NS27963. Accordingly, the Government may have certain rights in the invention.

### FIELD OF THE INVENTION

The invention relates generally to the field of muscle growth and/or prevention of muscle atrophy, and more particularly, to the identification of specific genes in muscle that when activated may minimize muscle atrophy, and which may be used as a biomarker for muscle wasting diseases or conditions. The invention further provides for a means by which muscle wasting may be prevented by utilizing agents that increase the expression or activity of these genes.

### BACKGROUND OF THE INVENTION

Although most skeletal muscle genes are expressed at similar levels in electrically active, innervated muscle and in electrically inactive, denervated muscle, a small number of genes, including those encoding the acetylcholine receptor, N-CAM, and myogenin, are expressed at significantly higher levels in denervated than in innervated muscle. The pathways and mechanisms that mediate electrical activity-dependent gene regulation are not understood, but these mechanisms are likely to be responsible, at least in part, for the changes in muscle structure and function that accompany a decrease in myofiber electrical activity.

In skeletal muscle, changes in the pattern of electrical activity regulate the expression of proteins involved in synaptic transmission, contraction and metabolism. Disruptions in electrical activity, resulting from trauma, immobilization, disease or aging ultimately lead to changes in muscle structure and function, including a decrease in myofiber size, termed muscle atrophy (Jagoe and Goldberg 2001; Glass 2003). The mechanisms that regulate muscle atrophy are poorly understood, but it seems likely that changes in gene expression play a key role in initiating and maintaining a muscle atrophy program.

Although members of the forkhead (FoxO) class of transcription factors have recently been shown to have an important role in regulating atrophy (Sandri, M. C. et al. (2004), Cell 117: 399-412; Stitt, T. N. et al. (2004) MolCell 14: 395-403), it seems likely that additional regulatory programs are activated following muscle disuse and govern muscle wasting. Atrophic myofibers, due to their smaller cross-sectional area, have a reduced capacity to generate force, but they neither degenerate nor undergo apoptosis. Indeed atrophic myofibers retain most of the structural features that are characteristic of normal muscle. In contrast, in a variety of congenital myopathies, muscle wasting is far more dramatic. After short periods of inactivity, muscle atrophy is reversible. Even after prolonged periods of disuse, myofiber degeneration remains uncommon, and atrophy can be partially reversed. Taken together, these results raise the possibility that muscle disuse induces compensatory mechanisms to maintain skeletal myofibers and limit atrophy in the absence of innervation-dependent trophic support (Murgia et al. (2000), Nat Cell Biol 2: 142-7).

There is a need for a better understanding of the genes and factors that regulate muscle growth and which prevent muscle atrophy or wasting. There is also a need to more accurately diagnose a patient suspected of having a muscle wasting disease. These needs are addressed by the agents and methods of the present invention.

All publications, patent applications, patents and other reference material mentioned are incorporated by reference in their entirety. In addition, the materials, methods and examples are only illustrative and are not intended to be limiting. The citation of references herein shall not be construed as an admission that such is prior art to the present invention.

### SUMMARY OF THE INVENTION

The present invention relates to the discovery of a target gene, runx1, that regulates growth of muscle. The invention further relates to methods for identifying potential agents that modulate this gene for maintaining muscle mass and for prevention of muscle atrophy and/or autophagy. Furthermore, based on the role of this gene in maintaining muscle mass and in regulating muscle fiber size, screening or diagnostic tests may be developed for assessing the presence or absence of this gene in patients suspected of having, or patients susceptible to development of, muscle wasting diseases. The present invention further relates to probes derived from the nucleotide sequence of the runx1 gene and to antibodies specific for runx1 polypeptides, either of which may be used in establishment of assays for detecting the presence of this gene in patient tissue or cellular samples for diagnostic purposes. Thus, this gene could be used as a biomarker for detection of muscle wasting diseases.

Accordingly, the first aspect of the invention provides for a method of stimulating muscle growth in a mammal in need thereof, the method comprising administering to the mammal an effective amount of an agent that stimulates runx1 gene expression or activity in a muscle cell. The invention further provides for the use of an agent that stimulates runx1 gene expression for stimulating muscle growth in a mammal in need thereof.

A second aspect of the invention provides for a method of inhibiting muscle atrophy or muscle wasting or muscle cell autophagy in a mammal in need thereof, the method comprising administering to the mammal an effective amount of an agent that stimulates runx1 gene expression or activity in a muscle cell. The invention further provides for the use of an agent that stimulates runx1 gene expression for inhibiting muscle atrophy or muscle wasting in a mammal in need thereof.

A third aspect of the invention provides a method for the prophylaxis or treatment of a mammal having, or at risk for having, a condition involving decreased muscle fiber size, or muscle atrophy, or muscle cell autophagy, the method comprising administering to a mammal in need thereof an effective amount of an agent that stimulates runx1 gene expression. The invention further provides for the use of an agent that stimulates runx1 gene expression for treating a mammal having, or at risk for having, decreased muscle fiber size, or muscle atrophy or muscle cell autophagy.

In a particular embodiment, the mammal is a human patient. In a preferred embodiment, the patient may be incapacitated and demonstrate muscle atrophy as a result of injury or age, and also exhibits a decreased level of the runx1 gene or runx1 gene expression compared to an individual who is not incapacitated. In another particular embodiment, the muscle cell is a myoblast or a satellite cell. In another particular embodiment, the muscle cell is in skeletal muscle, in cardiac muscle or in smooth muscle. In yet another particular embodiment, the agent may be compounds such as small synthetic or naturally derived organic compounds, nucleic acids, polypeptides, antibodies, fragments, isoforms, variants, or other materials that may be used independently for such purposes, all in accordance with the present invention.

In another particular embodiment, the muscle growth is the result of an increase in the size of the muscle fibers present in said mammal.

In yet another particular embodiment, the mammal has a condition, which involves muscle damage or muscle atrophy/wasting or muscle cell autophagy. The condition may be a skeletal muscle disease. The skeletal muscle disease may be a myopathy or a dystrophy. The skeletal muscle disease may result from a neuropathy.

In yet another particular embodiment, the skeletal muscle disease may be the result of a neural condition or may result from an injury. The injury may be a nerve injury.

In yet another particular embodiment, the muscle cell is in a patient with a cardiac muscle disorder. The cardiac disorder may be a cardiomyopathy, an ischemic event or damage resulting from an ischemic event, a degenerative congenital disease or any cardiac trauma.

In yet another particular embodiment, the muscle cell is in a patient with myasthenia gravis or a congenital myasthenia gravis syndrome.

In yet another particular embodiment, the muscle cell is in a patient with a smooth muscle disorder. The smooth muscle disorder may be arterial sclerosis, a vascular lesion or a congenital vascular disease. In yet another particular embodiment, the congenital vascular disease involves muscular damage.

A fourth aspect of the invention provides a method for identifying a candidate compound for regulating skeletal muscle mass or function, comprising:
a. contacting a test compound with a cell containing the runx1 gene, or a target of the runx1 gene;
b. determining whether the test compound results in upregulation/activation of a runx1 gene, or a target of a runx1 gene, or a corresponding gene product;
c. selecting a compound that upregulates/activates the runx1 gene, or the target of the runx1 gene or corresponding gene product and further determining whether the test compound increases muscle mass or function in a skeletal muscle atrophy model system in innervated or denervated muscle; and
d. identifying a test compound that modulates muscle mass or function as a candidate compound for regulating skeletal muscle mass or function.

In a particular embodiment, the runx1 gene has a nucleotide sequence that is greater than 80% identical to the sequence of SEQ ID NO: 1. In another particular embodiment, the runx1 gene has a nucleotide sequence that is greater than 90% identical to the sequence of SEQ ID NO: 1. In another particular embodiment, the runx1 gene has a nucleotide sequence that is at least 99% identical to that of SEQ ID NO: 1. In yet another particular embodiment, the runx1 gene has a nucleotide sequence corresponding to the nucleic acid sequence of SEQ ID NO: 1. In yet another embodiment, the target of a runx1 gene is exemplified by any one of SEQ ID NOS: 3-31 and in FIG. 8. In yet another particular embodiment, the cell containing a runx1 gene is a eukaryotic cell. In yet another particular embodiment, the eukaryotic cell is a muscle cell or cell line, or any other eukaryotic cell which contains runx1, or has been genetically engineered to express runx1. In yet another particular embodiment, the cell further comprises a reporter gene operatively associated with a runx1 responsive element and measuring cellular runx1 level involves measuring expression of the reporter gene. The method of determining whether the test compound results in upregulation/activation of the runx1 gene or the runx1 gene product is accomplished using an assay for measuring runx1 RNA or protein expression, and the assay is selected from the group consisting of an immunoassay, an RNase protection assay and an assay wherein a nucleic acid probe is used for detection of the runx1 gene.

A fifth aspect of the invention provides a method of determining if a subject is at risk for developing a muscle wasting disease, the method comprising:
(I) measuring an amount of a runx1 gene or a runx1 target gene or a corresponding gene product in a tissue sample derived from the subject, wherein the runx1 gene or the target of the runx1 gene or corresponding gene product is:
   (a) a DNA corresponding to SEQ ID NO: 1, or to any one of SEQ ID NOS: 3-31 or a nucleic acid derived therefrom;
   (b) a protein comprising SEQ ID NO: 2, or a protein encoded by the nucleic acid sequence of any one of SEQ ID NOS: 3-31;
   (c) a nucleic acid comprising a sequence hybridizable to SEQ ID NO: 1, or to any one of SEQ ID NOS: 3-31 or their complements under conditions of high stringency, or a protein comprising a sequence encoded by the hybridizable sequence;
   (d) a nucleic acid at least 90% homologous to SEQ ID NO: 1, or to any one of SEQ ID NOS: 3-31 or their complements as determined using the NBLAST algorithm; or a protein encoded thereby; and
(II) comparing the amount of the gene product of runx1 or at least one of its target genes in the subject with the amount of gene product of runx1 or at least one of its target genes present in a normal tissue sample obtained from a subject who does not have a muscle wasting disease or in a predetermined standard, wherein a decrease in the amount of the gene product of runx1 or at least one of its target genes in the subject compared to the amount in the normal tissue sample or pre-determined standard indicates a risk of developing a tissue wasting disease in the subject.

A sixth aspect of the invention provides a method for screening, diagnosis or prognosis of a disease in a subject, wherein the disease is characterized by muscle atrophy selected from the group consisting of myopathies, dystrophies, myoneural conductive diseases, traumatic muscle injury, nerve injury, cardiomyopathies, ischemic damage, congenital disease, and other conditions resulting in muscle wasting, the method comprising:
(I) measuring an amount of a runx1 gene or target gene for runx1 or their corresponding gene product in a tissue sample derived from the subject, wherein the runx1 gene or runx1 target gene or corresponding gene product is:
   (a) a DNA corresponding to SEQ ID NO: 1, or to any one of SEQ ID NOS: 3-31 or a nucleic acid derived therefrom;
   (b) a protein comprising SEQ ID NO: 2, or a protein encoded by any one of the nucleic acid sequences of SEQ ID NOS: 3-31;
   (c) a nucleic acid comprising a sequence hybridizable to SEQ ID NO: 1, or to any one of SEQ ID NOS: 3-31 or their complements under conditions of high stringency, or a protein comprising a sequence encoded by the hybridizable sequence;
   (d) a nucleic acid at least 90% homologous to SEQ ID NO: 1, or to any one of SEQ ID NOS: 3-31 or their complement as determined using the NBLAST algorithm; or a protein encoded thereby; and
(II) comparing the amount of the runx1 gene product or the target of the runx1 gene in the subject with the amount of runx1 gene product or the target of the runx1 gene present in a normal tissue sample obtained from a subject who does not have a muscle wasting disease or in a predetermined standard, wherein a decrease in the amount of the runx1 gene product or the runx1 target gene in the subject compared to the amount in the normal tissue sample or pre-determined standard indicates the presence of a tissue wasting disease in the subject.

A seventh aspect of the invention provides a diagnostic method for determining a predisposition to, onset of, or presence of a muscle wasting disease in a subject, comprising detecting in the subject the existence of a change in the level of a runx1 gene, or a runx1 target gene or corresponding gene product, as set forth in SEQ ID NO: 1 through SEQ ID NO: 31, the method comprising:
a) obtaining a tissue biopsy from the subject;
b) permeabilizing the cells in the tissue biopsy;
c) incubating the tissue biopsy or cells isolated from the tissue biopsy with one of the following:
   1) an antibody specific for the runx1 gene product, or for the gene product of the runx1 target gene;
   2) a nucleic acid probe specific for the runx1 gene, or for the runx1 target gene;
d) detecting and quantitating the amount of antibody or nucleic acid probe bound;
e) comparing the amount of antibody or nucleic acid probe bound in the biopsy sample in the subject to the amount of antibody or nucleic acid probe bound in a normal tissue or cellular sample; and
wherein the amount of labeled antibody or nucleic acid probe bound correlates inversely with the predisposition, the onset or the presence of a muscle wasting disease in a subject.

An eighth aspect of the invention provides for the use of a pharmaceutical or veterinary formulation comprising the runx1 nucleic acid or polypeptide as defined above formulated for pharmaceutical or veterinary use, respectively, optionally together with an acceptable diluent, carrier or excipient and/or in unit dosage form. Conventional pharmaceutical or veterinary practice may be employed to provide suitable formulations or compositions.

Thus, the formulations to be used as a part of the invention can be applied to parenteral administration, for example, intravenous, subcutaneous, intramuscular, intraorbital, ophthalmic, intraventricular, intracranial, intracapsular, intraspinal, intracisternal, intraperitoneal, topical, intranasal, aerosol, scarification, and also oral, buccal, rectal or vaginal administration.

The methods for diagnosing or determining the susceptibility of a subject for a muscle wasting disease include, but are not limited to, the use of specific DNA primers in the PCR technique, the use of hybridization probes and the use of polyclonal and monoclonal antibodies.

Even more particularly, the present invention relates to the use of the runx1 gene or part of the gene, cDNA, oligonucleotide or the encoded protein or part thereof for preparation of primers or antibodies as a means of detecting the runx1 gene or gene product in a human subject or in a tissue or cellular sample obtained from the subject.

The invention relates to prophylactic or affirmative treatment of diseases and disorders of the musculature by administering agents that induce or potentiate expression or activity of the runx1 gene or gene product.

A ninth aspect of the invention provides for a biomarker associated with and/or predictive of a muscle wasting disease or condition comprising a runx1 gene or gene product, a target gene of runx1 or its corresponding gene product, or a nucleic acid sequence hybridizable to a runx1 gene or a runx1 target gene or their corresponding gene products, under conditions of high stringency. In a particular embodiment, the biomarker comprises the nucleic acid of SEQ ID NO: 1, or any one of SEQ ID NOS: 3-31. In yet another particular embodiment, the biomarker comprises the amino acid sequence of SEQ ID NO: 2, or the polypeptide encoded by any one of the nucleic acid sequences as set forth in SEQ ID NOS: 3-31. The biomarkers may be used alone or in combination.

In yet another particular embodiment, the biomarker is used to detect the presence of, or the propensity for developing a muscle wasting disease or condition, which is a skeletal muscle wasting disease or condition. In another particular embodiment, the skeletal muscle wasting disease is a myopathy or a dystrophy. In another particular embodiment, the skeletal muscle wasting disease or condition is the result of a neural disorder, a neuropathy or a nerve injury. In another particular embodiment, the skeletal muscle wasting disease or condition is the result of an accident or injury. In another particular embodiment, the muscle wasting disease or condition is characterized by muscle damage, muscle atrophy or muscle cell autophagy. In another particular embodiment, the muscle damage, muscle atrophy or muscle cell autophagy occurs in skeletal muscle, cardiac muscle or smooth muscle. In another particular embodiment, the muscle damage, muscle atrophy, or muscle cell autophagy in cardiac muscle is the result of a cardiomyopathy, an ischemic event (eg., an episode wherein tissue such as cardiac tissue is deprived of oxygen for a period of time due to an occlusion, which results in cell death or damage), a degenerative congenital disease or any cardiac trauma. In another particular embodiment, the muscle damage, muscle atrophy, or muscle cell autophagy in smooth muscle is the result of arterial sclerosis, a vascular lesion, or a congenital vascular disease.

The role of the runx1 gene in muscle maintenance and growth and its potential for use as a biomarker was identified in a mouse model as described herein in Examples 1 through 4. It is envisioned that the preferred biomarkers, including the nucleic acid of SEQ ID NO: 1, or SEQ ID NOS: 3-31 or the polypeptide encoded by the runx1 gene identified in SEQ ID NO: 2, or the polypeptides encoded by the target genes of runx1, as set forth in SEQ ID NOS: 3-31 may be of diagnostic or prognostic use in a clinical setting. Assays detecting this gene or gene product, or variants thereof, the protein or polypeptide or fragments or variants thereof, may be used to assess the presence of a muscle wasting disease or condition or may be used to predict the propensity for developing such a disease or condition. Such assays will augment existing diagnostic methodologies and allow identification and monitoring of patients. They will also facilitate the development of therapeutic agents directed at such diseases or related conditions, while potentially highlighting new targets for such intervention. In addition, these biomarkers may have predictive value in other chronic/acute disease states in which contributing factors or resulting events in common with muscle wasting disease or conditions occur including for instance, but not limited to stroke, various inflammatory conditions or aging.

Other objects and advantages will become apparent from a review of the ensuing detailed description and attendant claims taken in conjunction with the following illustrative drawings. All references cited in the present application are incorporated herein in their entirety.

### BRIEF DESCRIPTION OF THE DRAWINGS

FIG. 1. Illustration of the Constructs Prepared to Inactivate the Runx1 Gene in Muscle. Mice carrying a MCK::cre transgene (Bruning, J. C. et al. (1998), Mol Cell 2: 559-69) were crossed with runx1f/f mice (Taniuchi, I. M. et al. (2002), Cell 111: 621-33) to generate MCK::cre; runx1f/f mice, as well as runx1f/f control mice. The positions of the distal (P1) and proximal (P2) promoters, the loxP sites (triangles), and the primers used to genotype the mice (arrows) are indicated.

FIG. 2. RNase protection analysis demonstrates that exon 4 is deleted in MCK::cre; runx1f/f mice. RNA isolated from denervated (4-day) muscles of runx1f/f control mice fully protects the 280 nt probe from digestion; a low level of protection is observed in innervated muscle, confirming a 50- to 100-fold induction of runx1 following denervation (Zhu et al. 1994). RNA isolated from denervated muscle of MCK::cre; runx1f/f mice protects 105 nt, corresponding to the sequence encoded by exon 5, from digestion; a low level of protection is observed in innervated muscle, demonstrating that the mutant allele, like wild-type runx1, is induced by denervation. Quantitation of these results demonstrates that 96±0.9% (m±s.e.m., n=3) of the runx1 RNA induced by denervation in MCK::cre; runx1f/f mice lacks the DNA-binding Runt domain.

FIG. 3. A lack of muscle Runx1 expression leads to increased muscle atrophy. Limb muscles were denervated for two weeks, fixed and embedded. 1 um-thick sections of innervated (Inn) and denervated (Den) muscles were stained with toluidine blue and were examined. The area of individual myofibers was measured.

FIG. 4. Runx1 mutant denervated myofibers are severely and unusually atrophic. In control runx1f/f mice (a,b), denervated (14-day) myofibers (b) are ˜30% smaller than innervated myofibers (a). In MCK::cre; runx1f/f mice (c,d), denervated (14-day) myofibers (d) are severely atrophic and 10-fold smaller than innervated myofibers (c). The size of innervated myofibers is similar in runx1 mutant (c) and control (a) mice. Bar, 10 μm.

FIG. 5. The severe atrophy of denervated runx1 mutant myofibers is not accompanied by excessive activation of the FoxO or NFκB pathways.

Following denervation, the extent of Fbxo32 (a.k.a. atrogin-1, MAFbx) and MuRF1 induction is similar in MCK::cre; runx1f/f and 23 control runx1f/f mice. As Fbxo32 is a target of FoxO and MuRF1 is induced by NFκB, these results indicate that FoxO and NFκB are similarly activated in denervated muscles from MCK::cre; runx1f/f and control runx1f/f mice. The level of Fbxo32, MuRF1 and Gapd expression in innervated (Inn) and denervated (Den) (3-day) muscles was measured by an RNase protection assay (Zhu, X. et al. (1994), Mol Cell Biol 14: 8051-7).

FIG. 6. Z-discs are disorganized, thick filaments are absent, and the sarcoplasmic reticulum is dilated in runx1 mutant denervated myofibers.

In denervated (14-day) muscle of control runx1f/f mice (a,b), the Z-discs (arrow) are aligned, the actin-only I-bands (I) and actin+myosin A-bands (A) are readily apparent, and the sarcoplasmic reticulum (SR) and transverse-tubules (T) are situated between the myofibrils. In denervated (14-day) MCK::cre; runx1f/f myofibers (c,d), the Z-discs are misaligned and fragmented (arrow in c), distinct I- and A-bands are not discernible, and the SR is severely dilated and extends from the Z-disc into the myofibril. Myofibers that had been denervated for one week were similarly, though less severely affected. Bar, 0.5 μm in (a,c) and 0.12 μm in (b,d).

FIG. 7. Autophagic vacuoles are prominent in runx1 mutant denervated myofibers.

The vacuoles in runx1 mutant denervated (14-day) muscle (MCK::cre; runx1f/f) are enclosed by two bilayers (arrows) that enclose heterogeneous contents, including mitochondria (a), myofibrils (b) and other membrane-enclosed structures (c,d), hallmark features of autophagic vacuoles (Gozuacik and Kimchi 2004). Autophagic vacuoles, *; M, mitochondrion. Bar, 0.12 μm.

FIG. 8. Twenty-nine genes are mis-regulated≧3-fold in runx1 mutant muscle.

The ratio (mean±s.e.m., n=3) of expression, measured on Affymetrix 430 2.0 microarrays (see Materials and Methods), in denervated/innervated (Den/Inn) muscles from runx1f/f control mice and from runx1f/f; MCK::cre mice was calculated. (a) Sixteen genes are induced or 24 maintained by Runx1, as the expression ratio is greater in control than runx1 mutant muscle; (b) thirteen genes are repressed by Runx1, as the expression ratio is less in control than runx1 mutant muscle. (c) We validated the microarray data by measuring expression of a subset of genes, identified as mis-regulated in the microarray screen, by RNase protection (Zhu et al. 1994). These data confirm that expression of these genes is mis-regulated in runx1 mutant muscle.

FIG. 9. Model for control of gene expression and muscle wasting by Runx1 in denervated muscle.

Muscle disuse, caused by damage to peripheral nerves or by immobilization, as well as nutritional starvation and cancer cachexia, lead to muscle atrophy. In wild-type mice, induction of Runx1 limits the extent of muscle wasting and preserves the structural integrity of myofibrils. In the absence of Runx1 induction, muscle disuse leads to the disorganization of myofibrils, excessive autophagy and severely atrophic myofibers. Runx1 activates and represses genes in denervated myofibers, and mis-regulation of these genes in runx1 mutant muscles is responsible for the severe muscle wasting.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

Definitions

The terms used herein have the meanings recognized and known to those of skill in the art, however, for convenience and completeness, particular terms and their meanings are set forth below.

“Agent” refers to all materials that may be used to prepare pharmaceutical and diagnostic compositions, or that may be compounds such as small synthetic or naturally derived organic compounds, nucleic acids, polypeptides, antibodies, fragments, isoforms, variants, or other materials that may be used independently for such purposes, all in accordance with the present invention.

A “therapeutically effective amount” is an amount sufficient to decrease or prevent the symptoms associated with the conditions disclosed herein, including diseases affecting the musculature, or conditions that result in atrophy of the muscles, such as that which occurs in elderly patients or patients that are incapacitated and are bed-ridden, or other related conditions contemplated for therapy with the compositions of the present invention.

“Treatment” or “treating” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted. In the present invention, the treatments using the agents described may be provided to slow or halt net muscle loss, ie. atrophy, or to increase the amount or quality of muscle present in the vertebrate, such as for example, by increasing the number and/or size of the muscle fibers. Most preferably, the treating is for the purpose of reducing or diminishing the symptoms or progression of a disease or disorder of the muscle cells, or to prevent atrophy of the muscle. Treating as used herein also means the administration of the compounds for increasing or altering the muscle cells in healthy individuals.

The term “muscle cell” as used herein refers to any cell which contributes to muscle tissue. Myoblasts, satellite cells, myotubes, and myofibril tissues are all included in the term “muscle cells” and may all be treated using the methods of the invention. Muscle cell effects may be induced within skeletal, cardiac and smooth muscles. Muscle tissue in adult vertebrates will regenerate from reserve myoblasts called “satellite cells”. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

“Muscle growth” as used herein refers to the growth of muscle which may occur by an increase in the fiber size and/or by increasing the number of fibers. The growth of muscle as used herein may be measured by A) an increase in wet weight, B) an increase in protein content, C) an increase in the number of muscle fibers, or D) an increase in muscle fiber diameter. An increase in growth of a muscle fiber can be defined as an increase in the diameter where the diameter is defined as the minor axis of ellipsis of the cross section. The useful therapeutic is one which increases the wet weight, protein content and/or diameter by 10% or more, more preferably by more than 50% and most preferably by more than 100% in a mammal whose muscles have been previously degenerated by at least 10% and relative to a similarly treated control mammal (i.e., a mammal with degenerated muscle tissue which is not treated with the agent that induces muscle growth). A compound which increases growth by increasing the number of muscle fibers is useful as a therapeutic when it increases the number of fibers in the diseased or atrophied tissue by at least 1%, more preferably at least 20%, and most preferably, by at least 50%. These percentages are determined relative to the basal level in a comparable untreated undiseased mammal or in the contralateral undiseased muscle when the agent is administered and acts locally.

“Atrophy” or “wasting” of muscle as used herein refers to a significant loss in muscle fiber girth. By significant atrophy is meant a reduction of muscle fiber diameter in diseased, injured or unused muscle tissue of at least 10% relative to undiseased, uninjured, or normally utilized tissue.

“Autophagy” refers to a mechanism for breaking down cellular components including organelles or long-lived proteins in a cell. “Muscle cell autophagy” refers to the breakdown of cellular components including organelles or long-lived proteins in any type of muscle cell.

“Dystrophy” refers to any of several diseases of the muscular system characterized by weakness and wasting of skeletal muscles. There are nine main forms of the disease. They are classified according to the age at onset of symptoms, the pattern of inheritance, and the part of the body primarily affected.

“Myopathy” refers to any pathology of the muscles that is not attributable to nerve dysfunction.

“Myasthenia gravis” is a disorder of neuromuscular transmission leading to fluctuating weakness and fatigue. A myasthenic crisis may give rise to a generalized paralysis and assisted ventilation may be required. It is one of the best known autoimmune disorders and the antigens and disease mechanisms have well been identified. Weakness is caused by circulating antibodies that block acetylcholine receptors at the neuromuscular junction. Although the disorder usually becomes apparent during adulthood, symptom onset may occur at any age. The condition may be restricted to certain muscle groups, particularly those of the eyes (Ocular Myasthenia Gravis), or may become more generalized (Generalized Myasthenia Gravis), involving multiple muscle groups. Most individuals with Myasthenia Gravis develop weakness and drooping of the eyelids (ptosis); weakness of eye muscles, resulting in double vision (diplopia); and excessive muscle fatigue following exercise. Additional features commonly include weakness of facial muscles; impaired articulation of speech (dysarthria); difficulties chewing and swallowing (dysphagia); and weakness of the upper arms and legs (proximal limb weakness).

“Myasthenic syndrome” refers to a disease characterized by weakness and fatigue of hip and thigh muscles and an aching back; caused by antibodies directed against the neuromuscular junctions

“Myoblast” refers to an undifferentiated cell in the mesoderm of the vertebrate embryo that is a precursor of a muscle cell.

The term “antibody” as used herein includes intact molecules as well as fragments thereof, such as Fab and F(ab′)₂, which are capable of binding the epitopic determinant. Antibodies that bind the genes or gene products of the present invention can be prepared using intact polynucleotides or polypeptides or fragments containing small peptides of interest as the immunizing antigen attached to a carrier molecule. Commonly used carriers that are chemically coupled to peptides include bovine serum albumin and thyroglobulin. The coupled peptide is then used to immunize the animal (e.g, a mouse, rat or rabbit). The antibody may be a “chimeric antibody”, which refers to a molecule in which different portions are derived from different animal species, such as those having a human immunoglobulin constant region and a variable region derived from a murine mAb. (See, e.g., Cabilly et al., U.S. Pat. No. 4,816,567; and Boss et al., U.S. Pat. No. 4,816,397.). The antibody may be a human or a humanized antibody. The antibody may be prepared in mice, rats, goats, sheep, swine, dogs, cats, or horses.

“Gene Product” as used herein, unless otherwise indicated, is a protein or polypeptide encoded by the nucleic acid sequences identified by the methods of the present invention, including but not limited to SEQ ID NOS: 1; or 3-31, a nucleic acid comprising a sequence hybridizable to SEQ ID NOS: 1, or 3-31, or their complement under conditions of high stringency, or a protein comprising a sequence encoded by said hybridizable sequence; a nucleic acid at least 90% homologous to SEQ ID NOS: 1, or 3-31 or their complement as determined using the NBLAST algorithm; a nucleic acid at least 90% homologous to SEQ ID NOS: 1, or 3-31, or a fragment or derivative of any of the foregoing proteins or nucleic acids.

“Diagnosis” or “screening” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

By “homologous” is meant a same sense nucleic acid which possesses a level of similarity with the target nucleic acid within reason and within standards known and accepted in the art. With regard to PCR, the term “homologous” may be used to refer to an amplicon that exhibits a high level of nucleic acid similarity to another nucleic acid, e.g., the template cDNA. As is understood in the art, enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplified nucleic acid (i.e., amplicon) need not be completely identical in every nucleotide to the template nucleic acid.

“Complementary” is understood in its recognized meaning as identifying a nucleotide in one sequence that hybridizes (anneals) to a nucleotide in another sequence according to the rule A→T, U and C→G (and vice versa) and thus “matches” its partner for purposes of this definition. Enzymatic transcription has measurable and well known error rates (depending on the specific enzyme used), thus within the limits of transcriptional accuracy using the modes described herein, in that a skilled practitioner would understand that fidelity of enzymatic complementary strand synthesis is not absolute and that the amplicon need not be completely matched in every nucleotide to the target or template RNA.

As used herein, the terms “nucleic acid”, “polynucleotide” and “oligonucleotide” refer to primers, probes, and oligomer fragments to be detected, and shall be generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including abasic sites). There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single-stranded RNA.

The “polymerase chain reaction (PCR)” technique, is disclosed in U.S. Pat. Nos. 4,683,202, 4,683,195 and 4,800,159. In its simplest form, PCR is an in vitro method for the enzymatic synthesis of specific DNA sequences, using two oligonucleotide primers that hybridize to opposite strands and flank the region of interest in the target DNA. A repetitive series of reaction steps involving template denaturation, primer annealing and the extension of the annealed primers by DNA polymerase results in the exponential accumulation of a specific fragment (i.e, an amplicon) whose termini are defined by the 5′ ends of the primers. PCR is reported to be capable of producing a selective enrichment of a specific DNA sequence by a factor of 10⁹. The PCR method is also described in Saiki et al., 1985, Science, 230:1350.

As used herein, “probe” refers to a labeled oligonucleotide primer, which forms a duplex structure with a sequence in the target nucleic acid, due to complementarity of at least one sequence in the probe with a sequence in the target region. Such probes are useful for identification of a target nucleic acid sequence for EN2 according to the invention. Pairs of single-stranded DNA primers can be annealed to sequences within a target nucleic acid sequence or can be used to prime DNA synthesis of a target nucleic acid sequence.

By way of example and not limitation, procedures using such conditions of low stringency are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency that may be used are well known in the art (e.g., as employed for cross-species hybridizations).

Procedures using such conditions of moderate stringency are as follows: filters comprising immobilized DNA are pretreated for 6 hours at 55° C. in a solution containing 6×SSC, 5× Denhardt's solution, 0.5% SDS and 100 μg/ml denatured salmon sperm DNA. Hybridizations are carried out in the same solution with 5-20×10⁶ cpm ³²P-labeled probe. Filters are incubated in hybridization mixture for 18-20 hours at 55° C., and then washed twice for 30 minutes at 60° C. in a solution containing 1×SSC and 0.1% SDS. Filters are blotted dry and exposed for autoradiography. Washing of filters is done at 37° C. for 1 hour in a solution containing 2×SSC, 0.1% SDS. Other conditions of moderate stringency that may be used are well known in the art. (see, e.g., Sambrook et al., 1989, Molecular Cloning, A Laboratory Manual, 2d Ed., Cold Spring Harbor Laboratory Press, Cold Spring Harbor, N.Y.; see also, Ausubel et al., eds., in the Current Protocols in Molecular Biology series of laboratory technique manuals, 1987-1997 Current Protocols,© 1994-1997 John Wiley and Sons, Inc.).

Procedures using such conditions of high stringency are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used are well known in the art.

The following one letter codes are used to represent amino acids:
S-serine, T-threonine, N-asparagine, Q-glutamine, K-lysine, R-arginine,
H-histidine, E-glutamic acid, D-aspartic acid, C-cystine, G-glycine,
P-proline, A-alanine, I-isoleucine, L-leucine, M-methionine,
F-phenylalanine, W-tryptophan, V-valine, Y-tyrosine, X-any amino acid.

The following one letter codes are used to represent nucleic acids:
A-adenine, C-cytosine, G-guanine, T-thymidine, R represents A or G, Y represents T or C, N represents any nucleic acid.

A “reporter gene” refers to a gene whose phenotypic expression is easy to monitor and is used to study promoter activity in different tissues or developmental stages. Recombinant DNA constructs are made in which the reporter gene is attached to a promoter region of particular interest and the construct transfected into a cell or organism.

“Cardiomyopathy” refers to a disease of the heart muscle that impairs the ability of the heart to pump. Cardiomyopathies (CMPs) are clinically associated with heart failure and an increased risk of sudden cardiac death. Genetic defects are common in all forms of CMPs (dilated, hypertrophic, arrythmogenic right ventricular, and restrictive cardiomyopathies). In familial hypertrophic cardiomyopathy (FHC) the vast majority of defects are mutations in sarcomeric proteins. In dilated and arrythmogenic right ventricular cardiomyopathy (DCM, ARVC) the genetic basis is less well understood.

“Ischemic damage” refers to the damage that occurs to cells, tissues or organs as a result of insufficient blood flow and oxygen to the cells, tissues or organs.

A “small molecule” or “small organic molecule” is an organic compound (or organic compound complexed with an inorganic compound (e.g., metal)) that has a molecular weight of less than 3 kilodaltons, and preferably less than 1.5 kilodaltons.

“Subject” or “patient” refers to a mammal, preferably a human, in need of treatment for a condition, disorder or disease.

A “biomarker” as used herein, refers to a specific molecule, the existence and levels of which are causally connected to a biological process, and reliably captures the state of said process. In the matter of the present invention, the runx1 gene or gene product, such as the nucleic acid or polypeptide, is envisioned for use in detecting a muscle wasting disease or condition, or for use in predicting whether a subject may be predisposed to such muscle wasting disease or condition.

A “target gene of runx1” or “a runx1 target gene” is a gene identified as being dependent upon runx1 expression and is exemplified by the genes set forth in FIG. 8 and as set forth in SEQ ID NOS: 3-31.

General Description

In skeletal muscle, changes in the pattern of electrical activity regulate the expression of proteins involved in synaptic transmission, contraction and metabolism. Disruptions in electrical activity, resulting from prolonged bed-rest, cast immobilization, trauma or disease, inevitably lead to muscle atrophy. The mechanisms that regulate muscle atrophy are poorly understood, but it seems possible that changes in gene expression play a role in initiating and maintaining a muscle atrophy program.

Although most skeletal muscle genes are expressed at similar levels in electrically active, innervated muscle and in electrically inactive, denervated muscle, a small number of genes, including those encoding the acetylcholine receptor, N-CAM, and myogenin, are expressed at significantly higher levels in denervated than in innervated muscle. The mechanisms that mediate electrical activity-dependent gene regulation are not understood, but these mechanisms are likely to be responsible, at least in part, for the changes in muscle structure and function that accompany a decrease in myofiber electrical activity.

As described herein, one of the genes found to be regulated by electrical activity is the runx1 gene. Runx1 is a DNA-binding protein, previously referred to as AML1, but its normal function is not known and its expression and regulation in skeletal muscle were not previously appreciated. Because of its potential role as a transcriptional mediator of electrical activity, expression of the runx1 gene in innervated, denervated, and developing skeletal muscle was characterized, and it was determined that runx1 is expressed at low levels in innervated skeletal muscle and at 50- to 100-fold-higher levels in denervated muscle (Zhu X. et al. (1994), Mol. Cell. Biol. 14(12): 8051-8057). Four runx1 transcripts are expressed in denervated muscle, and the abundance of each transcript increases after denervation. In previous studies, C2 muscle cells were transfected with an expression vector encoding runx1, tagged with an epitope from hemagglutinin, and it was demonstrated that runx1 is a nuclear protein in muscle. It was also determined that runx1 dimerizes with core-binding factor beta (CBF beta), and CGF beta is expressed at high levels in both innervated and denervated skeletal muscle. PEBP2 alpha, which is structurally related to runx1 and which also dimerizes with CBF beta, is expressed at low levels in skeletal muscle and is up-regulated only weakly by denervation.

Studies presented herein were conducted to determine whether an increase in runx1 expression may be causally related to morphological changes in skeletal muscle that accompanies muscle disuse, notably muscle atrophy. As shown by Applicants herein, the runx1 gene has now been identified as playing a role in growth of muscle fibers as well as in inhibition of muscle atrophy or wasting. Currently, there is no useful therapy for the promotion of muscle growth or for prevention of muscle atrophy. Such a therapy would be useful for treatment of a variety of muscular and neuromuscular diseases and disorders.

Accordingly, since runx1 gene expression and/or activity relates to promotion of muscle growth and/or inhibition of muscle atrophy, any means of intervention that would maintain or increase runx1 expression or activity in disused and/or diseased skeletal muscle may minimize muscle atrophy.

Furthermore, other genes that are downstream from runx1 may be regulated by runx1. Thus, it is possible that intervention that would increase the expression or activity of these downsteam genes may likewise ameliorate muscle atrophy. In addition, increasing runx1 expression or activity, or increasing the activity or expression of genes that are downstream from runx1 in normal muscle, may increase muscle mass and function. Furthermore, it is possible that other genes upstream of runx1 may act to turn on the runx1 gene or may enhance its expression. Agents that act in this fashion are also envisioned by the present invention. In addition, it is also believed that there is a role for runx1 expression in muscle other than skeletal muscle. In particular, runx1 may play a role in heart muscle as well as smooth muscle. Accordingly, conditions or diseases in which heart muscle or smooth muscle is atrophied or destroyed may be treated by agents that increase runx1 gene expression or activity.

Thus, as shown by Applicants herein, runx1 is an unusually attractive target for pharmacological intervention in muscle wasting conditions and/or diseases, or for building muscle in normal individuals. Thus, it would be advantageous to develop a means of enhancing the expression of this gene through use of specific agents that are capable of activating runx1 gene expression. These agents may be effective in the treatment of patients who are subject to muscle atrophy due to old age or to a medical condition that leaves these patients bed-ridden. These agents may be small synthetic or naturally derived organic compounds, nucleic acids, polypeptides, antibodies, fragments, isoforms, variants, or other materials that may be used independently for such purposes, all in accordance with the present invention. It is a straightforward task, through conventional pharmacological investigations, to screen for agents that could activate this gene or enhance its expression. Appropriate animal models, such as that described herein, could be used to test for such agents in vivo.

Accordingly, one object of the present invention provides for a method of stimulating muscle growth in a mammal in need thereof by administering to the mammal an effective amount of an agent that stimulates runx1 gene expression or activity in a muscle cell.

A second object of the invention provides for a method of inhibiting muscle atrophy or muscle wasting in a mammal in need thereof by administering to the mammal an effective amount of an agent that stimulates runx1 gene expression or activity in a muscle cell.

A third object of the invention provides a method for the prophylaxis or treatment of a mammal having, or at risk for having, a condition involving decreased muscle fiber size, or muscle atrophy by administering to a mammal in need thereof an effective amount of an agent that stimulates runx1 gene expression.

In a particular embodiment, the mammal is a human patient. In a preferred embodiment, the patient may be incapacitated and demonstrate muscle atrophy as a result of injury or age, and also exhibits a decreased level of the runx1 gene or runx1 gene expression compared to an individual who is not incapacitated. In another embodiment, the patient may be scheduled for a surgical procedure and may be administered an agent that enhances runx1 gene expression prior to or shortly after the surgical procedure. Thus, prophylactic or therapeutic use of such agents that enhance expression of runx1 are envisioned by the present invention.

In another particular embodiment, the muscle cell is a myoblast or a satellite cell. In another particular embodiment, the muscle cell is in skeletal muscle, in cardiac muscle or in smooth muscle.

In yet another particular embodiment, the agent may be a compound such as a small synthetic or naturally derived organic compound, nucleic acids, polypeptides, antibodies, fragments, isoforms, variants, or other materials that may be used independently for such purposes, all in accordance with the present invention.

In another particular embodiment, the muscle growth is the result of an increase in the size of the muscle fibers present in the mammal. However, it is possible that agents can be identified that activate runx1 and that result in an increase in the number and/or size of the muscle fibers in a mammal.

In yet another particular embodiment, the mammal has a condition, which involves muscle damage or muscle atrophy/wasting. The condition may be a skeletal muscle disease. The skeletal muscle disease may be a myopathy or a dystrophy. The skeletal muscle disease may result from a neuropathy.

In yet another particular embodiment, the skeletal muscle disease may be the result of a neural condition or may result from an injury. The injury may be a nerve injury.

In yet another particular embodiment, the muscle cell is in a patient with a cardiac muscle disorder. The cardiac disorder may be cardiomyopathy, ischemic damage, a degenerative congenital disease or a cardiac trauma.

In yet another particular embodiment, the muscle cell is in a patient with myasthenia gravis or a congenital myasthenia gravis syndrome.

In yet another particular embodiment, the muscle cell is in a patient with a smooth muscle disorder. The smooth muscle disorder may be arterial sclerosis, a vascular lesion or a congenital vascular disease. In yet another particular embodiment, the congenital vascular disease involves muscular damage.

The present invention further relates to methods for the diagnostic evaluation and prognosis of a muscle wasting disease in a subject. Preferably the subject is a mammal, more preferably the subject is a human. In a preferred embodiment the invention relates to methods for diagnostic evaluation and prognosis of muscle wasting disease using either antibodies to detect the runx1 gene product, or alternatively, using a nucleic acid probe specific for the runx1 gene sequence, which may be identified in cells or in tissue biopsies.

Screening Assays

Another aspect of the invention provides for methods for identifying agents (candidate compounds or test compounds and the like) for regulating muscle mass or function. Furthermore, methods for identifying a subject at risk for developing a muscle wasting disease are also envisioned by the present invention. In addition, methods for screening, diagnosis or prognosis of a disease in a subject, the disease characterized by muscle atrophy selected from the group consisting of myopathies, dystrophies, myoneural conductive diseases, traumatic muscle injury, nerve injury, cardiomyopathies, ischemic damage, congenital disease, and other conditions resulting in muscle wasting are also contemplated by the present invention. The muscle may be skeletal muscle, heart muscle or smooth muscle.

Examples of agents, candidate compounds or test compounds include, but are not limited to, small synthetic or naturally derived organic compounds, nucleic acids (e.g. DNA and RNA), carbohydrates, lipids, proteins, polypeptides, peptidomimetics, antibodies, fragments, isoforms, variants, or other materials that may be used independently for such purposes, all in accordance with the present invention.

Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. No. 5,738,996; and U.S. Pat. No. 5,807,683, each of which is incorporated herein in its entirety by reference).

Examples of methods for the synthesis of molecular libraries can be found in the art, for example in: DeWitt et al. (1993 Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, each of which is incorporated herein in its entirety by reference.

Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310), each of which is incorporated herein in its entirety by reference.

A compound can be obtained by a number of means including from a commercially available chemical library such as is available from most large chemical companies including Merck, Glaxo SmithKline, Bristol Myers Squibb, Eli Lilly, Novartis, Aventis, and Pharmacia UpJohn. In addition, compounds known to interact with runx1 gene or gene products can be used as potential starting points in drug design.

Potential drugs can also be synthesized de novo or obtained from phage libraries. Phage libraries have been constructed which when infected into host E. coli produce random peptide sequences of approximately 10 to 15 amino acids (Parmley et al. (1990) Gene 73:305-318, Scott et al. (1990) Science 249:386-249). Once a phage encoding a peptide that can act as a potential drug has been purified, the sequence of the peptide contained within the phage can be determined by standard DNA sequencing techniques. Once the DNA sequence is known, synthetic peptides can be generated which are encoded by these sequences. The effective peptide(s) can be synthesized in large quantities for use in for example, in vitro, in situ and/or in vivo assays to determine their effect on runx1 expression and/or activity. These peptides can also be tested in mutant mouse models such as the one described herein for evaluating their effect on muscle tissue mass or muscle fiber size and/or number.

In one embodiment, agents capable of stimulating the expression of runx1 are identified in a cell-based assay system. In accordance with this embodiment, cells expressing the runx1 gene sequence are contacted with a candidate compound or a control compound and the ability of the candidate compound to enhance expression and/or activity of the runx1 gene is determined. If desired, this assay may be used to screen a plurality (e.g. a library) of candidate compounds. The cell, for example, can be of eukaryotic origin (e.g., yeast or mammalian), but is preferably a mammalian muscle cell or cell line, or any other eukaryotic cell which contains runx1, or has been genetically engineered to express runx1.

In addition, the cells can be genetically engineered to express a recombinant runx1 gene, or a homologue thereof, or the cell may further comprise a reporter gene operatively linked or associated with a runx1 responsive or regulatory element and wherein measuring cellular runx1 level involves measuring expression of the reporter gene and/or a recombinant nucleic acid sequence. The nucleic acid sequence encoding runx1 is set forth in SEQ ID NO: 1. The corresponding amino acid sequence for the runx1 protein is set forth in SEQ ID NO: 2. In certain instances, the runx1 protein or peptides derived therefrom are labeled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde, fluorescamine or by in frame fusion to a variety of fluorescent proteins:enhanced green fluorescent protein and derivatives to enable detection of an interaction between runx1 and a binding partner. The ability of the candidate compound to interfere directly or indirectly with runx1 protein binding to its binding partner can be determined by methods known to those of skill in the art, for example, by flow cytometry, a scintillation assay, immunoprecipitation or Western blot analysis.

In another embodiment, the runx1 protein may be used to produce antibodies specific for the runx1 protein. These antibodies may be polyclonal or monoclonal antibodies. They may be chimeric antibodies or single chain antibodies. They may be Fab fragments or soluble components thereof. They may be human or humanized. They may be produced in other animals, including but not limited to horses, goats, sheep, mice, rats, rabbits and guinea pigs.

In another embodiment, agents that enhance the expression of runx1 and also increase muscle growth as shown by an increase in muscle girth or muscle fiber size or an increase in muscle fiber number are identified in an animal model. Examples of suitable animals include, but are not limited to, mice, rats, rabbits, monkeys, guinea pigs, dogs and cats. The expression of the runx1 gene or gene product may be monitored in these animal models by using labeled antibodies to the runx1 protein or by using a labeled nucleic acid probe specific for the runx1 gene sequence or a homologue thereof. Tissue samples may be obtained by biopsy and the runx1 gene or gene product levels assessed by methods described herein and by methods known to those skilled in the art. A comparison can then be made between the levels of the runx1 gene or gene product and the muscle girth in the animal model. In addition, the mouse model described herein in the examples may be utilized and the structure of the muscle fibers can be assessed by punch biopsy after treatment of the mice with an agent identified by the screening methods also described herein.

Drug screens can be performed by any number of means including using high throughput techniques and biological chip technology such as exemplified in U.S. Pat. No. 5,874,219, Issued Feb. 23, 1999, the disclosure of which is hereby incorporated by reference in its entirety.

A test compound is identified as a drug candidate if an increase in the expression of runx1 gene or gene product is identified, and if the test compound then demonstrates an increase in muscle mass or muscle fiber size or number in the animal models described herein.

One high throughput drug screening assay employs eukaryotic cells, such as muscle cells or cell lines transfected with runx1 having a nucleic acid encoding a reporter green fluorescence protein (GFP) under the control of a runx1 responsive element or regulatory sequence. The cells are then robotically plated and grown in 96 well plates, and different chemical compounds (and/or different concentrations of the same compound) can be added to each well. Compounds leading to increased fluorescence due to GFP expression are then identified as drug candidates. These drug candidates can then be further tested for their ability to bind runx1 gene or gene product by in vitro or cell culture assays, and/or in a non-human transgenic or other appropriate animal model. Non-human transgenic animals can also be used to determine dosages and/or toxicity levels.

In another embodiment, a means of assessing an increase in runx1 gene expression is accomplished using a nuclease protection assay, for example, an RNase protection assay. In general, nuclease protection assays (NPAs), include both ribonuclease protection assays (RPAs) and S1 nuclease assays. Both are an extremely sensitive method for the detection, quantitation and mapping of specific RNAs in a complex mixture of total cellular RNA. The basis of NPAs is a solution hybridization of a single-stranded, discrete sized antisense probe(s) to an RNA sample (see Ambion® The RNA Company product catalog). The small volume solution hybridization is far more efficient than more common membrane-based hybridization, and can accommodate up to 100 μg of total or poly(A) RNA. After hybridization, any remaining unhybridized probe and sample RNA are removed by digestion with a mixture of nucleases. The nucleases are subsequently inactivated and the remaining probe:target hybrids are precipitated. These products are separated on a denaturing polyacrylamide gel and are visualized by autoradiography. If nonisotopic probes are used, samples are visualized by transferring the gel to a membrane and performing secondary detection.

Labels

Biological reactions that do not directly involve enzymatic catalysis such as those exemplified herein, or a binding of an antibody to a protein substrate, such as in a Western blot or ELISA, can be detected indirectly via the use of an enzyme label. Indeed, all of the proteins/peptides (including antibodies and fragments thereof), nucleic acids, and compounds employed in the methods of the invention can be labeled. Suitable labels include enzymes such as those discussed below, fluorophores (e.g., fluorescein isothiocyanate (FITC), phycoerythrin (PE), Texas red (TR), rhodamine, free or chelated lanthanide series salts, especially Eu3+, to name a few fluorophores), chromophores, radioisotopes, chelating agents, dyes, colloidal gold, latex particles, ligands (e.g., biotin), and chemiluminescent agents.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, currently known and available counting procedures may be utilized. In the instance where the label is an enzyme, detection may be accomplished by any of the presently utilized techniques known in the art including ultraviolet, visible, and infra-red spectroscopy, circular dichroism, magnetic circular dichroism, fluorescence (including measuring changes in fluorescent lifetimes and fluorescent anisotropy), bioluminescence, luminescence, phosphorescence, mass spectrometry, NMR, ESR, amperometric or gasometric techniques.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453. The proteins/peptides of the present invention can be modified to contain a marker protein such as luciferase or green fluorescent protein as described in U.S. Pat. No. 5,625,048 filed Apr. 29, 1997, WO 97/26333, published Jul. 24, 1997 and WO 99/64592, published Dec. 16, 1999 all of which are hereby incorporated by reference in their entireties.

Suitable marker enzymes include, but are not limited to, alkaline phosphatase and horseradish peroxidase. Other labels for use in the invention include magnetic beads or magnetic resonance imaging labels.

In another embodiment, a phosphorylation site can be created on an antibody of the invention for labeling with 32P, e.g., as described in European Patent No. 0372707 (application No. 89311108.8) by Sidney Pestka, or U.S. Pat. No. 5,459,240, issued Oct. 17, 1995 to Foxwell et al.

Proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

Transgenic Animals

The drug screening methodology of the present invention can be evaluated with transgenic, knock-in, knockout animals or other mutant animals generated specifically to take into account the particulars of modification and expression of the gene under study, e.g. runx1. In one embodiment of this type, the knock-in animal is a mouse. In another embodiment the animal is a knockout mouse.

The present invention also contemplates use of non-human transgenic or knock-in animals that comprise cells that express runx1 of the present invention. In any case, the transgenic, knock-in, and knockout animals of the present invention can be used in drug screens and the like.

A transgenic or knock-in animal can thus be prepared that expresses a recombinant runx1 or a fragment thereof. Such transgenic animals can be obtained through gene therapy techniques described below or by microinjection of a nucleic acid (such as a bacterial artificial chromosome (BAC) that encodes runx1 for example, into an embryonic stem cell or an animal zygote. Microinjection of BACs has been shown to be successful in a number of animals including rats, rabbits, pigs, goats, sheep, and cows (in Transgenic Animals Generation and Use ed., L. M. Houdebine (1997) Harwood Academic Publishers, The Netherlands). Alternatively, a yeast artificial chromosome (YAC) that encodes runx1 for example, can be used. In a preferred embodiment the transgenic animal is a mouse.

Alternatively, an animal model can be prepared in which expression of the runx1 gene(s) is disrupted. Gene expression is disrupted, according to the invention, when no functional protein is expressed. One standard method to evaluate the phenotypic effect of a gene product is to employ knock-out technology to delete a gene as described in U.S. Pat. No. 5,464,764, Issued Nov. 7, 1995; and U.S. Pat. No. 5,777,195, Issued Jul. 7, 1998 (both of which are hereby incorporated by reference herein in their entireties.)

In yet another aspect of the invention a knock-in animal is made. A knock-in animal is prepared in an analogous manner as a knockout animal except a variant/modified exon or gene is substituted for the exon or gene of interest through homologous recombination rather than disrupting the gene. Knock-ins and transgenic animals can be prepared with runx1 or a fragment thereof.

A specific gene-targeting strategy can be used that utilizes a replacement vector containing a particular point mutation and a neo gene flanked by loxP sites to construct the mutation in mice. This procedure is known as the Pointlox procedure (Giese et al. (1998) Science 279:870-873). The point mutation is introduced by polymerase chain reaction (PCR) mutagenesis into a restriction enzyme fragment containing the exon encoding the target amino acid. The restriction fragment of the wild-type genomic clone is then substituted with the mutagenized restriction fragment. A PGKneo cassette flanked by loxP sites is inserted into an adjacent restriction site. After transfection of embryonic stem cells and selection, targeted clones are identified by Southern (DNA) blot analyses. Because the PGKneo cassette could interfere with the expression of neighboring genes, it is removed by transient transfection with pBS185 for example, a plasmid containing Cre recombinase DNA. Chimeras, generated by injection of targeted ES cells into blastocysts, are mated with C57BL/6J mice, and crosses of F1 heterozygotes yield a Mendelian distribution of F2 offspring. The mutants (knock-ins) are identified by PCR. PCR products can be digested for 2 hours at 60° C. directly in PCR reaction buffer without purification, using a selected restriction enzyme (New England Biolabs) based on the creation of a corresponding restriction site by the point mutation in mutant (knock-in).

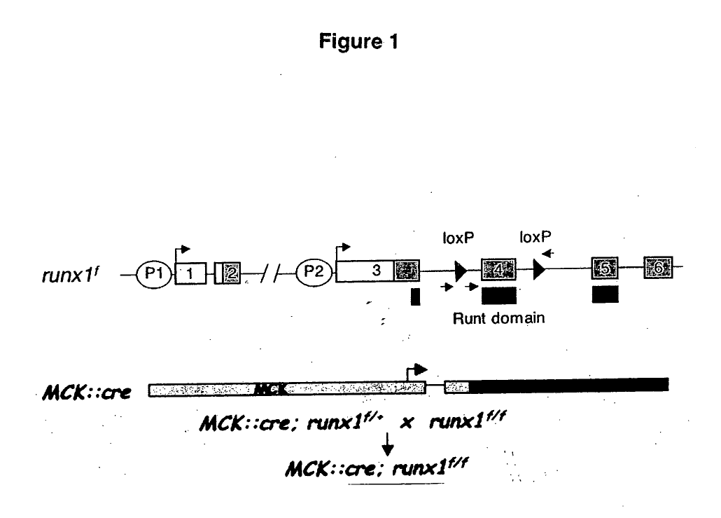

Because inactivation of runx1 may lead to potential complications or defects that can lead to hemorrhaging, it may not be possible to study the role of runx1 in adult skeletal muscle by analyzing simple runx1 mutant mice. Accordingly, in a preferred embodiment, the animal is a mutant mouse model wherein runx1 was inactivated only in muscle, by crossing mice carrying a floxed allele of runx1 (the loxP sites flank the DNA-binding Runt domain) with mice carrying a muscle creatine kinase (MCK):cre transgene (FIG. 1). Moreover, although runx1 is expressed in developing myoblasts and myotubes (Zhu et al. (1994), Mol. Cell. Biol. 14: 8051), MCK is expressed poorly during embryogenesis and at high levels only postnatally. Thus, using this strategy, potential complications that might arise from deletion of runx1 during myogenesis is eliminated.

Although a transgenic/knock-in/knockout mouse is preferred other rodents such as rats and rabbits, or mammals such as pigs, goats, sheep, and monkeys can also be used.

The examples described in the present application demonstrate particular embodiments of the invention. For the purpose of carrying out the experiments described below aimed at examining the specific role of runx1 in regulating muscle growth or for inhibition of muscle atrophy, mice in which runx1 was inactivated only in muscle were generated by crossing mice carrying a floxed allele of runx1 (the loxP sites flank the DNA binding Runt domain) with mice carrying a muscle creatine kinase (MCK)::cre transgene (FIG. 1), as described above.

Use of Antibodies against Runx1 Protein for Diagnostic Purposes

One aspect of the invention provides a method of using an antibody against runx1 or a runx1 gene product, or against a target gene of runx1, or its corresponding gene product, (eg. protein or peptides derived therefrom), with the runx1 target genes being set forth in FIG. 8 and in SEQ ID NOS: 3-31, or nucleic acids encoding runx1, or encoding a runx1 target gene, to diagnose a subject having or predisposed to having, a muscle wasting disease. As runx1 is essential for maintenance of muscle mass and for muscle growth or alternatively, for inhibition of muscle wasting, it provides a general biomarker for disorders in which muscle wasting is characteristic of the disease. This includes, but is not limited to, dystrophies, myopathies or neuromuscular disorders wherein one of the characteristic outcomes is atrophy of the musculature. In addition, patients who are elderly or in patients scheduled to undergo a surgical procedure, there is a need for treatment with an agent that could be used prophylactically to prevent such muscular atrophy or to aid in a faster recovery. Once again, the state of the muscle in terms of its mass can be monitored in patients that are bed-ridden due to age or illness and following such diagnosis by the antibodies to the runx1 gene product can be used with the agents identified by the methods described herein.

The diagnostic method of the invention provides contacting a biological sample such as a biopsy sample, tissue, or cell isolated from a subject with an antibody which binds runx1. The antibody is allowed to bind to the runx1 antigen to form an antibody-antigen complex. The runx1 antigen, as used herein, includes the runx1 protein or peptides isolated therefrom. The conditions and time required to form the antibody-antigen complex may vary and are dependent on the biological sample being tested and the method of detection being used. Once non-specific interactions are removed by, for example, washing the sample, the antibody-antigen complex is detected using any immunoassay used to detect and/or quantitate antigens [see, for example, Harlow and Lane, Antibodies: A Laboratory Manual, Cold Spring Harbor Laboratory, New York (1988) 555-612]. Such well-known immunoassays include antibody capture assays, antigen capture assays, and two-antibody sandwich assays. In an antibody capture assay, the antigen is attached to solid support, and labeled antibody is allowed to bind. After washing, the assay is quantitated by measuring the amount of antibody retained on the solid support. In an antigen capture assay, the antibody is attached to a solid support, and labeled antigen is allowed to bind. The unbound proteins are removed by washing, and the assay is quantitated by measuring the amount of antigen that is bound. In a two-antibody sandwich assay, one antibody is bound to a solid support, and the antigen is allowed to bind to this first antibody. The assay is quantitated by measuring the amount of a labeled second antibody that binds to the antigen.

These immunoassays typically rely on labeled antigens, antibodies, or secondary reagents for detection. These proteins may be labeled with radioactive compounds, enzymes, biotin, or fluorochromes. Of these, radioactive labeling may be used for almost all types of assays. Enzyme-conjugated labels are particularly useful when radioactivity must be avoided or when quick results are needed. Biotin-coupled reagents usually are detected with labeled streptavidin. Streptavidin binds tightly and quickly to biotin and may be labeled with radioisotopes or enzymes. Fluorochromes, although requiring expensive equipment for their use, provide a very sensitive method of detection. Those of ordinary skill in the art will know of other suitable labels which may be employed in accordance with the present invention. The binding of these labels to antibodies or fragments thereof may be accomplished using standard techniques such as those described by Kennedy, et al. [(1976) Clin. Chim. Acta 70:1-31], and Schurs, et al. [(1977) Clin. Chim Acta 81:1-40].

In accordance with the diagnostic method of the invention, the presence or absence of the antibody-antigen complex is correlated with the presence or absence in the biological sample of the runx1 gene product. A biological sample containing elevated levels of said antigen is indicative of a muscle wasting disorder in a subject from which the biological sample was obtained. Accordingly, the diagnostic method of the invention may be used as part of a routine screen in subjects suspected of having a muscle wasting disorder or for subjects who may be predisposed to having a muscle wasting disorder. Moreover, the diagnostic method of the invention may be used alone or in combination with other well-known diagnostic methods to confirm a muscle wasting disorder.

The diagnostic method of the invention further provides that an antibody of the invention may be used to monitor the levels of runx1 antigen in patient samples at various intervals of drug treatment to identify whether and to which degree the drug treatment is effective in restoring muscle cell proliferation or muscle growth. Furthermore, runx1 antigen levels may be monitored using an antibody of the invention in studies evaluating efficacy of drug candidates in model systems and in clinical trials. The class of runx1 containing antigens provides for surrogate biomarkers in biopsy samples to assess the global status of muscle cell proliferation or muscle fiber growth. For example, using an antibody of this invention, runx1 antigen levels may be monitored in biological samples of individuals treated with known or unknown therapeutic agents. This may be accomplished with cell lines in vitro or in model systems and clinical trials, depending on the muscle wasting disorder being investigated. Increased total levels of runx1 antigen in biological samples during or immediately after treatment with a drug candidate indicates that the drug candidate has a positive effect on muscle gowth. Likewise, no change in total levels of runx1 antigen indicates that the drug candidate is ineffective in promoting muscle cell proliferation or muscle growth. This may provide valuable information at all stages of pre-clinical drug development, clinical drug trials as well as subsequent monitoring of patients undergoing drug treatment.

On the other hand, the diagnostic method of the invention also provides a surrogate biomarker to assess rapidly the muscle growth response in clinical situations where administration of a muscle growth promoting drug is therapeutically indicated or in situations where a growth promoting drug is abused for achieving growth of muscle cell mass without a therapeutic intention (eg. in abuse of growth hormone or erythropoietin for competitive sports) In both situations, rapid measurement of runx1 can be done in various tissue or cellular samples.

Detection of Runx1 Nucleic Acid Molecules

In another particular embodiment, the invention involves methods to assess quantitative and qualitative aspects of runx1 gene expression. In one example, the decreased expression of runx1 gene or gene product indicates a predisposition for the development of a muscle wasting disease or neuromuscular disorder. Alternatively, enhanced expression levels of the runx1 gene or gene product can indicate that there is increase in muscle growth or muscle cell proliferation. Techniques well known in the art, e.g., quantitative or semi-quantitative RT PCR or Northern blot, can be used to measure expression levels of the runx1 gene. Methods that describe both qualitative and quantitative aspects of runx1 gene or gene product expression are described in detail in the examples infra. The measurement of runx1 gene expression levels may include measuring naturally occurring runx1 transcripts and variants thereof as well as non-naturally occurring variants thereof. The diagnosis and/or prognosis of a muscle wasting disease in a subject, however, is preferably directed to detecting decreased levels of a naturally occurring runx1 gene product or variant thereof. Thus, the invention relates to methods of diagnosing and/or predicting a muscle wasting disease in a subject by measuring the expression of an runx1 gene or gene product in a subject. For example, the increased level of mRNA encoded by an runx1 gene (e.g., SEQ ID NO: 1), or other gene product, as compared to a non-muscle wasting disease sample or a non-muscle wasting disease predetermined standard would indicate the lack of a muscle wasting disease in said subject or the decreased risk of developing a muscle wasting disease in said subject.

In another aspect of the invention, the increased level of mRNA encoded for by a runx1 gene (e.g., SEQ ID NO: 1), or a runx1 target gene, as set forth in SEQ ID NOS: 3-31 and in FIG. 8, or other related gene product, as compared to that of a non-muscle wasting disease sample or a non-muscle wasting disease predetermined standard would indicate the stage of disease in said subject or the likelihood of a poor prognosis in said subject.

In another example, RNA from a muscle cell type or muscle tissue known, or suspected, to express a runx1 gene, or a runx1 target gene may be isolated and tested utilizing hybridization or PCR techniques as described above. The isolated cells can be derived from cell culture or from a patient. The analysis of cells taken from culture may be a necessary step in the assessment of cells to be used as part of a cell-based gene therapy technique or, alternatively, to test the effect of compounds on the expression of the runx1 gene. Such analyses may reveal both quantitative and qualitative aspects of the expression pattern of the runx1 gene, including activation or suppression of Runx1 gene expression and the presence of alternatively spliced runx1 gene transcripts.

In one embodiment of such a detection scheme, a cDNA molecule is synthesized from an RNA molecule of interest by reverse transcription. All or part of the resulting cDNA is then used as the template for a nucleic acid amplification reaction, such as a PCR or the like. The nucleic acid reagents used as synthesis initiation reagents (e.g., primers) in the reverse transcription and nucleic acid amplification steps of this method are chosen from among the runx1 gene nucleic acid reagents described herein. The preferred lengths of such nucleic acid reagents are at least 9-30 nucleotides.

For detection of the amplified product, the nucleic acid amplification may be performed using radioactively or non-radioactively labeled nucleotides. Alternatively, enough amplified product may be made such that the product may be visualized by standard ethidium bromide staining or by utilizing any other suitable nucleic acid staining method.

RT-PCR techniques can be utilized to detect differences in runx1 gene transcript size that may be due to normal or abnormal alternative splicing. Additionally, such techniques can be performed using standard techniques to detect quantitative differences between levels of runx1 gene transcripts detected in normal individuals relative to those individuals having a muscle wasting disease or exhibiting a predisposition toward a muscle wasting disease.

In the case where detection of particular alternatively spliced species is desired, appropriate primers and/or hybridization probes can be used, such that, in the absence of such a sequence, for example, no amplification would occur.

As an alternative to amplification techniques, standard Northern analyses can be performed if a sufficient quantity of the appropriate cells or tissue can be obtained. The preferred length of a probe used in a Northern analysis is 9-50 nucleotides. Utilizing such techniques, quantitative as well as size related differences between runx1 transcripts can also be detected.

Additionally, it is possible to perform such runx1 gene expression assays in situ, i.e., directly upon tissue sections (fixed and/or frozen) of patient tissue obtained from biopsies or resections, such that no nucleic acid purification is necessary. Nucleic acid reagents such as those described herein may be used as probes and/or primers for such in situ procedures (see, e.g., Nuovo, G. J., 1992, PCR In Situ Hybridization: Protocols And Applications, Raven Press, NY).

Mutations or polymorphisms within a runx1 gene can be detected by utilizing a number of techniques. Nucleic acid from any nucleated cell (e.g., genomic DNA) can be used as the starting point for such assay techniques, and may be isolated according to standard nucleic acid preparation procedures that are well known to those of skill in the art. For the detection of runx1 transcripts or runx1 gene products, any cell type or tissue in which the runx1 gene is expressed, such as, for example, muscle cells may be utilized.

Genomic DNA may be used in hybridization or amplification assays of biological samples to detect abnormalities involving runx1 gene structure, including point mutations, insertions, deletions and chromosomal rearrangements. Such assays may include, but are not limited to, direct sequencing (Wong, C. et al., 1987, Nature 330:384), single stranded conformational polymorphism analyses (SSCP; Orita, M. et al., 1989, Proc. Natl. Acad. Sci. USA 86:2766), heteroduplex analysis (Keen, T. J. et al., 1991, Genomics 11:199; Perry, D. J. & Carrell, R. W., 1992), denaturing gradient gel electrophoresis (DGGE; Myers, R. M. et al., 1985, Nucl. Acids Res. 13:3131), chemical mismatch cleavage (Cotton, R. G. et al., 1988, Proc. Natl. Acad. Sci. USA 85:4397) and oligonucleotide hybridization (Wallace, R. B. et al., 1981, Nucl. Acids Res. 9:879; Lipshutz, R. J. et al., 1995, Biotechniques 19:442).

Diagnostic methods for the detection of runx1 gene nucleic acid molecules, in patient samples or other appropriate cell sources, may involve the amplification of specific gene sequences, e.g., by PCR (See Mullis, K. B., 1987, U.S. Pat. No. 4,683,202), followed by the analysis of the amplified molecules using techniques well known to those of skill in the art, such as, for example, those listed above. Utilizing analysis techniques such as these, the amplified sequences can be compared to those that would be expected if the nucleic acid being amplified contained only normal copies of a runx1 gene in order to determine whether a runx1 gene mutation exists.

Therapeutic and Prophylactic Compositions and Their Use

Candidates for therapy with the agents identified by the methods described herein are patients either suffering from a muscle wasting disorder or are prone to development of such disorder. In addition, elderly patients having limited mobility or patients who are bed-ridden due to a surgical procedure or who are scheduled to have surgery may be candidates for therapy with the agents identified by the methods described.

Patients suffering from such disorders such as dystrophies, myopathies, myasthenia gravis or myasthenic syndromes are candidates for such therapy. In addition, patients suffering from a heart condition whereby heart muscle is destroyed or patients suffering from a condition whereby smooth muscle is destroyed are also candidates for such treatment.

The invention provides methods of treatment comprising administering to a subject an effective amount of an agent of the invention. In a preferred aspect, the compound is substantially purified (e.g., substantially free from substances that limit its effect or produce undesired side-effects). The subject is preferably an animal, including but not limited to animals such as monkeys, cows, pigs, horses, chickens, cats, dogs, etc., and is preferably a mammal, and most preferably human. In one specific embodiment, a non-human mammal is the subject. In another specific embodiment, a human mammal is the subject. Accordingly, the agents identified by the methods described herein may be formulated as pharmaceutical compositions to be used for prophylaxis or therapeutic use to treat these patients.

Various delivery systems are known and can be used to administer a compound of the invention, e.g., encapsulation in liposomes, microparticles, or microcapsules. Methods of introduction can be enteral or parenteral and include but are not limited to intradermal, intramuscular, intraperitoneal, intravenous, subcutaneous, intranasal, epidural, topical and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent. In a specific embodiment, it may be desirable to administer the pharmaceutical compositions of the invention locally to the area in need of treatment.

Such compositions comprise a therapeutically effective amount of an agent, and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In a specific embodiment, it may be desirable to administer the pharmaceutical compositions of the invention locally to the area in need of treatment; this may be achieved, for example, and not by way of limitation, by local infusion during surgery, by topical application, by injection, by means of a catheter, or by means of an implant, said implant being of a porous, non-porous, or gelatinous material, including membranes, such as sialastic membranes, or fibers or co-polymers such as Elvax (see Ruan et al, 1992, Proc Natl Acad Sci USA, 89:10872-10876). In one embodiment, administration can be by direct injection by aerosol inhaler.

In another embodiment, the compound can be delivered in a vesicle, in particular a liposome (see Langer (1990) Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the compound can be delivered in a controlled release system. In one embodiment, a pump may be used (see Langer, supra; Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. (1983) Macromol. Sci. Rev. Macromol. Chem. 23:61; see also Levy et al. (1985) Science 228:190; During et al. (1989) Ann. Neurol. 25:351; Howard et al. (1989) J. Neurosurg. 71:105). In yet another embodiment, a controlled release system can be placed in proximity of the therapeutic target, i.e., the airways, thus requiring only a fraction of the systemic dose (see, e.g., Goodson, in Medical Applications of Controlled Release (1984) supra, vol. 2, pp. 115-138). Other suitable controlled release systems are discussed in the review by Langer (1990) Science 249:1527-1533.

Effective Doses

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to unaffected cells and, thereby, reduce side effects.

The data obtained from cell culture assays and animal studies can be used in formulating a dose range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to optimize efficacious doses for administration to humans. Plasma levels can be measured by any technique known in the art, for example, by high performance liquid chromatography.

In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. Normal dose ranges used for particular therapeutic agents employed for specific diseases can be found in the Physicians' Desk Reference, 54^{th} Edition (2000).

Muscle cell treatments may also be achieved by administering DNA encoding the runx1 polypeptides described above in an expressible genetic construction. DNA encoding the polypeptide may be administered to the patient using techniques known in the art for delivering DNA to the cells. For example, retroviral vectors, electroporation or liposomes may be used to deliver DNA.

The invention includes use of any modifications or equivalents of the above runx1 polypeptides which do not exhibit a significantly reduced activity. For example, modifications in which amino acid content or sequence is altered without substantially adversely affecting activity are included. The statements of effect and use contained herein are therefore to be construed accordingly, with such uses and effects employing modified or equivalent gene products being part of the invention.

The present agents that enhance expression of runx1 or the runx1 genes or gene products themselves can be used as the sole active agents, or can be used in combination with other active ingredients.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of Constructs to Inactivate the Runx1 Gene in Muscle

The wild-type runx1 allele was replaced by a floxed runx1 allele in which loxP sites flank exon 4, which encodes most of the DNA-binding Runt domain. Mice carrying a MCK::cre transgene (Bruning et al. (1998), Mol. Cell 2:559) were crossed with runx1f/f mice to generate MCK::cre; runx1f/f mice, as well as runx1f/f control mice (FIG. 1).

### Example 2

### RNase Protection Analysis to Demonstrate that Exon 4 is Deleted in MCK::cre; Runx1f/f Mice

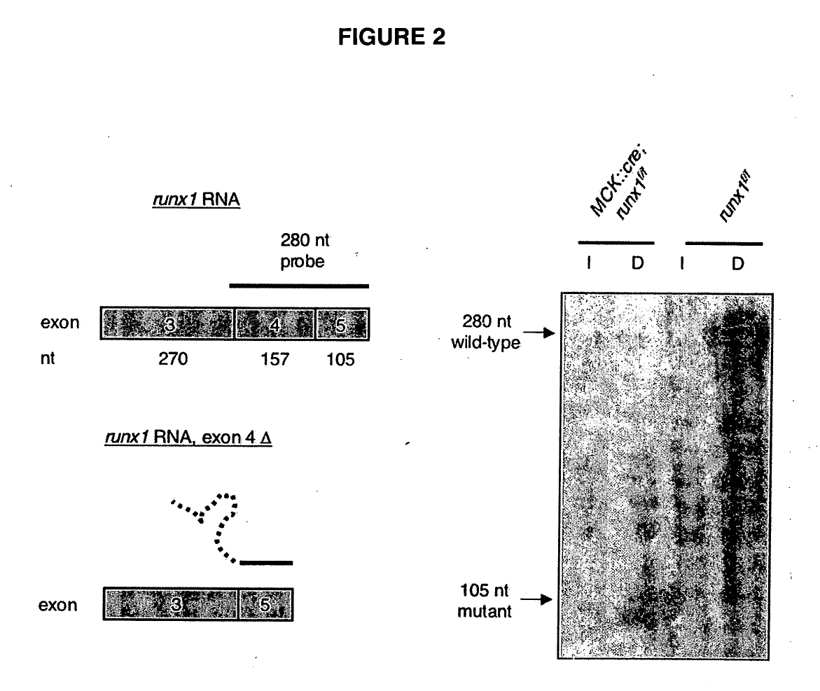

A uniformly labeled single-stranded RNA probe, complimentary to the sequences encoded by exons 4 and 5, was hybridized to RNA isolated from innervated or denervated muscle of MCK::cre; runx1f/f mice, or runx1f/f control mice (FIG. 2).

Results

RNA isolated from denervated muscles of runx1f/f control mice fully protects the 280 nt probe from digestion; a low level of protection is observed in innervated muscle, confirming a 50- to 100-fold induction of runx1 following denervation (Zhu et al. 1994 Mol. Cell Biol. 14:8051). RNA isolated from denervated muscle of MCK::cre; runx1f/f mice protects 105 nt, corresponding to the sequence encoded by exon 5, from digestion; this mutant RNA is induced, like wild-type runx1 RNA, by denervation. Quantitation of these results demonstrates that 98% of the runx1 RNA induced by denervation in MCK::cre; runx1f/f mice lacks the DNA-binding Runt domain (FIG. 2).

### Example 3

### A Lack of Muscle Runx1 Expression Leads to Increased Muscle Atrophy

Limb muscles were denervated for two weeks, fixed and embedded. 1 um-thick sections of innervated (Inn) and denervated (Den) muscles were stained with toluidine blue and were examined. The area of individual myofibers was measured.

Results

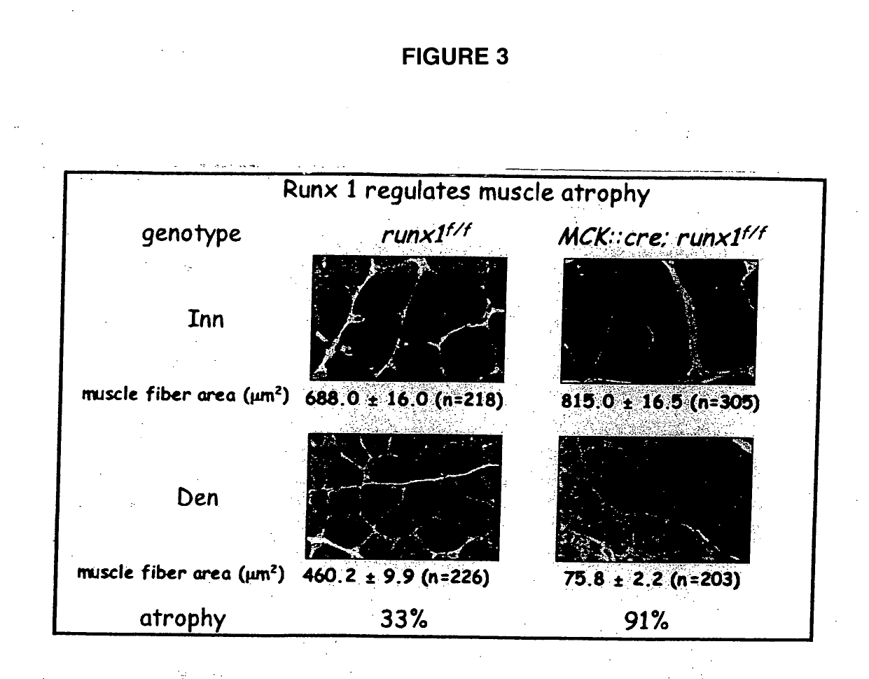

After two weeks of denervation, myofibers atrophy by 33% in runx1f/f control mice and by ˜90% in MCK::cre; runx1f/f mice (FIG. 3).

### Example 4

### Runx1 Prevents Wasting, Myofibrillar Disorganization and Autophagy of Skeletal Muscle

Materials and Methods

Preparation of Constructs to Inactivate the Runx1 Gene in Muscle.

Runx1+/−mice were generated by crossing runx1f/+ mice with CMV::cre mice. The wild-type runx1 allele was replaced by a floxed runx1 allele in which loxP sites flank exon 4, which encodes most of the DNA-binding Runt domain. Mice carrying a MCK::cre transgene (Bruning et al. (1998) Mol. Cell 2:559) were crossed with runx1f/f mice to generate MCK::cre; runx1f/f mice, as well as runx1f/f control mice (FIG. 1).

Hindlimb muscles of adult mice were denervated by cutting the sciatic nerve. Mice were perfused with fixative (1% glutaraldehyde, 4% formaldehyde in 0.1M sodium phosphate, pH 7.3), and dissected muscles were immersion-fixed for a further 1 hr. The fixed muscles were treated with 1% osmium for 1 hr, stained en bloc with saturated, aqueous uranyl acetate for 1 hr, dehydrated and embedded in Epon. For light microscopy, cross-sections of two week denervated muscles were stained with toluidine blue; images were captured on a Sony DKC-500 camera, and the cross-sectional area of individual myofibers was measured using NIH ImageJ. Muscle wet weight and myofiber size from three mutant and control mice was measured. For electron microscopy, longitudinal sections of two week-denervated muscle were stained with uranyl acetate and lead citrate.

Runx1 RNA expression, in innervated and denervated gastrocnemius muscles, was measured by RNase protection (Zhu, X. et al. (1994), Mol Cell Biol 14: 8051-7); a uniformly labeled RNA probe, complimentary to the sequences encoded by exons 4, 5 and eighteen bp from exon 3, was hybridized to RNA isolated from innervated or four-day denervated muscle of MCK::cre; runx1f/f mice, or runx1f/f control mice. Expression of MuRF1 (AK052911, nucleotides 172-471), Fbxo32 (NM_{—}026346, nucleotides 256-444) and Gapd were measured by RNase protection (Blagden, C. S. et al. (2004), Mol Cell Biol 24: 1983-9).

Affymetrix mouse genome 430 2.0 microarrays were probed with cDNA copied from RNA isolated from innervated and three-day denervated tibialis anterior muscles from runxf/f control and MCK::cre; runxf/f male mice. Experiments were performed on three separate microarrays for each of the four experimental conditions. The chips were scanned with an Affymetrix GeneChip Scanner 3000, and the raw data were processed with Affymetrix GCOS software. The signals were normalized and analyzed by dChip (Parmigiani 2003). We calculated the ratio of expression in denervated/innervated muscle for runxf/f control and MCK::cre; runxf/f mice.

Results from the microarray screen were validated by measuring expression of selected target genes using an RNase protection assay: Myh2 (NM_{—}144961, nucleotides 1650-1910), Myh3 (XM_{—}354614, nucleotides 2104-2372), Aqp4 (NM_{—}009700, nucleotides 445-895), Accn1 (NM_{—}007384, nucleotides 1406-1672), Thbs1 (NM_{—}011580, nucleotides 298-548), Chrng (NM_{—}009604, nucleotides 696-1244) and Scn5a (NM_{—}021544, nucleotides 76-611).

Results

Inactivation of Runx1 Selectively in Skeletal Muscle

Mice lacking Runx1 die during mid-embryogenesis (E12), due to hemorrhaging in the central nervous system (Okuda, T. J. et al. (1996), Cell 84: 321-30; Wang, Q. T. et al. (1996) Proc Natl Acad Sci USA 93: 3444-9). To study the role of Runx1 in skeletal myofibers, we circumvented this embryonic lethality by generating mice that carry a loxP flanked allele of runx1 and a muscle creatine kinase (MCK)::cre transgene (FIG. 1). The runx1 allele contains loxP sites that flank exon 4 (Taniuchi, I. M., et al. (2002), Cell 111: 621-33), which encodes the major portion of the DNA-binding domain as well as sequences that are essential for interaction with CBFβ, a Runx-interacting protein that is required for Runx1 activity in vivo (de Bruijn and Speck (2004), Oncogene 23: 4238-48). Splicing from exon 3, the first coding exon in skeletal muscle (data not shown), to exon 5 or exon 6 alters the reading frame, resulting in a truncated protein. Thus, deletion of exon 4 is likely to result in a runx1 null mutation (see below).

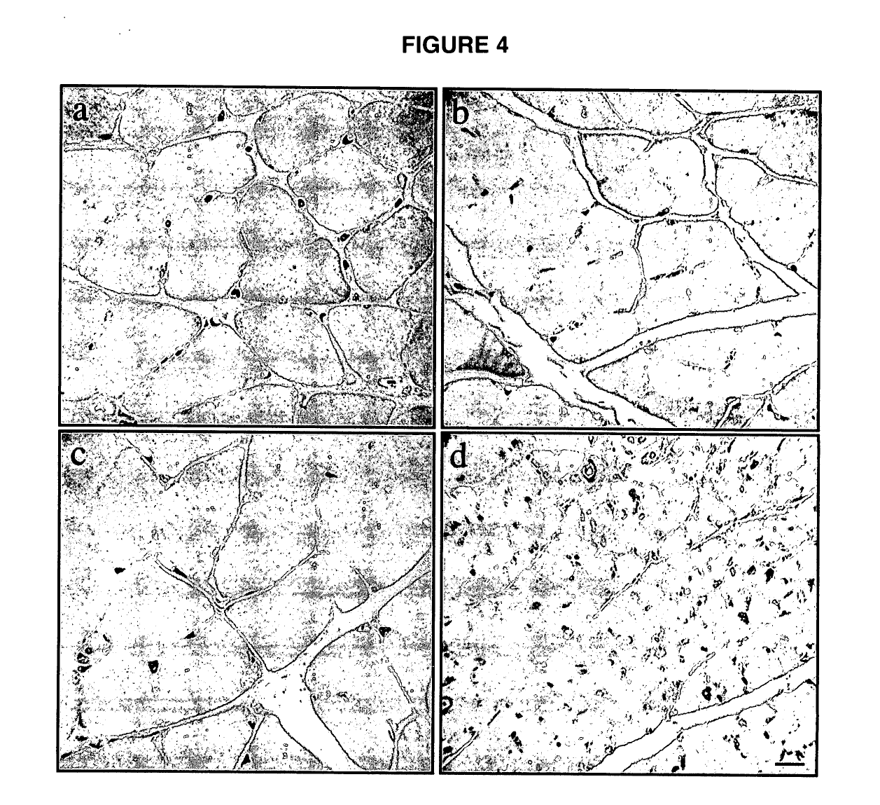

Runx1 is expressed in developing muscle but down-regulated by innervation (Zhu et al. (1994), Mol Cell Biol 14: 8051-7), which begins at E12.5. Because the MCK regulatory elements confer only a low level of Cre recombinase expression in embryonic skeletal muscle and substantially greater expression after birth (Bruning et al. (1998), Mol Cell 2: 559-69; Nguyen et al. (2003), J Biol Chem 278: 46494-505), maximal Cre expression is attained at a time when skeletal muscle runx1 expression has already declined. Since severe defects in myogenesis lead to neonatal lethality, this experimental design minimizes the potential for neonatal lethality if Runx1 has a key role during myogenesis. Indeed, MCK::cre; runx1f/f mice were healthy and fertile and born in expected numbers. We assessed the effectiveness of Cre-mediated recombination by measuring runx1 RNA expression in innervated and denervated muscles from MCK::cre; runx1f/f mice and runx1f/f control adult mice. We used an RNase protection assay, which detects both wild-type and mutant runx1 RNAs, and found that 96% of skeletal muscle runx1 is inactivated in MCK::cre; runx1f/f mice (FIG. 4). Moreover, these data demonstrate that runx1 expression is not dependent upon autoregulation, as suggested for runx2 (Drissi et al. (2000), J Cell Physiol 184: 341-50), as runx1 is induced to the same extent in runx1 mutant and control muscle.

Denervated, Runx1 Mutant Myofibers are Severely Atrophied

In wild-type mice, peripheral nerve damage (Zhu et al. (1994), Mol Cell Biol 14: 8051-7), as well as limb immobilization (Bodine et al. (2001), Science 294: 1704-8), lead to an increase in Runx1 expression and muscle atrophy. To determine whether Runx1 regulates muscle atrophy, we denervated hindlimb muscles in mutant and control adult mice and examined innervated and denervated muscles by light microscopy two weeks later. FIG. 4 shows that denervated, runx1 mutant myofibers are severely and unusually atrophic. In the soleus muscles of control runx1f/f mice and runx1f/f− mice, which are heterozygous for the mutant allele, a two-week denervation period leads to a ˜35% decrease in the cross-sectional area of the myofibers (innervated runx1f/f, 690±236 μm2, m±s.d., n=218; denervated runx1f/f, 460±150 μm2, n=226; innervated runx1f/−, 684±259 μm2, n=489; denervated runx1f/−, 401±142 μm2, n=434) and a corresponding decrease in muscle wet weight. In the soleus muscles of mice lacking Runx1 (MCK::cre; runx1f/f), denervation leads to a 10-fold decrease in myofiber size (innervated, 702±252 μm2, n=877; denervated, 82±29 μm2, n=524) and a similar decrease in muscle wet weight. In addition, the interstitial space between the denervated, runx1 mutant myofibers is unusually compacted, indicating that the muscle extracellular matrix is also defective. We noted no signs of apoptosis, as myofibers were neither TUNEL-positive nor stained with antibodies to activated caspase 3 (data not shown). Other muscles, including the tibialis anterior and gastrocnemius muscles, are also affected, and similar changes in myofiber cell size were evident in denervated muscle from myf5cre; runx1f/f mice (data not shown). In contrast, innervated control and runx1 mutant muscles appear similar, indicating that the low level of Runx1 expression in innervated muscle does not regulate myofiber cell size (FIG. 4). These results indicate that Runx1 is required to sustain denervated muscle and to minimize atrophy.

The Severe Wasting of Denervated Runx1 Myofibers is not Accompanied by Excessive Activation of the FoxO-Mediated Atrophy Pathway

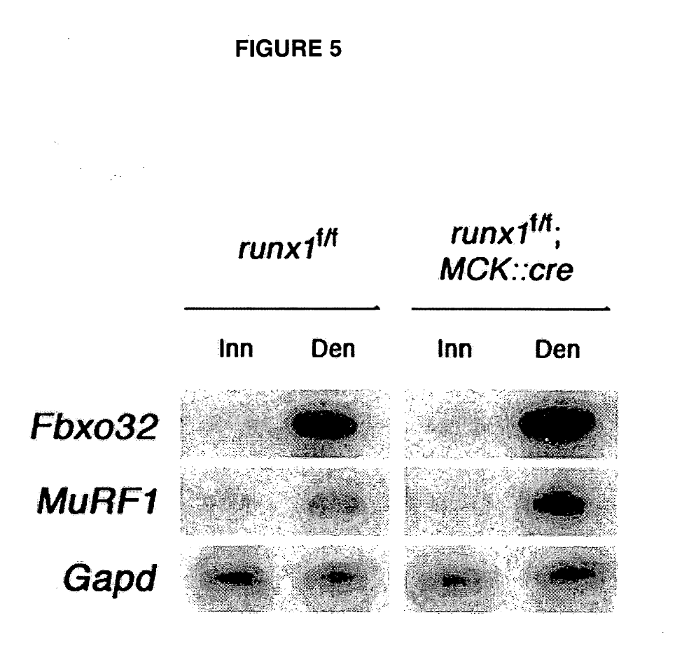

The pathways that regulate atrophy are poorly understood, but muscle disuse leads to a reduction in phosphatidylinositol 3-kinase (PI3K)/Akt activities and a decrease in FoxO phosphorylation, triggering nuclear import of FoxO and activation of FoxO target genes (Sandri et al., (2004), Cell 117: 399-412; Stitt et al. (2004), Mol Cell 14: 395403). To determine whether the severe wasting of denervated runx1 mutant myofibers is due to excessive activation of the FoxO-mediated atrophy pathway, we measured expression of a direct target of FoxO1, atrogin-1 (MAFbx; Fbxo32) (Bodine et al. (2001), Science 294: 1704-8; Sandri et al. (2004), Cell 117: 399-412), an E3-ubiquitin ligase, in innervated and denervated muscles from control and runx1 mutant mice. We found that denervation causes a 5.3±0.4-fold (m±s.e.m., n=3) increase in atrogin-1 expression in control mice and a 8.6 ±0.4-fold (m±s.e.m., n=3) increase in atrogin-1 expression in runx1 mutant muscle (FIG. 5). Thus, the severe wasting of denervated, runx1 mutant muscle is unlikely to be due to excessive activation of the FoxO pathway. Atrophy can be induced by NFκB without inducing expression of atrogin-1 (Cai et al. (2004), Cell 119: 285-9). Instead, NFκB induces expression of MuRF1, an E3-ubiquitin ligase, which like atrogin-1, is induced by denervation (Bodine et al. (2001), Science 294: 1704-8; Cai et al. (2004), Cell 119: 285-9). To determine whether the severe wasting of denervated runx1 mutant myofibers is due to excessive activation of this NFκB pathway, we measured MuRF1 (Trim63; Rnf28) expression in innervated and denervated muscles from control and runx1 mutant mice. FIG. 5 shows that denervation leads to a 4.7±0.7-fold (m±s.e.m., n=2) increase in MuRF1 expression in control mice and a 9.5±0.1-fold (m±s.e.m., n=2) increase in MuRF1 expression in runx1 mutant muscle (FIG. 5). Thus, the severe wasting of denervated, runx1 mutant muscle is unlikely to be caused by excessive activation of this NFκB pathway. Although Atrogin-1 and MuRF1 may contribute to wasting of runx1 mutant muscle, other pathways are likely to be responsible for the striking decrease in myofiber size in denervated, runx1 mutant muscle.

Runx1 Induction is Required to Prevent Disused Myofibers from Undergoing Autophagy, Myofibrillar Disorganization and Severe Muscle Wasting

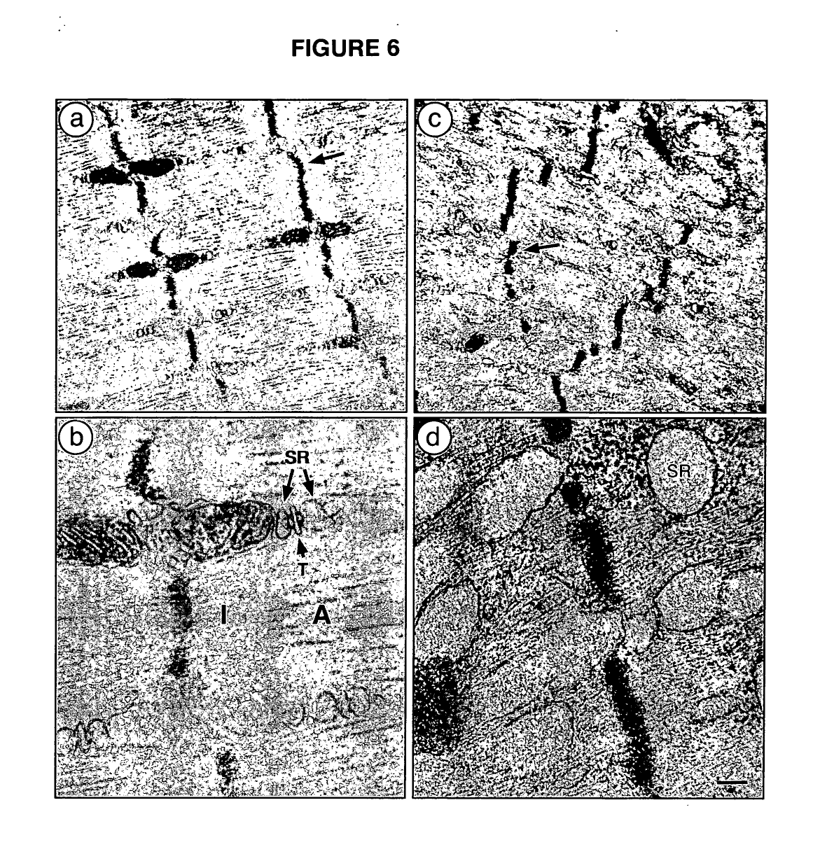

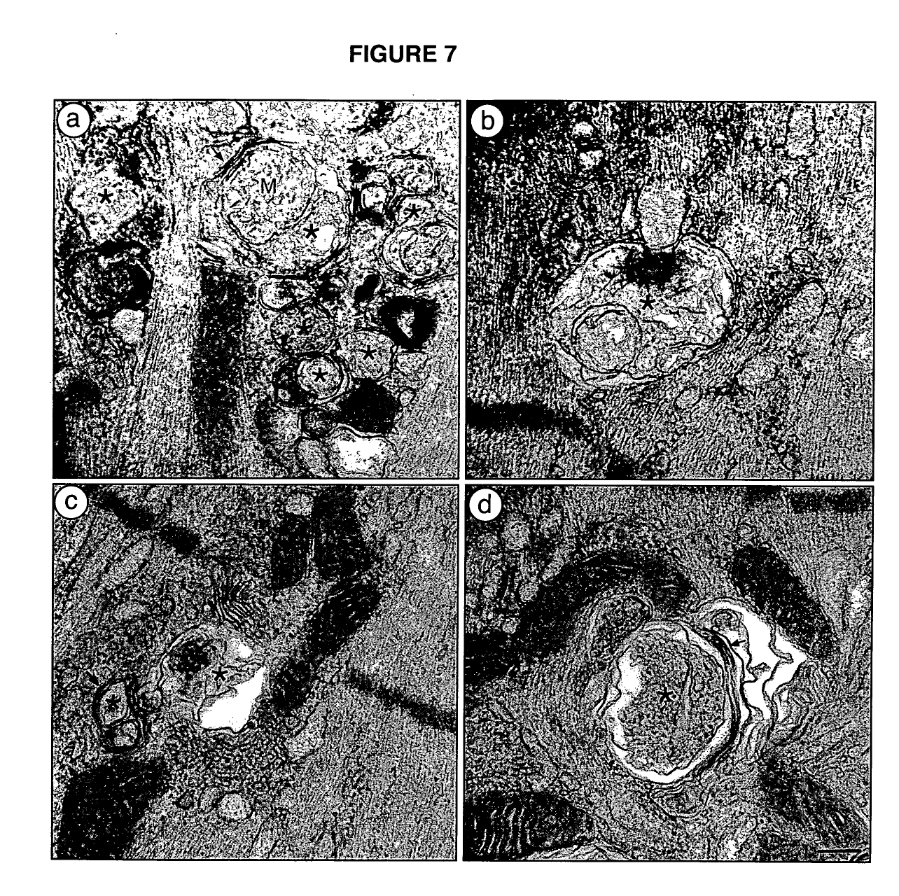

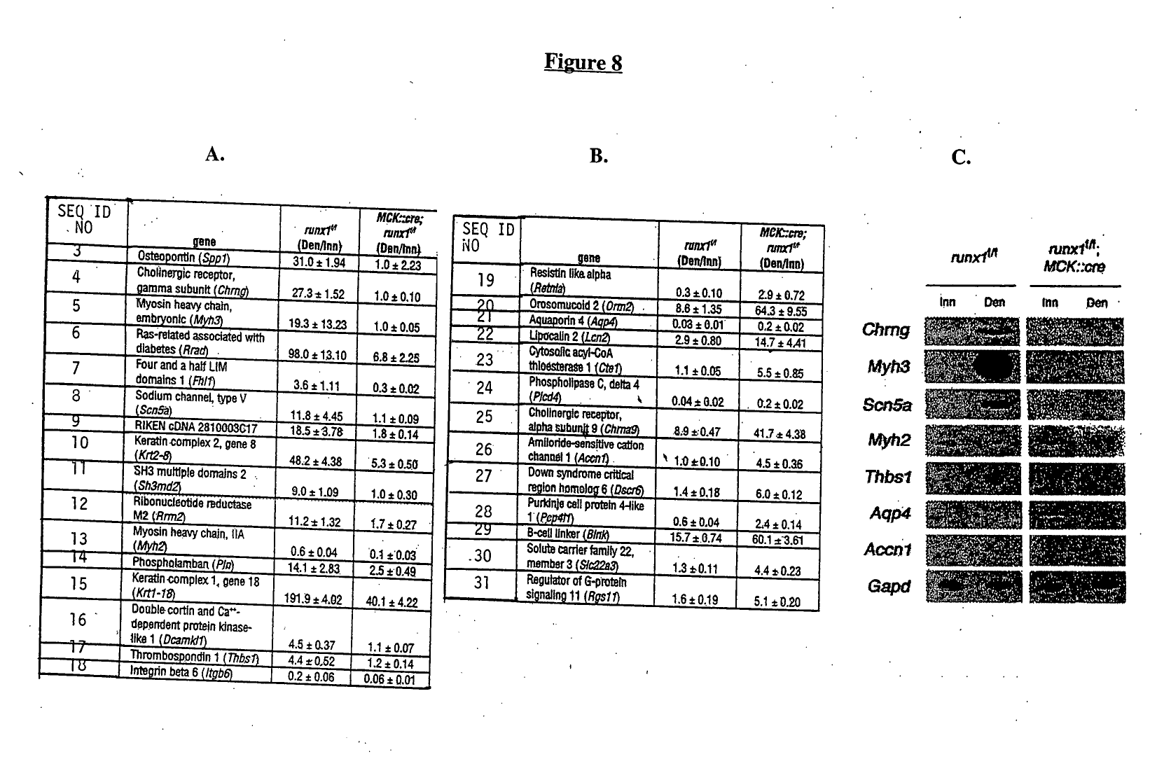

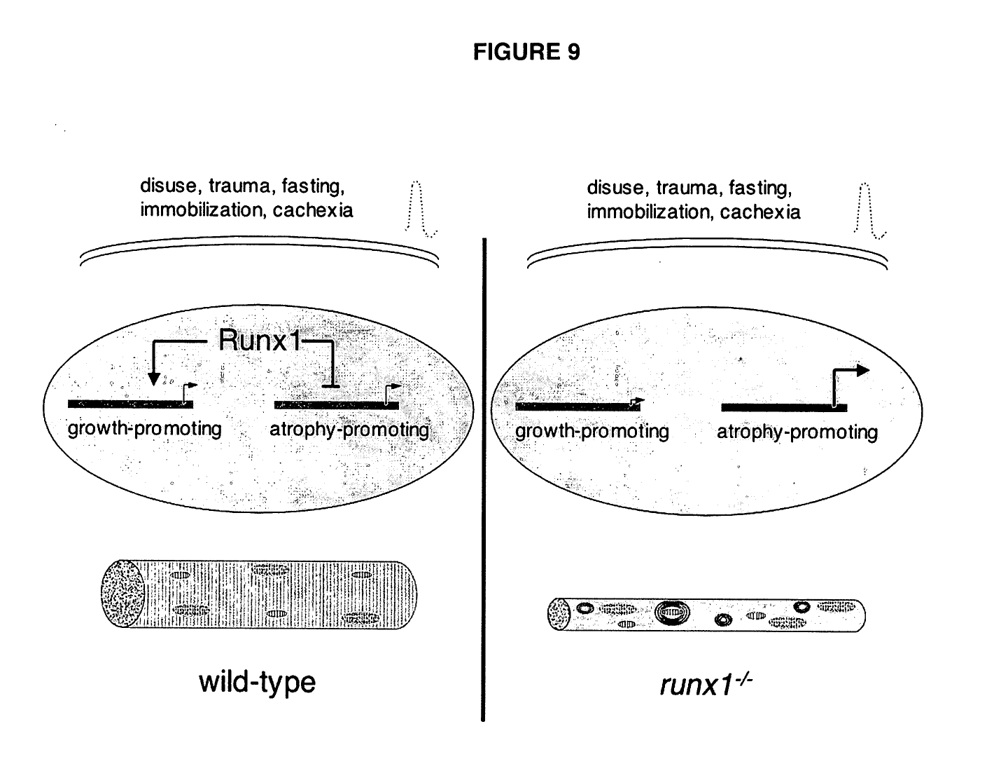

In wild-type mice, disused myofibers have a reduced capacity to generate force, due to their smaller cross-sectional area, but these atrophic myofibers retain most structural features that are characteristic of innervated muscle. We examined runx1 mutant and control soleus muscles by electron microscopy to determine whether denervated, runx1 mutant muscles display additional signs of muscle wasting. We found that denervated, runx1 mutant myofibers have structural aberrations that are not evident in atrophic, denervated control myofibers but are reminiscent of structural abnormalities found in a variety of congenital myopathies (FIG. 6) (Engel (1999), Ann Neurol 46: 681-3; Nishino (2003), Curr Neurol Neurosci Rep 3: 64-9; Selcen et al. (2004), Ann Neurol 54: 804-10). First, in denervated, runx1 mutant muscles, the Z-discs are misaligned, fragmented and irregularly spaced. Second, the distinctive A- and I-bands, readily evident in innervated and denervated muscles of control mice, are not apparent in denervated, runx1 mutant muscles. Instead, the myofibrils appear to contain only thin filaments, presumably composed of actin, and to lack thick filaments composed of myosin. Third, the sarcoplasmic reticulum in denervated, runx1 mutant muscle is severely dilated and extends from the Z-disc into the space normally occupied by myofibrils. Fourth, scattered throughout denervated runx1 mutant myofibers are double- or multi-membrane vacuoles, which enclose heterogeneous contents, including mitochondria and additional membrane-enclosed structures (FIG. 7). These features define these organelles as autophagic vacuoles (Gozuacik and Kimchi (2004), Oncogene 23: 2891-906), demonstrating that Runx1 is required to prevent denervated myofibers from undergoing autophagy and indicating that excessive autophagy is responsible for the severe wasting of denervated runx1 mutant myofibers.

Twenty-Nine Genes, Encoding Channels, Signaling Molecules, Structural Proteins, but not Transcription Factors, are Mis-Expressed in Denervated, Runx1 Mutant Muscle

A loss of muscle activity leads to a reduction in signaling that promotes muscle growth (Murgia et al. (2000), Nat Cell Biol 2: 142-7), but disused muscle does not undergo autophagy. Taken together, these findings suggest four potential mechanisms by which Runx1 prevents autophagy. First, Runx1 may partially counter-balance a loss of muscle activity by inducing the same genes that are induced by muscle activity. Second, Runx1 may induce a different set of genes that compensate for the loss of muscle activity and likewise promote muscle growth/maintenance, thereby preventing autophagy. Third, Runx1 may be required to repress genes that promote autophagy. Fourth, Runx1 may induce genes that encode for structural components of the myofiber, and incomplete or partially assembled structures may lead to a stress response that provokes autophagy. To identify genes that are dependent upon Runx1 expression and regulate muscle structure, we screened oligonucleotide microarrays with RNA from innervated and denervated muscles from wild-type and runx1 mutant mice (FIG. 8) and SEQ ID NOS: 3-31. To increase the probability of identifying direct targets of Runx1, we probed microarrays with RNA from skeletal muscle that had been denervated for three days, one day after runx1 expression is maximal in wild-type mice but prior to overt structural changes in denervated runx1 mutant muscle. We validated the microarray data by measuring expression of a subset of genes, identified as mis-regulated in the microarray screen, by RNase protection (FIG. 8). Only twenty-nine genes are mis-regulated (≧three-fold) in denervated muscle lacking Runx1, suggesting that only a few genes are responsible for the dramatic muscle wasting observed in runx1 mutant mice. To assess the reliability of the microarray data, we measured expression of six mis-regulated genes in innervated and denervated muscle from wild-type and runx1 mutant muscle by an RNase protection assay. In each case, the RNase protection data corroborated the results from the microarray analysis, confirming the reliability of the microarray data (FIG. 8). As most genes are unaffected by Runx1 expression, including genes that are known to be dependent upon innervation, including myogenin, MuSK and the acetylcholine receptor α and δ subunit genes, it is unlikely that Runx1 simply compensates for a loss of innervation. The twenty-nine mis-regulated genes encode ion channels (five genes), signaling molecules (fourteen genes) and structural proteins (four genes) but not transcription factors, indicating that the identified genes are good candidates for direct targets of Runx1 (FIG. 8). Sixteen genes are not appropriately up-regulated or maintained in runx1 mutant denervated muscle, suggesting that Runx1 activates their expression. Thirteen genes are expressed at unusually high levels in denervated muscle lacking Runx1, suggesting that Runx1 represses their expression. These findings are consistent with other studies showing that Runx family members can activate or repress target genes (Kramer et al. (1999), Development 126: 191-200). Further studies of these Runx1 target genes should lead to a better understanding of their roles in muscle wasting.

Summary

Disruptions in myofiber electrical activity, including denervation, lead to muscle atrophy (Jagoe and Goldberg (2001), Curr Opin Clin Nutr Metab Care 4: 183-90; Glass (2003), Nat Cell Biol 5: 87-90). Although innervation provides trophic signals that are critical for muscle differentiation and growth, atrophic myofibers neither degenerate nor undergo apoptosis but retain most of the structural features that are characteristic of normal muscle. These results suggest that myofiber size and differentiation are only partially dependent upon innervation or that denervation induces compensatory mechanisms that limit the extent of myofiber atrophy and wasting. Our results demonstrate that Runx1 induction is required to prevent myofibrillar disorganization and severe muscle wasting, indicating that muscle disuse indeed induces pathways that compensate for a loss innervation, thereby preventing myofibrillar disorganization and limiting the extent of muscle atrophy and wasting (FIG. 9). Moreover, our experiments demonstrate that these compensatory pathways depend upon Runx1 induction. Despite the importance of Runx1 in leukemogenesis, only CD4 has been identified as a bonafide target of Runx1 in hematopoietic cells (Taniuchi et al. (2002), Cell 111: 621-33). Among the Runx1-dependent genes identified in our microarray screen, we identified several genes, including acetylcholine receptor α9 subunit, osteopontin, orosomucoid 2, lipocalin 2 and amiloride-sensitive cation channel 1, that were not known to be expressed in skeletal muscle. In addition, the microarray screen identified multiple genes, including aquaporin 4, keratin 2-8 and keratin 1-18, that were known to be expressed in muscle but not known to be regulated by innervation. Moreover, the screen identified multiple genes, including embryonic myosin heavy chain, sodium channel type V and acetylcholine receptor γ subunit genes, that were known to be regulated by electrical activity but by unknown transcriptional mechanisms.

The work presented herein demonstrates that Runx1 is required to activate and repress gene expression in mammalian skeletal muscle, and the data indicate that these Runx1-dependent genes are direct targets for Runx1.

Autophagy is responsible for the normal bulk degradation of long-lived proteins and organelles, but the program can be over-activated by a variety of stress stimuli, presumably to facilitate cell survival during periods of acute stress (Klionsky and Emr (2000), Science 290: 1717-21). The pathway for constructing autophagic vacuoles and delivering vacuoles to lysosomes requires the sequential action of a set of genes, identified in yeast and conserved in mammalian cells. The products of these “autophagy genes”, mTOR, class III PI3K, two ubiquitin-like protein conjugation systems and a cysteine protease, act in a concerted series of post-translational modification steps to form double-membrane autophagosomes and to transfer autophagic vacuoles and their contents to lysosomes (Klionsky and Emr (2000), Science 290: 1717-21; Gozuacik and Kimchi (2004), Oncogene 23: 2891-906). The transcriptional changes that initiate and attenuate this autophagy program are poorly understood. Our findings indicate that Runx1 has a role in restraining pathways leading to autophagy, as a failure to up-regulate Runx1 in denervated muscle results in severe muscle wasting accompanied by hallmarks of autophagy.

Although it is currently unclear whether excessive autophagy promotes or prevents cell damage and leads to improvement or worsening of disease outcome, the presence of autophagic vacuoles is a prominent and characteristic structural feature in a variety of congenital myopathies (Engel (1999), Ann Neurol 46: 681-3; Nishino (2003), Curr Neurol Neurosci Rep 3: 64-9; Selcen et al. (2004), Brain 127: 439-51), neurodegenerative disorders (Shintani and Klionsky (2004), Science 306: 990-5) and cancer (Gozuacik and Kimchi (2004), Oncogene 23: 2891-906; Shintani and Klionsky (2004), Science 306: 990-5).

Myofibrillar myopathies, characterized by pathological defects in myofibrillar organization and accumulation of autophagic vacuoles, can be caused by mutations in Z-disc proteins, desmin (Goldfarb et al. (1998), Nat Genet 19: 402-3; Munoz-Marmol et al. (1998 Proc Natl Acad Sci USA 95: 11312-7), alpha B-crystallin (Vicart et al. (1998), Nat Genet 20: 92-5; Selcen and Engel (2003), Ann Neurol 54: 804-10) or myotilin (Selcen and Engel (2004), Brain 127: 439-51). Moreover, Danon's disease, which is also typified by the presence of autophagic vacuoles, is caused by mutations in LAMP-2, a lysosomal membrane protein (Nishino et al. (2000), Nature 406: 906-10; Tanaka et al. (2000), Nature 406: 902-6). The genes responsible for most congenital myopathies, however, are not yet known. Our findings demonstrate an unexpected role for electrical activity in regulating autophagy and raise the possibility that reductions in muscle activity could cause or contribute to these static muscle wasting diseases if expression of

Runx1 or Runx1-target genes were compromised.

TABLE 1

| SEQ ID NO | PubMed Number |
|---|---|
| 3 | BC007016 |
| 4 | NM_005199 |
| 5 | AF104922 |
| 6 | NM_004165 |
| 7 | NM_004468 |
| 8 | DQ052007 |
| 9 | NM_145144 |
| 10 | NM_031170 |
| 11 | NM_020870 |
| 12 | NM_001034 |
| 13 | NM_017534 |
| 14 | DQ046231 |
| 15 | NM_010664 |
| 16 | AL157694 |
| 17 | NM_003246 |
| 18 | NM_021359 |
| 19 | NM_020509 |
| 20 | NM_000608 |
| 21 | NM_198098 |
| 22 | NM_005564 |
| 23 | BF197852 |
| 24 | NM_032726 |
| 25 | NM_017581 |
| 26 | NM_001094 |
| 27 | DQ040121 |
| 28 | NM_006198 |
| 29 | NM_013314 |
| 30 | AL591069 |
| 31 | NM_004296 |

What is claimed is:

1. A method of stimulating muscle growth in a mammal in need thereof, the method comprising administering to the mammal an effective amount of an agent capable of stimulating runx1 gene expression or activity in a muscle cell.

2. A method of inhibiting muscle atrophy or muscle wasting in a mammal in need thereof, the method comprising administering to the mammal an effective amount of an agent capable of stimulating runx1 gene expression or activity in a muscle cell.

3. The method of either of claims 1 or 2, wherein the mammal is a human.

4. The method of claim 1, wherein the muscle cell is a myoblast or a satellite cell.

5. The method of claim 1, wherein the muscle cell is a skeletal muscle cell, a cardiac muscle cell, or a smooth muscle cell.

6. The method of claim 1, wherein the muscle growth results from an increase in size of muscle fibers present in the mammal.

7. The method of either of claims 1 or 2, wherein the mammal suffers from a condition involving muscle damage or muscle atrophy/wasting.

8. The method of claim 7, wherein the condition is a skeletal muscle disease.

9. The method of claim 8, wherein the skeletal muscle disease is a myopathy or a dystrophy.

10. The method of claim 8, wherein the skeletal muscle disease results from a neural condition or an injury.

11. The method of claim 10, wherein the injury is a nerve injury.

12. The method of claim 8, wherein the skeletal muscle disease results from a neuropathy.

13. The method of either one of claims 1 or 2, wherein the muscle cell is in a patient suffering from a cardiac muscle disorder.

14. The method of claim 13, wherein the cardiac muscle disorder is selected from the group consisting of a cardiomyopathy, ischemic damage, a degenerative congenital disease and cardiac trauma.

15. The method of either of claims 1 or 2, wherein the muscle cell is found in a patient suffering from a smooth muscle disorder.

16. The method of claim 15, wherein the smooth muscle disorder is selected from the group consisting of arterial sclerosis, a vascular lesion, and a congenital vascular disease.

17. The method of claim 16, wherein the congenital vascular disease is characterized by muscular damage.

18. The method of either of claims 1 or 2, wherein the mammal is a patient who is incapacitated as a result of injury or age, and experiences a decreased level of the runx1 gene or runx1 gene expression compared to an individual who is not incapacitated.

19. The method of either of claims 1 or 2, wherein the muscle cell is found in a patient suffering from myasthenia gravis or a congenital myasthenia gravis syndrome.

20. The method of either of claims 1 or 2, for the prophylaxis or treatment of a mammal having, or at risk for having, a condition involving decreased muscle fiber size, or muscle atrophy, or muscle cell autophagy, the method comprising administering to the mammal an effective amount of an agent that stimulates runx1 gene expression.

21. A method for identifying a candidate compound for regulating skeletal muscle mass or function, comprising:
a. contacting a test compound with a cell containing the runx1 gene, or a target of the runx1 gene;
b. determining whether the test compound results in upregulation/activation of a runx1 gene, or a target of a runx1 gene, or a corresponding gene product;
c. selecting a compound that upregulates/activates the runx1 gene, or the target of the runx1 gene or corresponding gene product and further determining whether the test compound increases muscle mass or function in a skeletal muscle atrophy model system in innervated or denervated muscle; and
d. identifying a test compound that modulates muscle mass or function as a candidate compound for regulating skeletal muscle mass or function.

22. The method for identifying candidate compounds according to claim 21, wherein the runx1 gene has a nucleotide sequence that is greater than 80% identical to the sequence of SEQ ID NO: 1.

23. The method for identifying candidate compounds according to claim 21, wherein the runx1 gene has a nucleotide sequence that is greater than 90% identical to the sequence of SEQ ID NO: 1.

24. The method for identifying candidate compounds according to claim 21, wherein the runx1 gene has a nucleotide sequence corresponding to the nucleic acid sequence of SEQ ID NO: 1.

25. The method of claim 21, wherein the cell containing the runx1 gene is a eukaryotic cell.

26. The method of claim 25, wherein the eukaryotic cell is a muscle cell or muscle cell line, or any other eukaryotic cell which contains runx1, or has been genetically engineered to express runx1.

27. The method for identifying candidate compounds according to claim 26, wherein the cell further comprises a reporter gene operatively associated with a runx1 responsive element and wherein measuring cellular runx1 level involves measuring expression of the reporter gene.

28. The method of claim 21, wherein the determining is accomplished by performing an assay for measuring runx1 RNA or protein expression, and wherein the assay selected from the group consisting of an immunoassay, an RNase protection assay and an assay wherein a nucleic acid probe is used for detection of the runx1 gene.

29. A method of determining if a subject is at risk for developing a muscle wasting disease, the method comprising:
(I) measuring an amount of a runx1 gene or a target gene of runx1 or a corresponding gene product in a tissue sample derived from the subject, wherein the runx1 gene or the target of the runx1 gene or corresponding gene product is:
(a) a DNA corresponding to SEQ ID NO: 1, or to any one of SEQ ID NOS: 3-31 or a nucleic acid derived therefrom;
(b) a protein comprising SEQ ID NO: 2, or a protein encoded by the nucleic acid sequence of any one of SEQ ID NOS: 3-31;
(c) a nucleic acid comprising a sequence hybridizable to SEQ ID NO: 1, or to any one of SEQ ID NOS: 3-31 or their complements under conditions of high stringency, or a protein comprising a sequence encoded by the hybridizable sequence;
(d) a nucleic acid at least 90% homologous to SEQ ID NO: 1, or to any one of SEQ ID NOS: 3-31 or their complements as determined using the NBLAST algorithm; or a protein encoded thereby; and
(II) comparing the amount of the gene product of runx1 or at least one of its target genes in the subject with the amount of gene product of runx1 or at least one of its target genes present in a normal tissue sample obtained from a subject who does not have a muscle wasting disease or in a predetermined standard, wherein a decrease in the amount of the gene product of runx1 or at least one of its target genes in the subject compared to the amount in the normal tissue sample or pre-determined standard indicates a risk of developing a tissue wasting disease in the subject.

30. A method for screening, diagnosing or prognosing of a disease in a subject, wherein the disease is characterized by muscle atrophy selected from the group consisting of myopathies, dystrophies, myoneural conductive diseases, traumatic muscle injury, nerve injury, cardiomyopathies, ischemic damage, congenital disease, and other conditions resulting in muscle wasting, the method comprising:
(I) measuring an amount of a runx1 gene or target gene for runx1 or their corresponding gene product in a tissue sample derived from the subject, wherein the runx1 gene or runx1 target gene or corresponding gene product is:
(a) a DNA corresponding to SEQ ID NO: 1, or to any one of SEQ ID NOS: 3-31 or a nucleic acid derived therefrom;
(b) a protein comprising SEQ ID NO: 2, or a protein encoded by any one of the nucleic acid sequences of SEQ ID NOS: 3-31;
(c) a nucleic acid comprising a sequence hybridizable to SEQ ID NO: 1, or to any one of SEQ ID NOS: 3-31 or their complements under conditions of high stringency, or a protein comprising a sequence encoded by the hybridizable sequence;
(d) a nucleic acid at least 90% homologous to SEQ ID NO: 1, or to any one of SEQ ID NOS: 3-31 or their complement as determined using the NBLAST algorithm; or a protein encoded thereby; and
(II) comparing the amount of the runx1 gene product or the target of the runx1 gene in the subject with the amount of runx1 gene product or the target of the runx1 gene present in a normal tissue sample obtained from a subject who does not have a muscle wasting disease or in a predetermined standard, wherein a decrease in the amount of the runx1 gene product or the runx1 target gene in the subject compared to the amount in the normal tissue sample or pre-determined standard indicates the presence of a tissue wasting disease in the subject.

31. A method for determining a predisposition to, onset of, or presence of a muscle wasting disease in a subject, comprising detecting in the subject the existence of a change in the level of a runx1 gene, or a runx1 target gene or corresponding gene product, as set forth in SEQ ID NOs: 1-31.

32. The method of claim 31, comprising the steps of:
a) obtaining a tissue biopsy from the subject;
b) permeabilizing the cells in the tissue biopsy;
c) incubating the tissue biopsy or cells isolated from the tissue biopsy with one of the following:
i) an antibody specific for the runx1 gene product, or for the gene product of the runx1 target gene;
ii) a nucleic acid probe specific for the runx1 gene, or for the runx1 target gene;
d) detecting and quantifying the amount of antibody or nucleic acid probe bound; and
e) comparing the amount of antibody or nucleic acid probe bound in the biopsy sample in the subject to the amount of antibody or nucleic acid probe bound in a normal tissue or cellular sample;
wherein the amount of labeled antibody or nucleic acid probe bound correlates inversely with the predisposition, the onset or the presence of a muscle wasting disease in a subject.

33. A biomarker associated with and/or predictive of a muscle wasting disease or condition comprising a runx1 gene or gene product, a runx1 target gene or its corresponding gene product, or a nucleic acid sequence hybridizable to a runx1 gene or a runx1 target gene or their corresponding gene products, under conditions of high stringency.

34. The biomarker of claim 33, wherein the runx1 gene comprises the nucleic acid sequence as set forth in SEQ ID NO: 1, and wherein a runx1 target gene is selected from the group consisting of any one of SEQ ID NOS: 3-31, or combinations thereof.

35. The biomarker of claim 33, wherein the runx1 gene encodes a polypeptide comprising the amino acid sequence as set forth in SEQ ID NO. 2.

36. The biomarker of claim 33, wherein the muscle wasting disease or condition is a skeletal muscle wasting disease or condition.

37. The biomarker of claim 36, wherein the skeletal muscle wasting disease is a myopathy or a dystrophy.

38. The biomarker of claim 36, wherein the skeletal muscle wasting disease or condition is the result of a neural disorder, a neuropathy or a nerve injury.

39. The biomarker of claim 36, wherein the skeletal muscle wasting disease or condition is the result of an accident or injury.

40. The biomarker of claim 33, wherein the muscle wasting disease or condition is characterized by muscle damage, muscle atrophy or muscle cell autophagy.

41. The biomarker of claim 40, wherein the muscle damage, muscle atrophy or muscle cell autophagy occurs in skeletal muscle, cardiac muscle or smooth muscle.

42. The biomarker of claim 41, wherein the muscle damage, muscle atrophy, or muscle cell autophagy occurs in skeletal muscle and is the result of a muscle wasting disease or condition, a muscle injury, or a result of aging.

43. The biomarker of claim 41, wherein the muscle damage, muscle atrophy, or muscle cell autophagy occurs in cardiac muscle and is the result of a cardiomyopathy, an ischemic event, a degenerative congenital disease or any cardiac trauma.

44. The biomarker of claim 41, wherein the muscle damage, muscle atrophy, or muscle cell autophagy occurs in smooth muscle and is the result of arterial sclerosis, a vascular lesion, or a congenital vascular disease.


SEQUENCE LISTING (31 sequences)

SEQ ID NO: 1 - DNA, 4295 bp, Mus musculus
ctcattcatagagccagcgggcgcgggcaggacgggcgccccgcggccggacccagccagggcaccacgctgcccggccccgcggctggcacttccatcctgagctccccgagacgcaagaaagtcaccctttgcggctctttctagatctgcgttggaccttcttgttttcctttcctttcctttttttttttttttaatttttatttttattttccctttcttacttttgaatgaaccggcttctaggctttggatgtttcaacttctaggctgtgaacttttctccgattgtttcttttgacaactgcgggagaaagtgttccggagttccaggcgagcggtgaagttgcgtgtgcgtggcagggtgcgtgggagtgagcgtgtgtaactcgagcctactggtctacggtgcctcctcccaggcccgctccacctgctgcggttacacggtgctcgctcgtgggtatttgtgcgccccagcagaaaaccagccaccgccgggcgacgttggaggaagatcatcttgccagactctgggctgactcactcaaccaatgtagacagtggctgacttccgaagagtgcgagtctgcctgtgtgtgtgacccaactgcggctcaactcccaccaaaccacaggatcagccacaaacttaaccagcgttcccaaaccagagtcctcagctgtgggaagctctaccctgagtgtcatcgagtttgacttttttttttttaagattagctacagatctttgtaaagcactgaccatttatttggccagaatttattgagtaatgacaagtgatcctatccagccttccaactactgaagctgattttcaaggctacttaaaaaaaaaaaaactgcagcaaacattaatggatttctgttgtgtttaaattctctacagattgtattgtaaatattttatgaagtagatcatatgtatatatttatatatacgagcacatacattagtagcacaatcttttaaagttacggctcttgcttttgagaaacgaagtgagtttttcatggtaagaggggcgctctgtatggaagacactcctaagttttgtattttgttgagactctaaacaaaactgacccgcaagaaagaaaaacaaactgacaggcaactaactggtggaacttccaaatctagtttttgctggtcattttttggttgttgttgttgttctgcgcataaatattttaggacgcgtatgggaattttgctccgggaccgtttgtaatagccaaagactgaacttcaactctcaaagcgaggctctgttgggcatttgactttgaattctggttgcccactttttaacgcactaagcggccagttgctaaccctgcttgggtgtgaggccgatccgtgtaactagatctcagtccgggactgatggtaatttagtctcctacccccacaaccctccggtagtaataaaggcttctgaacttgtatgttggtctcccgggagcagcttgcagaagatccgagcccctgtcgccgtctagtaggagctgttttcagggtccttactcaatcggcttgttgtgatgcgtatccccgtagatgccagcacgagccgccgcttcacgccgccttccaccgcgctgagccccggcaagatgagcgaggcgctgccgctgggcgccccggatggcggcccggccctggccagcaagctgaggagcggcgaccgcagcatggtggaggtactagctgaccaccctggcgagctagtgcgcaccgacagccccaacttcctctgctccgtgctacccactcactggcgctgcaacaagaccctgcccatcgctttcaaggtggtggcactgggggacgtcccggatggcactctggtcaccgtcatggcaggcaacgatgaaaactactcggcagaactgagaaatgctaccgcggccatgaagaaccaggtagcgagattcaacgacctcaggtttgtcgggcggagcggtagaggcaagagcttcactctgaccatcaccgtctttacaaatccgccacaagttgccacctaccatagagccatcaaaatcacagtggacggcccccgagaaccccgaaatgccaggcagatccagccatccccaccgtggtcctatgaccagtcctaccagtacctgggatccatcacctcttcctctgtccacccagcgacacccatttcacccggccgtgccagcggcatgaccagcctctctgcagaactttccagtcgactctcaacggctccggacctgaccgccttcggcgacccacgccagttccctactctgccgtccatctccgacccgcgcatgcactacccaggcgccttcacctactcgccgcccgtcacgtcgggcatcggcatcggcatgtcagccatgagctcggcctctcgctaccacacctacctgccgccgccctaccccggctcatcacaggcgcaggccgggcccttccagaccggctcgccctcctaccatctatactacggcgcctcggccggttcctaccagttctccatggtgggcggagagagatcgcccccgcgcatcctgccgccctgcaccaacgcatccaccggcgccgcgctgctcaaccccagcctccccagccagagcgacgtggtggagaccgagggcagccatagcaactcgcccaccaacatgccccccgcgcgcctggaggaggccgtgtggcggccctactgagctgagcgccatcgccatcgagggactgggcctgccgtccatgcacagaccccgccaggagggcccttggaggccaccaggaagaatcccggagggaaactgtgaatgcttctgatttagcaatgctgtgaataaaagaaagattttatacccttgacttcactttttaaccacgttgtttattccaaagagtgtggaatgttttcggttcggggtggggaagacgcagcccatcctgtttggcatctatttcttatttcggagttttcttttccgcaccttatcgattgcaaaaatgcctgtttgcatctgggtggtcatttatttttaagtgtgtatagatttgagcttgcttttttttcttcctttgaccaactcaaagaaataaaattcccttctctgtaaggtttatttaacttttagactttcatgtagctttggggttttatttgtgtttggtttttgtttttgtttttatttttaaagagacagctacagctttgggtcattttttaactactgtatttccacaaagaaatccctagatatttatgtatcttgatgtttgaacatttacatatgtgttgatacttttttaattatttaaatgtacttatattaagaaagatatcaagtactacatttttctttataatagccaaagttaaatattattgcgttgaagatgtctggaaaaaaaagagatcgcttggttaactagaaatattgtttacattaaactccctttatgttattcaaacaagttggtaggtaacgcagcaatgtttttaattggattgtagacactgagggtcactccaaggtcagaagtacaaaattttctgctaggctcaacaaatagtctcatacctggctccttcccttcaaaaagagaggcaaactctgtcctgaaagggttcagagaggtgccaaggatttgctctgaagaggatttcattttggcctggagatatacttgccccaaggcctcctcattctggcatgctttatcacagagctcaaccaagtaagctgttggtcaggggtttacttacatagtatttacatagacccaaaccactgaatgtgatttttaaattgccttccattaatagtacccgttcattgatgaaaaccaaaacttgaggctgtaccccaaagatccaaatagaagagttaagaccaggtgtctttgaggcctaaaggctgagttttaagagagtgtaccccaaaagtctgaaggagccggtttccttctcccagtcttagtggaatcagtcatgggaggcagatgccacgcccacctgtgcaggatgctcctcagaagctgccccttcaccagcatcttctcccaccaggccgagcccctgacctttggggtgcatcagtgtgatagatcctggtctctgcagtccgccatggctacggttcagatgtgcatcgtgtcactgtaaatgtaatggtactgttgttacagtggaggacttggtcaaaatccagttgttctacaacgtatgaagcctaaccgctggttctgacatacatgtgctcaaaatga

SEQ ID NO: 2 - protein, 453 aa, Homo sapiens [sequence omitted]

SEQ ID NO: 3 - DNA, 1486 bp, Homo sapiens
agcagcaggaggaggcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaaaaaaaaaaaaaaaataaaaaaaaaaaaaaaaaaaaaaaaaaaaa

SEQ ID NO: 4 - DNA, 2187 bp, Homo sapiens
actgcagagagctgaggcaccatgcatgggggccaggggccgctgctcctcctgctgctgctggctgtctgcctgggggcccagggccggaaccaggaggagcgcctgctcgcagacctgatgcaaaactacgaccccaacctgcggcccgcggaacgagactcggatgtggtcaatgtcagcctgaagctaaccctcaccaacctcatctccctgaacgagcgagaggaagccctcaccaccaatgtctggatagagatgcagtggtgcgactatcgcctgcgctgggatccgcgagactacgaaggcctgtgggtgctgagggtgccgtccaccatggtgtggcggccggatatcgtgctggagaacaacgtggacggtgtcttcgaggtggccctctactgcaatgtgctcgtgtcccctgacggctgtatctactggctgccgcctgccatcttccgttccgcctgctctatctcagtcacctacttccccttcgactggcagaactgctcccttatcttccagtcccagacttacagcaccaatgagattgatctgcagctgagtcaggaagatggccagaccatcgagtggattttcattgaccctgaggccttcacagagaatggggagtgggccatccagcaccgaccagccaagatgctcctggacccagcggcgccagcccaggaagcaggccaccagaaggtggtgttctacctgctcatccagcgcaagcccctcttctacgtcatcaacatcatcgccccctgtgtgctcatctcctctgtcgccatcctcatccacttccttcctgccaaggctgggggccagaagtgtaccgtcgccatcaacgtgctcctggcccagactgtcttcctcttccttgtggccaagaaggtgcctgaaacctcccaggcggtgccactcatcagcaagtacctgaccttcctcctggtggtgaccatcctcattgtcgtgaatgctgtggttgtgctcaatgtctccttgcggtctccacacacacactccatggcccgaggggtccgcaaggtgttcctgaggctcttgccccagctgctgaggatgcacgttcgcccgctggccccggcagctgtgcaggacacccagtcccggctacagaatggctcctcgggatggtcgatcacaactggggaggaggtggccctctgcctgcctcgcagtgaactcctcttccagcagtggcagcggcaagggctggtggcggcagcgctggagaagctagagaaaggcccggagttagggctgagccagttctgtggcagcctgaagcaggctgccccagccatccaggcctgtgtggaagcctgcaacctcattgcctgtgcccggcaccagcagagtcactttgacaatgggaatgaggagtggttcctggtgggccgagtgctggaccgcgtctgcttcctggccatgctctcgctcttcatctgtggcacagctggcatcttcctcatggcccactacaaccgggtgccggccctgccattccctggagatccacgcccctacctgccctcaccagactgagccaaccaaccactgtggggcatgtgggagtcacacacgtgggtcacactgagtcttatcagccacgttctcctactgaggtcctaagtgtgctctttgggaagtgcccttcaggactgtgtgagccaaacagccctgagaaaagctggggaaacagtctgagctggagtccgagagtggttgggggtgggccgtggctagtgtcctgctgcagtcagcacacacgtgggattggctagctcatcctggcaccagccacccctccactcagtgcactcccctcacttaggcaaagcattattcattcccatcagtctgaagcccgaaggactgttttgtataataccttcggacttgggactggctccccttttacaagttctccctgaaagagggcagtcacaagaggtgtgaagagtagcagccgatgctctctccaaagcagggcagcagcccataccagctggcatctcccccccgtgccttctgggtacaataagcacccaattctcaacagccccagtggcctttccattcatgtgcatttttctgccactgaccacaagacgatttcctgagttttgtaatcctctttttttttttttttttttt

SEQ ID NO: 5 - DNA, 2823 bp, Homo sapiens
agattcactggtgtggcaagttgtctctcagactgtacatgcattaaaattttgcttggcattactcaaaagcaaaagaaaagtaaaaggaagaaacaagaacaagaaaaaagattatattgattttaaaatcatgcaaaaactgcaactctgtgtttatatttacctgtttatgctgattgttgctggtccagtggatctaaatgagaacagtgagcaaaaagaaaatgtggaaaaagaggggctgtgtaatgcatgtacttggagacaaaacactaaatcttcaagaatagaagccattaagatacaaatcctcagtaaacttcgtctggaaacagctcctaacatcagcaaagatgttataagacaacttttacccaaagctcctccactccgggaactgattgatcagtatgatgtccagagggatgacagcagcgatggctctttggaagatgacgattatcacgctacaacggaaacaatcattaccatgcctacagagtctgattttctaatgcaagtggatggaaaacccaaatgttgcttctttaaatttagctctaaaatacaatacaataaagtagtaaaggcccaactatggatatatttgagacccgtcgagactcctacaacagtgtttgtgcaaatcctgagactcatcaaacctatgaaagacggtacaaggtatactggaatccgatctctgaaacttgacatgaacccaggcactggtatttggcagagcattgatgtgaagacagtgttgcaaaattggctcaaacaacctgaatccaacttaggcattgaaataaaagctttagatgagaatggtcatgatcttgctgtaaccttcccaggaccaggagaagatgggctgaatccgtttttagaggtcaaggtaacagacacaccaaaaagatccagaagggattttggtcttgactgtgatgagcactcaacagaatcacgatgctgtcgttaccctctaactgtggattttgaagcttttggatgggattggattatcgctcctaaaagatataaggccaattactgctctggagagtgtgaatttgtatttttacaaaaatatcctcatactcatctggtacaccaagcaaaccccagaggttcagcaggcccttgctgtactcccacaaagatgtctccaattaatatgctatattttaatggcaaagaacaaataatatatgggaaaattccagcgatggtagtagaccgctgtgggtgctcatgagatttatattaagcgttcataacttcctaaaacatggaaggttttcccctcaacaattttgaagctgtgaaattaagtaccacaggctataggcctagagtatgctacagtcacttaagcataagctacagtatgtaaactaaaagggggaatatatgcaatggttggcatttaaccatccaaacaaatcatacaagaaagttttatgatttccagagtttttgagctagaaggagatcaaattacatttatgttcctatatattacaacatcggcgaggaaatgaaagcgattctccttgagttctgatgaattaaaggagtatgctttaaagtctatttctttaaagttttgtttaatatttacagaaaaatccacatacagtattggtaaaatgcaggattgttatataccatcattcgaatcatccttaaacacttgaatttatattgtatggtagtatacttggtaagataaaattccacaaaaatagggatggtgcagcatatgcaatttccattcctattataattgacacagtacattaacaatccatgccaacggtgctaatacgataggctgaatgtctgaggctaccaggtttatcacataaaaaacattcagtaaaatagtaagtttctcttttcttcaggggcattttcctacacctccaaatgaggaatggattttctttaatgtaagaagaatcatttttctagaggttggctttcaattctgtagcatacttggagaaactgcattatcttaaaaggcagtcaaatggtgtttgtttttatcaaaatgtcaaaataacatacttggagaagtatgtaattttgtctttggaaaattacaacactgcctttgcaacactgcagtttttatggtaaaataatagaaatgatcgactctatcaatattgtataaaaagactgaaacaatgcatttatataatatgtatacaatattgttttgtaaataagtgtctccttttttatttactttggtatatttttacactaaggacatttcaaattaagtactaaggcacaaagacatgtcatgcatcacagaaaagcaactacttatatttcagagcaaattagcagattaaatagtggtcttaaaactccatatgttaatgattagatggttatattacaatcattttatatttttttacatgattaacattcacttatggattcatgatggctgtataaagtgaatttgaaatttcaatggtttactgtcattgtgtttaaatctcaacgttccattattttaatacttgcaaaaacattactaagtataccaaaataattgactctattatctgaaatgaagaataaactgatgctatctcaacaataactgttacttttattttataatttgataatgaatatatttctgcatttatttacttctgttttgtaaattgggattttgttaatcaaatttattgtactatgactaaatgaaattatttcttacatctaatttgtagaaacagtataagttatattaaagtgttttcacatttttttgaaagacaaaaa

SEQ ID NO: 6 - DNA, 1443 bp, Homo sapiens
gtggctgcagcagcagcggcggcggaaaccctaaagtccgagtccggactacgagtgcgtggcctcctaatccggatcctagtcctgagcgtgtctgtgtgcgagtggacggtcccggacgcgatgaccctgaacggcggcggcagcggagcgggcgggagccgcggtgggggccaggagcgcgagcgccgtcggggcagcacaccctggggccccgccccgccgctgcaccgccgcagcatgccggtggacgagcgcgacctgcaggcggcgctgaccccgggtgccctgacggcggccgcggccgggacggggacccagggtcccaggctggactggcccgaggactccgaggactcgctcagctcagggggcagcgactcagacgagagcgtttacaaggtgctgctgctgggggcgcccggcgtgggcaagagcgccctggcgcgcatcttcggcggtgtggaggacgggcctgaagcagaggcagcagggcacacctatgatcgctccattgtagtggacggagaagaggcatcactcatggtctacgacatttgggagcaggacgggggccgctggttgcccggccactgcatggccatgggggatgcctatgtcattgtgtactcagtgacggacaagggcagcttcgagaaggcctcagaactgcgggtccagctgcggcgtgcacggcaaacagatgatgtgcccatcatcctcgtgggcaacaagagcgacctggtgcgctctcgtgaggtctcggtggatgagggccgggcctgcgcggtggtctttgactgcaagttcattgagacatcagcggcattgcaccacaatgtccaggcgctgtttgaaggtgtcgtgcgccagatacgcctgcgcagggacagcaaagaagccaacgcacgacggcaagcaggcacccggaggcgagagagccttggcaaaaaggcgaagcgcttcttgggccgcatcgtagctcgtaacagccgcaagatggcctttcgcgccaaatccaagtcctgccacgacctctcggttctctaggtcccacccgctcccactatggtgggagacgaacggaagggttggtgggctggcccagccaactgccccgggtgcctcagagcaggctcagactctgggtccctcggagctgccagccgggcacccccaacctcatggtcatggacagatagacagtgctgccctgcgaagtggctctcaggggccagtgagggctgggcccacagagatgcatgcgcaggctcatatgcgtcccaagcagccgcagcgcagccgccgggcaggcctgcgtgccgggagaggactctgccttttttcacagcccgggtgtgcctgccctggagggaggctcttcagtgcggtagctacttgtttacatgcagatttttgtaataaaggctatttcctgataaa

SEQ ID NO: 7 - DNA, 1738 bp, Homo sapiens
ggcgggcccgcgctcacttcctcggggaggggttggggccgcgctcccactcgcccacacggtccgtctgcagtcggcagcctcgccggcagctcgctcggcccccgcttggcccggcccgcaggtaggagccgccgagggacgcggcgcccggttcccggccccgcctgcgccgcgcgctccccgctcccgccgcccgcttcgagggttctctccccttgccaccatgagcgagtcatttgactgtgcaaaatgcaacgagtccctgtatggacgcaagtacatccagacagacagcggcccctactgtgtgccctgctatgacaatacctttgccaacacctgtgctgagtgccagcagcttatcgggcatgactcgagggagctgttctatgaagaccgccatttccacgagggctgcttccgctgctgccgctgccagcgctcactagccgatgaacccttcacctgccaggacagtgagctgctctgcaatgactgctactgcagtgcgttttcctcgcagtgctccgcttgtggggagactgtcatgcctgggtcccggaagctggaatatggaggccagacatggcatgagcactgcttcctgtgcagtggctgtgaacagccactgggctcccgttcttttgtgcccgacaagggtgctcactactgcgtgccctgctatgagaacaagtttgctcctcgctgcgcccgctgcagcaagacgctgacacagggtggagtgacataccgtgatcagccgtggcatcgagaatgtctggtctgtaccggatgccagacgcccctggcagggcagcagttcacctcccgggatgaagatccctactgtgtggcctgttttggagaactctttgcacctaagtgcagcagctgcaagcgccccatcgtaggactcggtggaggcaagtatgtgtcctttgaagaccgacactggcaccacaactgcttctcctgcgcccgctgctctacctccctggtgggccagggcttcgtaccggatggagaccaagtgctctgccagggctgtagccaggcagggccctaagccagggctcctggacccaggctttcccataccacgggcccaggactgtggctccttttctaaaccacctctgggactcagctccccccgccaaaaaaaatgggtctccttctgggctccaggattgtctccccactccagcatccccaaactggtactccctgacccaggcccccaatcctgggctcttacagagcctccatgagtcaagccccctccccacacctggactccagaattcaccctctcccctgcagtctgggttcccagactgagtcctctccccaaatcagggctctagacccgagccctccaaacctggactctgggacttaggcccccttaaatctagacttctctttataggtttcaggtctcctatgggtgcctgggaagtccttgaaagtggactgttctcaggcttgacctgccccaccccatccccgcggttgaggctgtgggggcagcagatcaggagcccactgataaggggccctagggtacagggtgctgcccagcaggtcgccaccgagtgtcttctcattttatttcagctccattttgcccatagatgggcagaggggtgagattggctcatcccccttccagattctgcaataaagcggtgtgaggaaaaaaaaaaaaaaaaaaaaaaaaaaaaaaaaaaaa

SEQ ID NO: 8 - DNA, 6096 bp, Homo sapiens
  misc_feature 4348, 4349, 4350, 4351, 4352, 4353, 4354, 4355, 4356,4357, 4358, 4359, 4360, 4361, 4362, 4363, 4364, 4365, 4366, 4367,4368, 4369, 4370, 4371, 4372, 4373, 4374, 4375, 4376, 4377,4378, 4379, 4380, 4381, 4382, 4383, 4384, 4385, 4386: n = A,T,C or G
  misc_feature 4387, 4388, 4389, 4390, 4391, 4392, 4393, 4394, 4395,4396, 4397, 4398, 4399, 4400, 4401, 4402, 4403, 4404, 4405, 4406,4407, 4408, 4409, 4410, 4411, 4412, 4413, 4414, 4415, 4416,4417, 4418, 4419, 4420, 4421, 4422, 4423, 4424, 4425: n = A,T,C or G
  misc_feature 4426, 4427, 4428, 4429, 4430, 4431, 4432, 4433, 4434,4435, 4436, 4437, 4438, 4439, 4440, 4441, 4442, 4443, 4444, 4445,4446, 4447, 4448, 4449, 4450, 4451, 4452, 4453, 4454, 4455,4456, 4457, 4458, 4459, 4460, 4461, 4462, 4463, 4464: n = A,T,C or G
  misc_feature 4465, 4466, 4467, 4468, 4469, 4470, 4471, 4472, 4473,4474, 4475, 4476, 4477, 4478, 4479, 4480, 4481, 4482, 4483, 4484,4485, 4486, 4487, 4488, 4489: n = A,T,C or G
atggcaaacttcctattacctcggggcaccagcagcttccgcaggttcacacgggagtccctggcagccatcgagaagcgcatggcggagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcatatcctttgcagttaatacctgctgagtaccccctgggggcccacgctggggatgtgcacctttccttgaccccacgcacgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaggatgaggagaacagcctgggcacggaggaggagtccagcaagcaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtg

SEQ ID NO: 9 - DNA, 2967 bp, Mus musculus
cttttcctccctgttgtccctggccttgtccggagtcgggctcataccatgtcggtcgcactcagcaacaggttccaaggagggaaggcgttcggtttgctcaaagcccggcaggagaagaggctcgaggagatcaatcgggagttcctctgtgaccagaagtacagtgatgaagagaacctgccggaaaagctcgcagccttcaaagagaaatacatggagtttgacctgaataatgaaggcgagattgatctgatgtctttaaagaggatgatggagaagctgggggtccccaagacccacctggagatgaagaagatgatctcggaggtgacagggggtgtcagtgacaccatctcctaccgagactttgtgaatatgatgctgggcaagcggtcggctgtcctcaagctggtcatgatgtttgaaggaaaagccaacgagagcagcccgaagccagctggcccacctccagagcgagacatcgccagcctgccttgaggacccacccggaccttccgggctgccatccctgcccctgcctctccctctgcgctctgccatcttgactcattgtgttaggtctcctgggttatgccccagtggactcattttttttttttctctatgtaagcacatgttacctgacttaatgaggctgggaatcccgagcctcaggcccctctgctccctgctgctctgccctctttgtacaaaagggctaacatcaaaccaaaaactggagagggcagggaggctgcaaacctcgttcccatgtttgaagagggcctgtccgtcagttcatctttctcagcttctaaattaaattcaggctgcaagctgctctcggaagatccagagcaggcacagagctccagggctccctagtgtcttagggacatgcttccagtgccgctggaccttctagcagcggctactcagtggctagacacacaactgattcccttttgttcttgcccttgtccattctcagcgacatgacgcaaggtggggaggaaagaacaacctttgggtacttcagaggaaccagaaggaaccgctcctttacagacagctcagagggacagtgtaatggcactgtcccttgctagagcagtagaggggtttggaggcagaggtgaggtcagtggtgtgagggagtcctactttcagggtggatgctgagggcaccagggtagaagtggagtcaagtgttgaacctcaaacagagaacaggctgtctgattgcacagtgccccaatccggctatttactgtcctgccctactctcccctcccttcactccctcagttatttatcgagtgtgtattacaagctttaagttctcttgctttgtctccatgtgtgctatgtccaactcctagctcccttttcaaatccctcaagtcctggagcttggggattagatggaggtaacatggcagattttagctgggccagtccttccatttcactgctggcctccaggggttaagtgccttgctgaaggttggtgggggctatcagagagtgtggactggagacccgctctaggctgcattggtcaaaggaaagggtagagagcagcatttcccagcctgatacttgaggaaggtgtggtctccacggaacaccttgggtcaggactcaccctcctgagggcctccaggttggcagtgagatttcagtaagcaccaagcctagctttgcagattgtcttccttacagcttcctgactggtcttcagagaccatttccaagaattcatgcagcaaagtatcaaatgtcttacccgagtggggaacctcagccttgaccctgcttgtgttctggaaccatttactctgggcactcaggacgagacagtcactcatcacacatatttatcaagcacctgctcaatgacacgagtccagaacccggcagtgaaatggactgatgtatgaaatttatgtctactggggaatcactcccagccaatctagccaatgactatgatatggagtgggggggagtgcaagaggtgagtaccatgtagtcccagggattccagctgggctcataatttggcaagtgtcagaggagcctctgtaaaaccttaagtggaaaagacagggaagcgttgagctaatgtgcttttcctgtggctcttgttccacagtccaatacccagggtgctgttcccacagcagtgagagagagagagagtgaagcaaacagggcatggaatggttagctccagttcgtcctctggtctcaaccagttcagttcaggagagagatcatttcgctatgactggctgattttcagccctaggagctgcttcaaatgcacagtctagttgaagtttaaaccccagcattcctccctataaatataaaatcctgtctaccctcaccccatcctttgacttatttttcttttaagattagcatctctgcccccatttcctcctgccttccttctctgagcgggctgggaccttggactgcattgggtccctattctatgtgacagaggaaggaagaatcagccggctcagtgggtccacaactcatgagcttacacaccacctccataaaggagtgtgccactgacctctgcagagcacagaaagaggcagtggtctgaagatcaatctatgggaagacttaagaatgaccctttaatggaggagttggcatatatttcaagttgtgtctgttggttccagggtttttaacctctctagttaagggcttagctttcttgggacatcggctgccttatttctgaaaaagaccaaatgtaactggtgtaaccagcagtttggggattgccaagtatggctttgccctgtgactcaaaagatgtttgtaaggtagatttgggtgagtgccattattgtgtgcatgtgtgtatgtatggatgggatgtggtctcctgcagactgaaataaactagagcaatttaaaaaaaaaaaaaaa

SEQ ID NO: 10 - DNA, 1714 bp, Mus musculus
cgaacctccgtcttcagctcactgccttcgctccagacttcaccatgtccatcagggtgactcagaaatcctacaagaatgtctactcggtcggacttctagctagcgctcgttcaggatcgtggactggatccgctagtactgcttctagcttttcgggtcgggcatgtcagctaggtagcagcttcggggaagcatggcacggtgtctctggcggctttggcggggctggtgtcgggggcatcacagccgtcacggtggatcagagcctgttgagccccttgcagctgagtctccccaacatccaggctgtgcgcactcaggagaaggagcagattaaatccctgaacaacaagttcgcctccttcattgacaaggtgcgcttcctggagcagcagaacaagatgctggagaccaagtggagcctgttgcagcagcagaagacgtcgaggagcaacatggacaacatgtttgagagctacatcaacaacctccgccggcagctggaagccctgggccaggagaagctgaagctggaggctgagcttggcaacatgcagggcctggtggaggacttcaagaataagtatgaggatgagatcaacaagcgtacagagatggagaatgaatttgtcctcatcaagaaggatgtggacgaagcatacatgaacaaggtggaactagagtcccgcctggaaggactgaccgacgagatcaacttcctccggcagatccatgaagaggagatccgtgagttgcagtctcagatctcagacacgtctgtggtgctgtctatggacaacagccgctccctggacatggatggcatcatcgctgaagttcgtgcccagtacgaggacattgccaatcgcagccgagctgaggctgaaaccatgtaccagattaagtatgaggaattgcagaccctggctgggaagcacggggatgatctgcgccgcacaaagacagagatctccgagatgaaccgcaacatcaaccgcctgcaggcggagattgaagccctcaaaggccagagggcatcgttggaggcggccatcgctgatgctgagcagcgtggggagatggccattaaggatgcccagaccaagctggctgagctggaggctgccctgcaacgggccaagcaggacatggccaggcagctgcgcgagtaccaggagcttatgaacgtcaagctggccctggacatcgagatcaccacctaccgcaagctgctggagggggaggagagcaggctggagtctgggatgcagaacatgagcattcatacgaagaccaccagcggctactcaggaggactgagttcatcctatgggggactcactagccctggcttcagctacggaatgagctccttccagcccggcttcggttctgccgggggatccaacactttcagccgcaccaccaaggctgtggttgtgaagaagattgaaacccgagatgggaagctggtgtccgagtcttctgatgtcgtgtccaagtgaatggccactgaagtccttgccagcctgagctcctgcagctgtatggcagagatcagggataatagagacccgtccgaggatcagccctaatcctctggccaaccttaggaggaatttcctatctgggatatcccaatggcttctgcctcccatccaaacccaattcagttgtattgtctaaaataaagccatcagccggctatgtc

SEQ ID NO: 11 - DNA, 5196 bp, Homo sapiens
gtgtgtgcgcgcgtgaggggggcagcagagaggagtctcccggtgccgccgcggcgtcagagacactgcgagcggcgagcgcggtggggccgcatctgcatcagccgccgcagccgctgcggggccgcgaacaaagaggaggagccgaggcgcgagagcaaagtctgaaatggatgttacatgagtcattttaagggatgcacacaactatgaacatttctgaagattttttctcagtaaagtagataaagatggatgaatcagccttgttggatcttttggagtgtccggtgtgtctagagcgccttgatgcttctgcgaaggtcttgccttgccagcatacgttttgcaagcgatgtttgctggggatcgtaggttctcgaaatgaactcagatgtcccgagtgcaggactcttgttggctcgggtgtcgaggagcttcccagtaacatcttgctggtcagacttctggatggcatcaaacagaggccttggaaacctggtcctggtgggggaagtgggaccaactgcacaaatgcattaaggtctcagagcagcactgtggctaattgtagctcaaaagatctgcagagctcccagggcggacagcagcctcgggtgcaatcctggagccccccagtgaggggtatacctcagttaccatgtgccaaagcattatacaactatgaaggaaaagagcctggagaccttaaattcagcaaaggtgacatcatcattttgcgaagacaagtggatgaaaattggtaccatggggaagtcaatggaatccatggctttttccccaccaactttgtgcagattattaaaccgttacctcagcccccacctcagtgcaaagcactttatgactttgaagtgaaagacaaggaagcagacaaagattgccttccatttgcaaaggatgatgttctgactgtgatccgaagagtggatgaaaactgggctgaaggaatgctggcagacaaaataggaatatttccaatttcatatgttgagtttaactcggctgctaagcagctgatagaatgggataagcctcctgtgccaggagttgatgctggagaatgttcctcggcagcagcccagagcagcactgccccaaagcactccgacaccaagaagaacaccaaaaagcggcactccttcacttccctcactatggccaacaagtcctcccaggcatcccagaaccgccactccatggagatcagcccccctgtcctcatcagctccagcaaccccactgctgctgcacggatcagcgagctgtctgggctctcctgcagtgccccttctcaggttcatataagtaccaccgggttaattgtgaccccgcccccaagcagcccagtgacaactggcccctcgtttactttcccatcagatgttccctaccaagctgcccttggaactttgaatcctcctcttccaccaccccctctcctggctgccactgtccttgcctccacaccaccaggcgccaccgccgctgctgctgctgctggaatgggaccgaggcccatggcaggatccactgaccagattgcacatttacggccgcagactcgccccagtgtgtatgttgctatatatccatacactcctcggaaagaggatgaactagagctgagaaaaggggagatgtttttagtgtttgagcgctgccaggatggctggttcaaagggacatccatgcataccagcaagataggggttttccctggcaattatgtggcaccagtcacaagggcggtgacaaatgcttcccaagctaaagtccctatgtctacagctggccagacaagtcggggagtgaccatggtcagtccttccacggcaggagggcctgcccagaagctccagggaaatggcgtggctgggagtcccagtgttgtccccgcagctgtggtatcagcagctcacatccagacaagtcctcaggctaaggtcttgttgcacatgacggggcaaatgacagtcaaccaggcccgcaatgctgtgaggacagttgcagcgcacaaccaggaacgccccacggcagcagtgacacccatccaggtacagaatgccgccggcctcagccctgcatctgtgggcctgtcccatcactcgctggcctccccacaacctgcgcctctgatgccaggctcagccacgcacactgctgccatcagtatcagtcgagccagtgcccctctggcctgtgcagcagctgctccactgacttccccaagcatcaccagtgcttctctggaggctgagcccagtggccggatagtgaccgttctccctggactccccacatctcctgacagtgcttcatcagcttgtgggaacagttcagcaaccaaaccagacaaggatagcaaaaaagaaaaaaagggtttgttgaagttgctttctggcgcctccactaaacggaagccccgcgtgtctcctccagcatcgcccaccctagaagtggagctgggcagtgcagagcttcctctccagggagcggtggggcccgaactgccaccaggaggtggccatggcagggcaggctcctgccctgtggacggggacggaccggtcacgactgcagtggcaggagcagccctggcccaggatgcttttcataggaaggcaagttccctggactccgcagttcccatcgctccacctcctcgccaggcctgttcctccctgggtcctgtcttgaatgagtctagacctgtcgtttgtgaaaggcacagggtggtggtttcctatcctcctcagagtgaggcagaacttgaacttaaagaaggagatattgtgtttgttcataaaaaacgagaggatggctggttcaaaggcacattacaacgtaatgggaaaactggccttttcccaggaagctttgtggaaaacatatgaggagactgacactgaagaagcttaaaatcacttcacacaacaaagtagcacaaagcagtttaacagaaagagcacatttgtggacttccagatggtcaggagatgagcaaaggattggtatgtgactctgatgccccagcacagttaccccagcgagcagaatgaagaagatgtttgtgtgggttttgttagtctggattcggatgtataaggtgtgccttgtactgtctgatttactacacagagaaacttttttttttttttaagatatatgactaaaatggacaattgtttacaaggcttaactaatttatttgcttttttaaacttgaacttttcgtataatagatacgttctttggattatgattttaagaaattattaatttatgaaatgataggtaaggagaagctggattatctcctgttgagagcaagagattcgttttgacatagagtgaatgcattttcccctctcctcctccctgctaccattatattttggggttatgttttgcttctttaagatagaaatcccagttctctaatttggttttcttctttgggaaaccaaacatacaaatgaatcagtatcaattagggcctggggtagagagacagaaacttgagagaagagaagttagtgattccctctctttctagtttggtaggaatcaccctgaagacctagtcctcaatttaattgtgtgggtttttaattttcctagaatgaagtgactgaaacaatgagaaagaatacagcacaacccttgaacaaaatgtatttagaaatatatttagttttatagcagaagcagctcaattgtttggttggaaagtaggggaaattgaagttgtagtcactgtctgagaatggctatgaagcgtcatttcacattttaccccaactgacctgcatgcccaggacacaagtaaaacatttgtgagatagtggtggtaagtgatgcactcgtgttaagtcaaaggctataagaaacactgtgaaaagttcatattcatccattgtgattctttccccacgtcttgcatgtattactggattcccacagtaatatagactgtgcatggtgtgtatatttcattgcgatttcctgttaagatgagtttgtactcagaattgaccaattcaggaggtgtaaaaataaacagtgttctcttctctaccccaaagccactactgaccaaggtctcttcagtgcactcgctccctctctggctaaggcatgcattagccactacacaagtcattagtgaaagtggtcttttatgtcctcccagcagacagacatcaaggatgagttaaccaggagactactcctgtgactgtggagctctggaaggcttggtgggagtgaatttgcccacaccttacaattgtggcaggatccagaagagcctgtctttttatatccattccttgatgtcattggcctctcccaccgatttcattacggtgccacgcagtcatggatctgggtagtccggaaaacaaaaggagggaagacagcctggtaatgaataagatccttaccacagttttctcatgggaaatacataataaaccctttcatctttttttttttcctttaagaattaaaactgggaaatagaaacatgaactgaaaagtcttgcaatgacaagaggtttcatggtcttaaaaagatactttatgtggttgaagatgaaatcattcctaaattaaccttttttttaaaaaaaaacaatgtatattatgttcctgtgtgttgaatttaaaaaaaaaatactttacttggatattcatgtaatatataaaggtttggtgaaatgaactttagttaggaaaaagctggcatcagctttcatctgtgtaagttgacaccaatgtgtcataatattctttattttgggaaattagtgtattttataaaaattttaaaaagaaaaaagactactacaggttaagataatttttttacctgtcttttctccatattttaagctatgtgattgaagtacctctgttcatagtttcctggtataaagttggttaaaatttcatctgttaatagatcattaggtaatataatgtatgggttttctattggttttttgcagacagtagagggagattttgtaacaagggcttgttacacagtgatatggtaatgataaaattgcaatttatcactccttttcatgttaataatttgaggactggataaaaggtttcaagattaaaatttgatgttcaaaccttaaaaaaaaaaaaaaaaaa

SEQ ID NO: 12 - DNA, 2500 bp, Homo sapiens
cccaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgcctccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggttgacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgttttgtactcacggcggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcataaaaaaaaaaaaaaaaaaaaaaaaa

SEQ ID NO: 13 - DNA, 6062 bp, Homo sapiens
tacgggtcctgctttaaaaagctccaagaactgtctcactcccaggctacatcttctcacttgctaacaaggacctctgagttcagcagccatgagttcagactcagaattggctgtttttggggaggctgctcctttcctccgaaagtctgaaagggagcgcattgaggcccagaataggccctttgatgccaaaacatctgtctttgtggcggagcccaaagaatcctttgtcaaagggaccatccagagcagagaaggaggaaaagtgacggtgaagactgagggaggagcgactctgacagtgaaggatgatcaggtcttccccatgaaccctcccaaatatgacaagatcgaggatatggccatgatgactcatctgcatgagcctgctgtgctgtacaacctcaaagaacgttatgcagcctggatgatctacacctattcaggtctcttctgtgtcactgtcaacccctacaagtggctgcctgtgtataagcccgaggtggtgacagcctaccgaggcaaaaagcgccagggggccccgccccacatcttctccatctctgacaacgcctatcagttcatgctgactgaccgagagaatcagtcaatcctgatcactggagaatctggtgcagggaagactgtgaacaccaagcgtgtcatccagtactttgcaacaattgcagttactggtgagaagaagaaggaagaaattacttctggcaaaatacaggggactctggaagatcaaatcatcagtgccaaccccctactggaggcctttggcaacgccaagaccgtgaggaatgacaactcctctcgctttggtaaattcatcagaatccactttggcactactggaaaactggcatctgctgatattgaaacatatctgctagagaagtctagagttgttttccagcttaaggctgagagaagttatcatattttttaccagattacatcgaataagaaaccagaacttattgaaatgcttctgattaccacgaacccatatgattacccatttgtcagtcaaggggagatcagtgtggccagcatcgatgatcaggaagaactgatggccacagatagtgctattgatattttgggctttactaatgaagaaaaggtctccatttacaagctcacgggggctgtgatgcattatgggaacctaaaatttaagcaaaagcagcgtgaggagcaagcagagccagatggcacagaagttgctgacaaggcggcctacctccagagtctgaactctgcagatctgctcaaagctctctgctaccccagggtcaaggtcggcaatgagtatgtcaccaaaggccagactgtagaacaggtgtccaacgcagtaggtgctctggccaaagccgtctacgagaagatgttcctgtggatggttgcccgcatcaaccagcagctggacaccaagcagcccaggcagtacttcatcggggtcttggacattgctggttttgagatttttgatttcaacagcctggagcagctgtgcatcaacttcaccaatgagaaactgcaacagtttttcaaccaccacatgttcgtgctggagcaggaggagtacaagaaggaaggcatcgagtggacgttcatcgacttcgggatggacctggctgcctgcatcgagctcatcgagaagcctatgggcatcttctccatcctggaagaggagtgcatgttccctaaggcaacagacacctccttcaagaacaagctgtatgaccagcacctgggcaagtctgccaacttccagaagcccaaggtggtcaaaggcaaggccgaggcccacttcgctctgattcactatgctggtgttgtggactacaacattactggctggctggagaagaacaaggaccccctgaatgagaccgtggttggactgtaccagaagtctgcaatgaaaactctagctcagctcttctctggggctcaaactgctgaaggagagggagctggcggaggggccaagaaaggtggtaagaagaagggctcttctttccagacagtgtctgcccttttcagagagaatttgaacaagctgatgaccaacctcaggagtacccatcctcactttgtgaggtgtatcatccccaatgagacaaaaactcctggtgccatggagcatgagcttgtcctccaccagctgaggtgtaacggtgtgctggaaggcatccgcatctgtaggaaaggatttccaagcagaatcctttatgcagacttcaaacagagatacaaggtattaaatgcaagtgcaatccctgaagggcaattcattgatagcaagaaggcctctgagaagctccttgcatccatcgacattgaccacacccagtataaatttgggcacaccaaggtctttttcaaagctggtcttctggggctcctagaggagatgcgagatgacaagctggcccagctgattacccgaacccaggccaggtgcagagggttcttggcaagagtggagtaccagaggatggtggagagaagggaggccatcttctgtatccagtacaatatcagatccttcatgaatgtcaagcactggccctggatgaaactcttcttcaagatcaagcctctgttgaagagtgcagaaactgagaaggagatggccaccatgaaggaagaatttcagaaaattaaagacgaacttgccaagtcagaggcaaaaaggaaggaactggaagaaaagatggtgacgctgttgaaagaaaaaaatgacttgcagctccaagttcaggctgaagccgaaggcttggctgatgcagaggaaaggtgtgaccagctaatcaaaaccaaaatccagctagaagccaaaatcaaagaggtgactgagagagctgaggatgaggaagagatcaatgctgagctgacagccaagaagaggaaactggaggatgaatgttcagaactcaagaaagacattgatgaccttgagctgacactggccaaggttgagaaggagaaacatgccacagaaaacaaggtgaaaaacctcacagaagagatggcaggtctggatgaaaccattgctaagctgaccaaggagaagaaggctctccaggaggcccaccagcagaccctggatgacctgcaggcagaggaggacaaagtcaacaccctgaccaaagctaaaatcaaacttgaacaacaagtggatgatcttgaagggtccttggagcaagaaaagaaacttcgcatggacctagaaagggctaagaggaaacttgagggtgacttgaagttggcccaagaatccataatggacattgaaaatgagaaacagcaacttgatgaaaagctcaaaaagaaagagtttgaaatcagcaatctgcaaagcaagattgaagatgaacaggcacttggcattcaattgcagaagaaaattaaagaattgcaagcccgcattgaggagctggaggaggaaatcgaggcagagcgggcctcccgggccaaagcagagaagcagcgctctgacctctcccgggagctggaggagatcagcgagaggctggaagaagccggtggggccacttcagcccagattgagatgaacaagaagcgggaggctgagttccagaaaatgcgcagggacctggaggaggccaccctacagcatgaagccacagcggccaccctgaggaagaagcatgcagatagtgtggccgagcttggggagcagattgacaacctgcagcgagtgaagcagaagctggagaaggagaagagtgagatgaagatggagattgatgaccttgctagtaatgtagaaacggtctccaaagccaagggaaacctagagaaaatgtgccggactctagaggaccaactgagtgaactgaaatcaaaggaagaggagcagcagcggctgatcaatgacctgactgcgcagagggggcgcctgcagactgaatctggtgagttttcacgccagcttgatgaaaaggaagctctggtgtctcagttatcaagaggcaaacaagcctttactcaacagattgaagaattaaagaggcaacttgaagaggagataaaagccaagaacgccctggcgcatgccctgcagtcttcccgccacgactgtgacctgctgcgggaacagtatgaggaggagcaggaatccaaggccgagctgcagagagcactgtccaaggccaacaccgaggttgcccaatggaggaccaaatacgagacggacgccatccagcgcacagaggagctggaggaggccaagaagaagctggcccagcggctgcaggcagctgaggaacatgtagaagctgtgaacgccaaatgtgcttccctcgaaaagacgaagcagcggctgcagaatgaggtcgaggacctcatgcttgatgtggagaggacaaatgccgcctgtgccgcccttgacaaaaagcaaaggaacttcgataagatcctggcagaatggaaacagaaatgtgaggaaacgcatgctgagcttgaggcctcccagaaggaggcccgttcccttggcactgagctgttcaagataaagaatgcctatgaggaatctttggatcagctagaaaccctgaagcgagagaacaaaaacttacagcaggagatttctgacctcacggaacagattgcagaaggagggaaacgtatccatgaactggagaaaataaagaaacaagtggaacaagaaaagtgtgaacttcaggctgctttagaagaagcagaggcatctcttgaacatgaagagggaaagatcctgcgcatccagcttgagttgaaccaagtcaagtctgaggttgataggaaaattgctgaaaaagatgaggaaattgaccagctgaagagaaaccacattagaatcgtggagtccatgcagagcacgctggatgctgagatcaggagtaggaatgatgccattaggctcaagaagaagatggagggagacctcaatgaaatggaaatccagctgaaccatgccaaccgcatggctgctgaggccctgaggaactacaggaacacccaaggcatcctcaaggatacccagatccacctggatgatgctctccggagccaggaggacctgaaggaacagctggccatggtggagcgcagagccaacctgctgcaggctgagatcgaggagctgcgggccactctggaacagacagagaggagcagaaaaatcgcagaacaggagctcctggatgccagtgagcgtgttcagctactgcacacccagaacaccagcctgatcaacaccaagaagaagctggagacagatatttcccaaatgcaaggagagatggaggacattctccaggaagcccgcaatgcagaagaaaaggccaagaaggccatcactgatgccgccatgatggctgaggagctgaagaaggagcaggacaccagcgcccacctggagcggatgaagaagaacatggagcagaccgtgaaggatctgcagctccgtctggatgaggctgagcagctggccctgaagggtgggaagaagcagatccagaaactggaggccagggtacgggagctggaaggagaggttgagagtgagcaaaagcgtaatgctgaggctgtcaaaggtctgcgcaaacatgagaggcgagtgaaggaactcacttaccagacggaagaagatagaaagaatattctcaggcttcaagatttggtagataaacttcaggcaaaagtgaaatcttataagagacaagctgaggaggctgaggaacaatccaacaccaatctagctaaattccgcaagctccagcatgagctggaggaggccgaggaacgggctgacattgctgagtcccaggtgaacaaactgcgggtgaagagccgggaggttcacacaaaagtcataagtgaagagtgatcatgtcctgatgccatggaatgactgaagacaggcacaaaatgtgacatctttggtcatttccctctgtaattattgtgtattctaccctgttgcaaaggaaataaagcatagggtagtttgcaaacaaaaaaaaaaaaaaaaa

SEQ ID NO: 14 - DNA, 159 bp, Homo sapiens
atggagaaagtccaatacctcactcgctcagctataagaagagcctcaaccattgaaatgcctcaacaagcacgtcaaaagctacagaatctatttatcaatttctgtctcatcttaatatgtctcttgctgatctgtatcatcgtgatgcttctctga

SEQ ID NO: 15 - DNA, 1386 bp, Mus musculus
tccgcggcggaactcctgttctggtctctcgcttcgctctcctctccagacaagatgagcttcacaactcgctccaccaccttctccaccaactaccggtccctgggctctgtgcgaactcccagccagcgggtccggcctgccagcagcgcagccagcgtctatgcaggtgctgggggctccgggtcccggatatccgtgtcccgctctgtctggggtggctctgtggggtccgcaggcctggcgggaatgggtggaatccagaccgagaaagagaccatgcaagacctgaacgatcgcctggccagctacctagacaaggtgaagagcctggaaactgagaacaggagactggagagcaaaatccgggaacatctggagaagaaggggccccagggcgtcagagactggggccactacttcaagatcatcgaagacctgagggctcagatccttgcgaattctgtggacaatgcccgcatcgtcttgcagatcgacaatgcccgccttgccgccgatgactttagagtcaagtatgagacagaactagccatgcgccagtctgtggagagcgacatccatggactccgcaaggtggtagatgacaccaacatcacaaggctgcagctggagacagaaatcgaggcactcaaggaagaacttctgttcatgaagaagaatcatgaagaggaagtccaaggtctggaagcccagattgccagctctggattgactgtggaagtggatgcccccaaatctcaggacctcagcaagatcatggcggacatccgcgcccagtatgaagcgctggctcagaagaaccgcgaggaactggacaagtactggtctcagcagattgaggagagtaccacagttgtcaccaccaagtctgccgaaatcagggacgctgagaccacactcacggagctgagacgcaccctccagaccttggagattgacttggactccatgaaaaaccagaacatcaacttggagaacagcctcggggatgtggaggcccgatacaaggcacagatggagcagctcaatggggtccttctgcatctggagtcagagctggcacaaactcgggcagagggccagcgccaggcccaggaatatgaagccctcttgaacatcaaggtgaagcttgaggcagagattgccacctaccgccgcttgctggaggatggagaagatttcagtctcaacgatgccctggactccagcaactccatgcaaactgtgcagaagacaactacccgtaagatcgtggatggcagagtggtgtccgagactaatgacaccagagttctgaggcactgaggcagagaaggagggaacccctgggaactgagggaccaataaaagttgagagctcactgg

SEQ ID NO: 16 - DNA, 76329 bp, Homo sapiens
atattaacctgcttcattgtattctaaatgccagaatatttaaaatagaattatattttagtctaggtttccaagtattttaaacactctgcttgaattccatggctgtggagtttattcacggtgcttttaatacacactcttgtacacatgcacacatacacagttacctatacttcaatccactcactttaatgaaattttatacattctaaatgctgaaatatttcaaattttagcttctagtaagataaatgatatctttaacaattatgcattagtaagaatacgagaatacaaattcattctctcaaaaatgactctatctataaggaatttttattgtctcttcttctcttgccccatatttggggaaaatattcccttggctactctgttcctcccattactagaacaattcaaattggggacaggacaaatgggggctgtgaacacagtgtaaagccagagctttgcttcagtggctcaggtctttactccaaaagaaatggtaacacacaaggcaaggatggatcacacatgagggaatatttggcatcctgtccagttttacatcctgattgaggacaatttgtgtgggactgacacaggccatttccacgcatatcctaaatttattaaacatggttattaaattatgtctgtctgtatttctcacaatatacagtatggataaaactactttctcatttcctttttcattgcaacctgatttaaaacagggatgtttgctcactaaattggttgagaaactagtggctaaatttgaaaccaccacctaaatttagacagtcaaaattgcccaccaatgaaacattctccacctcctatccacaatacaaccattgttcttctatgtgtgtccttatttttgaccaaagctgtcaattaaggtaagtcccaacaattctctaaaacatggaggagagaaaatgttcaaataacaatattatgatcagggcttccaaagggtacaaacagctacaaaaccataaaacacagctaaatgtgaaagtcaacactaccaagccagaggctttcaatgaattccctcttgaaagaggtctctagtaggcatctaatggagccaccaatatggttctgactcacacactctgcatccctgaagaaagggatcataacagaaactcaaggcatttattttaaaggaagttagagcaagaaatcaaaagaattttttgtctgttctgttccattttaataacttttctttctaaatgaattctggtgagaaaacattcaaacacatttaaggtggtctaatacccaattattgagaattattcactttcttctacataagcatatatttgttcactcaaattaaatctgtggcccacttaaaatcagatggagtaacatcactgcacttaatatttagagggaaaaaaagtttgttttacatcccacttaactggaaagcagttaaaataagaaagccattaattctttaaaaaaattcttgaacttccttttagaaaatgttagaaagtaaagatacagtcatttacagcatttaaaatgggaaaataaaaggtaaatttgggaaagtgtaattgaccatgaaaaaatgttaatttcctcagagagttaagacattacatccacaaaaagaaagagagtattctgaaaaataaacaccaaaaaataaagaacttttaaagctcctagaaatttaaaaaaaaaactttattaaaaggtaaaattaatttctcagtaagtcagaaaaaaattaagacatagaaaatatgacaaactttaaagatcaatataggaggttcaacatctgacttatacaagtttcaaagaagagaaaacaaagagaaagaaattatcaatgaaataatataagaaaaattaacagaacaaggcagtaaagatttaaagtgcctaaagagtgcccagtacactaatgataaaagattctcaccaatacatatcattcgcaaaattatttcacaatatcagagataatgaatagatcctacgggatgccagaaagaaacaaataaaaataggtaatatagaagtaaatatgagggagaatagcatcagattttataattgcaacactggaagcaacaagacctttgaaattctgctaaaagttcttaaattgtataaaacatcataagtgagagttgaataaaaacaaatattgcagatgaacaagttctcacaatttttattttacatgtatcctttgttaggaaaccatcagagaaactcttccaaaaaatgagatacattccccaaaaactagaaagtagtaaaagccaaaaggaggttccctcttgatatagtctggatatctgtcccctccaaatctcatattgaaatctgatcccccatgttggagaggggcctagtgggcggtgtttgggtgataggggcagatccatcatgaatggcttggtgccctccctgcagtaatgagttcacatgcaatatggttgttaaaaggagtctgcaatttccttcccctctcttgctctctgtctctctccatatgacatgaaggctccccttcaccttctgccaagagtggaaacttcttgtggcctcacctgaagcagatgctgctgccatgcctcttgtacagcctgcaaaatcatgagccaaataaacctcttttccatataaactccccagtctctgatattgctttataacaatgcaaaatggactaacacactcatgaaaatggtaaagggaagtccagtttttattttttcttaaaactgtactgtggagatgatgactgtacagaaagcaaagcattcaaccctacctaatgcataggggaatgtaggcttccaaaagaattttctgaaggaagtgtaattggtagatcatctgatacatttaagaatatggaaattaatattgattaatgtttaacatatctcaagaaacagataggtacatagaaaactcagcaaaattttaaaaatgagtcaactaataactccaagcaaaaagagcaaattttgcagcaaatgaaataataatagtatactacttgactctgcaataaaatgtaaacactactgactactaaaaattcgtgatatggatgcaggagggtgacagagggaaattgagaaagataacaagtttttatcaaaaacgttaaaaatctaaacatgaaaaatcaagaaataacaaaataaacatatttttttaagaaacgtggaggtaaatacccaagatatttttgagaaaatggaagtctagggaacaggactttcaaggaaagatgggacaggtaaagcctctgctttgttatacatctttggtactatttgatttctaagtacattatttcttactttgctacaaataaaaataaatatataccttataataaaatcaaactgacctcagaagcagtaataatattctatttcttaatctgagtagaaggattcatgacttttttcaaaattatactgtggaaattttcaaacatgcaataaagttgaaaaaatgtttatagtgaacacctgtatacccaacactcagaccctgccattaacattttaccatagtcacctcatcacatatccagccatctacccatgctctatctattcaaaatccatcttctatatatatatatatatagaagatatatataataatcgtctaatatatatatgtctatatatgtgtgtgtgtgtgtatatatatatatatatatatatatatatatatatatatatatatttcaaggtaaattgctaacatcagtgaactttcccctaagtacattagcaaatgtataaatgttttagaagaagcttttaagaatcaaaaaaatcaagcaaattattgaacaaattaaaaattggaatataatatagtcttctcagtcaaaatttgactttgcatatttgagctttacaaattttgtcatcaggtcgggtgtggtggctcacgcctataatcccagcactttaggagcccaaggctggcagatcacttgaggtcaggagttcaagaccagcctggccaacgtggtaaaaccccatatccactaaaaacacaaagattagccaggtgtggtggtgaacgcctgtagtcccagctacattaggatgctgaggcagggggattgcttgaacctgggaggcagaggttgcagtgagctgagatagcgccactgtaccccagcctcagagacagagcaagactccaagtcaagaaaaaaaaaaaaagttcctcagagccaagttcagtgctcaataaagggtcccagtaaaggtatgttgaagtaaatcaacactttaactttcttagtacagtttaggttaattcatgacccaattctaggaaagaagactggcttctattttgcagtgttgcttttttaaacaaaccctaagtttacaattaaatgataagattcaggaagaatatctaaaaaaaaaacctaaatgttattttggtaaagaaagttatgagaactggttaggatgaacctttactttcttttctaataagaatttctctccttgcttaataagcttgcagaagcaaacatacactctactgtggcttaacatttacttaagattatatgactaacttaaatgtcacaatgcaaattactgaagtaattgaaatagtacttaatgggtctctaatcatccaacagcatctgagttaatacataaactaacacacacagctaaacagcgcctgaacagtaacaattttatgactgtgatttaacagacaatgtcacatataacacattccttaaaatacttccatgacttggtgatactttctagatccataaatcacattctcaagaaagccaaaaaagtaaactggttgtttgaaatttcattaaccctttgctctgaaatctgatggctgtgccagcacagagcacaggaggaataggtaatagcaaagaaactgaagtgggacattccacaaagtggctgcatccaatctcttcacagataaatccaagactcagagccctctgcggagtgtgttatgaccccattcctgtaaagctaatgatttttcatttcccatttttagtaactctcgacttaaaatatatgaaataactaacttcaaggagaaggactgggactaatgagaccagaacaacagaatgaaattgaaatttgaaactccaaagccatagatacagcacataaaggccagcactggtggccaagtgagtgaagtcagaaccaagtttgcataagtgaaaatcagctgtttcctgggcagctgaacttcatgacctacatcacacttcactctttctaaatagtctggcataagaggaactcgagcaactcaattgcaagaaaacacataaaccaattaaaaaacgagcaaagcacttgaaaagacatttttcaaaaagtgacatacaaatggccaacaggtatacaaaaatatgctcaacatcagtagtcatcatgaaaatgcaaattaaaactacaatgagatatctcacacctgatagaatggctactatcaaaaagacaaaagataacaagatttggcgaggatgtggagaaaagggaaccccttgtacactgttggtgggaatgtaaattagtacagccattaatagaaaacagtatggaggttcctcaaaaaattaaagatggaaccaccctactatccaagaatcccatttctgtatatatcaaaggatatgaatcagtgtgtcaaagagatctgcactttcatgttcactgcagcattattcacagtagccaaggtatggaagcaatctaagtgtcaatcaatggatagatacatgggttaaaaacatagtatatatatctgcatgtacacaatggaatcctatttagccttaaaaaaagaaggaaatcctgtcattagcaacaacatggatgaacctggaggacattatgctaagtgaataagccaggcacagaaagacaaatactgcatgatctcagttacatgtggaatctatagtagttgaactcatagaaatggagagtagaaaggtggttaccagagattggaagagggaattgggaatatgttggttaaaagatacaaaatttcagttaggacagaagtataaattcaagagatctattgtacatcatagtgactataatgagcaatagtatatcaaatacctgagggctgggtgtggtggctcatgcctgtaatcccagcactgtgggaggctgaggcacgaggatcacttgaccccaggagttcttgaggctgcagtgagctaggatcgcaccactgaactccagcgtgggcgacagagtgagaccttgtctctaaaaaaccccaaaccaatatatcgtatacttggaaactgctaggagagtaaactttaagtgttctccccacaaaaatatgatatgggtgtaaagtaatgcatattttaatttgccagatttagtcattccacaatgtatgcatatattaaaacatcatgttgtatatcataagtataatttctgatttgtcaattaaaaattaaaaagtagtctgtgaatgccaagctggagccaactgatatatttagcattggatatatgctaaaactggaaacctttcactagggacaaagcatttctgataaagttcttgataatattttaaatgttagcccaagaaagaaacaactaaaacagtataaaaatattcttttatattgtttaggaaatcaataactattatatatattgacaggcacctaaagacaatattatttttctttacacaaagtaatactgtttatattttcctttgcaagtttatggaatgcagattacatttaaactttctgatctcaaaatacaccgctattataaagaatgtatattctcagtcttgggtttccctcagtgtggtaaagtcctgagtccctctgaaccatttggcagactgtctcactcaactgaataatttccaggttatttgtcaccttaaaacaaatgaggctgtacattcaattcagtgatgtccaagaataacacactagaaacagcactaaattggaaaataagagactttactttcagctttactaataagctgtgtgctcctgggaaagtcagcttctctctttccttgacagtgatggtcaagatcctgcatagtgtgcaagtttacgactgccattattcttgagcattggtaagctggggtcacagcacctgtgggcaaatgctgcaaaagcctcaggactccggattctggctccttccactctggaaatagctttggctttggaaatctatccagtagcattgctgggctggaagtggagggaggctgggagtggtttcacatgtaatttcctacccatgatgatttaccttatctcctcatcttatctcattctgtttcttttttctccctttaaactctatctcatgacttccatgcactttccatcttattctccttcacatcactttccaaagacaatagaaaacccaagaaaatgaaactaagtattgcagaaaaaaggaagcctttgttttctggacctcctgattatgagatgcaggattgaagagcctgtgtactattttagattgtttagtactgcacctgaccatatcaagtaagtgtggctgcttaagaaacatgagcatattctgatttttgctaagaatgtgtcaatccatccccaaattctaatgacctgaaagagtaaatgcatatctgaaagcctctaacatgtctctttcaggagacatttaatgcactgtggcaatctggttgttgcatagatcccttccattaaagtattattttctaaaataaactttcccattctttgtccattttattttccaatattccaagtcctctaagacagtttcctggctgtcccatgtggtctaacagaatccttttccaattactatttttaaaattagataaaaattcatgttttctatcttaattatctgttacccttttatatgatcttttgttaaatgtctcaaaaattttatcccaaagatggcaataaatctgttccactattcaagttcataatttaattttctttgaaatcatatttgcagttttgcaagtattttaaaaaatatgtataagtccttacccttataaaattggctatggaaagagagcaaaagtaacaataacagtggagttgtcagaataaaaataaaatacaaagactctaatatattatgttaaaatcattcaatacaaatatgactgaaaaccagcagagtgctgaaaaatatttaacaactggctttttgctgaggtgggtggaatccctgatttgtaatgactgctgatttccctggtataaatgctccctccatgacatccatagctaatttacagctaccaagtgtttaacaactggctcagaaaattctggaaattttaacagtcagctcaagtgagctggtacacgccagctccagcacatagttggagcctactatgtttaaagcactgttaaactttcattttataactgaataatactttaacatataactgctatcatcattagaatctttgggcctattaatagttgatgtttaataatatcttttacacaaatagctctggggagtgaggaaaaatcatatttcttagtcaacctcaaatatcttgatatactaaaaagttaattgaagaaagccaacatgagttaaataagcaatcattgaacagaatttttaacacagccatagtgtactactctagtgtgtgctgatggatgttaaattcttggtggacttgaacaataacaatgatgtagctgaaattaaattcaacagagagggtcagggagaattgagtgccaaccagcaccagaagatcacaaaatcctaatttggtttgcaacctttaagaatcccaatgccaattcccttatcctgccataaaatctacccattcaggcagttgtgacatgtgaggattaggccccattatttcattcgttttttaaatgtgctttccctttataccaaaggattaacttaatctcgtccttcattatgcatgaggttcaagttctccttcaaattataggatttgcactagtgcacaccatctccaccggtgtcccttgtgattaacctgattcagttcatttctctatttactgagtgtgagcgtcttctccatatcaggagctgtgtcaggattcaaagatgctcaaggacatcccttgctctctggtcatcaagtggaccagttcagaaaccagtcaggaatgaggagtgccagtaggtcatgtaaaaaacatgaaaagttaaacttggcctgtaagcccagtgacatgtcaacaagccattcacaacaatgatacatcagccaatgagaagtgatgctacagaggctaaaacacatttcactccaccccaaacactaaagtgcttaagggccaaagtacaacccatttcctttcgtattatttactacaaaataaatgaatagatatttaagaaattttaaaaatgaggagaagataaaagcaccataatcaaaacatttttctgatctccccttcttagacactaatccttccagctcatgtttgcataatctttaaggattcattttttgcctatctggtcatcaaggtccctggagactcttcagtgtttcaacacatcataatgaaaaggactgatgacttctcaaccaccaaacctcaccagccatctcacttgaaaaagtagcctcataatcactttactgctcaaccatattcacactttcagatgccaaaagattattttcctttatgtacaagtcaatttttttttatttctttaacaacacataagtacatttgtcttgctttattcttgctggtttagattataaaaccctctgtgtggtcttttaatccttaccctcatgttcatgggcactctcttttgttaataaccatgacaatttttttgaaattcactcgtcttttcattctgttcattcagtcactaaatctcatctaacacatggcactggttttctctctgaaattaccctgatttaagcgaagaatggtagcttgttttacagtagtgaaaaatcaaagtaaagttgagaatgaacactaaggtactattcagcctcacctttctttggactaaaagtacaatgcccctttttttctcagtagcagctttctgaacttcaatactttccttattatagtgatagccatctagaaagctatgtttaccgattaataaaagaagaaccttagaagggaatgaatctgttcagaaaaaggccctatgctatgtagaagggaaaagatctttaatgaaagttaaattaccttcttagaattaaaggctgccattcacaggaagcctgagccccccaggtgatttggaatacaactccactttcatcactatcactttagagatggcaagtaatagctgctactttattttaaaaatatcccccaccattcaaacctctttttacttgagaaactcaacttatatctcatcacggaacttgctctcatcactgagcttatatggtgcaattagtaattgccttttgagatgtttccttttatctgggcactgaataactcactttcttgcttgctatgataacagaactgtattagatgacgagaaaatcatctcactactcttagcaatgacaagatcattaccctacagaaattcaaagaagaatttgttttataaggagcaaagtagagaggatatcatgtgagaaagaaaggcaatattcctgggtcactcagcaagtccaggtagcaaagagcctcaaaatctgagtaataaaaatatagaaatgaccaaccttaggttgataattcattcattctgtgaaaatccaaggagctctaagactctaggacaaacactaggaataggtagttaaataagacacgaattttgctcataaggggttacatagtctcttagaaagacagacttgtaaacaaacactgaaaacatgagaagtaaaggagacgtattcaaattgctgtgaaattgaaaagccaaaagaatcaaagctcaacccaaaaggatgaaaagaaaactgtacctctaggcttagaaagtactgctccatgtcattatgtgatggatgggtaaatgagtgcatatgcacacagaagtcatagaagctagcctgtaggagagacgtctatgaacccctagacaaaccacactccccacccaaacactcattctgtccttgaatctgctgccaaccttctgctaagtagtctctcagcctgagtcctatgactgagcttctttgaggatggggtgcttacaatctctgaggcagtcactaccttgggaagttccatttgttttcccttgtatttagctgaaatctcttttcccttagcttcaaccaattggcatcagttatttcctccaaattctgttagagagtgcattccaattactaaagatgctaagtaaacaagtattaaaaggaatgctatatttaaggctgcctgatctttctacttactatgaaatgcaacacacattgattgagtgcctactaaatgaaagaactgtgtgaggtactaggcggccactggaataagagaacaaatctgcctacaagaagctcacagtctagcgagcggggcaggtatgtaccttcataataaggggcagaacaggattatcatcacagagaaagacaagtcaagtagaacaagggctcagcggatgcagagatcactttctacttagtaaatctggaaatagccatggagaaaggaaaatgcaaaattaacagagtcaaccaatacttatttaccgtctactatgtgctaggtagtgtttgggggactgaggttacagcagcacatctaacatttgaggtctttacttccaaagagattagattttagagaaaggagacatagacaagtgtgacatatatttcacaatgtgtgtgtatatagtatcacgatatatatatatataaattatatgtattatatattgtgacatatatatgtcatagggtgataatgataaaggggacagtgctgccgggtggggggcaggtttctatttcagataagaggaacacctcatatggggacttctaagcacaaacattaaggaagtgaataagcaagctatgcagatatctaggggaagagataggtcttgaagatgagatctggtgttacccaggagactctggaatgaaaagcacacactgaggcataaagagaggaagctacctgacacaaagggaaacagcatgtgctgcagtttggtgtaatgcaggattagtgtaaaagccatggaagataaaggatagaaaagcagaattggatagcaattctccatgacacttctacatgtctgatgacagcttttgtcttagattatctttgcaaatttgtgggtcgagcaaacagccttggaagatagagacaatgtcttttttcagggtaaagggcatatttgtttcatcaccaggataatgtccttggctggggcaaaggcttggtagcaacccttataagatagggggctttctaagcttggaatttctcagcagtgacacatcatcagtatatgcccaggaatccccgaggctactccacatctccagcatgggactcatggggaaaaccagtacaaacatggagctcatgttgcctgctgtgctgtaataaagtccttcatttctgacccaggagttttctatcttttgccagtatccatgaaaatgtgacagtctaactttttagcttgcaaataaagtaaatctcaggaccttcacaacatagctgggctattattatgctaataactaaacactgagttgagaaacttccattctacttgggagaggggtaggggaagtacagagaaacaaccaaccaaacaaacagacacttgatagaatgtcaggtagtcacaaatgaacaataaagtaagataaaaggacagagtaatggggatgctattttagatatggtgatcaggaaaggcctctatgaggaggtaaatgtgagcagagacctgaaaaataaaatgaagcagctgcttgctatggttatgtctatttaagagataaggaaacagaagctaggagtgtgtatcttgctgaggtcttgtccacactaacgagttgtgcatttgtcacagcttacacaactaacaagctggggacttgttacacaactagttacacaactaacaagttgtatatttgccactacataaaaagtagtgcggtggggatatgaaccatggcatcatgaatcctgccacactatgcgttctgtatatactcatctctaaggggtaatgagcactcaaactatggagaatgcagtaggagttatgagaaggagcagatctgaaaaacagcaagagacagatttcacaaaactggtcactgggttcgctgaaaaggcaaataaagagattagagaggactcagagcctttaagcatggatggccagaagaacagccgttcccagaaaccagggctcaaggatgaggggctgagagggagggagaatgggtaaatggataaataatgacttgattttcgacataccaacctggacgcattagtagaatagcctttgaaacttcccctccgatttgagattcatggactttgctcctctgccctctgttgtgacttttattaatctataaactctagttctaccccagcccctgggccctattaaaacaccggcaagagaaaatgaactgtctcgtgctgactattcctaaccagctggctttctttcaataaacagggagtgaactcagtgtctattctctcttactaaattttccatttttcattccttttggagatttcagaagaataaggggaaacctccccgcattttccccagtgcttctttttgacgggttcctggagaacaccactgtactgctgttcaatttatttttcttacttatattaaatataacaaattcagacaccacactgtcataaatccatgtcacattttatccctttatgttgactgcaggatttttaaaacttcattctaacttctttaaagcttgccagagtgcactgatgttgaggtcagtaataggtcttttatttctatttttaagcagttctgatttattataggatttttaaaacagtattaaaaatataaaaaggcataaacttttaaaaattaactgtttattaaaaataaactttaaaaaattaaccatataatgattgccattctaactggtgtgagatgatatctcatagtggttttgattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattactgggtatatacccaaatgactataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggaggggggaggggggagggatagcgttgggagatatacctaatgctagatgatgagttagtgggtgcagcgcaccagcatggcacatgtatacatatgtaactaacctgcacaatgtgcacatgtaccctaaaacttaaagtataatttaaaaaaaaaaaaaaaaaagaaagaaattccagagcacacaaaaaaaaaaattaaccatataatgaaaatatatgtatatgttaaatgtatgcataacaataaatatgtatgcatgtgtgtgtatataatatattctccgtggtggcaggtacctgtagtcccagctacttgggaggctgaggcaggagaatggggtgaacccaggaggcagagcttgcagtgagccgagatcgtgccactgcactccagcctgggcgacagagcaagactgtgacaaaaaaaaaaaaaatatatatatatatatatatacacacacacacatatatattctccttttttacacaaatggcattatattatacagactttgaagattagaagatactgttttaaaactaccaccaaggactcacccttaggtaggcatgcacatgtgagtttatatacacacacacacatacatatgaaatatagtcaatatttaaaattttattatagtgtaaaagtagttatatctcagcatttgatgatgatgatgatgacgatgaggatgatgactctgtaaaccacatcagtgatatgttctccagatgaatcctgtcttttagtaacaaatcagaaccatggatagcaacaccagtcgtcccaccccttttccttcccttcaaaatgatttattctttcaaaggagtaacttcaccccaatctctactttcctgtctttaaaaaacctgaaggtataagtgaggatttaagtgccccagagggcttaatcttgaaaaatattagcgactgttacttaaatctatgtaacaccacatttgggaatgagcactatttaaggcagacatcaatcagttattcaatcaggttgattagcaaaaaataaaaattccattgtaaacctcttagggcagagagatgaactgcaagttttgcttgttaataataccatttaaaagagatctgggtctttgttcaggccttgttttgtgtgagacacccatgaaaaattatttgtagtttcagcctgtgcagggttcagttgaggttcagattcaggatattaaatttatggaatagattaaagccatgctctgcagccagcatggcttttagtttgcagaatgaaacatgggggtgtagagagagggatttcaaagctctgtattcaggctgagcttatcttttttcctatgaccactatgctttaagaagagtttagaaagtcggccagtcatgaatgcttgtctattaaacatatgcttctgggacaaagaccacacagaatgtgaggatgcatactatgacttctgccctcaaaatgcttatatattttgggggggataagacaagttgtatataaacagtcactgtttttcatctttgcacatggccatattctgaacaataacagtaaatatcaagagaataattcatcagtgctctaataggacattcagaggagagaggaaacctgtgggctgctggtactgagagacataatgaagcagatgggattagaactgggtttttaaaaaagagtggaattttgacaagcacacattaaggatccatctctgtgaaggaaaatcgagtacaattgccattcactggacagagttgtcctccctgtaacactggccacaagcgtccaagccaggacagcattctgagagcctgatggactgagaaagggaacttttcccagagtaaccaagggctaggctgtgtgaggagcaagaatgaattttagtagggaatgagaaccagatattagtgccaagggcaggatcaagactcaggactgagagggagatctaattagaaggcaaattaagaaatgtgaagctaacatcagaggtaagtctggagtaaggcacttagtgaggaaggatgaagcaattctagatataggctgagaacaaggtgaggatcttagaggtccacagctataagaaacatctaaaatgggattgcggcagccaggaaagatgtaaagatattataaaattagacccaggatttgctatttgaggggttatctgaagggaaagaagtagaaaaagtcgtttcaaagatttgaggctcagaagaatgaagacccccactgatatggaaggccactgtgagaactcgattgagcatttgattttgcttatgttgaatttggcatccaggccaacatataatttagtggggagtatatgctcagtagggattaattcagagctggaactccctgaatagatcaggaatgacatccaatatcccttatttttctcaggaataagttagtaatgatcagaatatctactggatttctcaattgacattcgtggccaaagagagtagctaaggtaaataaaattgattctaatacttttcttaaaaaattccacctgcaaaacgctacctttataggaaaaatactgctctgtgggttgagaattgttagaatgacctctccaaatcagatggcaaatcatgctttgcaaagctcatcatcacagtgctccatccatcatagtgctttggccatacagcttcatttacattgagaaacaaacagaattagcgtctgcgcctcaggaaagtgccattgtgtataaaagtattttctttaagtcagatcaggataaaagctttctcttttcaattccctacagtactgctccactttctcatccattacctccttcacatagtaatataaacacacactttactgctgcatggagaaatattaatgctagaaacttggcatttaaataatacctttctcccaataagccaaaagcagtttataaggcactattctatgaaatcatcacagtgggcctggaaataaaatcaagatctttgtccctccagagactattttctccactaaaccccactggccaactgccagcatcaggggaaagaaatacaagcacaaaagaaaaccacttactttgacttgagggatttgactctggaccgtagagagcttgcaccatagaaagtgggtagccagaactaatggtggggtctgactggttttccagtgtaatccttaaccccaaataccacaggaagtgcttaaacaaatatgctacaagtagttggaataaaagaaaacccctcttgcttatattgttctaacccagctattcccttctgattttcagaataccctaaagcctgaaggagaatttctattagtatatcatgtttttcataacaaagatgacttcacttaaatctcttttttttttttttttctttgagacagagttttgctcttgtcacccaggccggagtgcagtggtgcaatctcagctcactgcaacctccacctcctgagttcaagagattcttctgcctcagcctcccaagtagctgggattacaggtgaacaccaccaagcccggctaattttgtatttttagtagagatggggtttcaccatgttggccaggctggtcttgaactcctgacctcaggtgatctgcccaccttggcctcccaaggtgctgggattacaggggtgagccaccacacccagccaaattttcataatctaagtagaatatcaatattcaattcacatgagctatctatacatgaacatgtgtttttggtctaaattccattttcttagtataacaaaatgcctattaatcaacaattcattcaatcatgtattatctactcagtactacgtcactctttcatagctgattgtctgtctttcaaataccaacttcccattctcagtggtacagtggccagaaagcatactttttccaattatatcagcctcatgatcagaacgattaatggagtcttctgtgagtatgcaaatagatgtacagatgtttatcaaacacagacattccgtattatacttgactgataaatacataatcattaattttgatttacatgctgttgtgtgtcctagcgtgtgtgttgactggcagagatttttaaaaatcttgctgcatcatacttcagtatgcactctgcaagtgcatacagcagcaaaataaccaacaaatagttaataatgaattaaatgagttaataaaaaaagatgtctggttaaaaaccagactgcaacaaaactttctctcaaaaaatttagtaacataaactgctaaagataagataccaatcaaagaactgtgcagttgtggccataactcagacaaccaaggatgcagacaggaagacccatagagtatttattctgtgccccaacagcccccagaatgccgtgtctgcaggaatgatccataggctggaagaaaaagtgatcattatgtcagatgtgatgaatgcaacacctggttaggaacaatgtgtgcaaaactgtacacagccatatgaggatggaagcatatggaactgatcagaacacaatcctggtctcaggtaagggggatggggtcacacagatcttttaaaagactcagaggaagcatgtggtgatggcgatgatgggggagttgagggaggagttgtgaaaccatgagcctggatagaattcttaaaactgcaatcagccgggcacaaaaacagtgcatttggagcaacagggaatgaagtaaccccactctggctttcttaaactgagaactggcatttcttctcatctatggccaatttcgagttacagccttgaagtaagccccagcacattcgagaagaaaaacataccccattacacatgtatttttatgataagtaaaaataaagtatctaaagacaaatatttactcccttttctaaaattagaagattagataggtagctgctgttcaaagcacataagtggtttcacttctgatggctgagagggcaataaaactgtaagcaaaacatttaaaaagcctcaattctccacaaatttccatttggattcttttgttttagttactttctctccttctacccaaagagtgactcacaaataattctagcagtatataccagtccacatgacacttaataaaaataatatatctggaaaaatggcaaaaatagtttctagtttaaatactgcaacagataggagtgaaatgacgaagtgtgaatataatttggctcaacttaaatataatttggctcaacttagtgtttgacagagagcccaacaaatcaatattccaaaacaaccaaacatttaatagcgttaccttgcatctactcaaaactaagtggcagccagagattttctggcattaaatcacagcatggtacatttcaatatccaccatcaatgtgttgtttttcataaaataccactgaagtcaccacaactcgttggaaaccccaaagtcccatttttcagacttttcatctgttaagcacctctctgagaaccctgtctaaatttggagtgctcgtcaggtacaattctgtttgacttctggttgtgattcacacttcagttgagcttcctgtcattttaaaatgtcacccttgtgggtttttatataaataaatgtgggtgttcccattttataaattttataaacaggaacacctacattaatttatatagtggctttgatgttcaagaaatactgtatttaactctgagatgctcagggtttacagactgtttttgaacttcactcatcaataatctacatataaagaatcaaacactcctaaatcatccccctttgtccaagcctcctgaaggccatatggatgttgcctggtatcccctggaatgtgcatcatgggggcagggacttactgtcttggtttttgctttattgtcagtgttgagaacctaaggttcacaatcaaacacaaaacaggtggagaacaacccacctgttaccctagacagaaaggcacctggctactcggcctaagcagtttctggtttgaatcccagctatcacttaatatacctctttaaaagttaaacctgttgatggatccttaaaatgaaaataatgacatcttgcagcagagcttttgtgagaattagagataattacataaagtggctgcctgacataaagatatccaaataatcgctatgaggatgacgacttgatagtgattaataaatgcaaactgtgatgtttcaaattaaaaagcaagagagaagagaactgaccacggataggcgtcattgtggaaacttcttccagactagactggatctaccttaaataaacattgaatatacaagccatcgttccaatttgataatccaacttgacaatatgttcaggtcaccatgatgtctactaatgtcaaaaggtcgataatcctgacccaagtgtcatctgcaaacttaatgagtatatttcctgagatgaccataatgcaaggtctttgtgtatttgtttgcggatgtctgtatctgatgtcctttggttgcttgcatgtaacatgctgccagtggcctcatgagaacaggacttgtgtctgtttaattcaacttgcctggagccaatacagcatcacgtgcatgtggaaatgaactgattctgatgttgatcatgattttcatcatctcagctatgataaagattattaacttttagcagaaatctcctccaatgtcaccagaataccacaaagtgcctttgggtttgccttttagatatgttggaaaagtctagatgaccattctggaggtccagttcttcctctcctatcccagggtcccagggatattacaagcgatgaagacaacaatgctgaagagccagagagaacgtgggaacttcttccctctgaatgtcttctctgcctcacaaggaagctaggccactagatcatggcttcagttagactctgaccattttcaaaatatataagagaaaaaaatatgcacagttactctgcttctcagtaacttcaaggccttgaggctcttatttttggagtaagtctggcatcagttacagaagaaaggagcaaagagacgtaagaagtatacaggtatgcccaatattacagaagtgaccacacctgccagaccactcacctttttgctagatgggaattagggaaatggtcagaataggagaatttgtttctttttacctttctaagaaaacgtgaaaagagctgaacagaattgttcttttattggacaataatatttggtgtatttttcctagtagctagtcaggaattcaacctccttaaaatgctttgaaataacagctttattaattagcatatactcagttcctagtactcagcttggcacagagaatgcaggcaataaatatttattacatgaataaattaatgatgagttgggattgtcataagaaatctccacaatccattaaaacagattactacatttttaaaacatgacctgaagggtaggttttggcactctgacaactgtaattagcctataataatcaggtaaaatgagaagtaataaaggtatgataaacatctcacactaaaagtaagcgaacactgggaagcgcctcacagtatttgatgaatactattcacacgtgatgttattttcttgctatgcacaagatttttattagaggcattcattgaatttaacacatcaggcaaaatgaagcatccactaatctgagaagtcacaaagctttaggcatcactgaatacactgcagcaaatgtccagaaggaaatatgtatggtgaactgttcatcgtaagtttgctcgtgtgcaacacaaatgccattttcctgcagataagacaaaggtccatttaactttagtccataagaaagtagggggtggcgtgggcgtggcggctcatgcctgtaatcccagtactttgggaggctgaggttggcggatcacgaggtcaggagatcgagaccatcctggccaacatggtgaaaccccatctctactaaaaatacaaaaattagctgggcgtggtggcgtgtgcctataatcccagctacttggaaggctgaggcaggagaatatcttgaaccagggggtcagaagttgcagtgagctgagatagcgccactgcactccagcctggcgacagagtgagactccgtctattaaaaaaaaaaaagaaagaaagtcaggggaaaagcaggaaatgggtaacttcatctttaccaggaataacagggtctattgtacaaaaaaaaaaagaagaagaagaaaggtaatgaaacatgaaagttcagtgcaaaaatcttcatatttattctataataagggtacctatctcctatcattagcattttattctcaagtaaaaacaggtcaccatgccattactttgacctcattaccttttcttaaacatgattaaataatgaaaggcaatggtactaataattcaaccatattcctaaacaatgctagaaatgttctccaagcactcttctgtaaaaaaatgctttctttggaattcttgactttaaacggactgcagatatttgagaatagttgaagaaaatatgcagtctatggaaagcatgaacacaaaatgaatggacttctcagctatctgtgccatctaatgtctaggatttcccagagaaatgatctgaattgaaatcccacctctaccacatactatgtgatcttaaattacttaacctctgagtgcctccatttattcatctataaaatgggcatagaaacagcacacatcttacagggttgttgtgaagagtaaatgagacatgatgttaaacatgtgggcacagaataaggactctgtaaatgttagctggtattccagtgacttggaatttgggtaattcttcctataggatcaaaaggaaaactggaaaggaagaataattcagcaagacaactcttcaaagactgatgtggagatccaaggcctcttagcttttaagaacctatctatattttcatatgatatatttcaatatccatcacaatctccagcatcccaccccaaatgtaagaaatatgtctgacaaaagaattactttgatagagtaaatagtagaggtttaaaccctcttatctctagaactataggaattgaacctaaccctgagaatccaaaattctccatgctacctgtcacaccacatcctaaaataaggtcagctagagactaaaggtgactaagcaaaggaaagactcatctggctaatttggcacattgttttaaaattgtccagaaatctaaatagccaacagttaaataatcattaacatgatattgtaaaatggaaaaaatacttatgccattatattacaacaaaagaatagaggatataaaattaaatatatagtagagtatctatgcacattctgagtcatttacacacaacggcaatggtaacagcaattgtaaagctcttggtgtaaaattatggctgttttttttattttcttcttcataccaaatgctatattttccaccttctaaaaacacacaataaaaaatacaagtcccagatatcagaataacaacaacttcatcgtgtctaatttaaatagcattttataatatgctaagagctacaatatacttagtgtcatttgcacctcaaataaccctatgaagtctgttgagcacataacgttcctgcaagatcaggcacctgagagaaagggtcaagtcacttcccatgtttaggcagccaggaaggagggaggctagaaatcaacccagggcttctccacctcttctcatgagagtggtctggaacagaacggccctggaatattagaattcatgggtagagctgccccattggtccttggccacaacacttctttcctgggctgtaaaatgagtctaaatgacagcattttcccaaacagattttgttacagtaagaaacaagatattgtttgaagtaatcactggtacatttctctctatggttttcattcacttagaggtgctgtaatgaacttagcatggcccaagtaaatcaggcacatacatcgtcttccatcatagtggggaggtctggaaagaacctgtgctacagctaaagaaactatcatcagagtgaacaggcaacctacaaaatgggaggaaattttcacaacctactcatctgacgaagggctaatatgcagaatctacaatgaactcaaacaaatttacaagaaaaaaacaaacaaccccatcaacaagtgggcgaaggacatgaacagacacttctcaaaagaagacatttatgcagccaaaaaacacatgaaaaaatgctcaccatcactggccaccagagaaatgcaaatcaaaaccacaatgagataccatctcacaccagttagaatggcaatcattaaaaagtcaggaaacaacaggtgctggagaggatgtggagaaataggaacacttttacactgttggtgggactgtaaactagctcaaccattgtggaagtcagtgtggcgattcctcagggatctacaactagaaataccatttgacccagccatcccattactgggtatatacccaaaggactataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaatcatcattctcagtaaactatcgcaaggacaaaaaaccaaccaccacatgttctcactcataggcgggaactgaacaatgagaacacatggacacaggaaggggaacatcacaccctggggactgttgtggggtggggggaggggggagggatagcattaggagatatacctaatgctaaatgacgagttaatgggtgcagcacaccagcatggcacatgtatacatatgtaactaacctgcacattgtgcacatgtaccctaaaacttaaagtataataataataaaattaaaaaataaaaataaaaataaaagaacctgtgctaacaacaggcctcttttcctttcttttgcatcataattcagaaactaaggccttaagcagccctgggtagagtggatcttggtgccacacctcagctctacctccttgaggacaatcatgctgccactgaaatgccagggttctttccactaggtttccaatctgcttcttggaggtattcccttctctcctaggagctgacacagcaggcagcatttcctgcaacaccctccaaaatcactatgctaaatgagaagagtctgctgccccccacagaacactaatctgctttgttttcagccagatgttttataggggacatctgtgttagatggaaaacaaaaagttactggtctacatattagggagcaaaaaggaatgatgtaaacgcacatgtataagtgttcaaatttttcaagtacagaaagaaaaaacttaagtattaagaacataaccatggactatgaccacaccgattaatattttaaaaatccacaagacatctttccagcattttatattgtgattcctgaaaatttacggttatcaaaaaacagagccaaatttgctaagcctgtaaagtcattctgttcctattattctggcagtaaatgacctggctgatttcatgctgggcaacaaagataaatttatctgtatctagttaaaatgcatttgacactgtacatagttttggctagctacactgtcttggtctcaagttaaaaataagaggccatgtaaatcaatcctaaactctttacacaaagccacagtaaaaagaactgcttggtattcagtgaatataatcctctctaaataaatcttaaactcagctttggagaacttctatgtaaaacttgtataatatagcttttacaggccaaaaattaaatccttagatttaacaaattctacttttaaacgttttctttggctgaatcttgatagagaacagtctttttttttttaacaaattctcataattcttttccaaaaattctatatttaataactagttaatctgatcaacaaagacagcttgaaaaatacttctcatgacctaagaaacaatagggcctaagacacacaactcaaattaaatgtgttctccacaataaacttctggattgctttaagaaaatgcagggggaaaatccataattcatctcactagtttcaacacagggcatttcaaatcctctttccaagaccatgatgggtgctccaaagtacgtagtctttcaaaacacactgaactggtttggatgagttgaaaactccaggttactgtgaaggtaagccaagaccaaggaaaaataaaataaaatttgttactgtttttaaatatagcagtcagataaaagttatagctttacatttaataatataatgtcactttagcatagtctaacgcatttttcagtgaatacagttttcacatcacattatagcaatttaatcttaataatcccatgagacacttagcaataatttctatttgtcagatgaagaaaccgaagttttgtaggaattgatcactgatgaagaaagaaactaggcacaaagctttgttggaaaagtacagtccagaaaataatgcttggaaaatattattctgaactctattaaactagagaaaaaggtttcaagggctctggattcttttaaaacacagtcctcttcctgaatccaagcgatattaaattaagtttaaattaagctaagtaactatgccaacatgcaacagaaataaagacaagaagtgaaaaaaccaagaatacaccactattcatggttcttccttcaatgagaacatttcttaatttgttaaaattgattttgtttttcaccctgaggctccacatatcaggccacttgaaagcaacaatgcttgctcataaggcaactttcagtcaccagggtggtgacaggaaatgacctgcttcatctcccctatattcatggtatgtttccttcccatcccctccacccatcaaattagactcctctccagaagtcaggtcatctttcccagagccgccacctcttcccacctggatccttgccttcactcaggttgtgtcacaacatcagccttatttttgtgttgctttggaattgcctctaaatgtctcttgtttggatgtccccccattactctctgagcagtgatagtactcaacttgttaggtgtacaccatagtgaaccacaatgcatataatctaaacattttaactgtcagaacttggtgctataccttaaaattaacactagagcaacagcatccatcagcgataggattgaactagaatgatcacatgtgctccagcaggatgccttgcctttagtgcaatcagccagtcaacaaagaactctttgagggccaactttatgtccggtgaagagtgaggttagaaatgaattttgatgcaagaggcttgtgttatgaaaaataaaataaaatccttaagtctccaaggaagctcagaatatgaccattcacatttgttgcataatgcctgaaaagttgaaaaaaaatatggttttttaaatccaaaaacatcctaaagtggctgcagtcccagtcctgacccagaggcaataacagcatcttccaaagaaaccatttgtttctttttaacaaggacgtaatacgagtttcaaaacttatctcacgggatggttctgagaattacaagcaacaaagcccatgaaagtcttttgaaaagggctgagtactctataaacgtaaactagtagtaacagtactggacttcgctattctacctctgtaaagggtgaccaagaggactcaaaagtgacccaagtcaccccagtttttggtcagagaatttctattaggttaggttagatatttatcaaacaaggtcacactaatttcaagtttttaccattaggttgttcttacttgcaaagtttctgcaaggattggctgaccacattctccttcacttttggtttttaccagctgttatgtatcattttttcccatctttatttatttttttaacttaattttttttcttttgagatggagtgtcgctctttgttgcccaggctggagtgcaatggcgcaatctcggctcactgcaacctccgcctcctgggttcaagcgattctcttgcctcagcctcctgagtagctgggattacaggcaactgccaccacgtccagctaattttttgtatttttagtagaaacggggtttcactatgttggccaggatggtctctctaactcctgacctcaggcaatccacccacctcagcctcccaaagcactgggattacaggcgtgagccaccgcgcccggcccccatctttataaagagacctaggagacatggtgttctggaagaaagagaaaaagatacttgcttggcctccaagctaggtcacttggagcaaaatgagtcagtctacaaggaaatttttttatataattgcataatagaatgcacatttctattcttctgcttgtagcattttaacacttcagaatttttgaccagaaataatatgaacatatctcgttacactgccactggagtgcttcattctatttggagcccaaatttcccatgaaattgaacacttaaaactccaaaaatatactaagatttctcttttcttagtatatacatgtaatagaagagatgtaatctctacaaaatacaacttcattttaaataaataaaattaacatttgtgtggcactttctatgtgattttataaatatctcatttaagcctcattctgttaagtattattatactcacttcagatatgaggaaacaaattcagcaatattaagtaatatgtacagatagtgagagatgaaatggggtagaaactcaagtatggctaattccaaatcccacattctttctactatgctatgttactccatatcaaagtataaattagtggctatcatgaaagctaaactttaccaggaagtatgggtgttagatgctggactaggcattttgagttatttcatttaagtctcataataactctaaaaggcaagaatgctactatctaaattttacaaattaggcaaccaaggctaagaaacactaaagaattttcccagttcacacagctaacaaggagcagaagacagactcacactccagtttgattgacataaagtgcatggtccttaccactagactatattgctttttggttgtagggaaaaatcttacattaattaattcaaacacaacagaactcgaattttgttgtaaaagctgagagcttgcaaatcaaaactggaaaaatatcagtaaagatatattgatcgagagaggcatgccaaagaaattaaaatctaaattactccatatagaccaaattctcgattttaataattcaaatatcagattgaagccattcttactaccctacttccttcccagcttcctctccatttcttcctgaaagacttgtctatgtttgcatcaatttctcctctaatttgttttcctaaactagctccctcacctatttccaccaagctgcctctgtcaagatcatcagacacccccaggttattctactcaatcatcacattttcaggactcatcttatctgagccattagcagtgtatgcacaagtgtccctctgtcccagatatgctttcagggcatgccattctgctgacttgattcctatctaaccagtcactccttttcagttctttatacgggtgccctttctttttcccatctcttaattttggagcaacctaaggtcagtcattgaaactcttcccttctctaatgacacgtatttccttggtgagctcatccggttttattgctttaaatattatctatatgctaatgtcttcctgatttatatcttcaggccagacttctttcctgaattccagcgttgtagccaactgcctatgcagtaacttctctggtacaactaacaagagatctcaaacttatcacatctaaaactgagcacatctcttcttctacccccagcctctagaaaatcactaacacatattaagtgatcaataaagctgttggactgaatagatacaatccagccagatgccaatgagtctcaaaagaagagtcacagccatacaccctgcaaccagcagccctaccttgttaaaaagagtcatccaaatccattacaaaaccacagtagttattttactacagtcatgaaagttggaagtttctatagaactggaaattatgtaatataaggtaatcacaaagaataatcattttctgttgtatcataatatataacatcaacaagatctcattataattcatgcgcattactacatggattctgggatatctcacctcaaatcatctaagatattactcgcttatccagaacagcctacgagaagcaaagtttaattctctactgtaccacttacagctactactttttaaaacagctctactgaggtataacttacatgccataaaattcactcattttaatcgtataattcaatgatttttagtaaatacatacaattggcaaccaccaccacaattcagttttagaacactttcatcaccttggaaagagtcctcatgcctgtttgcagtcatgctatcctcctacgtccagccccagacaaccactaatctatctgtctttatggatttgcctttttagaaaacagatactatttttaagggcctcttatgggatagatacctaagtaaacatttcacataggctatctcactcccataacaaccataaggtatcattatttgcccaattctagagataacgaatatgaggttcagagagggtaagacaagagtctcacttaactatgtgggtttacaaaaacaaaaaacaaaaaacaaaaaaaaactacctctaaaagcaggtatctgaagatatctgacagataaaacaatgccatgaatttgcacaatgctatcacccaaaacaaacaaggaagatgaatggtgtcctgttagggtttaattccatcaccacctgctgtaacacaaatcagccctccagacagccaaggaggatggctctgtctggctcacacctgaactatggaaatggagcttgcacatgtccttaacacttatttgaaaaatgaggaccttgctacctatgatctcaaagatttgcacatcattgacctcctcgtgcagcatacatccacaaaacagacatgtggaccatgaccacagacagatccgcacctgaattactatgcctaaaagcttataaagtcatgttcaaaaatcggtataactcaagttagggatgtatttctataagaactataatatctgttgtatgccaatacagaaaatgtgggttacagttctttgttttttgttttaattggataaccatttagttatggtacatttacatgtactataatctctagttcttcaaaccagtatattagaaaatgtacatcagacacagtctggtatcagtgtcatgctaccacactgatttgcaaggatgaacaattcttagcttctttcattattgctcactgctaaatttaagaaggaagacctcccatcttaaaaacagagaagacggccgggcgtggtggctcacgcctgtaatcccagcactttgggaggccgaggcgggcagatcgcgaggtcaggagatcgagaccttcccggctaacacagtgaaaccccatctctactaaaaatacaaaaaattagcagggcgaggtagtcccagctactcaggaggctgaggcaggagaatggcgtgaaccctggggggcggagcctgcagtgagctccagcctgggcgacagcgagactccgtctcaaaaaaaaaaaaaaaaaaaaaaaaaaaaaaaaaaaaaaaaaaaaaaaaacagagaagacaatacagtttatttgggatcttattattgcagaaacagattatccgttgttgaataaatcattgatagcaagtgactgaaaaaatagagttgaggacaaagatgctctaaaatgctacacaattctcagctcctatagacacctatagctttggtaactgccaggagaataaagatcaggaagagccatagtaatccttcaggctgaaaatcaagatggaggccccccataagcgggtcctaagagtcccactgcctgttactgccaagaatgaagtcactcagtagcagactcgcagctccttatgctttgtgcagccaaggctgctccttagcaaccaatgtcgacaactgggtttccaaggaaaccagaaagcccaatttataaaactatgaaacaaagcaactgtaaaggcaacactagacacagaaggtaactctattcttaccattgctactgaatttctgccagtgaagtggtacaaatattttatttctaaatgtttaaggggggaaaaacccaatctttaaagtagaccacattactccctgacctccacatctttctgtagcaggatggcaaatacatttttaggattcaaatgagggatagcatcaaaaatttgtgtctcaaaaaaattagatgggttattgaaggtgaagaatattttcagaatcatctgcgtggttttaaattccttccagatcacaaggataattctattgactctgctcatgtataaaacaagtagatatatttgccaggctcttttccattagttctgatacataattactatggcttcctgaataacacactttttatcacgcagggggtaatagaagcacagagttaggggaaaatatgaatgaggatgaaataaataaggcagagatattacttgtatgcgacatctgagtattttgggaaatcgagcatatgtcagatgccaccaactgccttataatttagggctcccagacaatttaaaaaagagaagtaaagaactatttctatgaaaaatttctctctattgactgcctcttaagatagatcattacttctttactggagagcctagggtgaattatttaacctatgagcctcaaattcttgtctgcaaaatgtgcatgaaatgaagaagagttaagtgagatgatatgtatgatagcttagctacagtgtagcactaagaaatgctagtgttgaccgggcatggtggctcacgcctgtaaacccagcactttgggaggccgaggcaggcagatcacctgaggtcaggagtttgagaccatcctggccaacatggtgaaaccccatctctgctaaaaacacacacacaaaaaaattagccgaacacagtggcacatgcctgtagtcccagctactcgggaggctgaggctggagaatcacttgaacctgggaggccgaggatgcagtgagctgagactgcaccactgcactccagcctgggcaacggaagcaggctccgtctcaaaaaaaatgctaattttgtcttcttgatccttccttcaatatatcttgcatttcttattctcatcatgctttaaaaaaatctgcaagattatttttcttaagagtcaaaagaatgaatagaaatcactgcacaaaaagtgtttcactgtgagaaaattatgttaaaaaatgtatgaggagaaataccaaatgaggtaaggttacccattcagcatgttaataatgtaaaagatgccctatttctcctgtctccttgactaggtggtccttgcaccaggaatttatgggcactttcacaattatcatggattttatttttaaatatcatcttcattttcccagtcttcctgacagaataaaaactgttcctgaaaactgctgaccactcagttctcaaaaataacttgagaattaatgcaactagtcatttgctgaattcctgaaaaagctctgattgagcttgagttctccttccatctctgctattgtatgtccttctgatcactcctgagaactctctctctacatattttaacagcagaagaacttttcaaaacttgttttccctggcagaatatagtaagaccctagtccttgtttcagtagtgaaattctattaattcaatgatttgtggactccttacgatctgcacagtgccgttcctgatgccagggaagaagagaagttccgattgcacccgccacctagtactgatgtgggtaatatctacccatgggccctggctctgccctctgaagcaatgtagactgagtcacctctgtcttctacataacgctttcccaaatgtttgaagatggacattctgtccaaaaaagaataatgggccaaaacttagattatagtttcttctttcacatattttaaagaaatatcaaatgtatcatgtaacctttctgaccttaccttccttgtctatataatagatatgataatgcccccagtggaaagctggagaggaggagggagaggagaaagagaggaggaggagaaaatataggtagaagaatagagaaagctgtgccacttactgagcacttactatcaatacctccccaagcactaatgcttctcaactacccagtggggtaatcactctgttctgtcccaattttacaaatggatacaccaagacttgcccaaggtgacactgctataaagtcttaaccactattttatctcctttctgtatcccatgctctgtatgccaaaaattacaacatattttgcaaacataattattttccagcctaaacaatcacagctccatcaactactcattatgttaccttatgtcatttggattcaaaaagttctcagaaatctacttgccccccttctagatgtcacctggtttgccagcatccccctggaaatgggtcacagaggcagtttggcaatttttgttaagagggtattaaataccaagtactctgctagagctggagacaggaaagatgaatagacagtccttgttctcaggcagccagaagtctattagcactaaagacttgcaagcagaaaatatatatataaagaaaatttctataataaagcacaaagaagatgtgatggtaggaacatgaaaggagagccagatgcagtcaacagagcaaacagattgatgacttgggggaatgcagtaagaccaccacctcccaagggccatacacgtttctacacatgtccctgttctgtggttgctttaacaaatcataacacatggctagcttattctgtatcaaaacctgcattattttccatgaattcagaatataaactattgccctggccaatgctgatgtcggggacatacatggagaatgtgcaatgtcatgaacattaatatgactttgtgagctctgatccttaactttggtgtgtcataatcattggattttctcactaaccagtaatttagtaagatagtcctctggactgaatatctgtgtctttccaaaatttgtatgttaaaatctaatacccaatacgacggtatttggagatggagcctttggagtgattaggtttagataaaatcatgagggtggagcccccatcatgggattagtgctcctataagacaaggaagagaccccagagcttccactctacgccatgtgaggacagagcaaaccaagaagagggtcctgaataagaactgaatctgccagcaccttgatcttgaacttcccagccttgagaactatgagaaacatatgtttgctgtttaagccacccagtctatactattctagaacagcacctcaaacagactaagacaataagcataccacttacatcttcttccaacacacagactaaaatgtggctctagataatgctcatggagtcaaagtctggggtgtgctactgaagcccctccaagcccttgatgatctgttatctgtagcagttgggtagagttcttcaactgagtagaaatctcccacaattctttcatttcagctaccagaaagccataatattgtaatgcattattatttaaagtggtttacaaattgtagatactgaaacattttcagggtgtccagagggatgagctatctccaaatgaagatgaagtatctgagcactggagcaagtggatggtgaaacaccaccagttgcagtctaagtgcagatgaaaagacaaagtcagattagctcttggtagacagactttcaaatggtcttattttatgtgaatttgaaatagtgacataaaacattaaaaggttgtcactttacacttccaattttacgtaatgtgaatcatttcctacaaaaaaaataggtctgatttttgtgaattttgcatttttactaggctttggaaggcaactccccctctaaaatgtgactcgctgcaccgagggaaggttgtatggagaagagaaaaccgagggggatggtggacagatactcagaaaaggagctctgcagtcagggagagaaaatcaaccaatgagcaggacatggcatcaaaacactttacattaaagaagctgtgtgtctcaatggtgcagaaaagcccctgtggctacatggctagagatgagaccatgtcagggcggttgtggaaatgcctagaatgttactctgcaaggtcacagtagatcatagaaagaggaaaacaacagatatgctggccatgtgcaaatggaggctgatccagcagctcaaaactaaagttactgcaagggataacaaagatgaaaagaaacttgtgcatgaggatatagaagccttgagaatttaatgagctgggcaagtagagaaaaggaacattaagaaaacctctgctggacacggtagctcatgcctataatcccaaaactttgggaggctgaggcgggaggatcgcctcaggtcaggagtttgagaccagcctggccaacatggtgaaaccctgtctctatttaaaatacaaaaaaattagccgggcatggtggcgggcacatgtaatcccagctacttgggaggctgaggcaggagaattgcttaaacccgggagacggagggtgcagtgagccaagttcgcgccattgcactccagcctgggggacaaaagcgagactccatcttaaaaagaaaaagataacctcaagttctgaagactagagtacttagtaccaacagcattatcaggaatattctgtttcttagagcaaggaaaacgaattgctacaatttaaaaaaaaattgtttcaagcattgaaagaatgctccatcaatagcggaagatatccaaaaggcaagcaatgaagggatcattaagagctattgacttgtgatgctgcacatagaagtagtagctgtaccttacccaatgcttggggagccaaatatgttagattctgaatttttcagactttagaaaaatggtgcagcacatacatcacacattacttatccttaccaatgacatctgggcagtggcctttgatcagcatattaacaattctgcagtgacatacacacatgttcacattaggtgggatcaatgatgactataaaagcttcatattatttgggtcaggttttgccaacaaaggagttatggaaaaaaaaatgtctgtttctacagcttttggagattgggaatagcactgagctggtatttgaggtaactgagagttaatgaagaaggaaggtgagaaaggagagcaaggcctagcttgtgcggataaccagtgttcgggtggagggcataagaaaggaaagccgaaaggtgagacaaagaggcagcaagaaaaccaagagaacacgatatcatgcaggatcaaaggagagagaaaagcaagctggaaatacctgtgaaatcaaaacatcaaaatataggaattagggactcaataaagcttctggtccatgtcgggttatcttacataggtcaaggatattacacatattcaagggaaatattcgtcaaggtcaacagcattttgaatctgatctgtcagtttccttcagtgggctgaaattgaattactaccttgctccaggtagtgctccatattgtacttcagtctacttccatcttggtcccgaaatattctatgtaaaaataatttattgattcttgcattatttcaatatgtagcctgcctttgattttgagttttacttaacctaggacaatgatctacaatataatgtttccagtcagatttccacagttatttttgcaacacacacggcttctgctttcactctctgtgatgaggaatccctttctgttattgggaagaaagagtttagactttacatcagtggttctcaaccaggggttgaggggtaattttgcctcccagaggacatttggcaatgcctaagaaacattttaattgtcacaactgggggaagggagtagggcattctgtcatctagtgggtggtgctagatgctgctaaacattctgccttgcacaggacaggccccaacagtacagacttatctgtcctaaaatgtcaatagtgcaggttcagaaactccgctttcaataattacagacttcggtgtttggtagaagttttgcattattcatatcacaaaataagcatttcaatgcatataaaaattcagcataacattaattaaatggttaaaatgaaagcaacttttaaaagaaaaaaattagaattaaataattattaacataatgaagctaagaattcaatttgactcaagtatatttccctcaaaattattcctttcctaacatctcattaaatactgtctaatatcatgaaggctgacaaaaacaatgtgaagtttgaagacagaatctcatcacttagtgcatcactaagtaactttttggccaagttataaaaaatctaatgaagcagaaaaaaaaattcaaagtcacatatataaaaaaaattaagtatgctgaaatctacttaaaaagaccactcatagttcaaaagaatagagggccaattagatggctttgaaatgcaaattttcattgttccattttaaaagtcatgcctaaggtggccttaaaaagcagttatttccaagaaatggcattcttggaaagtttcattgttcgggaattctaacctgtgtcacgtagaaaggactggaaactgtcatctctacctcctgcactcaaccccagcacacatgcgcatgaaggtctcacaacttataaaggattgcatcaaaccattgcttgaatatgctctctaacttcttctgggaaaaataacatttattcaacttaagtagaaaaattgtggagataaaacagagcgaccgatttacacagccattctttagagaaagttttgttttcctttaacatttcaattgctatagtcaaggggagtaagtaacaacaacgctataagaccacaatgctttgaagagaactttagcatcctgcaaaccaaggcattggatatgcactatctaatgagggtggagaggccagcaatccaccccctccagataaccttatctccagccttgctattttagcaattatggctgacccttgaaaaacacaggtttgaactgtgcaagtcaacttatacgcagatttttttcaatagaaattatgagtgtgcctgcctctcctgcctcccctttcacctcctccacctcttctgactctgccatctgtgagacagcaagaccaacacctcctcttcctcctcctcctcagcctactcaaggtgaaggcagtgaggataaagacctttatggtgatccacttccacttaacgaataggagttatattttatcttaataatattttcttttccctagcttactttattataagaatacagtatataacatgaaatatgtgttatgttaattgactgcttatgttatcggtaaggcttctggtcaacagtaggctattagtaattaagttttggaagtgtctaaagttatacatatatttttaactgtgtgggaggttgattcctctaatcccatgttgttcaagggtgaactgtacttgatatgtcatttgtcatttaaattctcaacaaagagttggatggatgagtcatttcccccagactgacggcatcatgaaggcctgaaggtgtcaccaactcctctcctccaacatcagctgaaaagatacagtcctcacttagaccaccctcagagagcacagtttgtaaaacttatgaaaatggaagttgtagaaagcactagaagttgctaaaatactttcatatgaaaaagactcaaatccacccactggattagacaaaaacctgagctgaatgagccccagctatctacctagaactttatcttcattcctcatttgtcttccttgctcccggtggagagaaggggatatacaaagagatggctgcagttagaaaaaggcaatagttgcctcagaaacccagctcctggtgagtcaaagttatggttacagggaatgggctctgtccacaaaagttgcttctttaaatatcttctgtgcataagaatctgaaatctcatttgaatttgccaaacacctgttcagtattaagcaatgtacccaccgattccaaactgctatttaaaaagtccttagggaatggagggtcaaagagtataaacaatctattctttattgggctataagaaagcaccatattaaagtagaaaaagaccaggttatcttgacagatcaagatggtggcctgtagcaaactctggcctttgcctctgatcacagcctttatgttctagtgacctcataggaagttctctaaaatacactaaaaatgtctagtttgtttttaactgtattacgtgttcacgtctctgaaacttctctttcatctcgattactctgttaggtccctcctcagttcaactccagttcccactttggtagatcagcctgtcgaacaaatgggatggcattcactacacaatgataattaaaatttaaatggctataagaaaacaagtcctctactgtttattggtgcccatacaatcatcaaaacataggtactgtaaatcttacaaatgtgactcaaaaagattgaaagtggaatagattctaagaaacaagcaattgtattattcacattccagctgatccatatcgccaaataacaaataaatcatccaaggaattcctccaggaacaatctttttccttgagttttcccattcattttttaaaaatttcaacaaatatttatggaagacccactttgtgacatccactgtggtgtaggtgcttagtaggggcttgctacccctaccattggtcttccattctaagctaggaagagaaaactttttctttaagaaacattctattaaacacaggattagtgatatatgaatatatatcctccaagtcaacaggttcatttcctcctggatttctctaagttactttgctgttgctatttttataacataattagaatgtgctagaaaaataatagaaatcggtcaaattgatactttcctttctcaaacaactttttatcaaaaggaagaaaactaaatgtatttgtcccttggctattaaaaatacattggagcaaacagacacttaggattagattatcacaaaggaaaagtcaaataattcctgaggcattcaaaaacatgcagaaatgtcagtaaacatccttgaatgatttttccccgccctggcagaatgaaaaggtaaggaaaaacactagatgcaagaatcagcagacagacagtagctcggtttgtcttccatttcccttatccccagacagttcatgattcttggttttagaaactggaccacagggacgacagctgagcgtctgatttttcaaatgactgacgtacaatcaccgggcacacaagggtcaaaccttgcagctatttaaaaaacacttatgtataatctgataaaaagaaatagctaaacagaaaagatataaacaaatttgaagtttgggaagtcttgttgaagaatcactaaactgacagcacagaagtgaaaagaactgaattataaaggagggggacaaaagcccagatctggaaatagtcattctatttcaagatttgtcctgaagaactgtactttacaagaagttaattacacttgtgaaagctacaatcattctgccattatctatactcccactaaatctctattagaaagactaccaacaagggatccgtatgtcacattaagagaaacagacaaaaccaagcaagtttgtaaaaactgatgaaagaacaacaaaccaatgcaaatggccttcgctactggttatatggcacccattttagctttttaacttcctaggatagacttatataagtactttcccatcagaaataaaattgttataccattttcaaaaaatatcatgtggttatataaatcaagatgagactttgaaaacaaaagtactccataaatatgtatacattgtctctcaaggaaaacaaatttctcattaccaccatgtggccaaagatagattactaataacagatgttttaagtatccactttagaaaatttaggtactataaatatgcccaaggaaactcaaagttagtatattattaaaataattatttttatctccaaattatgtaaatcaaatacactaattttttctccccagagaagtttgaaacaaatgcctatatgctctgaaagttgtctctggattcttttaactttaatgtttatatttaaaatacatttatgctgtaaacaattcaaataatataaagcaaatggaccctttgtagattccaatcccactttctttctcagaggtaacatctcctatcagtttattgtgttttttttccagacctttgtctaaatgtatataatccaaaggttgggtttttttagaattatgaaaaaagctatactgcacatattctgcaaattgctttagtagagataaaaattactaacctgaatacagtttggaagccagctctgtgccactgtccccaaagacaaaaatgaccatcaataactagctttaaagcttcaaccatgaggttcccatgactgctcatgtttgcagtaattttccctaattttttctatcagaaatcaagcttctaaattaccacacactattctttgtgactgaggaaatagctaccaacaccctgaatgttatcaagcaaaattatagttggggtagaccgagagaataccagtctgccattaagcatagcgagctccctgggagggaaggctggagaggatttgaaaagggcaaaaagagagacacatgggcagaccaagagaccccctgactcctgaagctctcccttcacctatccttggattcccctttcttggcattctggcaactttgaataaacagtcaggaacccccagacaaatatttcattgcatattgcttattttcaattagttacccaataataaaatagattacttctgtgaagaaatctttaaagaaaagtctctccacaaaccaaatcttgtggcactcaccacagggttagacacttttaataaaaattattattaatctgcatgagatataaaaggttcatccttccttggctacatttagaccctctctctcattttcttccttcccccatccaactcagcagccagggtcactgtttacccatctgacatcactgtttgccagctattagcattatttaattagctccttcgacaaaccaagtcaatgtcaggagctgtttctgtgacttaaaacacaacctacaccagccctctggagtctgctctgtaccaccaggggtcccctgggctccagaacacctcagctgaactcaggtgtcaggacaaagcacacaggagagagaatcagtcccaagagaagtcagcctcagacaacattactggctggcactacagctggaaataaaatgtttcctcaacagctttttttttttttttttaatattttaagttctgggatacatgcgcagaacgtgccagtttgttacataggtatacatgtgccatggtggtttgctgcacccatcaacccgtcatctaggttttaagccccacatgcattaggtatttgtcctaatgctctccctcccctcgcccctgcccctaaaaggccctggtgtgtgatgttcccctccctgtgtccatgtattctcattgttcaactcccacttatgagtgagaacatgcggtgtttggttttctgctcttgtgttagtttgctgagaatgatggtttccagcttcatctaggtccctgcaaaggacatgaactcactcttttttatggctgcacagtattccatggtgtatatgtgccacattttctttatctagtctatcattgatgggaatttgggttggttccaagtctttgctattgcgaatagtgctgcaataaacatacgtgtgcatgcatctttatagtagaatgatttataatcctttgggtatacacccagtaatgggattgctgagtcaaatggtatttctggttctagacccttgaggaatcgccacactgtcttccacaatggttgaagtaatttacactcccattaacagtgtaaaagctatttctccacatcctctccagcatctgttgtttcctgactttttaatgatcgatcgccattccaactggcatgggatggtatctcattgtggttttgatctgcatttctctaatgaccagtgatgatgagctttttttcatgtttgctggctgcatgaatgtcttcttttgagaagtgcccggacagctgtttaaacactctacatgatagagactgtttacatgtctacacaggtggactgcaaagtacacaacaaaacagggcatttatcttcaagcacaggtattgctaaaaggaaaaataaacccttgcaaaggagtctgtgatcttttcgcttatgttgtaattaacaaagagttaggagatagtgttaggtattcgtttggtaatcaatttctgagtatctcttagagtcaagcattgtggagcacagaaacatgaaatgacccatcctgtgactgtgagaaatttgcagcctagtggcagtttggacgtgcaaagatgtgattggaaaacaggccatgtggattgctggcatgcaggaaatgctctgaggatatccatacctttttcccaccaagaagactcctggaggcttctccctcctctccactctcttctaacaagggacaggtttgattgggagtgggcaaaggtcagttggattcccaatactagaaacacctatcctgctgtggatacactattctcccaagccaaggataagactaagacccctgggaaacaaggggaaggcagggaaggtggtcagagacgaagagggcaagaggcacaaaacctgtggctttgtttttaccaaaaatgttaaggaagtcttgggtagcactagagtgcccatgagcataggatgctctggctgggtctaaacaacaatgttttttaaaaccatgatgtgggaaaatggccaagttacatgtggttttcctgtttcttcatcattgaagactaaaatccacctgtggaaaccgatctctcctgtatagtgggaaaatactttacatcctagtggttatgcaaaggttaagtgactgctggattctggaatagcaggttgatgactaggaggagtgagctgtccacataataattaaagtgtgtttcagatgaggtgtggctggtgctgcagaccatatcctttgaggaaaactccctctgcagctgaagaggaggcactgagggctgagtccagtgagggatgagaagggcactcccattgtgagcaggtgagctgcccgctttcagtgccagagaagaagctggcttctgtacttcaggagcagcctcatgtagggtccagtcttaaggctgttctctcattcttcctttatgtaaaagccttctggaaagcttaaatgttaggatttggcctcagctatgctgtgggaaatgaaaggacaggctcttttctgtattgagaaagaagtggcatagggtagagaactacatcctgggaaagcaggttgtaagatggcaaatgtgccctacaggacagcaggcagcctcgtgccccagaacaccagtggagtggcagtgccagccagtgggtctgcgagacgtgccggggttcccgatgtaccttgagattccagggagggtggaaccttaacattagtggtagaaaaagatctatctctcttgacaaaccaaaatactttattctactgcaaatagtccccatgacaaccacatttccatttatgtcttagctcataataacccaagaacactcactattcttagcctacctttgtagcacaaccatttcttcagacatttcactccttcccaccactaatctctgtttcaatggtcctctggattctaaacttttctgaatacttttttaaaaaacagatgaaataccaataaccaataaaagaaacaaaattttattctatcttttttttaaaaaaattctctcaatctacttatcatcattggtgcaatttctatattcaagttggaggtgaaaagtctgaaattagttcaaaatggcttttgccagactcattaaaatgcttcggaagcggttttaattttttttctagcgtcttcaatgggcctgatttctgccccatttcgaatcattcttcctctgtacaatgtcacatatctgcgaataaatatgtaagctctcctctacttgatgagaaaacctcacattacaaactgttgggcaacccaggagaaacaggccagatggcctagagacctgctggcctgctaggcctgaacagaagcacaggctttcctcaatctatgtgctttgtgcagttaatataactctatttttggaaacataaccttggttccaagttcagcccatgtttctttcctgtgcagcactcaactattccactctctcatgacacttctctgcaggaccaccctcgaacaggaactccgggttgtccattgcaaagtgttcccatttatttaaagtttcccatcactctcttctttccacctccattctctggcaaaagcatcttcttttttctttgcaccaagaaatatacaacaggcagttctggtcatttactttctccaacttccttttatgtttctgagtcactgaatcttgcctcacaccaataacatctagctctaatttgatttttttcctccaatatttcaaatacaaaacaagacaaaatcttgtttcctttggtacaagagtggctccaacaatagctaatgcgaagtgttgatggcactttgatctccatttggcagcatgagggaaccaaagctttttactctcctggtgactaatcattatttcacatcaattggagaggattaacaaatcttagtaatccagatgtgcgtttgcaattcctatggtccccaagtgtgccacatgcattgtgggaataagcattaggttaataacctagcactatttaaaaaatttaatgcactttgccttcataaactacgacatgccctccaatacattataaagcaagccttactttagagtggggtgacataacagttgtaggcttatcgatgtggaaaggaaagttatttgacatttattgttctggcaaagtataaagacaaaaagaaaaatagttttctttgggtctggacattcagatctttctggatgaaaattctcagagcagcaaatgtaaggcctacagtccgataaggacgcacatcctctattcacaggcatattttcaaaatacccccgaggagcccttcttcttagaggagtcattgaaaagatcagctggagggcatccaggcagacagaatgggatggcacagacacttagtattagaagcaacaagctgtttggagaactgggacttagggtgtacaggggaaaatggcaagaagtgaggctggaaagggagaagaggccaatatctccaaggattttacacgccacgctgggacaaaagctctgttcttagaaagagctctctggaggtagcacagaacgtaggtaggagaggaaagagacaggaagcaaagagatcagctagggaacttctgcaagggtccaggcccaccgtgagagcttgaacgcatgcgtggagaagggtaactggattcccaaaacatttctgggcagatcggaggagccactgaatgactcactggatacgggggcgaatgctctcgtgaggtcagtgaaagctcagagaaacagcaggggagcacaatgaggatctcatgatcatgagcttggtcttgtgtcccagcccccaccctaccccactccaccccatgcaacagccagtccctctcctgcactgttgtcacatggctcagtacaatctccttgggcacatagcccatggcattataaaccactggttaaaatgctggctgcataaacatcttcttttgagaagtgtctgttcatgtccttcacccgctttttgatggggttgttcttttcctgtaaatttgtttaagttccttgtagattctggatattagacctttgtcagatgggtagattgtaaaaattttctcccattctgttggttgcgggttcactctgatgctagtttcttttgctgtgcagaagctctttagtttcattagatcccatttgtcaattctggcttttgttgcaattgcttttggtgttttagtcatgcagggcttaaaacctagatgacaggttgataggtgcagcaaaccatgatggcacatgtatactatgcaacaaacctgcacgttctgcacatgtatcccagaacttaaagtaaaataaaaaaatttaaaaaataataaattttaaaaaatgctcatcttctgggctgtactctgagtgtctcagggcagggaccataactcacccatcttcatatcctcaatacttggcacgtaagggcttgttatgtgatgaatgactttcaacttttaatctacatttggccatcaaaatggaaaataacccaaacttcagagaagttgcttctttcttctgtctgcttctctatacatttataaagttagtccagaaaatttatttcagtcatgacaccccagaggtataaatatatgtgatatatgagtcaaggtagataaagaaaatagccaccattcctagcgtgatatgtgtcatatgataaagcacttgaagttgacagtaagaaggctgctgtgtgtcttgttcatctgggtcctccctcccactcccaacgatagctatccatttcccccactactggcatcattttctaactactttttcacatttatctgtgttcctagttcctatgccctgagcacaacccctccccaaatccaatacagctgtcatcattctggacaaattgtcttgctttcctagcccagggcaatgttctatgttgtttattttgcctagaatgtcctctcatcatcttttctacttgccaataccttatagtattttattaaatgaattattttatttaattacttagcagcacgtgatactgttgtcagacagtccttattttatttcattacttagcagctatgatactgttgaccatatacaactccaaatctgctttatttttctgaccggaagacaactaatgtctaagagttctggctcttgaggttttttttttatagctgtgtgatatcacacatattatataacccttctaaatctcagtttcctcatctgtaaaagagaaaaaaatagtacttaccttctagagttggtgtgaggattaaattttatcatgcatatgaagtgcttaacacagttcctagcacaacggcaaatagctgaatagctgttagttgttatgcttattatattatttaacttttaatataactactgtctaccctgctgcccaaggcatgtctcttatccgagaggcgttttctttttttttttttttttttttgagacagagtctcactctgtcgcccaggctggagtgcagtggcgggatctcggctcactgcaagctccgcctcccgggttcacgccattctcctgcctcagcctcccaagtagctgggactacaggcgcccgccaccacgcccggctaattttttgtatttttagtagagacagggtttcaccgttttagccgggatggtctcgatctcctgacctcgtgatccgcccgcctcggcctcccaaagtgctgggattacaggcgtgagccaccgcgcccggccgcgagaggcgttttctttccttttccttttcttcacgtcttcctgttccttcccatatccaatcagttactaaattctatgaattctaccgtctaaatatcctttgcatgttaggcaagagcccaggctgcacaaaataggtataatctgaattttagaagcttgaaaataagacaagtcaaaaaaaaagaaaaaaatggctttggagaggaaagtattgttaacatcactgataattaggccttcccgaataagagacaaaaataataacaatacttcaaaactattaacagactgcagcttgaaatccttacttctatgccaagcatcagactgttcttgaaatccttacttctacatcaaaccttcattatgtaactggtcctataattggaggaaggactgctcacatagctaactttgaaaattggcagtgtaccatatttgcaacagattggcaaattatttaagaagtcggtaggaataagaaagtgggtatatttttcattttgattgacagaagtcatcatgtgggtaaaaccatcacttcattcagtactaccaggtcttggatctgtgaaaggatatttgctacagtgaatctcccagccaagggctcccatggtggctacaaatcacctacttaagtttccatatagtagaccatgacttccatgtttttaaatgaaaacagcaccgtgtgattaaagtaccatatattatatacataaattcttttaatgaacaaacatttaccaagcacctggtcaaagcaaagtaccatgctagagacccctgaggacctcgatattcccaaagtcattgctagcacattatcattcaatcacctgactacaatccatgtccctatgtttccagtcctttttttttctttcaggaattccttcacagaactatttagcctctcaaataccctaaggaattttagaataattttgacattttttagtgatagttatttaaaataaaatatttaaaattagaaatacattaatagagcttagatagtatttgtggaatataaagacaaagtttgttcttcccaatcaacctgtcttacatattttcaggtaattttaatttatatggcaaatatatgatttaagcacaaaaaattattattattattttgagacggggtctcaccatattgtccagtatggtatcaaactcctggactccagtgatcctcctgcctcatcctccttagtagatggcactacaggcacccagccaaaaatttttttaatgactagaccttatttggcattagagtcaagtctatattaaaatgtagacaaattaaaatatgttacagcattttttaaaaatctgccttttttgacctgatataaattttgcacccctccacaaatcatcttgtttagtcatagtcagacagttttaatctcagtaaataaaagctacattggattcaattatcattgtattttctttttattattattttcttaaatgctagaaacaccttaacgtgctttataaaagctggcattatcgtcatttatattctgctccttgttcacctcctatatttaaccaatggttatgaatacaaaattaacaacagatagttagaataaaaccaatttaaagcaatttaatggtcaaaaccaggagctatcagctgttggtgttctgttgcgttattaaaccaactcagaaatgcggactcttacttgatccccactaactgccagtcatgggtagttgctatacaccctgtggctgtaaaatcaattcctttttaaggaaaaagtttctaagcaataattatactttacttacagtttttaaagggtattaagacaaatgtcgttactgcaccacattcctaaaccccctagtggctccagagctatcagccgggaagcactttacattttgaaaatgtattgctcaggacaaaatgatgaagagccctggagagcatctgctgtgaaagaagaaacaatttcgggccaggaatatgatttcctgtaagtgaaataatcacatgagaagaaaagtgaaattatctcacacttactgaaggctcaagtgatggggaaaagttaagatggaaaagaaaaatgaggagcaaaaaggaagcaggagtgaggaagcaggtgtgaggaaggaccacatgtctatgtcttcccttatgcacaagcactagactttttgttccagtaaagaatttccagcaagtgacagaaagcaaacccatgcccagttgaactgaacaatttctctatgaacagaaaatgttttgtcaagaaccagaaatttcttttgataatcagtcatgaatgaacacaggtggcagaatgtatcctctgatatctctctatctctctctctttgttttttttttttgttttttttgttttgttttgttttgttttgtattttttgagacagaatctcactctgtcgcccaggctagagtgccgtgatgtgatcttggctcactgcaacttccgcttcccaggttcaagtgattctcatgtctcagcctcctgagtagctgggactataggcatgtgttaccacatccagctaatattttgcatttttagtagagacgggtttcactgtggtggtcaggctggtctcgaactcctgagctcaggcaatccgcccgcctcaacttcccaaaatgctgggagtccaggtgtgagccactgtgcccggctctctcatatctttctgggctcaaactcccaaatgtttccaaaaaaccactacttaatgcaagaaaattaaagatgatctaaaacaagacttgatttatgtcaccggataagtaaatatcatggacatataaattccaccaccccttcccccaaaatatgagagtcaaagactgaacactgctgagaaacatagtagaaaaattaaatgctacctcatctgagaagcataactttcttgaggatatggccatagcagtcataagattctcaaaggggaatgcaagccccaaaatttaagagccattgttgtagataaaataagaaaaactaaattaaagatagaggattttgtcttaaattcctggaattaaaagaatttttccatttcattgatattattttctgttgtcagctttagctaaaggaaattacaataaaccatataataaacaaactaaatcactattttatttaatttcaatttaataataagacctatataataaagcccccaaaagagagggttaaaaactataaacaagttccattggttttgaaagggttttttgtatgtaattttctttaatgtagctaaccaccaccaaaacccctttggttctttggagagacagattaccaagaaggaggtcagaaagggagaaaaatctgggaagcaatctcagttcctcactaacctcagaactatgcctagatataagcccaaggcctttgaatctcagccagagaccagcttttctttcccatcttgtaagaggcttcacaggggagaccggtccttgtttttggtttagagatggccttgaacaagaaaaagaagataagtatacactgaatacacaatcttttctggaatccaaaggccagcagcatcttaacactgaagtacaaaggcattttcaatgatggttcagtgaacataatcccccaggtgcagctttactgtacggcagaatattaaaatgcacaattaagtctctttccttgagaagaaagaaataggtaatcttttaaaagtcctcaaagatcttgaatagcaaagtgatgaaaggacaagtgggaaactcaccatgaagagggaatgacagagattatgaaataattggatctcatccaaggaacagacaaagcacatggaagccatattttaactatgaaatgcaaaaagatatacacagcttgaaatatgttaatatcatgtgaaacctaatcagcttataaatggtattttggactgtaacaaagcaaatgtcaccaaatggggccccaagaaggtaggagagaagaatagaccccaaggaagcatccctagggcttagaagacagtaattattccatcctgccatgaacatgaaagctttcttctcctggatacctaagtctggagtgagctcagaaatgccgtatgtgcaaagagtgcaaagataataagtggctcacagtgctgaaactggctacataacctctgtaataagttacttataaaacctcaataaaacccagagctagtcaaaccgagctggagaggatggaggctacagttacaggttaaaggcgatgaacttagtgaatccataattacacaaatgacttagaaggtagaaaggagacttttctataaaagcagatagaaaaatgaggtcaacatttaaaagctgatgtgactaataagcagatcagtggctgctgggctaggggtgggagcccagattgactgcaaatgcagatgaaggaactttcttggtcaaagtgtctaacactggaatgtggtgatggttgcatgattctatgcatttattaaaatcattaaataatatacttaaaatgggtgctttttgtagtacgaaaattatagcttaataaagctgtaatcattttttattttaaagttgacatgtttttaaattttgcgatattcacactctataaaatagttgtactgtatatctctgtgatgtgttagttacattaaaaatgttctttttccactattgggaatataaatttttataataatctaaagtaagattcttaagtttttaaaattactctaaatggattattttcataatcattaacaatccaaataggaatcaaaatagcaagaaacacttagaagaaattgaaagaagacatttcagtagctttctcaacatttactgtcttttggcaacccagtctaacccttttaacagggtagtatattaatctcaatgtgtttaaagactgacaaatcacaggaatttgataatttcagaagaaaagtatttttaaactccagcaggcacaccaataaagctcagcaataaattgtgagcattcttaccaaaaaaaaaaagatgcacatgaattttatatacttaaagtatttggaaaacactatataatgtcagatatgacactgtggttaagaatgtcagtctgccttgaccaggtactaatcacttgggccagttactttccccctgattctgcatctgtgaaagggagataacaatagtatctactcccataatactgttaatggggtaaaaaatcgaaattttagttagtgcctggcatacggtaaacattcaagaaacactagctgctattctacatttattgatattatccctgggctggattcaacatacaagatgattttattcttttctacaaagcctcacccccagggacagatccggtcctttaccttcctggaatgccaattactcagcaatcttcacgtaaactggagaaaatgtttccagctgttgtaacagaaaatgtcttccaacttttgattcattacacaaggtgttatcactcattttctggcacagagctggtgcactatcccagaactgatctgacacactttcttcagctgatgtcaaaccaaattataaggcaaataggacaaacgaaaagtcacatgaacagaatccatttacagtggggtcattcacactggtccttccaggtgagagaagcactgagagagagagatttgcaattatttaaatacatagcataaagcttcacttttgtcttcaaaaaaaaagtaattttaagacctagaaaacacagttgccaatcctcaaaatagatccacatttcctctggttcttcctctatttctcttgtgccccaaaatgtaggtatgtgggtgtaaacatctccaaagtcaagttctcgtgcattttgataatcttgtaaagttttgaaagtaacttccctctcacgttaactgatggtagaaaatgaatgggaactttacacacctgattcccaatacatcttaatgggaaagtgcaggctcataaaactgagggctaggacatttagtatttattttaagggcttctaaaatatgccacaagcttagagtacagggactaattttaatggaaatctgtttattagcatgctggtacacaaaagtaggcttttaatattatttggatttttttttttttttgagagagtttcgctcttgttgcccaggctggagtgcaatggcgcgatcaaagctcactgcaacctccgcctcctgggttcaagcgattctcctgcctcagcctcctgagtagctggtattacaggtgcgtgccaccacacctggctcatttttgtatttttagtagagacggggtttcaccacgttggccaggctggtctcaaactcctgacctcaggcaatcagcctgcctcggcctcccaaagtgctgggattacaggcatgagccaccgcgcccagcattatttggattcttaatgtgctagaagtgttattaattcaactataaaataaaacacatctttcagttatttaaagtacgaaggcaatgttacagtgtttcggcagtcacattttcaaacagtacaaggacatcctcaaataagattaactgtatcaatcatctcatactcccatgaacatacaaatgtgaaaatgcaagtttctctagtaatgaaacttgcagtgagtctgaaaggactcttcccttaggattctgttgtgaggagttctcccaagtcttccactgcccactgaaagaaaactaaggtattcccaatgcctggcacagaatccatgctgaatcgatgtttccggatctaacaagtctacaaggacttgcacattttaaacattaaaagacatttatgttaaaaagctaaagcacaatcatcttaataaaacccaggtgaaactcttgatccaatttggcaaaaccaaagacaactccgtgaatagtctgttcacctaacaggctttgtaacaggtctttgttttttatggtcactgtcaataattggcactctgtacaatacaataatacatgagattagattttctccaactgaacgtatcccagaggcatcatagcatgatcttcagaaattctcccttttcctataaactgttgccaaatgcagcaaggatttaatcaatggatgtgtttttttctcaatacacagtggctaagtctaccaatttgacacacaatgtaaataaagagaaaagcaatttttgctatataagaaaacgattggccgggcatggtggctcacacctgtaatcccagcactttgggaggctgaggcaggaggattgcttgagtccaggtgttcgaggccagcctggacaacagggcaaaaccctgtctctacaaaaaaaaaaaaaaaaaaaaaaaaaaaaaaaaaaccacacacacacaaaattagccaggcatggtggtggacacctgcagtcccagctacttgggaggctgtggtcagagaattgcttgggcccaggaggtggaggctgcagtgagccaaggtcacaccactgtactctagcctgggtgacagagtgagatcttgtctcaaacaaacaaacaaaaatgatgatgttaccaggaaacaaaatacaataagcctgcgctattcctttcacaggagaccaattccagaaatggcctgagaaatatgtcagtaattttactcttcccaggggcactgtatcaaccaacgacagaaacgaaccttttactggttctttgtatacagaccccacttatttctagggcttattattttttccctaatttagagctataacctcagaaatggaattgaccttcccattcaaaaaaggccctgttaatggggtcgctgggcattgtatggctttttgcaaagccctgtctgccctaagtcatgggcagcccctgccagctgccagctgaattaaataggcgacaccccctcagtctcttaacctcccaggctgtgttataagaaaatcactctaattctgatcactaggtggttccttcacaccagtttatcccatccattgttgttttccatagtcaagtctgcagtacgtaagactgttggccactaaatccttgggcatgtctagaaaattattatagctggaaattattttcaaaagttacccctccattcctgcacaccttaaaaacagcccacatctttccgggcatcgtccatgttgggaattgtctcatgcgtagggactccatctgataatagttgaagatctaaaatcggttctcaggaccagttaatgacttccattcattcccacagaagacacccggaagcagcacagggatcatattcagtttgtgcagcgtattgaggggagatacagacaacttctaacagcatgcacatgcactttgtggcattaatctcttcctcgggatccgtgtttccacagtgtttgttcagccttcctgactctttaacctacgtttattctccatacaggaagccattcaccctccacgtgaggcagcccactgaggctagattgcagtgagaacacatttcacactaatgcactggctgctttccagaaaattgctgctggcaatcatatcttgccatctgatgtcaacattggcattgagaggatgctaataaaactgcgaaatcagctaaaactaatgtttaatgcatctaccacgcaacaacagccggggttcgccgtctttagggctttgaattctgatgttccattcatactcttcagtctcctgagcagctcttctgaaacttaaatgtgaatatacacagcctgagaatcttgttgaaatcacattctgactcagtaggtctgtgtggagcctgagggtccgcatctctgcctgctccctggtggccttgctgctgctgtcccgggaccactcttggtactgaggtttttatttatttaattttttttttcttttgagacagagtctcactctgtcgcccaggctgggctggagtgcaatggcaccatctagcctcactgcaacctccgccttcaagtttcaagggattcttctgcctcagcctcccgagtggctggtattacaggcacccaccaccacacctagctaatttttgtattttattagagatggggtttcaccgtgttggccaggctggtctcgaactcctgatctcaagtgatctgcctgcctcagcctcccaaaattctgggattacaggcatgagccaccgcacctggccgatagtgaggtttttaagaatactgatgaagcatacacctgtgcacacagcacgactctttacaacttatttctgttgtgtaagcagtttctagctcccagactgtcactctgaaaaggcatcagaaggaaaatgtctagtctaatctttttttttttttttttaagctagtgcttaccttgagttattttcttcctggaagattttttttttaataaaaaaaatcaattttatgttactatctgccaaggcaactttggtttatggtcttccaagtagagaaaaagggaatagaaagatctcttgtaggggcactcctagccctgtaatctacttgttaaaggatgagccaaaagttaaaactaagaaatgtcatttaacaaattgtaagcatctaatgagggtagtcgtgaatacagaaatcaacaaagcactatcttctttctcccagatattttggtctcacacaggtgtacaaacagttctaaggcaaagcagaagtgattatccacagaaagagaactaccttgtgtttttccctcagggaggaactcataaagatcatggtgactgtgctcccattgacctaaaaagcctgttacctggcatgtacaaaacaagactggacttatggaatcagacacgttttgaaaaatgtgactttttttctaagaggattctagacaaagcagatggagtctgagataattttaacctccaataactgattacactgaatagcacctcaagaattgctactttacatgctgtttgcttctcgatcgactttggaacatgattactatcagccgtaagtcagaagatagacaaaagaggctctacagacaagtcacaagacaggaaagcacactgggaaacttggcttcaggggatctgaccctggacaatgaaataacaaagttagagagggaatggcttgatacgtttcccacataaatgtgcagactcatgtgaaggtgccagcaaggagccaaactgcacacagagtgggatctgtggggttttgttgttgttgttattgttgtttgtttgtttgtttttaaagaaaaagacagggccaggtgcagtggctcacgcctgtaatcccagcgctttgggaggctgaagctggcagatcacttgaggtcaggagttcgagaccagcctggccaacatggtgaaaccttgtctgtactaaaaatacaaaaattagctgggtgtggtggcacgcgcctgtaatcccagctacttgggaggctgagacaggagaaatcacttgagcctgggaggtggagaggttgtactgagctgagactgcaccactgcattccaacctgggagacagaacaagacacctgtcttaaaaaaaaaaaaaaaaaaaagacaaaggaaaaaagaccgaaatatgagcaagggtcatcttctcaatgcgactatgggtaattttaacttatcttaatattttcccatttccttcaaaattctttacattgattatatattaatcttacaaaggacaagaccaattaaaaaataaatctcccatctgtccttttgtttctaaggcatgttgtttacaatatagttcctatttaaaattggatttttaaagttccatttaatttctgaaacagacttctttcatttaatttagtttcaaggatttaaaaactaatcaggaaaatctggtgcaacatattatagtctgccaccacagacacccttgaatactgcaggtttgtgtctctgcaataattaggccagatgcttttcatgtgcatctcctattggcccaccaccaatttttatatccatgggacatattgatcagttcttctctttattcactggatggctaaggcaatgtcaggaaggtttacagcaattctcctgactcagacggcgagatgtttctaacagaatatctattgttcccccacaaaatgaaagtgctttcaatagcgaaaatgatctaaaattggcagatgcgttctttttattttctatactagagtctttcatgaagaagccctagctagaaacaaccatcatggtgcggtgtgcatggtaatgtagtgtgtggccacgctgcagagggcaccatcatcatttacagctacacatgaccacagcccagggtgaccaaggcagctcctatatggccatgacccgtaaggaccttgctgaatactggagccatatgctaatgccgagtggatagaccacagcaaactggtcaatcatccagctacttagtcattcagtcatttaccaggtcaagctctggagactcaaaggccaatagaacacctttcttagcctcatcctataaaatcgaaaggaggagacaggcattacaatagaccagcataatgaagatttgcatggctacatacaatgtacagtgaacaggctccaagagagtgagttgtcaattttatatggggaagataggaaatatttcatagacaaggccacctttgagctgaatcttgagaaatgagtaggttcttgctttgcagagctggggttcagtgagattaccttcaattatcaatgtaatcatcaatataaatatatatagcaacgagtccaatgccaggcacagagaaggtattcaacaaatgatactaataataatattactttctcggtgggatggattgccaatagaaagatgcctttaaggaatgacctgtaccattgaagaagaaagtgaataaaatatctgcatcgcattcacaactgactgacaataaatgggatgataaaaatacagttgttagtagttcaggtacagcagatcagttgcaatggaaactatgcaaaatgaccaattctacattcatggtattatacaactacaagggtcttacagttttaagaaagcctggaatatgagaacagaagggagaaggactgatatttttagaggacctaccatgtgtcaagccaaggcctttatatacatattttgtcaattaattatcaccccaatattatgaggtatgcattattatgccatcttaaagaagaggaaattgagattcagagagtttaactgatttgccagaggtcacactactaatgagtattcaagtcaggatttgactttgtattagttaagtgtctcatttcgaattccatgctctttttattatatcatgaagcatttccatgtgaaaagccacatttacaaaggaaaatatgattagtcattataaaaggataatttaaaatagtgagtccctttggcacattaaaatatgatttttttccaaaaaaaatttgttaagtgtcagcttttccattataacaactaccaacaattgggtaccaatcacagtgctaaatttgccatgtgaattgtttcatttagtcctcatgacaacttatagagcaggcattattatcattattattattcacaatatacagatgatgaaactgaggattatcagggtgtgaactatttccaataggaggataaatttcactatgcccagagtagaggacagatttgaactcatctataattctatggaactaatgtttttcctctctgtcatgttatcatgtatatctatgcttaatttatagcatacataagcaaatttcctcattataagtaaattcaatatacagaaattttaaacttaactaatcagtaagttgggtaggctaacttaattttccaggcttggatgaaaaaccacaagctagattttccagactgaaatctcagtccaggctcagctgaatttttttgtttgtttgttgcagggataactgactgaccagctgactttaaagtccaagttctttccatcatcccaacatgctcctgccagacagaaaaggctcacaggccgggtcagggctccagctgtcatagaagcaagagaactcaccaccagaaggcaatcacaaagccagtagacttgtaaacgtcccccaaatgctctcagcacactatggaagggctttagaaaacctatcttcagtacagcagtggaacactaagagaacttagaattcattttacagcgagctagaatgcaatttacagaaaactaacttttgctaaaatgaatctaagccttaaaatatacatcactgcaggtacccataattgatcacatcctcagaggagctcaaagtgaaaaaaaaaaggacttaaatcttcccaatttggaggcactaacatctacataaggacaggtatagctcatgagtcctaaatattgaccaaggatatgtgacctaaaatagtacattctattctgaacactttaattctgtaagtgaagtgtcattacgattcttaaacttctttttctttgaaatcatctatttccatcaattcatagctctaataaatcatgaggatggtttaacatgtgctctgagcttttgtagagaattttaatttgacattttaaagtgtaagctaaattacttgtatcaactgtttttttactccaatcgcaaacaaaactaaaaagtattacattcccttttgctacataggtagtgtagcaaatcaatagtacacattctttataccaagaatgaagggttttaatatgcttgcagaagttaattagaactgtcacagaaaagttctactaataaaaaaggaaaaataccactgctgctaatagctacagaaaacctcctttcccattccaaataaatcctatcagattctaaatttcttacattttgtctggaaacctctagtttccacattttctatgcttatcaactcctatagccaaggtcagatgcaataataatttgtaccacttttttaacttgtaagaggaagacatatcttaagcactctaaacccaagagtcgaccaaattatttttacttcagattagaattagatattaaaggccaaaaagaagaaaataaagaccattttgtgatatctgattaatagattcctattaatcatcattaccatttcctgtaatcaagtaaactcctatattaaacacctaaaataaatggcggcaattattgactcatttacaagcatcttttgagctttataacaggcaatgaggattcatgatcaaaagatacagagaagatgtaatgatggattaatttatcaacagctactgggatgtcctcaaaatggcagttatgatgctttctcatggcttaccatcgaaatcagtttatggctacccatacttcagctgagtaattcctaatgttttccatattgggaaaacaataaattgtttgctcactctagcatgctagcagtggtagattttttttttttttttttttttttttgagacggggtctcactctgtcgcccaggctggagtgcagtggcgcgatctcgactcactagcagtggtagattttataaagtacatggcctatgtgaaatgtctataagataaaaatataacatctctgggtttttttctcttcactaaagcatgctgcaatgttgtacagatctttgatgttgaaaagtagataagatagggatgtgcatttattttctacttaaagagcaatccagtttgtcattgctgtatttcagtgggaaggggatcattgggaagtgccctatctatgttctttatagtataaatcacctacttaggcactagctgttctatgtttctaaggacggattcaagaaccacagatttagcagctcaagaaagccagatttgcttttccataaaaggagtcaggagatatgcaagggtatctttgtagcaaatctggtaacttttagtcttattttaagcacttaaaaaatagttatactccaaatggcctctgggagtaactgcttggcaggttaagtgaataacccacagcaaagttccaacatccagagcttggacttgggcaagaggaagagactctcagactctaagatcctgcagatcctccccttggacatgggttcaggggacatgattttaactgttagctagaaaatgtccctttttcttctttggaggtctttaaccaaggcccagatttaaaaaaaaaaaaaatgcatcactgaaattaaggcatggtaagtgcttccttgaactgccagccatgccagcaaaaggactgatgacctggtgagcagtcaccctgaaagttgcaaggaagaggaggaaggtcggggcaaccatttgctgaagcttcagtgcccctgaataaagaatggagttgaaatcattagaaagggtttgctcacacataggctgcttatgaaccacaacggtcatcttggccaattgcagcttcctgcaaaatcttttctagattgtgcagcatttccaaaacgtttatttccttatctcagtctaaaaacatcaaaaaaagtactgttcaatgaactctttgcttagggctaacacaaacccgagaggcaactgagcaaggagtggtggtagaaacaagtgagaacagcatgtggtctgtgccctccggtgtccgtctcctctgggtaagatattaaacagcaggctccggctttcatggttccattgtccaaaatcccagcgtcaacagttttcatgccatggatacatctgtaggctgccccaaggcaatctacaaaaccatcttcctctctacgcatttactcaaagcactcaagcaattcattttttaaacaattctatttcctcaacccaaaaacatgaaattctttactgggcactggaattcccatattgtttatggagttgatgtgagggttcagttaaactattttttaaaaaggtgggacaagtaatcttagggtggcatcttgttggcagcatatggaccccgatgccaccacaggcaggccaagatggtagcagtgcaggtgccaccccactatggccaagcgttattccacaggacagcgtggaggaaactgatttattctcagaggcttatcaagcctcagtctctgccagggttgtcaaaaattctactctaacaaactgcttatgggacagatttctcttttgagccaaaaagattctaaagactcctggggtccatgaggaagcatctttgttctcactgggaacccaacgaataagtctttatgttctttccactttatcatccaattcctctgcagcttttcactgatcctgctatgaagagaacatgccaacactaggggaaatgccacagcagcaaactgaacgcttgacaaaacaacagttgagtacaacagtcagcagaatgactgcagggaaaaaaaggacagagcagaaatgtttcagatgatattctttggacaaattactcccttgacaattctttctcaatacatttacatcagtttggtgccttaacataatctaaagtggtacagtggttaccagcacaagctctggagcaagaccagtttaaatcgtagctgtgtggtcttgtgcatgtttttaacgagttctgtgccttagtgttctaatctgttaaatggggataataagcaagttgttatgaggattaagcatgctaatacatgcaaagcactttgaacaatgctcaataaagctatcatcattatagttcaaaattgctattgcatgtatcatcatagttgacccacagcactctataaaacacaggtaatcattttatctgtgtttcactgatgaggataatgaagctcagttaagtgttttgctaaaaacataaggaatctacggctgagccaagacttgaatcctggttttttgacattaaacctcggattccgtcccatgactttggattatttacatgtaaaccacagtaaactagccattatcaaatattaattctagacaaattcagtaaggaagaaaacaactgagaacatcatacctattgtagggatttattcctgaaataaacaaaattaaataatcagctgatggcaaattttagctaaagtcattaaagccgatttttagattcagagtagcattttggaactcccaaaattctgaagtgcagcagggagagggaacatttatccatatgtattctttgctctaatgagacactttataaaaatatttatttgaataggtaaatatttatgaaaatagatgaactcccatcttgagaaaattcaatatatgaaaattcagaactacaagatgtttgaataactcattagaaatggagccagtgaagggcttacaaaatgtgtaaaggtgaagtttgccatcttattgctactataattcacaagccagcatgaactttctggtcattgatagatttacgacatttaggctacagactcttaaatatagaagactaagtctcattcattagagaatctcatccattagaatccttatccgcatattacttagggcaagggttataagccaagcaaaagatggtaaatacaaatgaacaactggcacatattccttctttcctaatgaattttataatttagagcatctgaatggatccctaagggaagtattctttcatgactaatactctttctaggattagcatctatttctaattcagaaagtttgaatgcgctagcctttaagaacttggaggagtcttctatttaataagatcaatcttttaattcatggatcattttaattttaatatttactatacatacaaatcattcaaattatccctcaaaaaaaattttttaaaaaagcattttgatagcaccacattaaaaactaaacttccagcaaagatagtgtgctcaaaagtataatctggtaaactctgaatgactagcaaatcatgctgtacatataatcaatttttttttttttttttttttgagacggaatcttgttctgtcacccaggctggagtgcagtggtgcgatctcggctcactgcaagctccgcctcccgggttcacgccattctcctgcctcagccccctgagtagctgggactacaggcgcccaccaccacacccaggtaattttttttttttattttgagtagagacggggtttcaccctgttagccaggatggtctcgatctcctgacctcgtgatccgcctgcctcggcctcccaaagtgctgggattacaggcgtgagccaccgtgcccggccctaatcaatgttttttaattcaactcaccatcaaggttctgtcaaacctgtacaaaaatagtccacccaagacaataaaaggtgatgcttcctttgggttcatctgaccaactcacattaaaatgaccacccatccaagtcaactgatgttcattctgtagctgaaaaacctccctttcattttaacactgaaaaatacaagatgcgtactattctagaaatatgcctttaaagctatccagatggaaatatgtcatttcttagatacatattttaaaatctataaaatgtaagtaaatctcactagcgaacactacaaagacttaaaatcataaaatatttaggtcaaagaaataagttagaattttccctactggtcagagtttactttcacacctcagtcattcattcttcaggctataagatctatgagaaaactgaaaacagatgacagaaaagaatcattatgttaagcaaataataaatgaacaagtagaaatcactgagtaattatttaatcttcaaagatatgcatttttttcttactttattcctatgaataatcctgctcaactcaaacttctgactgttctagggccagcaagtacatatttgttgatcaagtgacccaggccctttaacaacttgagcaaaatgtatgctttagccacgtacaccctccgactccctggttcttgccttaaagtcaaagtcacatcacaatatgtctcttacctgtttcccatccaacgtgtacaggcgtttcaccactcccgagtccagcttgatggcatcggtgatatcggtgaggacctgctcaaaggaatgagccgttttcttgttcagcagaatcctgacagctttccgtggcttcacgccacttctgatgatggtgaccagcttgggccgaatgaaatccttattctctcgcacctctgaagggcttcctttggcagtggccagtgaagacactgcccgagaagccgaggtggtcttgacgttcaccgaccagttggggttcacattcttggtgtactccagtttcttgaagggctctatggagccacatacataactctctcctaaggaagagagaaatatatttaaatttatactaaaatatgcccatttcttttttcctacttatcctctcttttgccttttctgcttaggggcaagagctagccctgacttttcaaacaatggaagtgtgattaataattagggaaaaagaaagatttctgattgctaatctggcactgtcagcaacagatccacatcctggaaaaacaaaacaaaacattattcctccatttcctcatcttaaggtcggtgatagaaatgctgatctacttgaaaagaccttagagaagtgtgaaagtgaatttaaaatagaaaatattctttgggaattagagatacttaaatctgcagaaaatttcaaaattggaagtacctccatttgctacctgagataatttaaacactaagaatgaatcagaatacagcatcttaaaatagtcatataatttacgaatttgatgagagttgatgctaaagattttgtaaagcccagtaaagttccaaaagcaagcaaataatcacccttcctttctctcatctatatagtgccaatcacagtgatccacacatactataaggtcaattcatacttgttcataagtaagaggttttagaagtgttcaacatacaaactacagcttcaaaaatgggctctaaaataaatataagtaaatgtttattaaaagtgactccttagcaggaaatcttttaatgaaaataatcagactattaagtagatgtagatcacacacacaaaaaagaaaaacatgaaccaagagacaacaaattgtaaaatgacgaattctaaatacatatttcataaagttaggtcaaagccttttgaaattaaagattttgtaatggctttttttctgtgttaataagtttttgtaactaggaataggtaaaaatgctgaaagaccttcataattgtaggtaaaatgaccgaggcctaatctaacacagcccattgacttttatctaagtccttgtattatttttaaattcacagtgcaagcatccaaggccaaagaataagtagtcacataaaaataactgaagcaggccttcaaaagttaaaaagagcagttatacatgttactgaacacttgttttgcagatgttcttaaaatattctctaatgtgtatacatacacattatgcctcactagaagttgtatgaatagtgcaagtttaaccaaccccatttctattcacctggcagatcatgacttgtctcagctatgccttcctcaggatttaagtaaatatcaaaaacttatatgtatttttgatattttcttaaatcctgagaaaggcatatttgggtactatagaaacagcagtgttttgggagttttcatggagtgatgacctgcacatgatagtgggaagagagaactggaaattcgggctcaagacctggcctctccaagcatgtgactttggacaagtccctcagatttctgggtggtagtttatttatctataaaacaaaacctcattaggtttttctgaagatcaaattagaagatagagatcaaagtgatctcaaatacaaacctgctatgtgagtgaaaatgacctgaggctacataccctaatcgtgcagtaagcctgaaagaaaaattcaaaaacaaaattactagaaatcggtaagcaggtcttatattgaacagttaccagtggacaccacgttgggtccatgtcaatgtgagaatgccctaacactccccagtgagacacacagcatgtaactgagctggcaaagctagcctctgtcagaattctgtgctaactgaagatatgcaattgtctgattatttataagtttctatactaagctaataactaaaagcaaaacaaagatacagagagcagtttacatttcaacaaaaactttaaaaatatattaaattagaagagggagcagttttaaagaacatagcaacatggagtgagtttgttaaaagagaaaataaacactttttaaaaatatcttcttagataaagcaccctgaaaatgtttaaaagcataagaaagttctccagttacactgagaaggcgttgctggtttggttcaagcaacaatgatcagaagaaatatgtgtttaatctgagctgtaaatgtgaggctcctctgctggataatcagatttaaagactggaatgagaatcttagagaaggatgtgagactccagtaacagatgatactgtgctaacaggaaagcaacagctaaaggaaggagaaggagctgccaccaaatccaagatcacacaccacagtcatttagagtctgtgacactattcaggaattcaggaaaggcccagatggatttccttatgctaataaatttccattgatttccttatgctaatacatttccattttatcatgaaaagaaatttccattaaatctttttaaatctatgtattaactatttttacagtaatcataacactgaacttctctctgccacaagagatgtggttcccgccttaatgcagttaaattaaagttggttgtattttctgattttgtaaaaaatagcttttcaattcatgggctaattatcatcgattattataaattaacaaacgtgattgttctgaatgggaagaatacatagttttgatttggtcaagtagctctagatatccagtgttcaatctcttccttagtgtgactgagagttaagagataagttataatcttgttaaaataatcatttttagagtgcattcaagtattgacatgaggtatgtgtgttaagtgccatttaaaaataacaagacagggccaatcatggtggctcacatatgtagtcccagctacttggggggctgaggtgggagaatcacttgagcctgggtggatgaggctgcagtgagccatgattgtgctactgcactccagcctgggcaacaaagcgagatcccatctctctttatttaaaaaacaaacgaaaaaacaaacaaaataacaagataaagtaagcttacaattactgtagtatacttcctcttacatacactaagccaccccacaccccgaagtcttttggtataacttctactgcaacaatgaaataaaagtgaatttatcttaaacaaacaaacaaacaaataaaaaaacacaaggtatttttgagcaaaacagcccaaaatttgctttggaagctttgcattacttcctataggtgaacttgaacaaggatggcatgattgcaaagctcttagcggtggtactagaaattgcaaacatcatgattttctggttggttatcatacaaccagtttttcttgtttgtttgtttgttttttcagtttgttcaagccctaaggattctatgataagtctgattcttcacttgaaatgtaatactctgggtgtctaagacactccaacaaatagcaagatgctgaagcagcatctggctgaattttgttgtggcatttaatgaaggccaaaaactaatctttgttgagagagataaagaaaatgacagtttttttaaaagtaaggaaataagcatgaaattatgtttggatccaactaatactaaaatgtcagaatatggtaatttctttcctgaaagattttagtattttcagaaagttggaggatgaaataatatctaagtaagcatactagattatttttaagatactgaagagttcctgtgtaaaaaatcacctatgtatttttatttttatgtttcattttttatgtttttgagacagagtctcactctgtcacccaggctggagtgcagtggtgtgatctcagctcactgcaacctctgcctcctgagtccaagcaattctcctgcctcagcctcctgagtagctgggattacaggcatgcaccaccacacccagtgaatttttatatttttagtagagacaggatttcatcatgttggccctcctggcctcaaactcctgccctcaagtgatccgcccacctcagcctcccaaagtgctgggattataggcgtgagccaccgcgactggcctgtatttttaaatgatcatactggccattattcttcaaagcataacaatacatttcctctaataagatttattattttaaaaatctgtgaagttaattctactttgccattcgcttccattatgaaattagaatatttccacttaaaatggttccctgggcactactgaaaactgacaatgtggtacctgttccctgggtgactgggcctgcagtgtgtcagctataaaagtgttcctccacgagactctatattgggggattctacaaatttgcctcttattgtgggagttttttcattggctaagagtaaattccagttctgatactacttaacttcagtctgaaaatgacctcatctccatccttcacattactagagctggttgttacctcctctaggcttcctttaagatcaatatgtggcatagtcaaaagcatccttaagtgaaggatatccgtaatatatttcatttttccagaggataaactttctgattaaaaactctggcaattaaatttcattattgtgagttatggcaaaatctaggaaaaactcaaatatgtattgaaacctctgcaactgaactgaattaaattacatgaaatattgtttacagaatagaatatcgtaaagtaccttctggatattacattttatgaggcaactcagatacgcacagcaacatttcataatctacctggaagtcactggcttaggatgattttcattgagctattaaaggcaagaaaaaatatttcccaaaggtgcttatgggtcattcttttgcagaagtcttccgtaacacaatagaacataaaagtaagcttatagaaaatgaacttgaatgtgtatgtcagacctgtggacatgtcaccttctcaaatccaaagcaacagaaaaacaacattaaggaaaaaaaaaatgagagatctaaactttttgaaattgaaaagtagagactcttttacttatttcttattataataagcatggcttattataatttacaaaaatacagggtacaaaaatagatggcaaaggataaaaacaaaacaaagaatatatggaaaacacatggagggtgcttaaatagtaagccaagaaatgcaagttaaagcagatttaagatgtactcttgtacctattataattgcaacaaaagaataatcaaaataaagtggtattgaggttgcagcaacaaaataatacaaggtgctaaaatatatgacaaaggataaacacacaataaagtataaatagaaaacaccattacagacattaaaatggtaataatcattttaaaaagtaacatggaaaactacttggagccacgaaaatgtttatacccataatctcattgctaaaaaattttgcataataacaaaacaacaaataacaaaactgtgacaagcacacacaacattaacaatgaaacctaaaaattaaaaacaacctcctaaatcctgagtcataggtgcaagacctaacttagccactctgggaagggaaagaaacctaagtcaggagactatcttcaaataggttagaaagcaagatgtgtctatgaaaagattccaccacaaggcagtaagtaataattgctaagtaagtggtctaggccatcctaattcagaggagaaagaagtcacttcccacttcccactgggctatctatgaaagggaaatggaattc

SEQ ID NO: 17 - DNA, 5820 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttataaaaaaaaaaaaaaaaaaaaaaaaaaaaaaa

SEQ ID NO: 18 - DNA, 4798 bp, Mus musculus
tgttttcattttagtcttagtgaaaggttttgaatttacagctcaagttacttttcaaagcagtgtgagtagttttacttaaaaaaacaaaacaaaaccaaaaaccccggcaagaaagactttcacggcttccagctttggtcttgtaaccctgaaggtaattcatttttaatctgtctgcacagtgaaaactgaaccgaatggggattgagctggtctgcctgtttctgctacttctaggaaggaatgatcacgtccaaggtggctgtgcctggggtggtgcagaaagctgctcagactgcctgctcacaggaccccactgcgcctggtgctcccaggagaatttcacccacctgtctggagctggcgagaggtgcgacaccccagcgaatcttttagccaaaggatgtcaactacccttcattgaaaaccctgtctcccgcatagaagtccttcaaaataagcctctcagcgtgggccgacagaagaacagttctgacattgttcagattgctcctcaaagcttggttcttaaattgagaccagggcgtgagcagactctgcaagtgcaggtccgccaaactgaagattacccagtagatctgtattacctcatggacctctccgcctctatggatgacgacctcaacaccatcaaagagcttggctcccggctggccaaagagatgtccaaactaaccagcaactttagactgggctttggctcttttgtggaaaagcccgtttctccttttatgaaaacaaccccagaggaaatcaccaacccttgcagtagtatcccctatttctgcttacctacatttggattcaagcacattttgccattaactgatgatgccgagagattcaatgaaattgtgaggaaacagaagatttctgctaatattgacacacctgaaggtggatttgatgcaatcatgcaggctgctgtgtgtaaggaaaagattggctggcgcaatgactcgctccacctcctggtttttgtgagtgatgccgattctcattttggaatggacagcaagctggcaggcattgtcattcccaatgatggactctgtcacttggaccacaggaatgaatattccatgtcaactgtcttggaatatccaactatcggccaactcattgataaactggtacaaaacaacgtgctactgatctttgcagtcacccaagaacaagtccatctgtatgagaactatgcaaaactcattcctggagcaaccgtgggactgcttcagaaggattctgggaacatccttcagctgatcatctccgcttatgaagaactgcggtctgaggtggaactggaagtgttaggggacacagaaggactcaacctgtctttcacagctctctgtaacaatggtgtcctcttcccacaccaaaagaaatgctcccacatgaaagttggagacacagcatccttcaatgtgactgtgagcgtatccaactgtgagaaaagaagcaggaacctcatcataaagccagtggggctgggggacaccctagaaatactcgtcagtgcagaatgtgactgcgactgccagagagaaatagaaactaacagctctaagtgtcacaatgggaatggctccttccagtgtggggtgtgtacctgcaaccccggccacatgggtcctcactgcgagtgtggtgaggatatggtgagcacggattcctgcaaggagtccccgggtcacccttcatgcagtggaaggggtgactgctattgtgggcagtgcatctgccacttatcgccgtacggaagcatctacggaccttactgccagtgtgacaacttctcctgtctgagacacaaaggccttctctgtggagataacggtgactgtgactgtggcgagtgtgtgtgccgggatggctggacaggtgaatactgtaactgtacaaccaacagggactcgtgtacatctgaggatggagtgctgtgcagtggacgtggggactgtgtctgtggcaagtgtgtctgcagaaaccctggagcctcaggacccacctgtgaacgctgtcctacctgtggcgacccctgtaactctaaacggagttgcatcgagtgctacctgtctgcagatggccaggcccaagaagagtgtgctgacaagtgtaaagccattggtgccaccatcagtgaggaagatttttcaaaggatacttctgtctcctgctctctacaaggagaaaatgaatgccttattacattcctaataactacggataatgaagggaaaaccatcattcacaacatcaatgaaaaagactgccccaaacctccaaacattcccatgatcatgttgggggtgtcactggcgatcctgctcatcggagttgtgctactgtgcatttggaagctgctggtatcatttcatgaccggaaggaagttgctaaatttgaagcagaacgctctaaggccaagtggcaaacgggaaccaatcctctgtaccgaggttccaccagcacttttaagaacgtgacctacaagcacagggagaagcacaaagcaggcctttcctcagatgggtagacagacctccttcggccagagaaagtcaactccgcttgcgttaaaggtttactgcactgtatggtttatcatgctttgtcgcttcaaaaggaggtcgacttaagataataacaggtcattcaacaaccagtcattcagggcatgaaggtgagacattgttcgctgattggaaacaatcaagaaaagaatatcccaagcaaagagatgaattcaagatcccttgaagaatataaactctgttgcattaatgcctcaaaaaaaaaaatcatcaaaacgattcccgggaacttgatttgcatttgaaaaatgttcagaattagaatctcatttgcttccggaatccagctacctaaagtctttgtctccaaaaaaatcccggagtccttatgcattgtctacccatacttgcagaaaaatctatttcctgaaggaatttgtttctttggttggtttggtttggttttgtttttttatttcctttccttttctttttttcttttttctttctttctttctttctttctttctttctttctttctttctttctttctttttcttttttttttttttttagaaagggtgtctctatggccattctggaactgaccaggctgcctggccttgaactcacaaccatggaactgcctgtctctgccttccatttcatgctcagagatcttaacttttatgtatgagaaatgagagtctgatttcccatcaactgaaagaatccagttctcccagagtgacttcccaagtgagagattgaaaattaagtcatgggcttgtactcttaaaagacatggacacaagtattttgtttgaaagaggaggaaggggtaatttataaaatgtgttgctagtaacactaaagatatgggtcctccgtgttcaattgccctctgatccccaagtttccctcctattattccacatttctcaattattgcactcaactacagtcttcaaaactccacaactgtcatacaaaagtatccactttttaaaaaatcactttaagttattcgcttgtcaactagaattttgagaatagaaaagaaacctagggtaacagaatcaaaggctacacagctttatatcaaatgtgtcaagactgtggaagtcccgcatgaagtctgccctacatgtaggacattcctatcccgtatgtaccacccagaacacacatcccagcttcaggaggtgggcatgccacccacttgctctaaaaagtaaaaccacttacaaaatctatgcaaggcaacaaacaaaaccggtagaagattggagggatagtctctggagggtttcatttcttatagttactgctgcattacaaaccactttgatctcactggatctccatagagatgctgagaggatgggcagaagaggtgggtacagactctcaatcttataaccccaagccctttgttctctctactgcacgagactcttttgtcagtaaaatgatggaaggattgtattagttatggtataactgtcttagtttgaaagtatactgacacaaaacaattagcacctaggcccccggaatgtggaccggttgctgtatttttgtgctgtgtgtgttgccttcactggatattaaggtgaatccactccctaggtgaacagctctgtgtttgtgagtggaggctttgacagctgtctgttgaattgtcagcttggtctcaaacctccttcactcctgccatataggtctgtgtacgtgtccacatcatctaggttctttcttttctggtttttaaatgactcaggtaaattagtctgttagggagtacacagctaaatttaaaagtcttaatgaatctcctttggatccctgaatggtccaagatcttggagtctggagcaaccagcagattcactcccagtgatgtggtgagactgctgaggatacacggggctgtttgtgctgtttttgtgttattgctgaaatgagatgttaggaaatagaagtttctctcatgtgtgtatcaggaagaatttgttctttctctaattaccgttttgacccaaggcaggaccttgaaagagccaaaacattaacagtagtgcttctgttcactgaagaattatgttacatggagataaaatggttgttttttttttttaaagtggttttattgtattttgcgttgacataaataaacataatttaaacaatgaaaaaaaaaaaaaaaaaaaaaaaaaaaaaaaaaaaaaaaaa

SEQ ID NO: 19 - DNA, 695 bp, Mus musculus
  misc_feature 4: n = A,T,C or G
ggantttggccctcgagsccaagaatttcggcacgagggggtactaggtcagcaatcccatggcgtataaaagcatctcatctggccaggtcctggaacctttcctgagattctgccccaggatgccaactttgaataggatgaagactacaacttgttcccttctcatctgcatctccctgctccagctgatggtcccagtgaatactgatgagaccatagagattatcgtggagaataaggtcaaggaacttcttgccaatccagctaactatccctccactgtaacgaagactctctcttgcactagtgtcaagactatgaacagatgggcctcctgccctgctgggatgactgctactgggtgtgcttgtggctttgcctgtggatcttgggagatccagagtggagatacttgcaactgcctgtgcttactcgttgactggaccactgcccgctgctgccaactgtcctaagaatgaagaggtggagaacccagctttgatatgatgaatctaacaaaaactgcagtctcaatttggaaatctgactcatgtgcctttaaatgtgttcatattgcccatttaccctgcttcttgaaatgcttcttgaaaaataaagacaaatttgcatgtgaaaaaaaaaaaaaaaaaaaaaaaccatttgcggcsscaagcttattacctttagggggg

SEQ ID NO: 20 - DNA, 844 bp, Homo sapiens
cagacacagagtaaacttttgctgggctccaagtgaccgcccatagtttattataaaggtgactgcaccctgcagccaccagcactgcctggctccacgtgcctcctggtctcagtatggcgctgtcctgggttcttacagtcctgagcctcctacctctgctggaagcccagatcccattgtgtgccaacctagtaccggtgcccatcaccaacgccaccctggaccggatcactggcaagtggttttatatcgcatcggcctttcgaaacgaggagtacaataagtcggttcaggagatccaagcaaccttcttttactttacccccaacaagacagaggacacgatctttctcagagagtaccagacccgccagaaccagtgcttctataactccagttacctgaatgtccagcgggagaatgggaccgtctccagatacgagggaggccgagaacatgttgctcacctgctgttccttagggacaccaagaccttgatgtttggttcctacctggacgatgagaagaactgggggctgtctttctatgctgacaagccagagacgaccaaggagcaactgggagagttctacgaagctctcgactgcttgtgcattcccaggtcagatgtcatgtacaccgactggaaaaaggataagtgtgagccactggagaagcagcacgagaaggagaggaaacaggaggagggggaatcctagcaggacacagccttggatcaggacagagacttgggggccatcctgcccctccaacccgacatgtgtacctcagctttttccctcacttgcatcaataaagcttctgtgtttggaacagctaa

SEQ ID NO: 21 - DNA, 2764 bp, Homo sapiens
agctctcagagggaattgagcacccggcagcggtctcaggccaagccccctgccagcatggccagcgagttcaagaagaagctcttctggagggcagtggtggccgagttcctggccacgaccctctttgtcttcatcagcatcggttctgccctgggcttcaaatacccggtggggaacaaccagacggcggtccaggacaacgtgaaggtgtcgctggccttcgggctgagcatcgccacgctggcgcagagtgtgggccacatcagcggcgcccacctcaacccggctgtcacactggggctgctgctcagctgccagatcagcatcttccgtgccctcatgtacatcatcgcccagtgcgtgggggccatcgtcgccaccgccatcctctcaggcatcacctcctccctgactgggaactcgcttggccgcaatgacctggctgatggtgtgaactcgggccagggcctgggcatcgagatcatcgggaccctccagctggtgctatgcgtgctggctactaccgaccggaggcgccgtgaccttggtggctcagccccccttgccatcggcctctctgtagcccttggacacctcctggctattgactacactggctgtgggattaaccctgctcggtcctttggctccgcggtgatcacacacaacttcagcaaccactggattttctgggtggggccattcatcgggggagccctggctgtactcatctacgacttcatcctggccccacgcagcagtgacctcacagaccgcgtgaaggtgtggaccagcggccaggtggaggagtatgacctggatgccgacgacatcaactccagggtggagatgaagcccaaatagaaggggtctggcccgggcatccacgtagggggcaggggcaggggcgggcggagggaggggaggggtgaaatccatactgtagacactctgacaagctggccaaagtcacttccccaagatctgccagacctgcatggtcaagcctcttatgggggtgtttctatctctttctttctctttctgtttcctggcctcagagcttcctggggaccaagatttaccaattcacccactcccttgaagttgtggaggaggtgaaagaaagggacccacctgctagtcgcccctcagagcatgatgggaggtgtgccagaaagtcccccctcgccccaaagttgctcaccgactcacctgcgcaagtgcctgggattctaccgtaattgctttgtgcctttgggcacggccctccttcttttcctaacatgcaccttgctcccaatggtgcttggagggggaagagatcccaggaggtgcagtggagggggcaagctttgctccttcagttctgcttgctcccaagcccctgacccgctcggacttactgcctgaccttggaatcgtccctatatcagggcctgagtgacctccttctgcaaagtggcagggaccggcagagctctacaggcctgcagcccctaagtgcaaacacagcatgggtccagaagacgtggtctagaccagggctgctctttccacttgccctgtgttctttccccaggggcatgactgtcgccacacgcctctgtgtacatgtgtgcagagcagacaggctacaaagcagagatcgacagacagccaggtagttggaactttctgttccctatggagaggcttccctacacagggcctgctattgcagaatgaagccatttagagggtgaaggagaaatacccatgttacttctctgagttttagttggtctttccatctatcactgcattatcttgctcattcttcagttctctactccctcttgtcagtgtagacacaggtcaccattatgctggtgtatgtttatcaaagagcacttgagctgtctgaagcccaaagcctgaggacagaaagaccctgatgcaggtcagcccatggaggcagatgcccttgctgggcctgggggttttccaagccctcagctggtcctgaccaggatggagcaagctcttcccttgctcatgagctcctgatcagaggcatttgagcagctgaataacctgcacaggcttgctgtatgacccctggccacagccttccctctgcattgacctggaggggagaggtcagccttgacctaatgaggtagctatagttgcagcccaaggacagttcagagatcaggatcagctttgaaggctggattctatctacataagtcctttcaattccaccagggccagagcagctccaccactgtgcacttagccatgatggcaacagaaaccaagagacacaattacgcaggtatttagaagcagagggacaaccagaaggcccttaactatcaccagtgcatcacatctgcacactctcttctccattccctagcaggaacttctagctcatttaacagataaagaaactgaggcccacggtttcagctagacaatgatttggccaggcctagtaaccaaggccctgtctctggctactccctggaccacgaggctgattcctctcatttccagcttctcagtttctgcctgggcaatggccaggggccaggagtggggagagttgtgatggaggggagaggggtcacacccaccccctgcctggttctaggctgctgcacaccaaggccctgcatctgtctgctctgcatatatgtctctttggagttggaatttcattatatgttaagaaaataaaggaaaatgacttgtaaggtcaaaaaaaaaa

SEQ ID NO: 22 - DNA, 845 bp, Homo sapiens
caggtgagcctctcactcgccacctcctcttccacccctgccaggcccagcagccaccacagcgcctgcttcctcggccctgaaatcatgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggctgagtgcacaggtgccgccagctgccgcaccagcccgaacaccattgagggagctgggagaccctccccacagtgccacccatgcagctgctccccaggccaccccgctgatggagccccaccttgtctgctaaataaacatgtgccctcaggccaaaaagaaa

SEQ ID NO: 23 - DNA, 424 bp, Homo sapiens
agcggccgcccttgtagcgtaaggttcccccaacattggccacagagccgttgatgacgacagcagccgtgatgcccttcaggaaagaggccatggaaaggcagagctcaccccctttggaaattccaagcagcccaactcctggaccttttacctcaggatgactgagcaagtagttcacagcttcttcaaagtactccagatggagcgtctccatggtcttggggaggtcttcatagttatagtaagccagagccatcacagcaaaacccttcccagccagcagactagcccgatactccagcaggccacctccagttccgaacatgtccacaatgccaggaaagggcccaggttctggcggcaggaagagcgtgcctcgcacccggcccgcgcgcaccggctcgcgccgcacccccggcgg

SEQ ID NO: 24 - DNA, 3107 bp, Homo sapiens
gccagctgctgtagaagaggggaggaaacaagccagtgcaaggggagcaaaagagaaaaggagccaggctgggcttcctgatcccacagcatcgcagagctcgggaggcacagctcacagacacaggaaacacaggactgctattctgctctcctgcccacggtgatctggtgccagctggtggaacagtgggtgatggcgtccctgctgcaagaccagctgaccactgatcaggacttgctgctgatgcaggaaggcatgccgatgcgcaaggtgaggtccaaaagctggaagaagctaagatacttcagacttcagaatgacggcatgacagtctggcatgcacggcaggccaggggcagtgccaagcccagcttctcaatctctgatgtggagacaatacgtaatggccatgattccgagttgctgcgtagcctggcagaggagctccccctggagcagggcttcaccattgtcttccatggccgccgctccaacctggacctgatggccaacagtgttgaggaggcccagatatggatgcgagggctccagctgttggtggatcttgtcaccagcatggaccatcaggagcgcctggaccaatggctgagcgattggtttcaacgtggagacaaaaatcaggatggtaagatgagtttccaagaagttcagcggttattgcacctaatgaatgtggaaatggaccaagaatatgccttcagtctttttcaggcagcagacacgtcccagtctggaaccctggaaggagaagaattcgtacagttctataaggcattgactaaacgtgctgaggtgcaggaactgtttgaaagtttttcagctgatgggcagaagctgactctgctggaatttttggatttcctccaagaggagcagaaggagagagactgcacctctgagcttgctctggaactcattgaccgctatgaaccttcagacagtggcaaactgcggcatgtgctgagtatggatggcttcctcagctacctctgctctaaggatggagacatcttcaacccagcctgcctccccatctatcaggatatgactcaacccctgaaccactacttcatctgctcttctcataacacctacctagtgggggaccagctttgcggccagagcagcgtcgagggatatatacgggccctgaagcgggggtgccgctgcgtggaggtggatgtatgggatggacctagcggggaacctgtcgtttaccacggacacaccctgacctcccgcatcctgttcaaagatgtcgtggccacagtagcacagtatgccttccagacatcagactacccagtcatcttgtccctggagacccactgcagctgggagcagcagcagaccatggcccgtcatctgactgagatcctgggggagcagctgctgagcaccaccttggatggggtgctgcccactcagctgccctcgcctgaggagcttcggaggaagatcctggtgaaggggaagaagttaacacttgaggaagacctggaatatgaggaagaggaagcagaacctgagttggaagagtcagaattggcgctggagtcccagtttgagactgagcctgagccccaggagcagaaccttcagaataaggacaaaaagaagaaatccaagcccatcttgtgtccagccctctcttccctggttatctacttgaagtctgtctcattccgcagcttcacacattcaaaggagcactaccacttctacgagatatcatctttctctgaaaccaaggccaagcgcctcatcaaggaggctggcaatgagtttgtgcagcacaatacttggcagttaagccgtgtgtatcccagcggcctgaggacagactcttccaactacaacccccaggaactctggaatgcaggctgccagatggtggccatgaatatgcagactgcagggcttgaaatggacatctgtgatgggcatttccgccagaatggcggctgtggctatgtgctgaagccagacttcctgcgtgatatccagagttctttccaccctgagaagcccatcagccctttcaaagcccagactctcttaatccaggtgatcagcggtcagcaactccccaaagtggacaagaccaaagaggggtccattgtggatccactggtgaaagtgcagatctttggcgttcgtctagacacagcacggcaggagaccaactatgtggagaacaatggttttaatccatactgggggcagacactatgtttccgggtgctggtgcctgaacttgccatgctgcgttttgtggtaatggattatgactggaaatcccgaaatgactttattggtcagtacaccctgccttggacctgcatgcaacaaggttaccgccacattcacctgctgtccaaagatggcatcagcctccgcccagcttccatctttgtgtatatctgcatccaggaaggcctggagggggatgagtcctgaggtgggcatttcacgggaagggttggtatgctggctttagacggggagaaacatctggaaggatgctcgagagaacaaatggaggtggtgaaaatcaagctttggattgtgcattcctaggcacaaaattacctcattcttcctaacaagcaatctgggacctgattttccaccttttttctcttttcttcccttcctttgttttcataagcctttggtatctttcctgcccttttcctttgtgtactctatactggagttcccttcttcctcttgctgtaggctcaatcccataccgacatctacaactaatctttcccatcaactctgtgtgaaggcaggttgcaactagaaattcagaggggcttggaatagagaaacctaaagaagcatcatcccctccatccccaacttcctcaaagcccaaagccaagggaaggataaatcaaggctcaaggcttccccagcaaagattagggaaagagacttgaccccaggactgtactacgactcttaagagaacactgcacagcactcaaagtcccccactggactgcttcctccttagccccactggtataaatacatctctctccaatttggcaaaaaaaaaaaaaaaaaaa

SEQ ID NO: 25 - DNA, 1892 bp, Homo sapiens
gaggctcaggaaaaagatgaactggtcccattcctgcatctccttttgctggatctactttgctgcttccagactgagagctgcagagacggcagatggaaaatatgctcagaagttgtttaatgacctttttgaagattattctaatgctcttcgtccagtggaagatacagataaagtcctgaatgtgaccctgcagattacgctctctcagattaaggatatggatgaaagaaaccaaattctgactgcttatttgtggatccgccaaatctggcacgatgcctatctcacgtgggaccgagatcagtacgatggcctagactccatcaggatccccagtgacctcgtgtggaggccagacatcgtcttatataacaaggctgatgatgaatcttcagagcctgtgaacaccaatgtggtcctgcggtatgatgggctgatcacctgggatgcaccggccatcaccaaaagctcctgtgtggtggatgtcacctacttcccttttgacaaccagcagtgcaacctgacttttggttcctggacctacaatggcaatcaggtggacatattcaacgccttggacagcggagatctctctgacttcattgaagatgtggaatgggaggtccatggcatgcccgctgtgaagaatgtgatctcctatggctgctgctctgagccttacccggatgtcacattcaccctccttctgaagaggaggtcctcgttctatatcgtcaacctcctcatcccatgcgtcctcatatcttttctggctcctctgagtttttatctcccagcagcctccggagaaaaggtctccctgggagtgaccatcctgttggccatgactgtatttcagctaatggtggcagaaatcatgccggcctcagaaaatgtgcccctgataggtaaatactacatagccacgatggccctgatcacagcctccactgcgttgaccatcatggtgatgaatatccacttctgtggggccgaggcccggccggtgccacactgggccagggtggtcatcctgaaatacatgtccagggtcttgtttgtctatgatgtgggtgaaagctgcctcagcccgcaccacagtagagagcgggaccacctcacgaaagtttatagcaaactcccagagtctaacctgaaagcagccaggaacaaagacctttccagaaagaaggacatgaacaaacgcttaaagaacgacctgggctgccagggtaagaaccctcaggaggccgagagttactgtgcacagtacaaagtgctgacgaggaatattgagtacatcgccaagtgcctcaaagaccacaaggccaccagttccaaggggagtgaatggaagaaggtggcgaaagtcatagaccgattcttcatgtggatttttttcattatggtgtttgtgatgactattttgatcatagcaagagcggattagtcacagatattggctttgctatctgggtagaaattaatacaattccctgtgatctattccaatgttcttccaagatttttgttcatctataatttaggggttatgttgtctgtgctttttatttttagcttcaaatgaatgtcgaagctatctgctctgttaaattaagcagaagatccaaaatttcaagggtaggaagatggaagaaatagggaaagagcccttttatagcccaccgtagtggtgggtgactggcctctagtttatataattatttacctctttggcagtacagataacaaaatacactttactggtaaaatttaaaacaaaaaaggcaaaacaaaacaaactcattttccccttagttcccattaaaatatgcatttgaaaaatgaaatagtcatatatatattagtgtgatttaagtctcaat

SEQ ID NO: 26 - DNA, 2747 bp, Homo sapiens
ctcttccacatcaccttggtgtctccctaaataaaaccagccctccttatcgcctggaaaaaatcaagagctagagtttgaatgggttttatataaacactcacccctgtcagcgtgcggctgggctctcaggataaactcacagcatctggcgcgatgcttgccttgcgttctctcccctgaacgtcaaggtttaagcagagcccgaggactgggagctcttctctgaaattcgatcaacctgaagccagttgcggaactgcacggggtcccgatggacctcaaggaaagccccagtgagggcagcctgcaaccttctagcatccagatctttgccaacacctccaccctccatggcatccgccacatcttcgtgtatgggccgctgaccatccggcgtgtgctgtgggcagtggccttcgtgggctctctgggcctgctgctggtggagagctctgagagggtgtcctactacttctcctaccagcatgtcactaaggtggacgaagtggtggctcaaagcctggtcttcccagctgtgaccctctgtaacctgaatggcttccggttctccaggctcaccaccaatgacctgtaccatgctggggagctgctggccctgctggatgtcaacctgcagatcccggacccccatctggctgacccctccgtgctggaggccctgcggcagaaggccaacttcaagcactacaaacccaagcagttcagcatgctggagttcctgcaccgtgtgggccatgacctgaaggatatgatgctctactgcaagttcaaagggcaggagtgcggccaccaagacttcaccacagtgtttacaaaatatgggaagtgttacatgtttaactcaggcgaggatggcaaacctctgctcaccacggtcaagggggggacaggcaacgggctggagatcatgctggacattcagcaggatgagtacctgcccatctggggagagacagaggaaacgacatttgaagcaggagtgaaagttcagatccacagtcagtctgagccacctttcatccaagagctgggctttggggtggctccagggttccagacctttgtggccacacaggagcagaggctcacatacctgcccccaccgtggggtgagtgccgatcctcagagatgggcctcgacttttttcctgtttacagcatcaccgcctgtaggattgactgtgagacccgctacattgtggaaaactgcaactgccgcatggttcacatgccaggggatgcccctttttgtacccctgagcagcacaaggagtgtgcagagcctgccctaggtctgttggcggaaaaggacagcaattactgtctctgcaggacaccctgcaacctaacccgctacaacaaagagctctccatggtgaagatccccagcaagacatcagccaagtaccttgagaagaaatttaacaaatcagaaaaatatatctcagagaacatccttgttctggatatattttttgaagctctcaattatgagacaattgaacagaagaaggcgtatgaagttgctgccttacttggtgatattggtggtcagatgggattgttcattggtgctagtatccttacaatactagagctctttgattatatttatgagctgatcaaagagaagctattagacctgcttggcaaagaggaggacgaagggagccacgatgagaatgtgagtacttgtgacacaatgccaaaccactctgaaaccatcagtcacactgtgaacgtgcccctgcagacgaccctggggaccttggaggagattgcctgctgacacccctcgagtcacccagcactccctccaaacagaccttgaggcccaagacccaggacaaggaacagcaagctcaggtgggatggccccagtgctggaaagaagcaagagccccctatgcacacattgcagactagctgcctagacctcgctccggccacgtccaacacgacgcatccttgggccccgccgtgcgtccctcttaggagagatgagtcacactctggaactgtccaagaacgaacctgccatcacatctcactgccagatgtataaagcacctgcatgctcagacttcttgtggcgccacctccacgtctgtcttgtacatgacactcctccacgcggtttccagtgtccacactgctgcccgtgcagtgggaccagattccaggtccaaagtcaccatgaggccaccctggaatcagaactgcacaatcaagagggaacccatgggactctctgctacattcagttcttgtgtcgtttgtgaaagttcttaacctgcccaaaaacccccttttccccaagctgcccatggggcttcggcgccaaaggtgacccgcgccaacctccctcccccccagtgcctatgacggcggcacagcagccagcgggtgggggacgcctgtgttcacccatggtgcccatgtcgttcttctctccctgtgacacagcttgtacagtctgattctttttatctggggtaggggggcttttatgtttgtccgatggagatttgttttgttttgcttcattttatgcttttttattttagttttgatgttctgaggtttgctttggtttttccattttctttggcatttatttattcgtgcttcaaatcacagtcatattaaaagctggtcttgtggaaaaaaaaaaaaaaaaaaaaaaaaaaaaaaaaaaaaaaaaaaaaaaaaaaaaaaaaaaa

SEQ ID NO: 27 - DNA, 469 bp, Homo sapiens
ccccgcgccgtggcgaccttggatccagacacctggagatgctgagctgaccagaactggaaggccgcttgaacctagggctgaccaacacacttttggatcaaagggagcctttgggtttcagcatcctgtaagagtctatttacccatgtcaaagcgtcaagaatacctgcggagttccggggagcaagtactggccagtttcccagtgcaagccacgattgacttctacgacgatgagtctactgagtctgcttccgaagctgaagagccagaggaaggacccccacccctccatcttctgccccaggaggtgggaggtcggcaggaaaatggcccagggggaaagggcagagaccagggcatcaaccaagggcagcgatcctcaggagggggtgaccactggggggagggtccgctccctcaaggtgtctcctcaaggggtggcaagtgctcctcatccaaatga

SEQ ID NO: 28 - DNA, 566 bp, Homo sapiens
gccagaaccggtggagcagcgacccctgagcagtgttctctgtgctgagcggcgggactgagctgttgagttagagccaacatgagtgagcgacaaggtgctggggcaaccaatggaaaagacaagacatctggtgaaaatgatggacagaagaaagttcaagaagaatttgacattgacatggatgcaccagagacagaacgtgcagcggtggccattcagtctcagttcagaaaattccagaagaagaaggctgggtctcagtcctagtgggagaaccccctcctagtccacctgaaaacaccaaattcaaccatcatctgtcaagaaattaaaagaacaacaccctagagagaagtcatccacacacaatccacacacgcatagcaaacctccaatgcatgtacagaaacctgtgatatttatacccttgtaggaaggtatagacaatggaattgtgagtagcttaatctctatgtttctctccattttcattcctcctgcaactattttccttgatgttgtaataaaatgaagttacgatgagtgaattcaaaaaaaaaaaa

SEQ ID NO: 29 - DNA, 1828 bp, Homo sapiens
acttctccctagagcaggggtgtttgccagcagcctgcactctcagaaatcagacttgagtggccggaacccttgagaccagaggcttaccatgctgctccctaggagggccaggaactgctgacgtgaccactggacagttattcgtgtctcttacaattaccaaacagaatggacaagcttaataaaataaccgtccccgccagtcagaagttgaggcagcttcaaaagatggtccatgatattaaaaacaatgaaggtggaataatgaataaaatcaaaaagctaaaagtcaaagcacctccaagtgttcctcgaagggactacgcttcagagagccctgctgacgaagaggagcagtggtccgatgactttgacagcgactatgaaaatccagatgagcactcggactcagagatgtacgtgatgcccgccgaggagaacgctgatgacagctacgagccgcctccagtagagcaggaaaccaggccggttcacccagccctgcccttcgccagaggcgagtatatagacaatcgatcaagccagaggcattccccacccttcagcaagacacttcccagtaagcccagctggccttcagagaaagcaaggctcacctccaccctgccggccctgactgctttgcagaaacctcaagtcccacccaaacccaaaggcctccttgaggatgaggctgattatgtggtccccgtggaagataatgatgaaaactatattcatcccacagaaagcagttcacctccacctgaaaaagctcccatggtgaatagatcaaccaagccaaattcctcaacgcccgcctctcctccaggaacagcttcaggtcgaaacagtggggcctgggaaaccaagtcacctccaccagctgcaccatccccgttgccacgggccgggaaaaaaccaacgacaccactgaagacaactccagttgcctctcaacagaatgcttcaagtgtttgtgaagaaaaacctatacctgctgaacgccaccgagggtcaagtcacagacaagaagctgtgcagtcaccagtgtttcctcctgcccagaaacaaatccaccaaaaacccatacctctgccaagatttacagaagggggaaacccaactgtggatgggcccctacccagcttttcatctaattccactatttcagaacaggaagctggcgttctctgcaagccatggtatgctggagcctgtgatcgaaagtctgctgaagaggcattgcacagatcaaacaaggatggatcatttcttattcggaaaagctctggccatgattccaaacaaccatatacactagttgtattctttaataagcgagtatataatattcctgtgcgatttattgaagcaacaaaacaatatgccttgggcagaaagaaaaatggtgaagagtactttggaagtgttgctgaaatcatcaggaatcatcaacatagtcctttggttcttattgacagtcagaataacacaaaagattccaccagactgaagtatgcagttaaagtttcataaagggggaaaaaaaagatcaataccattgcttcagacactttcccaaagtttctccttttgagaaaaagtcccaaaacttcatattttggattatgaatcatccagtaataaaatggaagatggagtcagctattgaagtggtcatccatttctttttaagaagctcatgtggacttgttctattgcctgacctgatgaactgttaatatctggtgaggttgagttatcatgctactaatattttccaaataaatatttttatttttaaaaataaaaaaaaaaaaaa

SEQ ID NO: 30 - DNA, 162013 bp, Homo sapiens
gaattcaaatccgggggccttgattgtaagtcagtccactttcctccataaagtgctcaaaatccttcaagcaagtccatgaaacatagtactcagatctctcacactttataacctgcagaaacctcgttgaaactttgtatttattttcttactatcatggtcctccaatgggctactttgtttcattttacggtgtcaggatccacagtcagccagactggcagatggggaggctgtgctttcatttggtgggaatggttctctagcctcctgatgtgaaagttgcaaaatggtcatttaaatctcatacaccaccttcgcatcttacaacttgagatagttctaagacactaagtcagaaatcaatgccaagggcacatccctttatggccagctcctaatttgtgtctcctgctcaaggcaaggcagtggatttatcacccacattgaacttcctcagtggagacttttctaatatccgaatctcggccttatcttgttgtcagatattgggcagaataaaatacaatgtaacgcagagaagcgagtctgaccccctgtcccaaatatttcaattcactgcccagggagtgtcttcaagattgatgctaggatttttcaatgcttgctaatatggtatggatgaaaaaaaaagagcttttccctaggctatcactcagagcagggcttcagaacacctttaaaaatagactgaatcacatttgtttttcctttccagatcattttgacttggaatcactttttcccttccagattacctggctgaatctataaaaactgaatccattgtttgtttttattagaagagtgtgtctttcccatactttctgaaacatcaacaattcaacaaaaattttatgttggcccttgtccctcagagtaactagtcctttctgctgatgacaaaagtgttttaatagtgtaataggtttttgttttgttttgttttttgacaaagtctcgctatgtcacccaggctggagagcagtggtgcaatctcagctcactgcaaccttgccccccgggttcaagcaattctcctccctcagcctcctgagtagctgggattacaggcacccgccaccgcgcctggctaatttttgtatttttagtagagacagggtttcagcatattggccaggctgacctcaggcgatctgcttgcctaggcctccgaaagtgctgggattacaagcatgagccactgcacccagcctaatagtttatttttaatggagtgaaagcaggactctaatgagtgaacagccaccccactaggtgtctgattcttggtctaggcagcagaatggccaaacagattcatgcagcaatgctttctggacctcagaacaaggaaaatatttacaataaggaccttcattatgagaatgtgcaagagtatctcctgctcctctctggcagctgatattttggattatcaacatcagttcagctgaagtttatttagcatttattttattctaggcactgggctaggctggggatagaaagatacaccagtctttctcaaaacagtgtgaagcttggcagggaaaattgcagagataatgtgatgacagtggctgtgggtgtgataaagggtaccaccctctgatgggacagaggagtgaaccagggcagaaagtacattgaccttaacctcaacctctgagtacaaggaggagtccaagaggaaaatatgagggaaaagatgttctattctcacaccctctcatcttcattattttctctgcacccatttattttgtgccctcaggagcagtggttggtgtgcattctcgggtcgcacacacagggtttggaatcaaaagacgagggttggagttcaggaattttcctttttcacaaacatctagggcccttttcagatggtccatggaccacacagagagacgagctgggtgtttcttaatgaggagcatgatgagaatataatgactgggagagaattgccagttccaccaagcacaccctggagactctcggaggggaacattatccctttatcctcagacaggtatcccaaatcttatcagatgaaatgctgctgtttattttcagagagctttgagctctccagggatgggcaactgataataaaatcctggaattaactggggagcgggggaagcccacagctgaaccacactttctgggagtcttggtaaaccttagaaaggagctttgggcaaaggtaggggactccaacagatgttgcagagacatctgtacttttggacccacgggttatttttggcattggcatcttagtagagtggaaagaatacacatgtcctttggtgtcagataaatcttgtcctaaatcccagctcctctattaacttgaagggcaagtcaactgagcatctgcctgagcctcagtttcctcagcactagaatgggtacaagaatgcctgccttagcaattggcttgacatatgcttgcaaagtgcctggtcataggtggtgctcagaacagttggttcctgtgaacacataaagacccttaagccgggtgggagtcaggagggagggaaggaacaggctatgggccagatttgaccctagagctgtaagtttgctgatccctggtctagcataataaatcactgataaatttgccttaaatataaatagtcaaagccttggccagttgtggtggttcatgcctgtaatcccaggagtctgaggccagcctaggcagcattgtgagactccaactatacaaaaatattttaaaatttttaaaaaattggccgagtgtggtgatgcatgcctgtagtcctatctactcagaaggttgaacccaggaggtcgaactgtgctgctgtattccagcctgggtgacagaggaagaccctgtctctaaaaaataaaataaaatatctcggacactaatttgctctaaaggctgagaataccgatactttcccactggaccccacaggtaggtcatatttcccagcttcccttgaagctagagaggccacgtgtctgagtcctggtcagtgatgttggggaagtgaatgtggaactgctaagcctggagccggagcaaccttcctcctgcagtacccggaggatggtggaactcttacacggaaggatatgcgttcctggaggcatgcgaggcaggcaggagccccacagctcccctccacaccaattcatctgcacaggaatatgggattgcgaataagaaataagctttattgtgttacacaactgaggcttgggagactgcttgttgcagctcttcctgttaggctgctctgactcatacagcatcttagtgtgcttcctggcagggcacaggtcacgaggctgtgggcacctgacaaaatggtatggaggctggtgcctctaagagcatcctcccctgggtctggtacacaaggtctctgtgcctccaccccatgtctccgcctatagaggcttttccctctcttagcccccggggctcccactctccaggcagtcaccctgggacatgtgccacagctgtcctccccttgggctcttgtgtgcgctgtttcctttcctttcctcaaaatacctgtctttcctcggccgggcacggtggctcacgcctgtaatcccagcactttgggaggccaaggtgggcagatcacgaggtcaggagatcgagaccatcctggctaacacggtgaaaccccgtctctactaaaaaaaaaaaaaaatacaaaaacaattagccaggcatggtggcggctgcctgtagtcccagctaccggggaggttgaggcaggagaatggcgtgaacccgggaggcggagcttgcagtaagctgagatcgcaccactgcactccagcctgggcgacagttagagactctgtctcaaaacaacaacaacaacaacaacaacaaaaaacttgtcttttctcatgaggctgttccgttttgtacctttacacacaccaattccagacacaccttccccactgctgcctctacccagggctgggcttggcttcctttatctgtctagctgccttccctctactcatcttgtgctcactgcctcatcagactctgaggtgctggagggccggcgctctgccctcctagcctaacgcagtgtttggcaaatatttgtgattaaattaatgaatgaaagagaaagggattccttggcacgtgtaatgctttttatgaccatggctaggccaaccctggaattggctttcagagattaacccaggccctctaccgatggtgttgcttcctgttgactcctaacgccgattccttgcctcatcctccgccttcatcatcttctgccttgcatccatgaggtgtgatcagtcacagggtgaacaatcatcggcggccatggccgcccgcagacctctggagcaggaggaaaacaggcctccactttacttgactcctatctgctcatggggtctgtgtccgcagaatcctaatgtagggggtgtgacctggaacatgaaagggacaggaaggaatagaactgctggctctttggggccataattttaatgtgctgacgtccattgtgcagaagagacttttcccagctgtcctggcaccacacagtagtctgtgtcatcaatcgagcctgctccagggcagctgggcacaggtgcaaggcccggtgcagggaggagcactgaagggcctgggctgagcagctcccagggtgcagcgctgtccccttcatggctatggagtggggagcaaacaccacctaccttccaaaacaaactaaaacctttccaacattctgcctcggttttttccccaccccctcttaaatcataaccaagaaaggacatgtatttatattctgtgaggaaaaagcaaagttttaatggtctttggtgtggctttttaaaatgataaatgactgttgagcttgtcaaatcatggctaatactgttttatctgggtgagatcagtaacagctttttaagggttaccatgctgttatttctcttcgtgttggtaataaagcaaaataactctgtggctggtacttgtgtgataatagagaaaattagtgaggggtgaagtttaggggcttagaagaaggtgaataaataaatgtaatttaaacaattctggggcaggcatccatcccaccttccttaacatctgctctagattctagtgccctgtagagtccaaggaggggagctgtccttgactgtgagcatagtggaaaccactggatggaggaagggtaggcacaagcctggacctgagaaacaaaggcacctattctgctttcaaattcagtcctgggagagcaggtgacctctgtcataggactgctatagggcaccagcagtggagataaccaagtgataagcaagtctttggctttccattaacagagaccactggtcccaacatggaaaagcgtcccagaagcctttcccagttgctgcgtccacctagcatacacacaggatggctctgatttcatgacacttctgccaacgggaagctttgggcaatcgacagcacaaaatgaaagacaacatgtgtgcttccaggctagtggggagggacgccgtaagctgatgcttctgagacagaaaccgaagggacaaggtgccaggagctcctatactaaaaactgtttgtagcacatttatggggattaagtggttgtggtgaaaacagcctgcacgtttaatattgatgacgttttaaaaagcaatgcactgcaaatatctgttgagaaatgaatgccctttaattgttcacaaatgagtatcttaaagaaaatacccatagagaagtcacctggatagttaaaaacattaactacccagggtagaggagaaaattagattgaaactaattcagattaatttggcatcaactttaaagaaatcaaataaataaatgtccaagttattagagtgtaagaaagtgttccaggaaccttttccacagaggttcttgatcttcctgtgtggcagttagtgggaaattatgtgacaaatttatcacatctcttgtcagctcctagcatccttgtaagattgctacaattccctatccagtctgtatctaatagagcatacataatgatttttattttaattccaaccaagttgaatacattataaaaccaaataaaattgtctatttatcaactccagagatcattagataataacaatttattgttgcaaagttttgtgaaaaaattatcaaattttagatccaaactggtttagtgtagtgtattgctaaggcaaataggtggctaagaacgtagacttcctagttttgtggatgcaagcaaatggctgaatttgagggtttttgagctctggactagcccagtcctgagatttcctggttttagtggggagacttaggtaaataaggcattcaaagtgcttgtcctgttgtctgagatatgtaagtagctggtgatgatcatagatgctgacaacagatactgagaaagttgactcagagaggcagcttgggcctggaaattcaggcaaatggcaagcccctagggctgagggaagcgtgctttaggcagggcaccaagattccaatactaaggacgtggccagggtccactctctggagctggtataaactggagcttagtaatgaaaactagggaaaggcccagggtagaggtcggagctaccagatttggcaagatttctcaagcccccgtggattcagttgtgatatagagtgaacaatccccccatccgggtggctctggggacaaatgagaaacatgcaggaaccgctgagcatgtggaaggtcctgggcacatgccagttccctccttcttgctctttgtcactaactgtggtgcccaagtcctccccttgcctggtctagtctggggttgctggatgtggactcactcagaaccccacacttctttccaattgcagtctgagcagagggacgctgggacagagccctggactttcctaaaaccgggaaatcccaggacaggactagaccagagctcagtgagggctgaagactcagctgtggggctgcagggcttgaactccatcgcatgttttctcatgagcctggaaatccttcaataaatatgaacaacaaaagtgccccaaaggtgctgtgttagaaaggcacccatctggctcatgcctgtattctagttgagtcaataaggcaaatttaccttatgttgtttgtgggcagttgcaatggtcctataggcaatgaaaacatcagatcacagtttgcttgtgttgctgtgagaatctgacctcaaagaaaatactcccccaacccaaatccaaatctatgaaatgaagacacagcagttgtggtggggcactaattatgaatgttgagcttcaggttagggttgaagtaaatttgggctaaagtgctttatatattgagcattccaattctcataacattgaatagacactaaaacttaatttacgaaagtagtttaatagccaatggggacaaaaatttattcttacagttatcaatactcacttcctgcttagaaacttgttttcttccttaattaccagaaaacagagtacatttaataatcaatcatactaaatttaaaattagggaaagagagcttatggtgttcagcaaaaagggaaatatcaaaaaagacatcttagcatctggattttttggccaaaaggagagcctgcagtttaattaatggtctacatcaaacaggatattttatgaagcaatagaaagctgtaattaaatctagccttctggatttgatacatcacaaaaccaggagataaaaacaacagaaaattggtgatagggttgaaactcaaccttgttcaagaagcattcacaatggaagctgttgacctatattctatatactttacatttcaacagatagtctatgtttgaacagtgatttttttatatgtgaagattttctgtaaagaactgtattaacatttgtaaacagagtccaagaaaaattaattatgactccttttttggatttcacattggtacagcatgacctttaactcattatcttattacaactattagaagaatagtaaaaacatatttcatatacttttcaaatatttataatcatagtattctttttataatagttttcaattcttatttgatagcatgctatacaattatttttccttactaaactttcatagacatttcacttaaatgatttttctttttttaaaccatataacctgtcaaagtataaaagacacatacaagcggccgggcacagtggctcatgcctgtaatcacagcacttcgggaggccaaggtgggtggatcacctgaggtcaggagttcgagaccagcctggataacatggtgaaaccccatctctactaaatatacaaaaattagccagccgtgatggcaggctcctgtaatcccagctacctgggaggctgaggcaggaaaatcacttgaacccaggaggtggaggttgcagtgagccaaaatcacgccattgcacttcagcctgggcaacaagagcaagactctgtcttaaaaaaaggatatatacaaggtacaaattatttgaattagtctatattgatctcccttttcattctctcagtttctctttattaaacctctttctcttaagaacataaacaaacaacaactactagaaaagctttaaagtccagcattagtcaaattgaagtactgtcctgacccagaaagcattatccaagaaagtgcatttcaaggccacctttcctaaaccagtctcaggaaggtccacgctctatttccctttgcaaaagcatttaagttaatgctgatatatgatttgtatttattaactcccccccaccccaaccaatgtagtcttttttaccttaacctattattgacttggtgacaagatatagagtgatcatggagctgggatccatgttcctatgtgtcatcagttagaagaatgaagaggccaagggagtcattctaatatttatatggaagaataaaggcccagaagcaggtaaatcattctaaaagaaggggtagggagtaagaggagggcttcctctaccagaaattaaagtgaaattccaagccaaagtaatcagaacagcatggtattggaacaagaacagacaagtggaccagtgaaaagatcagagcacttggagactgactccacccaggggaacatcatatacaataaggatggcctcataactccatggggagaggatggattgttggctaactgatgttgagaaaagtaatgactcatcagatatggagaatgatcaatctgcctatcaccatgtacaaaggtggactcagggtgaattgaagacttaaatgtcaaagtaaaaggtaatattaacaaaggaacatacagagaaacatgacaaggtcagggaaagactttttaaagaaaagtataagccaaaaatggattatctagattatatcagatttctgcctcatgacagataccatcagcagagttaattaagtgatgacaatgatactgagtagagccatgctactctaatcaagggtcgttatgggagttatacaatgcatattgtacaatgcatattgaagccaaaggcttcaataatgttcactgtgtgctcaacactgttgtagacacttgacatgtgttcaactcacttaatccctacccacttcatgaggcatgtgccatttttgtagattttttttagaggaggaaaggaaggtacagagagattagataatttgtccagaatctggagctgcaggataggagacttggaaaagaaattgcaatgtctgaaccaacaagtaattaactgacatctctagaacaggaaaagaagttctgtaaatcaacaagtaaaagatagcaagtcttgcagaggaatcagcaaaggatacagatagttttcatgagaagaaacttctccaaaaggcgactaagcacatgaagaaatgtgtgtgtctggggagctttccccttctaatactaggtcagtttctgtgtggtaacttgagaatacaatgaaacatcgtttaaagtaacctgtggcatgttactggtaccttgcaggctaaatacatcctaagtgtgctaatactgcagataccttgatccccttaggacagttgacccaagccttcccaggagctgtttacctgggggtgtcacctggctgcaaaacagggatgaacaatgcacagatttcaggggtcacccagggatgctgcgacctcagaacacgtcacatggaagatgcctctcagtttccatctgaaaagcctggtattaatccaataattcaatcaccaaatttagtagttgcattccccagattttcttttgggcaatccacagtttgtagaatgcttttctgtgttcccaggttcagaatttcagggattgcttctgtcttcaaggagcacttcagcttcctggttaggaagagaaggtagcagacgtgtagaaaaccctctgcgagactctgctctggcagttttatgggtaggcgcaggcctgcctgaaaccaaacgctttcctttttccttcaccctttcccgttgttgtctttttggttcctttggtatggtagtgtctccatgtgagtttccagggaagcagaggctgaagcgagggcgtgggagctgtttcatgcattagactctctggggagcagccacagggaggggactgagggacaccgcgggatgtccagagaggccggtccccagcgggtgggggcgggccagacggtgcggagggggagaggaggaagactgtgtccagggcatgagccttctggcattgtgggttcccccacgctccagcgccggaggggacccaggcggcaccagcgcgttaggctgcccccgggagcagtgctgcaggagccccgagctcgaggtggtgccggcagccgcgcgctcagagctccaacagggctgggccgcggccaaggagccaggagggtgcacgagaacgtcccatccagggacccacggatacttcgttcctgccagggtctcctttgggccgacagagttgtctctaattgagtgcaggcagccccggagcccgccgagagctgaggctgggctgggctcccggggactcgggcagctatttcaggcagacgcggggcatcgcgccccaatgcgcacagtgttgccgaggggcagaaagagagggtctcagcctaagccaggtggaaggtccgagaagaggctggaagggggcgatggtccagcaggagctgggatgaacaagggctggaagagcaggtgggagttccaggtgagggaagtgacctggaagcggagcagaggtcgcagctggcccctgctgggcctagtctcgcttctgtcgtgaccccttgtctaggaagatcccatcgcgaaggcctcatgagtcccacgtgaccggcacgcctgtgaggcaggaagctcagagtccaggccccgtgcccctctttgggggctagagcttctcccaggtgcaggcagcctgttcacaagagcggggccctcctgggtgaggcctcaggctccggttgtcacccctggcaggcgtgtgcccagcttagatccacctgcagctggctctgaacgctggatggggctgggttggggttttcctgtggcctggggtgagtcccagcaagccaccaccatccttgcccactccccacaggacagagcatagaccctgttcctaatcctcccaggccacactccgcagaatgctgcttctctgtttccaagcgcctgcatgtcttgcatcccctgccaccatcttttgtccgcctgaaggggcagccccataaaccttacttagacatgtccctgcccccagcacaggggctgccttcaagaccgcctttgtctgtggggtttttccttgatgactgttcagcatgccggccccactttcttctgcttgcatctggggttctcccaggctgaccagctgtccccacttgctgggagtgaaggggtttcctgggatacgaactttcagtattaaaaccaggagagtcccagacaaactgggacaattggtcaccctaactgacccacaagtagaaaaatgattcataaaatgtataataatgaaaagctgtgaattatttgcatgtataatagagggagaacatggttaaataaatgtggtgtagccatttgaaatagctactacagggatgaggaaacaatacctataaaataacacagaaatgttgatgacatattaaggaagaagaagtctgggttatacatttgtgcaaactgtatgatttcaaccatatttgtacactccatgtggtagatgggttgttggaaggtaaagactggctgcataggggccctaaaggtgagagcttttcaatactgctgcttacgctttcttcttaactaattcagatattattttgccaaatgcagagccaggaggtgtgaagcagccttcatggtggcaccacatgtgacacagatgagtagatgggtgcagcgttgacaataactgcagtcatattgttcatcttgatgggatttctgagtaacaagtcaaagatgcagaaattaaagtatacaaattggattgggttttcagtaaaggaaaaggcaccaaatcgtttctcagtaaggcacagtaaagggaggctatcaaaacctattgttagtaaaagtacctattgatgatctcttaagcaaaattgttacctagttgtccacgtgaaggaaaggactctctgaaaagagggggctgtttagagaagaatcaagcctctggcaccagaggggactccccagttaggaaactgttgtaacaaaaggactccatcgctcagagcaggatcagatgataaatcaggctgaaatggtataagaaacctacaattaccttcctccatatgaataattgaaaaggcaatggcttcatttaggttgtaatctgacttacagcttttaatagctatgctctgttaaagtgtacacattatcttttaccttattgagtttatattgctttatatcaatataatgatttttttactatgtaaaaaaatacacttacatagtaagtatatttacatagtaaaatcaataattttttttactgtgtagcaaaatacacttacttgattcatgaaccattaagtttttaaaatctgtgtgagataatgtattctctgggtttattttgactactgaaaatttctagtcacaagacattatagctagatacattccaggtgtttttaaatgttaaactgtcttcagcatcagaaatggcctcatgaggtaagtcattattttcccctattttcaaggatggaattgaggctgggagcagttttgtgactcacccaagttaagatgtcatgaaaaattgtttccttgcaaggtctgccacaccctaaaccaatgtcctgtcccaatccacctgacttgagggctgttagattttagatgtcagaatcacatggagggcttgtggaaccagactgctgggcctcactggagtttctgatccaatgggtctggggtagggcctgaaaatttgcatttctaacacttgcagttcccagatgttgccgttgtcacaggtcttgggaccacactttgaggatcactggtctagaaggaggcttgtattagctagtgatttttttcattgtgatgatgatggctaaccttacacttgaacaaaaaatgtggctttctttaaagaaaatcatcagaagttcagttctttaaaattcagctactactcactgacatggtgtattaccctgttctcatgctgctaataaagacatacctgagactgggtaatttataaaggaaagaggtttaattgactcacagtacagaatgtttggggaggcctcaggaagcttacgatcacggtggaaggggaggtaaacacttccttcttcacatggaggcagcaaggagaagtgctgaacaaaagggggaaaagtctcttagacaaccacgagattccatgagagctccctcactatcatgagaacagcagcatggggtaaccacccctgtgacacaattacctcccaccaggttcccccatgacacatggggattatgagaactacaattcaagataggatttgggtgggaacacagccaaaccatatcattccactcctgtcccctcccaaatctcatgtcctcccatctcaaaacacaatcatgccttcccaacagtcccccaaattcttaacttattccagcattaactaaaaagtccaagtccaaagtctcatctcagacaaggcaagtcccttcaacctatgggcctgtaaaaccaaaagtaatttagttacttcctagatacaatggggatacaggcattgggtaaatacagccattccaaatgggaaaaattggccaaaataaaggtgctacaagccacatccaggttacactgatgcaagagatgggctcccacagccttggacggctctgcccttgtggctgtgcaggatacagccccgtcccagctgccttcatggctggcattgagtgtgtggcttttctaggcacacggtgcaagctgtcagtgaatctaccattctgggttctggaggacagtggccctcttatcacagctccaccaggaagtaccctagtagggactctgtctgagggctctgaccccacatttcccttccacactgccctagcagaggttcttcatgagggctgtgccccacaatacacatctgcctggacatccatgtgtttccatacatcctttgaaatctagatggaggttctcgaacctcaattcttgtcctctgccaacccccgggaccaacaccacatgaaagctgccaatgcttagggctgagacctcctcagcctggattccattgtccactatcagcattttggtcaaagtaattcaacaagtctctaggaacttccaaactttcccacatcttcctgtcttttgagccctccaagtctctaggaagttccaaacttttccacattttcctgtcttcttctgagccctccaagctgtttcaatctctgcttgttacctagttccaaagttgcttccacatttttgggtatccttgtagcagcaccccattctctggagtaccaatttactatattagtccattcttatgctgctaataaagatataaccaatactgggtaatatataaaggaaacaggtttaattgactcatggttctgcagggctagggaggcctcaggaaacttacaatcatggtagaaggggaagtaaacatgtccttcatcacatggtggcagcaagaagtgctgagcagaagggggaaaagctccttagaaaaccatcagatctcgtgataactcattcactattatgagaagagcagcatggaggtaactgcccccatgattcaattaccttccgccaagttcatggggattatgggaactagaattcaagatgagatttgggtggacacagcaaaaccatatcacgtggtttcttttactgaagcattttcccctccactgcaacttgcatacctgtattgcaactttcaagtgctataaaaatagcacatacccccatcaggcattgatagcacctccatgtgcagattttgaaggtaactgaaacttcctctgccctgacaggccatgctgctgctcagtgtcttccgctgctgtctccaagtggtgttcttgtcatcctggaagccctgccttgggccatgcctgaaatgcactaatatgctcatttgtgaggtagtacagagtggtcagggcatccaggcacatcatggatgatggggcactgttacgggtgctgggacactgccatgaacaagaggcccaggtccctcccctgtacctctgtagtcggtgccagcctcatgccattcagctaccttcacccataatgtccctgcaccctctctgttgagtctacctcctcttcctcctccatccagcctaggtctctggcccagttcttcagcccatatcttccagtggccccactgcccttcccactggcctgcctgggctgagagcactgctggagacaccactcccagtacatgctgctctgtcctctctagttcccggtgtcctgatattgcctgatagtcatgttgagtgtcctgacgtccactctggctctccagactctgttctctgccaacttccaacctgcccctccccactcataccctctccccctactcttagcaaaccactcactctccacttccctgagaaaatagaaaacttggaataatacgcttcttcgatgtcctgcaccaggacaatcaacttctctatctctgctcccatagcttccttctaaccccacagcagaagagttataatactgccccctgttgaaagcttattttccacctgtgctctggtccccacccctcctggcttctcaggtgatttccacctgcctaccacacccttgatgcctgtcttcagtctctccttcactggtgttctttgcttgtccacccttaaacctgcttaagttcacctgtccacgcttaaacctgcttaagttcacctgctatttaaagcagtgtcccttatccctgtgttcctctgcagctgagcacctgtaattccctgtcataggcacattcatcatctctacattctcaccttccactcactcctcaacctagtgtggtctccatcatccccgccacaccacagaatctgtgcttcccagggtctctgatgaccttgaggttgctgttcactcacctcctcacccctggtcttatgtatcacaccaaagtgtcagctgtgcaaaagccaggaataggattcctactctgtagggacccctgtggctggcgcaggtagctgccatagtcaccggttcatatttcaccgttctgctcatttcgtgctccctggtacatctcttatttcactgcttgcatattctgtccaagctctgataaagccaaacacatttaacagatggacacaactggccatgcttcaggcttctgattcacccatacttccaggagccatgaatctgaaatctggagctggtctgtagaaagcattctatttagtgaaatgttgaaggaattttaggtcaactatcatgtatttcattgtctccaatgccataatttccaagtgcctacttgaacagtagatactttcaggggattttcatatcaatgattgcattttgccattgttctttattttttcagtaatgctccatctgtaaacaacccaaatttcaaagcctgacatagtgtattgacaaattattcctggatgcctgtttttatcaggccaaaatccatagttattctaaaatggatatcatacaaataatgctgctgtagcatgctaacattttccagactattatgaaaatgtttagctcatttacaagtcatgggaaacaatatcggaatttgctactgtgtagaaccattactatagcacttccagtgatgcctgcttgtgatctttattgcccactttatagttctgtattttcatctccattttttaatccttaaatatacaaagtaaaatgtaaatatcttttttgtgggtcttttagaattattttttagcattagcacttgtaaatttactggaagtttcaacaatttgtatttcatgttagataaaagtaaggaagctttcaaagaaaagtctaacaaatataacacatgtacccacctccccaaatgaaaaaatgttagcattttgttgtattttatttagatttttttgcagagaaataagccattatacatcaagttgaaattctacttactattgttgtgcgacaaattatctcaaaacgtagtgatttaaaacaaccatttttttttactcacagattatgagagtcatgaatgccgaacaggcacaagaaggagtgtttctttctgctccacaaagtgcagcttcagataggaaggcttagaggttgggatgacccaatggccagggcaggagccgtcagggtgtcttaatttactgatttggcagagagtagtgctgtcagttgggccctcagttagctgcaacaactgcacatgatacctctgtgtagcttgggcttcctcctagcatggtggctgcagacgtcttacaaagtggttcagagccccacaggtgtgttctataatagctcacaaggcagaagctgcattgcctttcaggacctagcctcagaagtcacactgcctcatttctgctgtattctgtcagttccaggagagtcacaagcccttcctggattcaagggaaagggaattagatgccatttcctgatgaaggtggggcaaagttttgaaagagcatgtgtgggggactcatgccatctttggatagtcccagtctccctcctcaaggacatcaactcttgtagtttgtctccagtccaaacatgtgtgggcactttccctgcatatgtgggtatccacgtgaatgtctcttcattgcttctccatctttctaaaaagtaatcacagagagtcatgaaatccagtgttagaaccaggcattgttaatgctcttatgttattattattcaagctgggctaggggaatgtggcattaataaaacaatatgtacattggatttagttatactatactctctattctaatactttgtaatcattagtaataaatagtagcatattacttcatgtaggattttgcaaagcttaaactattttcacatttgttccttcagtccaggcttcctaaaaataaaatataataagagtcttgagacttaagttcatattttgcatgaatcagataaaataatagatagtaaaatgctttgtaaactgtgcagagctattaagttgccccttatcattaagtttttaagtctgattttctgatattgtaatcctgtcctaggcttcctctcatttctttgctgtaatctttgcttctacctcatttcttgtgtccaaatacaatcagttattcaaggcctggctcaaatgctgcctagaagcctttcctgacacagacctcccagcttacctcctctccctcctctgtgcttttatcgcattctgtgtttctctggtagattatcttttgtattctatttcacgtatcacatcactggggctcacctggaagcttctagcaaaccaaaccccagtctctgtagcccttagtgcacaacaacatgcctcccacccaggagcaggctgtgaaagtctttttattttttaattattatactttaagttctgggatatgtgtgcagaacatgcaggtctgttacataggtatacatgtgccatggtggtttgctgcacccatcaacctgtcacttacattaggtatttctcctaatgctatccctcccctagtaccctaccccctgacaggccctggtgtgtgatgttcccctccctatgtccatgtgttctcattgttcagctcccacttatgagtgagaacatgaggtgtttggttttctgttcccatgttagtttgctgagaatgatggcttccagcttcatccatgtccctgcaaaggacatgaactcatccttttttatggctgcatagtattccatggtgtatatgtgccacattttctttatccagtctatcattgatggacatttgggttggttcccaagtctttgccattgtgaatagtgccacaataaacatacatgtgcagcgtgtttctagtagagtgatttataatcctttgggtatgtacccagtagtgagattgctgggtcaagtggtatttctggttttagatccttgaggaatggctacactgtcttccacaatggtggaagacaatcccaccaacagtgtaaatgcgttcctatttctccacatcctctccagcatctgttgtttcctgcctttttaatgattgccattctaactggcttgatatggtatctcattgtggttttgatttgcatttctctaatgaccagtgatgacaagcttttttttcatgtgtttgttggccggataaatgtcttcttttgagaagtgtctgttcatatcctttgcccactttttgatggggttgtgtttttcttgtaaatttgtttaagtttgtttacaagtgggatgacagtgtaaagtctcccactattattgtgtgggagtctgaatctctttgtaggtctcgaagaacttgctttatgaatctgggtactcttgtattgtgtacatatatatttaggatagttagctctttttgttgcattgatcttttactattatgtaatgcccttctttgtcttttttgatctttgttggtttaaagtctgttatatgaaactctgggatgaaaattattgtcattcagaatgtcgaatactggcccccactctcttctggattgtagggtttctacagagagatccactgttggtctgatggacttccctttgtgggtaacctgacctttctctctggctgccctttacattttttccttcatttcaaccttggtgaatctgacgattatgtatcttggggttgctcttctcaaggagtttctttgtggtgttctgaatttcctgaatttgaatgttggcctgttgtcctgggttggggaagttctcctggataatatcctgaagagtgttttccaacttggatccattctacccatcactttcaggtacaccaatcaaacgtaggtttggccttttcatatagtctcatatttcttggaggctttgtttgttccttttcatgcttttttttgctgatcttgtcttcatgatttatttcattaagttgatattcaatctctgatatcttttcttctggttgattgattcggctattgatacttgtgtatgcttcacaaagttctcatgctgtgttttttagctccatcaggtcatttatgttcttctctaaactggttattctagttagcaattcatataaccttttttcaaggttcttagcttccttgcattgggttagaacatgctcctttagctcagaggagtttgttattactcaccttctgaagcctacttctgtcaattcatcaaagtaattctaattctccatctagttttgttcccttgctggcaaggacttgtgatcccttggcgagaagcagtgtcctggcttttggaatttttagcatttttgccctggtttttcctcatcttcgtggatttatctacctttgatctttgatactggtgaccttcagatggcgtttctgtgtagatgtcctttttgttgatgttgatgctattcctttatgtttgttagttttccttctaacagtcaggcccctctgctgcaagtctgctggagtttgttggaggtccactttagaccttgtttgcctgggtatcaccagtggaggctgcagaacagcaaagatttctacctgtttcttcctctggaagctttgtctcagaggggcactcaccagatgtgagccagagctcttttgtatgaggtgtttgtcaacccttgctgggaggtgtctcccagtcaggaggcatggtggttagggaatcacttgaggaggcagtctgtcccttagcagaacttgagtgctgtgctgggagatctgctgctatgttcagagctggcaggcaggaacgtttaagtctactgaagctgtgcccacagctgccccttcctccaggtgctctgtcccagggagatgggagttttatctataagcccctgactggggcttctgcctttctttcagagaggccctgcccagagaggaggaatctagagaggcagtctggctacagtggctttaccaagctttggtgagctctgtccagttcaaacttcccagaggctttgtttacacggtgagggggaaaaccacctactcaagcctcagtaatgatggacacccctcccccaccaagctcgagtgtcccaggtcgatttcagactgctgtgccggcagcaagattttcaagccagtggatcttagcttgctgggctccataggggtgggattcactgagttaaaccacttggctccctggcttcagccccctttccaggggagagaacagttctgttttgctggaatttgaagcaccactggggtatgaaaaaaactcctgcagctagctcagtgtctgcccaaatggctgcccagttttgtgcttgaaacccagggtcctggtggtataggcacccgagggaatctcctgatctgcacgttgtgaagaccatgggaaaagtgtagtatctgggccagaatgcatggttcctcacaggatggtttctcaaggcttctcttggctaggggagggagttcctcaaccccttgcacttcctgggtgaggcaatgtcccaccctgcttcagctcaccctctgtgggctgcacccactgtctaactagtcccagtgagatgagctggtgcctcagttggaaatgcagaaatcacccaccttctgcatccacctcgccgggagctgcagaccagagctgttcctattcagccatcttgccacctgtgaaagtcttttgaatggatgaatgaataaatgaatgaatgaactgttttttcactggcttcatatgctcatgcctcactaaggttgtagaaaatacaggatcttgaatactttaaagtggcaatgtcttgtgagacagacatcatccctgctgtcccaaaattacccagcgtgtgtggagaggcccctggctttggagatggggggcatttcccttgactcccacctcctcagcaccacagggctcatctctggggacctggctttccagttagagcagacctggatcaagaagggccacaggaaagagaaaagaaagacttggcagtgggagggcagggcgttcaggcctctccagggaagggtgccaacctctggacttagcatgatgtttttaaaagaaatgaatgtgtgtcttgtctctaccttcaacttgtaaggcaagtgcagtgccagaggctggggtatctctgctgcccaggtgcactccacagagacagagcaagagcacgtgcagactgtgctgtccaactcgcatagaaggctgacaaccacttccgtcctaatggggaaagtgaagttggcaaggcagaaatgaccacatgctcacccaaaaccttggtagcagcatgtctttccagaaattttgcaagccagatctggaaatttgatggctttgctcctctttttcctgaaagaaatggaaggaatcaggctcatgaaaagccagatttcacctccatgcacacaaccaggacccttgccttcctgaaatgccccacaggctttcacagaatagatgcagtcttagcagtgggcagcttgcttgttgtctggcctaagtctgagccaagataaagacatataaactgccaaggagacactctattccagaaccttctcttctttgcaaagcaaacataattttaagatcaagatgcataactcttgtaatcaaaagcttacacatttcctagcccccccaccaccctccacccccaagcaatgggttctaagcaatataaactgccaaagtggtgtgtagagggaaataaaaaccagactggtaagataccaacttttttataccagtttcagaaagtgcttatgcagagcaagtgtgggacaatattcaattggcaaaaatatgatgaaagtagctcacttaagaaaggaataaagtcttgtttattgaaagaacagtgacaagtttttttaaattctgaaagtcttgcatatttcctgcactgtatatttacctaagtacaagtgttttaagaatgatctcttgagcctgcttaaatcaagaacgcatatttcagcaatttgaaaaaaattgcatttctttgttcatggctgttctcggggttgagaggtatggacccaggagtcagaggtctgagtttaggcttccacctgctaactagcttggaacctctgactaggtttttaaaccctctaagtcttgggtttctcacctgtagaatgaggataataatcatgtctacctcacagtattgttgggaggacttaatgagatgtagagaaactaatctaaagcctggctcacagaaaggtaatgtgaagtctcaaaaaaagtgaattttacaatagcaaagacatggaaccaacccaaatgcccatcagtgataaagaaaatgtggtacatatataccatagaatactatgcagccataaaaaggaacgagatcatgtcctttgcagggatatggatgaagctagaggccatcatcctcagcaaactaacacaggaacagaaaaccaaacaccacatgttctcactcataagtgggagttgaacaatgagaacacatggactcaggcaggggaacaacacacaccggggcctggttggcgtgggggcaggggggtggtgcggagaggggtgaggggagggcgaacaccaggacaaagagctaatgctcgtgggtcttaaaatatagatcatgggttgataggtgcagctaaccaccatggcacacgtatacctatgtaacaaacctgcacatgtatcccagaacttaaactaaactaaaatttttttttaagtgaattttagccactagattttagagttgaagcatgccatggtacttgtgattaggcatctacctgtgtatttattcacctgaacacctggatgtgtagaataaaaattgctcaattgttgaaggctgctgcatcgctctatgctacttgttctgttaaagtatgttcagggagccagcccagagaggcacctccttttagctctgtgaggccttaaagctgggtcaccatgaagtccctgtatatatattttttgttttaataactccaagtaaccaaatgaaaacaattatttataaaatgtattaagaagtaaatgttatcaatttgggactttgcacaactattattaatgtgacctcaggtcccatgacaaattagaatggcaatcctgggtgagaagttaattttgtcctgaagctggcagaatcaacatgcaccccagttgtgggataggcctgttctctcactccatcacatccaacacctggcaaggacagcttttggtatgctaaagaatttgggaaagatgctgttggtgtaggggcggggagactagaaacaagatctttccatgggcagagctcttcagagcttgataacaaagtttggtctgcagcctttgtcaaataattataaactctccagttgaaacagggaaatgtgggctggcgtggtggctcacacctgtaattccagcactctgggaggctgaggcaggtggatcacttgaggtcaggagttcgagaccagcctggccaatatagtgaaaccccatctctactaaaaatacgaaaattagctgggtgtggtgatatgcacctatagtcccagctactcaggaggctgaggcaggagaattgcttgaacccagaaggtggaggttgcagtgggctgagatcatgccaacacactccagctgatagagtgagactctatttcaaaaaaaaaaaaagaaagaaaaacagggaaacgtttgcaaagcttccatctccaggttgacttcagtcaataatcctattgtcttaaggctggagtggacttccttgaagctctttgcccagaaaaccttgccaaaactctcctaagattattaagagttttctcgagtctcttttcctgattccaatcaactggaaatgttatgaaaataattctgtggtcaagtctcacttactcttgctctcaaacacaatccaccaaaagaacaaaacacaaagtaaaagtggatgcaagacacaagaggaagaggggccctctgcagaggctgctaagcaaacgtaggtttggctcaaagatttttgtttctttgaatcagttcactatttttaacctgtggaaatttgttcactcattctaacagtagcttttgctttgtgaaattggtttatttagtgttatgttcctcctgtaaaccttttcattgtatgtttttctcctaatctaatggtaatcctctcagtgatatgagtaacctggtgtttgtatttaacactccttcaacattcacagaatcagaaaatatcagaacaagaaagccccagccttcaagattgtttgatttgtgttccctaattttatgtgtgaacaaaactgaggctgtgaagattctagaataatatgttgagctcaaaaatatggtagtagaattagaatatagctttctaatatttgtataaaagtcaatgatatttagaagtttaaaaaagatacacattaaactgggaaaatactaaatatttaaattctgtaggctagttttcatattctggaaaggtggattttcatatggcactggtgctggtgtttcatcaggtcagctgtctatgtcataagagggtgttcttattagaaaatgtaggctttggcaagctttaaaaggtacacgtgttactgcatgttctgacgagattttgaaaatctttggcaactgtgctttccaagtggagaaaaagggctaccataatgttgaaagacggaaattaaaatctaatcagatatggtggcccttatcctccaccttagaatttcttttcgtggaatgtctggtggacagtaatatatagaggctcctcgacttaggatgggatcacatccagatacacccattgttaaattgaaaatatcataagttgaaaaatgcatttaatatacctaatctaccagacatcatagcttagcccagcctactttaaattgctcagaatgcttacactagcctgcagtcgggcaaaattatctaacacaaagcctattttgtaataaagggttaaatagctcatgtaatttattggatatagtatcagttgtttccccttgtgatcgcatcactgactgggagttgtggctcactgccacttcccagcatatatgactagcctggaaaaagatcaaaattaaaaaacaaagtacatttctactaagtgcatatcactttcacaccattattaagtcgaaaatcgccaagttgaaccatcctaagttggggaccgtctgtagatcaggcaatccttgacattttatggcggcagattgccaaaagagattggagtctagaagtcctggaagcagccccaacaattacacagaactcagcatcatgccctcacatcaaggcaaggagggagagttgtccctatttttcctttccttgaaagggatagtgaaactcattctgaggtttttaaaaaatgtactttttaatgcttttcctctatctgttttgctctcccttgataccctgtctacatcatgatggctgtggcactgctcctgctccttacatggggtcttagaatgcacaaagctgcccaatatcactttctctgtatttgaggaaaatttctagtagcaaaagaatatcctctgacactcttttcaaaatgccaactaaatgtgatagaactactttttgcaacagtgaagtcccaaagtctctttcttcctgatgggttaatttagtaccttcctttttccaaatcaaagaaccataaaagtcagtgaaaatctgcattattattatcctgtgtgtttagttttgctttttagtacttaccattattaaacacttggcccatttttggctaattgttgtgtaattccaaatacaaaccagtcaccatggattattgcagttagagtcatcagaaagcaaggagctatgactcatgctctgttcttgccatgtttttctgatttgtatttcatttcttacctgtgctcacaagtcacatgaagatcaatacatctgaaatatatatgctgactatgtcaccagattttatttctcccatcattgaatctggacaggcttctttcaaaagtgttgagtcatttttatgtctttatcacactcttctaaagtccttaaaactcctaattatttcagaggattattgtcatataaggtatacaaaatcaattatatgaagaagttgtaatcactataattaaactgaggcttatctagacagatcctgtgtcatatttttcacataatgaggctaaaaactttgtggagaaaataatggatatttatagaatatactcaagtgtatttattttagaaggtctgttagacatttcacaaaagaatctgtaatctacagaaattaccaaattatgttacataatattttaaaaacttgttttagaataagttggaatgtgaggtttataaaacaactggaataataaactgaaattataatttaatttttatacccagtgcatttatttttcttcctatgacttgactatagtttaaaattcttataggtttagtatcccttttttgaaaacaggttttcaatgtgtttctgtaacaataatgaaaagtaaaggcatgtaatctcttttccaaaataaaagattggtttgagaattatgatcatatatacaatgtactaaatgcaaccttttatgatgatgatataaaagattcacagactcacaaatcatatgccactcaactaaaatactaagagaacataactttatctttataaaacatatacataggcataaatacatagacaaaggtctagaaaaatatgcatatcagtgtggaaatgcaagtgatgataatgatttctgttgttttcttctaatttctatagagtcactatgctatttaaattttcaaaaaattaagttatgtgcaataatctttttaaaattatttgttttttaatcagctttttaatctgatgacatagaatgataacgtagtaaatcatagtttctgaaccaaaaaacagggacacatagactgagaagtgccaagttcattttagagattaaaaatacaacggcattgggacttttctagaaaattcaagatatgtggtcacaattgaggtgttcagcttctagagcgaactgaaccagatatctgctaaggctctgatagagaacagctggaagctcagaaaggtaaacataattcaaatattggtgaatatgccagtgcagccgagaaggtgacaggatttgaaaaatgtgccaggacacaatgagaaaggccttggaataaatacaaaaagcccaaactttattctgtaaaagtggggagctactgaaggttttaatcaaagcagggataatctcatttccaaggcaaaaaaacaaaaagaggcattccttcctaataccacatgtaggagctgttcttattctatggtggattattactttagtagtatcttataaagacagtgcctgaaggtcttttatctgtacattgatgtaacacttttcaaaagcatatcagcagacacatttttggaagcatgcattctgaaacttgaacttcaaataggagttatggctgatatcatggagtaaggtaaaacagaagctgactcctctccagtattcaaggctgactgttgtctctggaactgagaatactgggaaaagtgtgagttggacactgtgcacaactctaattaacactgttctaaagggaaagtatatgaatatattgtttaaacactaatagttaaaagttatactacaatatcttcaaggtttaacacataaattgagacacacacttaattaggcctaagaaagaagtggaatgtaaactcgttgattttattttcataaatgatttattttacaactatgaagaggaataatagaggccttttattagcaaagagattattatgatgtctattttcagccatgatttttctaccaatggaaagtggaactgaaaacctacttagggtgatgtgttggggttgggatcaaagagactactataactgtcctacctctgagattccagcatcacatgtggtgacaataacacttcatttagttatatttctagtgagtccagtggaatggagaaaattttttaagttaaaaattctaaaggacatatgtataatggtaggaattatttagtcttcttaataaaataaatcatttattttagaattattttgtagtctgaaagcacatttcctataatttttaaatgtgaaagcaggcaatattatcttgtaggtattgtattatattacaagagccaaagcatgctgattcccaatttcatccagtatgacttgtcttagaaaaagaaataaaaatgcagatccatttattcacttattaactgagtacatttcctgaatccatgtgcacgttgggtgataggaagctgaggccctgctggctaaagatgtaagagaacagagcctaaaactggcacaccagctggaaattaggagggcaagtttccctagaaatgggagaaccacagataggatgaaccccaaaatctgaatataaactctttccaaatacttggctgaccaccaacttatgcaaacataagggagacttcagtaggccagcctgaaacaaagcaactgcagaagctgaaaaatttagggatttgtcagttgctgcatatcacaggagagcagagcttgtagtttgagtccaggtcagctaacttcttaccagaacaataagccaatgatcaccaggagagcacaatagaactcagaaaggctgcagtgactattatctactagctttaggccctgtcctctgatatatcaagctagtaagacatgcaaggaaaccaggaatgtgataatgcctggggctgggagattatggaaattgtcagtcacccaaaactgactctaaataggcctccatgttgaatttagtagtcaaagacttcaaagcacctattgtagatatgctcaaccaaagggcattaaaagaattaaaggaaaaattgtcttaatgaatgagtagatagggaatctcaacagagaaattacattattaaaaaaatggaaattccagagctgaacaagtacaataattgaaataacaaacttgctaaacagtctcaatagcatattggagatggcagaagcaagaataagtgaaattgacaatagatttttaaaaatggcccaatcttaagaatagaaagaaaaaaatacccacaaaaaacactatcaaggagtccagcacacaaaatccaagctctgcctgtgcattaggcttcaggacatgagggaatggcagtgaattagggctgcactatgccaggaacactcatttaccacaactggatttgtcagcagttcgagtgaactttccttaggctaaattatttcctaagtttcaagtgaaactaaatatcatttgaaagacttttaaaggcagtctgcccaccccactccaactcaccccaatttccactaacaactacttcaaatgtaattgtcagaaagagcaccccactccactgcaccccaaatcagcctccgatagatattgcatctgaccctggggggcccttcccgctttcctcttgctatgctcactacactttatttccctcaatgaaaactccttccccaccctcctaatgtgtgacttcacattctatacatagaatttctttttttttccaaagtgaatatagttttatatattttttaactacataaaaataaaacacaatcaatacaaaaagaaatggataaaacgcaaatgtgaaaacaaactcaccaatcaccaaccactcagaactttaggtcagtttaataggtgtcctcttctgacatttcagtgtccagtctgccctccttatccatggggtctgcatccacagatttatccaaccatggatagaatatatgttttaaaaagcaaaaaaaaatatataaataaaaaccaatacagtataacaactatttacatagctatgttgtattaggtattgtaaataatctagaaataatttaaagtgtatggaaggatattcataggttatatgcaaatactacatcagttttggtgggggggatggagtctcactctgtcacccaggctggagtgcagtggtgccatctcagcttactacaacatctgcctcctgtgttcaagcaattctcctgcctcagcctcctgaatagctgggattacaggcgcacaccaccaggaccagctaattcttgtatttttagtagagacggggttttcccatgttggctaggctgatctcaaactcctgaccctgtgtgatctgcccacctcggcctcccaaagtgagccaccacgcctggctcaaatactatatcattttatatcagggacttaaacatctgtggattttggtatccaaggagggtcctagaatgaatcaccagattatggtcatctcctgaggatgattgtatatacataaattcggggtgttttacaccaatggaatcatatgcccagttttataatctgattttaatttttactcagaatgtggatatcagtccaaatcaataaatatagacaataattgacttagttcagcagagaaactggaatctcacactaagggtgaggaaggcgggagacaggagcattcagggaggggcaactcacaccttagagaccaggaaagactcaggtgtttctaagccaggggagccagtggggctgaacacaagagaatcgtgcaagctttaagaggagcatccagatccccagatctccggttcctccaggcagccccaggggtctgggaggctgggtgggaaggagatctaagtgctccctccatgacctgctgctcgactcccaccttccttccaccctcagaatttctctgtgggcctcattgtgactccgctcccctccttcccacccaggggaagccatctccacatgatggactttatctgtgtgcctcatcatgtctccactcacctctttcccatgcagggaaagcaggctccgcacagtgggctttctctgggctcctcttcttgtctcacagtctcctcctctgtggcttatgctctcaggttcctggtgcatttatcaaaccagcgctccgggtcctgctcactctcctctggctgccagtgggcagctccctgcctcccagtgttcttaagttcagcagttataattttcatctccaatgattcctttttgcaggatgctcctttttcatgattaattgctctcattttgtcaatgcagcagactcttgtgtctcactgaaagcatgaattcaggtttttagttacttcttgtttcttgtgtactgttgtgcatggaatcattttctccagttctgctcttctgaagaattgtgctgagccctgctgccctgcctggccagcatcttctcttcatatctgtagatggacagagttgttcggtgtagcaggtgccctgcgttctgtcatgggagacacagtgccttgttcaattctttcacttcccagttactgtattttttctggttggtggggctcagggggagatatccaagggttcaggagctctggccacttttccttgtgagagtttgatggggctgaacaagacagttagggaaggaggtaagccctcccttcaggtgctcagcccctctgacctctgtactgtctgccccgtggcctccagtccctcaacacacacctgcacccaggaccaagcttcctcggccctactgtgcgcagctggacccatgggctcctgcacaactggaccccagaatcctccctgcagccttcctctgccttccagccgacgcaggagctgttagtgaaggggaacagaaaaatggatccataaatccttgcttacctccctcgcaggctctcttgcctgggacagatgacatggctcctgcctgctctgctctctgtcaaaacttccccaagtattgagtctgtgggcatgaccattctctagtttctatctgtggcttggaatttgtgttaatgtattggttgggtcctttcaattggaatggggagagagaggaatccataaatctgtgactcagtgacctttaggaattggaagccaaggtctatttcttatgtcttattttgttattgtgcatagcagtgtctctcaactgtttgaatccaggcccctggagatgaatgtggactgtcagtggtgtggctttttccacctgcaccttctgcagagcagggtgcaccttgagcaccttgagcacgggcaccttctgcctgtgcacctgtgcacatgaaggagcctcttggggtcttgctgcctttggggcttttgctttcgcctccagctccttatacttctgcccatatatctcactctctgttttccagcctgtcttttctgttttctgaagtctgccctttccccttcatggttaggtgaaggaaggggaaatagaaggaaatatttgaagaggaaaggttgtggcactgcagggtatttgtgactttgtccccaattgcattcttcaagtgcagttcttggtttttagggtatctctttgagtgagattatctttaagtcttttgtccttattctgagaaaatcttataaagtttccctatatcttttcatagcctggatttctcagaaaacacatggagaagcaaagcttctgctaggttgctgtgggaaggggaagtataggcccaggcgggcagagtgagggaaggggagtggggcagagtgggcaggaaagtggacacatgtgggtccccacagagccctccagatcccatgcatagagcagctgctgccaccagcagcacagacatcttccaagggctggcagagccagggcacctgctctccaagaagggcagaggtcggcaggcatgcacagcacttaccccaaggcatggtgtcattattgctgttgctttggagcctcttatgggactggaatctgtggagaggatggagagagtaatgcatctggaatggacctcctggctccttcctatttctcatcttgatggtcacactgcacacttcccaggccgcttggccacctcactgtcatgcagccaccaggacagccagatccctgctccaagcactgcaggaggagtcctcctccagcagggtacaacctggcactgcagctgctgtgactcatgcgcatggcatcaaggcagccatgcccaggcccagctcttacctctggggaagatagacagctgacaggtcaggacacagactttggtggcaactgcccgagcacccacatagcaagttgccgaggccaggggcatgcagagccaggaaaacctgggaagatgcataaacaggcccagcccaactttgctttctgtaggtttcaggttggcagaggaaagcaaataagtgaagaaagagtggtgggggaggagcattggacagagggaaggaggccaaagacaaactcttctcactgcctgtaagtgagcaggagtctgacccccggcttttctcccagagcctgcttctggctttacagttatcactgccacactcatgagcagtatttccaatactgcacagcaagaggggctcccaaggaaatacatgtagacccaatctcagcttaggcaaaaagatttctttgctacaggatttgtcagaaccttaaaatgtcaatgtattggaatgtctgcattcagaagggctatccgagacattatactaatgtatgatggtctccactcatgtttttggaggcctcatgggagtagtgtttataggcgtcaccttaggaaatgctgccctagacttttctgactgtttgccagccccatcaggctttggatctcgtatcttcagaaatgctgtcagtattttccaaggtaaaaactagtaaagaaaaccaaacaaaaatccagaaatgtgctcagttaatagttctcctagtatcttcccagaatagcatgtgcatttcacccttgtcctggagataatgtctattacctggattataatctggaaatgaattctaactccttttattatgggcatgaccttgtttctgtactcccagcctccaaccagcaagaaaatgggggtctcagtcccacagccacaaggactgggattctgtaaatgccccagatgagctgggaggcagattcctccccagagcctgcagatggagttcagcctgcccctgacaccttgcgtgggagacactaacagggagaagagctgagcctcccccagccttctgacctacagcaccatgtgatcatgaatatgtgttattctaagccactaagttggtggtaatttgttatatagcaatagaaaacagatgcattaagtttgtctacaatgcccatgcctgcagtcatattctttggcttcctagaaggtgaccagcaatacatgtcagagtgtacaattagaaaaaaaaatacaaacaatacattatgttgtggttctcaaacgtgagtgtgcatcagaatcccctggagaagtgtattcaaacacagatttctgggacccacccctagaaattttgactcagtgtgcccgaggtggatcccaagaatttgcatttctggtaagttcccagatgatgctgttgctgctagaccaggaaccacactttgagaacactgtgttaagtcatgagctctccttttgaaaataatgatttactaagttgctgtatgtttctcaatctctactttaattgcttattttatgataaaaaatatttctataaaatgctcatagaaatgcctcttattgtgagtccagtccccattaagttgcctgtctacattgtgcttctgaatttagtcatttttccctttgggactgttctatacaccaccttattcactatttttattttcttacaaataaactttacaggaatcagcttaagaagctaacagcaattatctgcaacaagaataagtatgatgaaaatagagatgttacctgattgtggttctttattagagagtaaaggaacagagaaagaattaccccaaagctgcattgtgcatcagctgctaagtcatggcctgcttgggcaggtacgagaccagtgttaggaaatgcaataatccagacaactggagctgcagggcactggctgtcagttacccccaaaccaaggagagaagccaaggggaagactcccctcttgggctcccaaagaggtgaatgtattagatagtcactggctagagcatccaatttagtcatctttccagaacactcacggtcagatgacatgatgatgaaatatctcgtatagaaggctgatcatctcacttgattaaaaagttgtgtgacctgatttttccatagaattggcccttaatcatgagccatttttttgtcttgtatttaatatcagtcttgtgccagttccttggaatatccaactgactgatccaatcaatgaacgagtgtttatggagagtctaccaagtgccaagcaggagaacatcaattctgtcagccagtccttggaataatccaacgtggccatgtgggaggccacggcaaaggatggccaagcaccttaactgacagggttctgttcttgttcacagtggggagggtaattctccaaaattcagtcctgccactgccactggagcaagtaaggggtgtcaggcaactgtcactgtgccctgatgtgcagcgtggcatgtccacttgactctgtgacacgtctctcctcctagcccactggtagagcaggtcagagattggtcccttcttgaccatttgtactcccagtgttgatgatactacacccagatggtcagagcgaggctgcaatttcagctcttaactcatgccccggcttagcactcttctctcttagtggaagggaaaccctggtagtgccaggtttatggctgcagggctgagagcaacaaaacccatccaggcagcaggacctgctggttgggctgacagccttggagactgtgttgaacctggtcagcactggctccagcagggatggggcagagtctgtatagtgaagtctaacgtggttggccttgggtgggattctgtgtgggtctcatccttttggatggaattgcattaaatcaatgcagtgactcaagatgtttagttcatgaaaaacagtagctatgcttgccaagtatttgtgcctttgattaacttacttagaactaattaatcaccacttataatgtatacttttttaaacagactcctccaaaatggggaggcatgaatttaaaatagagtagccctattcagatggctatgatccacttgttaagcactccttagcctgagtggcctaaaaatcggagcctggggcaaaagcttatatattaattcttgatcaagggctgccatcctagggaaggaagaggggggaaagcaagagcggcaggaaggagggagagcaattcagagcgaggaaccccaagccagccacaactccacagccaaggcactggcctcattccccagggagctctgcaaagtctagtggaatatctgtgtctcagaacagccatggagggggttgggctccctcttctcctgtgctccatcttttactggtcagagtctgtcctgagagacagtcccatcatggctaggttgtcgggtcctctctgcttgggaagccagtatcttgcgggtccaggctgacacctgagaggtgggaaaaagatgtacactctctccagggggctgcaactggggaatgagatgagtgagccaccagggagatgcagctgtgtggaccaggactcagcacaaagctcatccctgaattccaaggagtgagaatgcagaagagagagctatcttccttgttgttttattaatctttgttttatgtgaaactgataggagtattcatttcctagggctgatgaaacaatataccagaaactgggtggtttcagaaaatagaaattgattcgctcctggttctggaggccagaagtccaaagtcaagatgtgcacagaaccatgctgtctcttgggctctaggagagggtctgttccatgcccctctcctggctccagcactgctgccagtccttggcactctttggctggtagatgcatcaccccaatctctgtctctggcttcatatggccttttttctggtatgtggctgtgtctctgttttctcttcttcaaaggatacctgtcattggactagggcccgctctgatacagcatgatttcatcttaactcattacatctacaaagaccctatttccaaaggtcaacttctgaggtttcaggtagacataaaatgtagggtgttgagggaggggggaagccagtcctataaaatacgctgaaattaactatcttccagcaggtaaatagttgataacctctatatcaactgcatattagactaaatcctatttaattggactggagttgggctcaggcattttttcatttacctcaagtaattctatatatatatattttattatcatcagcattatatcactgattataatgcaattggtaacatttattggacagttactgtgtctcaggcactgggctgagtactttatgtatattataccaatgactcatcattccagttcttcatcttacagataggaacacaagagtcaacaatgttggaaaatcgccagaggacacagacctgggagcggccaagtcaagacatccaggctgtctggctccagaactaggactcgtctttgccaagagaacccagaacatatttagaacctttcatcttcttctcgtctttagacagtctcagttatttagagaaattgtaaacattttaagatgcgtaactgcaagccttcactacaaagaaataaaacagcctggagagagaggggctgacgtatcatatgtatcttgtacaacctgaggatgagtcaccaagggcagaagttaataaatgacaggaattattattcaaacagatttaatactttcatgtctcccaatttcacaagtttagaaaaaaagtgatgcatttagaagacatctctccctcctatttcgaagcttagctgcctaacctacagtaaagcctgatagttatgaaccttggagaacagcttctatgacaagtgaggatctaagagcctatatctcataatgatgcctctctgcattaaactaaatcaccccattctgggcaatcattgcagatcccctggtttcctgtcttggccctggacccacactcccttccccatcaaaggtgggatggggagaagcagctgcccagcttaaaacagtgtacttcccagtcatgtgccaggcagaaccttgttggcacccacgtttctgtctctgtggctggacctgctcctcatcttgtcttcagtgcccttctttttcctttgatttctacctaaatccagctcattgtgggaccttctctggtccaaccttcttcaagtctccagctgctcttacctccatgggccatttctttgtttgaaattatcttacatttagtcgattccattctgtttagatctcagctgctctacatttgcttcacagaaattgaccttggctcccaaactatataacaagtacctcccagggacagaaacaaggtgtgtatatgcctggtattcctcccagccttctcggcataatgccaagcaaacaaaattggagtgatctccaacatacctgataattactctttatacctgtagtataagaagtgttaactttttgacattaaatgaaggtgcccaaatagtgccatcagatcatgttaaaattttttcatggtgacccaaattaataatgacatcctttttcctcatggaaacccaaaccaaattattatttatgattactataatttttagccattattaggctctgttatggtactgtgtggttgattaaaaatggaagttgcccatggaggtctacatgtcattgttttggccagtgcaaaatgtagccaggagcacaccacacctgacagtatcagtgattattattattattattattatttttaatactgatgaagagtttagtgactcctgatttcaagctaagagctgattaggaaaggttccattgacaactcatccctagaaaattaaaacagaagcacatagatattgcaatttgacttcagagacttccctttctcctagaacaatgcacaccgcaaatgagggctatttccgatggcttataactaaagtaaaatactgtgatatcgtctaatcttagaatctcttaagtgagaatttgttcaagagggctaacctagaacccagtggaaaaatccagctgatggtcacctgggtccattccctatggtctgaggagcaaatgaggcccctctcctgagtttatttatttatttatttattttatttatttatttattattattatactttaagttttagggtacatgtgcacaacgtgcaggtttgttacatatgtatacatgtgccatgttggtgtgctgcacccattaactcgtcatttagcattaggtatatctcctaatgctatccctcccccctccccctaccacacaacagtccccagtgtgtgatgttccccttcctgtgtccatgtgttctcattgttcaattcccacctatgagtgagaacatgcggtgtttggtattttgtccttgtgatagtttgctgagaatgatggtttccagcttcatccatgtccctacaaagaacatgaactcatcattttttatggctgcatagtattccatggtgtatatatgccacattttctatgccttttgaatacttgcattattttctgccttgcttcttacctttgacctttgtaaatgggagattatttgttgctcttgttttaaggataatttagaaatgacaactttagcatatatctaataattacttgccccagcatttcatcattacaactcaaattaacataatgtctgtttggattttaacaaagaagaaaagctaagaaaaaccttcacttggaaacccttcaaaagtttctcatttggctacagttactcaggctttctgcaaaacgaagcaatgttatccttacattacaccaacagttgtttgagtcttttatcagactgactttctgctcatgccaaaaggcgtcattttctttgtgtaaaagtgactttgaagattaataagtaacagaatgggtacacatggatttatgaacatttaaaattgcttaatgtttttgcatgtgtatactttgaaaaaaaataggagatgccccagttgttctacatagattgccttaggagaaggacataaatgtgcgtcattgattcagacttaagtgaattgttggtgatccacaatgatctcccattgacaagaggtgggtttttggtctcccaaggaactacaacaagcttttagtactttatatcagaaaaaagtacttagcctgtgaagctcatggctttggtgcccgctaatctctctttaaacacgaaagatacatcgcaggcccagagctgttgcatcagcaggcaggcagcaagctgagagctgattaggaagcgtcagcccctagaggcagaggtctgccccctgactctatgagcaagagtcaacatcaagcccttcaatccttgggcctgcagttttccctgtacaggaaacacaagcgtgtgtggatgtgtgtgcatttctttagaaacacataatttgtttgttgctttattttctacctttgaaagatggcaagatcccagtcacaaaaacagaaaccaggctaggtattttaaccagagagggatttaataaaagcgttaggatttgagctgttttggaagagctgaagaagaaggagaggacaggggtcaggaaggacagttcctgcagtgtcctgccctgctgggtggccattacaaagctggcagctgtggggcagtatccctttgcccccatgtggccaggcacacggggcactggctgactcccacagccaccactgtcaggattggagctggagctgctgccacagccagaagcagaatgtccactacctccctcctaccttctgattttgcactagctccctactggtacaacctacacagaaccctgccagtaaaggaatctgagaaatgtagtcccctgaggtacagaaaacacagaagggcaggcagtatctggcccagatgcctatcataaagctttagaaaatgttgtgttatcgctgcactttaaccattgaagtatttattttaaattgattcttatttaaatatcaaatataagagaaatcctatttgactatgccttttatgggacagtcaagaagataatcagtattatattaaagaatattttatgggtttaaagaatattttgggggcattttaattcacatagttcaaaagcagctgaatacatgtattaaaaggcaagctcttgataggccttgctatgtcatctacgaaaagcactcctgtttgcctctgctagctaactcagctatttgaagtacagatggatgaagcaaattctactgtttgggtttttatatttgacactggttgatacttttccaaagcaaatgttgcatttccgctggtagtttgatttcgctgtttgctttctgaaattctatgtacttttattttagtacatagaataactatgcctgcttaattccaatgagaaattatttcagataaataatcatcaacttgtttacttaaaataaaatttttgtacctcgaaaaaccaaaattgataataaaaatcctttaaaatgaccatgattacaactattatgcatatctgtaaatctctttacttcttacatcttgactgatcatctcagcaactctgtgagaagccaactactactgcctccaaattttttccaactatgttaggtgacaggtcattagctgtaaaaccaaggcaagaagttggactttataagtaaatacgatagtttacttatgaatatgtatttatgagactcctcaaaattcatattccaaccacattaattttctcttcatgctagagtctaatgcagtggttagactgcagcatgaaactggggtaagcataccacttggagatttgcaaagatgatcacaggaattgtcggcctggataattttaagggagccagttttcagatcctcaacctacattcgtactctttcccaaaacaaattcgtctaagaatatgtcccttttaaggatgcaggttcctacccaaaattgtacttttcccactttgcaaaattaatcatgcctctctctcatctttctctgaggcattgacttgaagttttaaaatcttcagggcaacaaacaatgggacaatttgacatattgttccagtattaagaaagtaactttcttaatgtaagactaattataagtaaatgtaaatgactgatgacttaattaaacaactttttgtaagtctgatggtttccatttttttccttagagcagaactgtaggagaacttacatgacatgctggctaataaaaaagtaaaaataatttggtgatagatcactgtgaatcttggcatacagctgagaaggaggttaaagaattgagtaacatttctgtagcaaaactcccttcattcccactgatttatttatatggaaaaatcttcttggcccataaatttataaaaatgaaaaacagaaatagaattaattttgagccctaactcattctagtaataaataatatatccactgatttcatgacctaactagaaaaacttagactcgcttttggccatggagacatacatttccaacaaaattttacttctgcataataattatttcttaaaatttattacatcttatttttttatcaattatgatctaaagataattgtaattataacccaagcagaaaaaatccaatatttagagacttattgccacaggaaaattttaaaaattaaatgtcaatttatatatgtaacttattgtacatagaagtatggtgacaaaacctataaaatattttctacacgaaatacattacattagaataaaagtctgtggaaatgaaaataagagctcaaagagaaaaaaattgatgtcaaatttcagactattaaagaacagtctgtttatagcaattggacacctgatagcatataatattcatactctattgcagccatcccttcctttttggcactagggaccagttttgtggaagacaattattccatggatggagggagtgggggctgttcggggagatggtttcaggtgaaactattctacctgagatcatcaggcattagttagattctcataagaagtgcgcaaccgagatttcatgcgccgcccacaatagggttcacgctcctatgagaatctaatgccgctgctgagctgacaggaggcagagctcaggcagtaatgctcgtggcccactactgctcacctccttctgtgcggtctggttcctaacaggccacaaactggtactggtccatgactcggtggttggggactcttgctctattcaatacgcttaaaagaatgatgttggccaggcgcggtggctcactcctataatcccagcactttgggagtttgaggtgggcagattacgaggtcaagagatcgagaccattctggccaacatggtgaaaccccatctctactaaaaatacaaaaattagatgggcgtggtggcgcacacctgtagtcccagctactcaggaggctgaggcaggagaatcgcttgaacccgagaggcagaggttgcagtaagcagagattgctccactacacttcagtctggtgacagagcgagactccatctcaaaaaaaaaaaaaaaaagaacgatgttataacaacatagtatactgaaaattacatcctttgcagttatttaaacaaaggatatttatgtgttaatgtaaacatacgtatgtgatacattttcaaaatttatttaaagagtatatgagcaaaaatctttacagatcattgatctatacaaaagataaggtgttttgaagatctgcattctggcatgtctccatgtgtgttttctttgttttctcagtttgaccttgtctgtgtcaatgcgtggatgctggacctcacccaagccatcctgaacctcggcttcctgactggagcattcaccttaggctatgcagcagacaggtaggtaccaatgtgacagccattttatgtcactataataccatgcattaatacggggagaaaaatgttttatgttaactattaaaacaatatttttgctaaattcgcaaacaattcttgattccatcattcatgacatccacacttggttggtttgactaattcttttcttttcaaacttcatctccttattccttgaacttcacaaaggtctgaggctgttcccggtaggactgagagcatcagaatgaaaacacagacctccaaagcaggaacatcatgactgggttgatgcactgttatgctgaatactacagaatcaatcaattcgatgtgcttcagtaacaacttactgattgtggttttattaatttagtcataagattagcctattccaaatttgtttcacctaatttttttttaaacgaaatccttgtattttattctacaaatgtaaaactctgtgaaaaaaatatttttccaaaaaccactaaaggagtttttttctattattttgtattcataaatgcaactgcccagtggcaggcatggtgagtgtgtatattcaagacatcatgctttctatttaaatattcttcttccttaagattattgatgctagctattgtattcatgtttaactctatttttaattccaattctaactatgaaaatacttgcagtaatttaatattcctctctatcccccttagcaggagtaataggaatatagactggaaattgctgactcatggctgtatctggcctaatattgttctggttaatccatctctgactcatggccatatctggcctaatattgttctggttaatcgagcccagcccatggtgggttcttgtgactgtaccattcagtgggtgatactaagtttacctttagagaactgcaaacagcaattaattagctaatagaacaacaaaaggccccatatcctgtagccaattgaagatctgagtttaatcagaccagtgcttcaaaatgggtagagccttgggtttcattttctgtcactttttctttctctttcatgtttctctctcgccctctccacagctgtcctttgcatgtgagtttctgctggatcctttcatgtgtgatgccctttcccctttttcttctttttatcgatgtccttttctcctgttctacttctagccctttcaagcttcatctttcctttcctaatttcccccacatgccactctttttttgctggctacaatttggaactgtgcagtttaaatatttatttattttgttttgtttttactctttctaatttggatattaggttttgcttcatctgtgttttttttctcttactcagtcaataaccatatctccaaactaaattaacgttactaaagtggggaatttccccttcctatattctcataagtgattgagcatctgtcctcatataggacttgctgccttggaggggaggggccagacctgggaaaaagaggagccatgaataactctgcttcctacatttggcttcttctcttcctccatatccatgatttatatatgtgaaggaagaacaagaaataacttaataggccatttgtcaatgagagtacagtgtaggaagggtggaaagtgaatataaaatctagattggggcttctggtttcctgttcagcctataaggagcttagaatttgccactcagtcttgacaacaagtaaaatgctgaacaaactgaaaaatcaataattcttctcagatccataagagaagtgagattacagggcaaactactaccttcagcatcaccccgcacccccaaccccactaaatagaaagacaggagaatacagagaatcataacacaggcgcagaaacctccttgagagagccagggtagataaacatgaactgtaattgatgaattcctggaggatcactgtggatgacctgaaggattaaaaactctagagggactcactcaaaggagggcccaagcttttgtgattttttttttttttttttttttttttgccacctgaagctctacaaggttccaaaggtgaatattaaggaaaatccctcatgctctggcagccagaggggaaaaggaacaattttgaaatatgccagaatatcgttcttaacaatgtctgccctcaggagaagatgtttaaccagagcctaatcttctggggttttctgagagcctcattgatctgggggaagggaaataccaactccagccccttctagccttccaagtggagaaagagaaacaccaaattctagcctcctctagctttcaacttggaagaagggaaatacccagctccagccccctctagcctttctcccctagttcagaggagagggatagagaagcatttgtgaagttcaccgtttagagacataggttcactaaaagactgagagctactcatagaaccacagaatgccttccctcccccaacacctcaccaccacattactaaaggtctatttatagcagttccgtttacccagtacaccatgtccacctatcaagaaaaaattacaagacatactaaaagccaaaacacacacacacacacacacacacagttgaagagacagagtaagcatcagaactaaattcagatatggcagggatgttggaatgatcaggctaagaatgtaaaacaactatgccaagaagtctgatgcataaagtagcatgcaagaacagttgggctatgtaagtagagagatggaaattctaaaaaaagaaaccaaatgaaatgctagagatcaaaaacactgtaacagaaatgaaaaacgttgttactgggcttattaggagattgggcatagctgaggaaagaatctctggccttgaggatatcccagtagaaacctccaaaactgaaaaaaaaaaaaaaaaaaaaaacccacacacacaataacaaccacctgaaaaagcagaacagaatgttcaataactgtgggacaattacaaaagatataatatatgtgtaataggaataccaaaaggagaataaagagagcgagaaatagaaaaaatattttaaacaataatgactgagaatttttcaaaattaatggcagacaccacagatccaggaaactcaaaggaaatcaagcaagataaacataaaaaagtatacctagttatatcgtagtcaaactacagaaagccaaagataaaggaaaaattctgaaagaagagtggggggataatgtcttactatagtaaaagagcaatgataaaaattacaccctacttcttttcagaaaccaggtaagcaagaagaaagtggaatacttaaagtgtcaagaaaaaaaatccaatctagaattctatactgtgcaaagttatcatttaaaagtgaaggagaaataaagactttgttagacaaaaattgagggaatttgatgccttgtaataaatgttaaaagaaattatttagagagaatgaaaatgatctaggttagaaatccagatctacataaaggaaaggcattggagaggaataagcaattgagaatatgtttcttattctcaattgatctaacagataatagtttattcaaaataatagcaacaatgtgtttggttgtgtatgtttatatatataagcatacataagcatatatagagtattttttgttataaggtactcatactactcatgaagtgatacagtgttatttgaaagtggatttgaattagttataaatgtatattgtaaactctaaagcaactactaaaaaaagattttttttaaaaaagcatgactgatatgctaaaatgaagaaaatgtggaatcctataaactgctgttcaatgaaaccacaaaagccaggaaaaagtgtggaatacaaacataagaataaaaaataagggaaacaaatagaagatgataacaaatacggtagacattaatccaaccatgccaatactcactttaaatgtaagatggtctaaatgagccaactaaaagacagagattttcacagtggatcaacaaacaagacccaaccatatatgttgtctacaagaaatccactttcaatatacagacacatatacactaatagtaaatggatggagaaaactataccatgatagcagtaatcaaagtaaagcagaagtagctatactattatcagcagagcagacttcagaggaaggaaagttatcagggataaaaacgggcattatataatgacaaaggggtcaattctccaagaagccaataatccttaatatctatgtgtttagtagcagagcacaaaattataagaggcaaaaactgtgataaaactggaatgagaaatagattaatccacgatttcactggagacttcaacatccctttgttggaaatggtcagattcaacaggtaaaaaacctataaagatataattgaactcaacagcatcatcaatcaactgtatctaattttcctctttagacaaccttatctaacaacagcagacttaacatttttttcaagttctaatgaaatattcacatgcaatattcaagataaatcacattctgggccataaaacatatattagcaaatttgaagaaataaaaattatacaatctctgctcccagaacatagtggaattaaactagaaattcataacagaaggatagatggaaaatccccaaatacttggggatgaaacaacacatctatagtaacacataggtcaaagaagaaatctcaagagtaattaaaaagtatagtaaacaaaatgaaaatgaaaacactcaagacttttggaatgcaacagaaacaatgtttaaagggaaatttatagcattgaatacatatattagaaaagaggaaatatataaaatcaataatctaagctttcaccttaggaaactggagaagagcatattaaacccaaggcaagtagaagaaaaaaaaatacaaattggagcagaaatcgataaaattagaaacaggaaatcaagggagaaaataaaaccaaaagctggttatttgaaacgatcaaaaaaaatcaataagcctccagtcagactaagaaaaaagaaagaggagagagagagaggttgaagacataaattactaatattagaaataaaagaggggacattttgacggatcccatggacattaacaggataataaagaaatactatgaacactctgtgttcacaaatttgataacctacatgaaatggaccaattccttgaaagattacaatctgccaaaactcacccaagattatatagacaatctgaatagggctatatctattaaataaattgaatcaatagttaattacttttcaaaacaggaagcaccaggcccagatgggttcacaggtgaatttgccaaacgtttagagaagaaactctaccaattccctaaaagctctttccaaagatagaagtagagggattgctttctaacttattctgagaccagcattactttaatacctaaattggacagacattacaacaaaagaaaactacagactaatatatttcatgaacataagtgcaaaaatcctcaacaaaaatttgcaagtagaatccaatagtgtataaaaaaaattatacaccacaaccaaatactgtagcataccaagtgcgtagtatcccaagtatgtaaaactggttcagcattctaaagtcaattaatgtcatccatcaatcacatcgacaaactaaggaagaaaaaatgacatgatcatatcaatagatacagacaaagtatttgtcaaaatccaacatctatatatgacaaaagctctcagtaaactagaaatataggggaacttcctcaacttgataaggaatgtctattaaaaaaactacagttaacatcatacttaatgacgataaactgtacagaagctttctcactaagatcagaaacaagccaaatatgtcatctctaataacttcttttcaatgccatgctagaagccctcgctaatacaataaataagacaagtaaagaaaataacaggtatactgattggaaaggaagaaataaaaactgtctttgttcacagatgtcatgattgtctatgtagaaaatcaaaagaattaacaacaacaacaaccaaaaaaaaaaactcctgaaactaataagtgagtatagtgaagttgcagggtacaaggctaatacacaaaagtcaactgctttcctatgtaccaacaatgaacaagtaggatttgaatttaaacacataatgccatttacattagcaccacaagaatacacaacacttagatataaatctaacaacatatgtacaagatttatgtgaggaaaagtgcaaaactctaatgaaagaaattagagatgatctaaataaatggagagatattcaacattcatggataggaagactcaatattgtcaagatgtcagttcttcccaacttgatgtatatattcaatacaatactaatcaaaatcctacagagttattttgtggatattgacaaactagttctaaagtttatatgtagagacaaaagacccagaatagccaacgcagtactgaaggagaagaacaaagccagaggactggcactacttaacgtcaagacttaccgtaaagcgacagtaatcacgacagtgtggtatttgtgaaagaatagacctctaccaatagttcagtggaataaaatagagggcctagaaatagacccacataaatatagtcaactgacctttgacaaagcagaaaacaaaaggtactggaacaactggacaaccacatgcaaaaaaaaaaaaaaaatgaatctagacacagaccttacaccctttaaaaaaaatggatcacagacctaaatgtaaaatacaaaactgtaaaactcctagaggatgggataggaaaaaaatctagatgaccttttagatacaacaccaaaagcataatccatgaaaaaaagaactgataagctggacttcattaaaattgaaattttcttgttgtgagagagacattatcaagagaacaaatgaaatgacaagacacagactgggagaaaatattaacaaaaggcaaatatttgataaagaactgttatccaaaatatataaagaactcttaaaacacaacaataagaaaataaacaacccaactaaaaagcgggccaaagatcttaacagatagctcaccaaagagagtatacagatgataataagcatatgaaatgatgctctacgtcatatatcatcagggaaatgcccattaaaacagcagtgagataccactacatatctattagaatggccaaaatccagaacaatgccaacaccaaatgctgactaggatgtggaacaacaggaattctcattcattgctggtgagaatgcaaaatgaaaggtgactttagatgacaaaattcttacacatttttgtaattcttacaaaattaaacatacttttactaaatgatccagcaattgtactccttggtattccaattccaacccaaaaaagttgaaaactcatgttcacccaaaaacctacacatacatgtttatagcagctttcttcataattgccaaaacttggaagcaaccaagatgcccttccataggtgaatggacaaactgtgatacatccagactatggaatattattccatgctgaaaagaaatgagctatcaagctatgaaaatacatagaggaaacgtaagtacttattactaaatgaagaaactgatctggaaaggctacatactataagattttaactatatgacattttggaaaaagcaaaactatggagacagtaaaaaaaaggatcagtggttgctagagggtggggagagggagggatgaacaggcagagcacagaggatttttagagcagagaaactattctgttagatactttaatggtggatacgtgtcattatacacatgtcaaaacacatagaatttgcaacatcaagagtgaacccaaatgtaaactgtggacattgggtgattatgatgtgtccctgtaggttcgtcaattgcagcaagtgtaccactcccatggggaatgttaacaaggagtatgctgtgcatgtgtaggggcaggggacagagtccactaaaaaaaattttagataaactaaggctacttccatctctactctgctgactcaagcaagatgctatatgtacatattttatggaaatttttatgaaaagatgtaatagaatcaaatctcttaatggacattgatagcaaacatttaccagacaataatttgacaccactatgctgaaccaattcacaccagtctctcagaatgttgtgcatgttaccttgttccttttcctaaactatttaataacacatatgccaatggtgaaaataaaatacaaaatttaagaacaactgacattggctgtgaactttgtagtttataaaacactttcacattaacatctcatttgatcctctctacaacctccctctagaaaaaggtgggaataaacaaaaacaagcaagcaaaaccttcgttttgaagaccttaggaattagcccaaggtcttatgactggtagatggttgaggtgagattcaaggcaaggccttctggccataacacaccattccttcttctttctgcctctctgtccttatgacttgacacataaaaaatgctagaaagtgtcacatctgtaccaccggggagactctgtgtgaaacaatagtttcaaggagggtcatcttttcagaaactgcaatcacaaaggatgcattctgtactttaaattttgtaattccagcctaggcaacatagggacaccctgtctctacaaaacatctttaaaataaaatgatataatgtaatacagtataatatggtatgatataatattttatgttatattttcccattgtgttgaagaaaatgtttaaggtgagctcttttcctgtctttctcaaaaaggtatggcaggatcgtcatttacttgctatcctgccttggtgttggcgtcactggggttgtggtggcctttgcaccaaacttccctgtgtttgtgatcttccgcttcctgcaaggtgtatttggaaaggggacgtggatgacttgctacgtgattggtaagacattcttacaccatcttctctatttggaaactagggccagatagccaacaaatgctagttaatcaaccttcctcttcctcatgtgtcctttaacaaaggaggtttcactgcagctacagtaattatttctgcagatgggagagctcagaggtaggattctgagtgctttagaaactaaaataccaccttactttgggagccaaatatagaatattataaagcactggggcaaactacttttatgtacttctacactgttataatatggtttctagctttgctttataagaaattaatttttataaaatctcagtagcttcttgaggcatttttgaggcatttttaaagcattttctctgcactgtgttatgtgatacagtttgagattactttcctgtgttgccaactctcctctttctgtacaagtgggaaactttttactatgggagaaataggttctaaaaatgagaagttcatttgtgacccttggataccttctatagccagaaagtttgaccttctggtgatggaagaaagaaaaagttcactttacttttttctcatgtcccttgggaaagccctggctactccttttggttccatttctagcttgggaccagccacttactttaagagtagtggtggatttctgaccaggaaaatgtgaattctcatcttagattattgctgcagatctttgcacttccactctcctctgtcactgctatacaatctccaagcagtgcgcattttggagacccctgaagacctttctgtgagtgggagaaggagagaagatgattagagggattggttgtcactgtctggaacagaaggagtaacattggaaggcaaggcatttttaaggagactcagcctaggtgaatcttcaaagttgtgatctgagggcaacaataaagtagcctatcccaggataatcagaatatgagaaaccaattagtgggaaggggcaggaaaggaaaagatttcagcaataaaatttcccatgtgatttttagagcatatcctgtcaatgaaaagtataacactgtggagagaattatcatttaatagaagttgtcttgtgataacgaaaagtctttgtagagtcttatatttataatgaatcttctaaggagactagaatgtgcatatctctagagatacagtgataaaatgtagccatcttaacatatttatgttttgataattacaattaccttttaaaataatcatcactgaagctttcctaaaaccaacacagatattcttcgtatttccctattgtacccagaagagtgctggtctgggtgcataagaggaacatggcaagtgcctgtgataaatggcagaggtgagagtgggagaaaagcatttagtatcaagaaggacacctccatcctttctgcaagtgtggaagcctccgtatctgagtgctagcgtgttctagcaatgctgatggatgtaacaggtgtaacatctctgtatttgtttgtttattttggagctgaccttgtgcttctgtgacctcttgtgtttgttttcctttagtgacagaaatagtaggttcgaaacaaaggaggattgtgggaatcgtgattcaaatgttctttacccttggaatcataattctccctggaattgcctacttcatccccaactggcaaggaatccagttagccatcacgctgcccagctttctcttcctcctttattactggtaatgtggttttggttatcatcatatttatactgattctgcagaaattagactccaaacagacatgaaaatgaaagcaataaagaaaattttcttttttttttttttttttattatactctaagttttagggtacatgtgcacattgtgcaggttagttacatatgtatacatgtgccacgctggtgcgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttatactcatttgggtatatacccagtaatgggatggctgggtcaaatggtatttctagttctagatccctgaggaatcgccacactgacttccacaatggttgaactagtttacagtcccaccaacagtgttttcaaacatctctaaaatggctacatgctttgagaatttttagagaattttaggatttaaagagagtatttgttgatgattttattaataattgtgaagaaaaatgaattaataaaataccagaacgcaaaattttaagtagggtactgcattatgttttgttttgtaataaatagcttcatgaaaactataaagtaaccaaccatatctgccagtatttcaacatgcaaattaaatgcaatgaagtctatttgtttctctgtgtgttctataaagcaaatgaatattactcagaaattctctgaggaataaggaaatgttaagagcatcaaaaaattacattatcaaaaagcaacagaaaagcatttgatttttgttgttgctgttttgagatggagtctcactctgttgcccaggctggagtgcagtggtgcgatctcggctcactgcaacctctgcctctcagattcaagtgcttctcctgcctcatcctccccagtagctgggattacaggcatgcaccaccacacccagctaatttttgtatttttaatagagacagagtcttaccatgttggccaagctggttgcaaactcctgacctaaaatgatccacccacctcggcctcccaaaaggctgggattacaggcctgagccaccacacccagccagcatttgatttttattggaatgaacatgttttaatgattcttctaatttaattttaatatatgggcaagtcagtttacataaatcagattcctaagcttttcttaatatttactaaatcccaattcttttcatatatggaatcaatatattgtaagatagcctggttttgcaagagcaatgtcatgttgattattttgatgagcaatgatccaattatcaagctagcatttttccccatccattataaccttgcaggaataatctgtatttcagggacctattatcaactcttattgactcctaaatgtatatctaattgcaagatccattttgatagaaaaactcaaggcaatagattttagcgtttgaaattcctagacataactcacaacagcctccttctttgccagggtggtccctgagtctccccgttggctgattactcggaagaaaggagataaagcattacagatcctgagacgcattgctaagtgcaatgggaaatacctctcatcaaattactcagaggtaatttctttcagtatgagtaacaaatattgctaaagctggtgaattgattttgttgcttcttgtgtacttaatgttgcttcttaaatttatatacaaaatatgcatattatatatatatatgtatgtatgtatatatgtatacacacacacacacactaccaaacatctgggaaatcattttcagtttgacttaactgaaaaaaatctagggaaaaatcataggacatattaaagaagaaatgattatgaagctctattagcattatagccaaggttaattgatcatttcagtacatattttaataattgcaccaagttacttgaattattatactttacttactgtaagccccataatattgaatcattcatcttgattgtacaaactgtaaaactttttttgacttttaatttaaatctttaagtttggccctgaaaaaaaatctgtttattcataccagagcttgagcagtaatgtaatatttcagcctctaaactcctatatgctggtctttctccctttcttcttttcttcctccattccttccttcttccaagaaaggctttttggatgcctctgcatcagtgtaccaagaaataggggagggccgggtgtaatggctcactcctataatcccagtgctttgggaggctgaggtgggaggattgcttgagcctagaattctagaccagcctgggcaacatagcaagaccccatctctacaaatagaaagttttaaaaactatgtgggcatggtggcacgcacctgttgtctcagctactcaggaggctgaagcaggaggatcatttgagcccaggactttgagactgcagtgagccatgatcacaccactgccctccagcctaggagacagatcaagatgctgtctctaaaaaagattttaaaaaaatgaaatgggacaaaaattaaaagcttttgccctttggaatatttgatatgcacttgaggatataacatatatttaaagcaacccataaataattagagaatagcatataaagaaaaatatgatgatgttttagctccaaagactaagtgactacagaataacagaaaggttgttataaaaggaatgttagagaagaatttttggatgatatgaatcttgatttaagactaggttgtcaacattgttttcttcatagcattcagtttgatttcacaggaaccttctgataagaacgttctgataagaactgggttttggggctgggtgcggtggctcacacctgtaatcccagcactttgggaggccaaggcaggtggatcacctgaggtcaggagttcaagaccagcctggccaacatggtgaaaccccatctctactaaaaaatagaaaaattagcgaggagtggagacgagcacctgtaattccagctacttgggaggctgaggcaggagaatcacttgaacccaggaggcagaggttgcaatgagacaagattgccccactgcactccagcctgagcaaaaagagtgaaactctgtctaaaaaagaactgggttttgttggttctattgtcccctccccagcccttgaagcttggtcatgtatatttggatttctgccttcttcctctctcttatattaagtggcaagcagtaggctcttttccaaaggtttcaactgaacagatgtcactggtgcaatggtgcaagtacattaatgatgtaccttgaggtttacagatcataaaatgtgtggtaaagataaagcatgatctttttaaaccactatgattagtccatccttgtcactctttgagtgaacacatttgagctgtgtgttccttttagggaaaaataatatacccctgtttcccagcttgcactcatccattgtctatagtcctcatagccaggtcaaagatctgatcatttgtattcaattttaatcatccagtgaataatctctgctcttttgcacacatagtccagaggtaagtccaaggttttgatcttgatttaaaggtctaaatatagaaatatatttaggaatatagaatatagaagaaacaaatatagaagggaatatagaaaaataggatcatagaaaataaataggaataaagaaaggaatatataaaaataggaatatataaagtgttctagaaaaataggcatctagaaaggaatatagaaaaataagaatatagaaaggaatatagaaaaataagaatatagaaaggaatctagaaaggaatatagaaaaaacagctgtgtgctggtagcttgtgaaatataatgttggattaaccaggcatgtcatgtgtgatgatagtgagtatcactgagagatgagtctgattccccgaagcagctgctgttacacatcccatgagtgtacagtttagcactgctcccacagacccactgacccagctttattcatgagcaccgtagagatcatgacaacaggtaagtagttcaatgttccacatggctgtgcttatgagagatcatgaactatgggatccagtggcttccaagtcattttaattttaatatggaaatttccaaacatactcaaaaatacaaaaatagttgaatgaatctctatgcatatccctcagctccaacagttaccaactcctgatcaatcttagtttttctatactctgcctagtttccctatccactgagtaggaatcaatccagccatcatagcatttcatcttgtaatattttcctctaaagtaggaacacataggtatctctggaactatgtgtgcactatcttcaggacactgctgaaggaggagcaagaactattcctgtggttcttaaaattcaatacaagtaatcacacctttgatccacctgtggggactgatccctcttgtgggagcaaagatggggccatggtatataaaatataaatataaaactaatatcaatgtcattgtcctaagaaaaataaccatgattccttaatatcaagtcagtgttccacttccctgattgtcctacaatgtttcaccctgtctatgtgcattagactcctcctgaagttatggattatgaattgttgatctctttgggtttatttaattcataggttcctgccctttttcatttcattccaactttgggaataaaaaaattcctggggtttttctcctgaagaatttccacagtctaaattttgttgattatatctgtaggatcctttaacacgtttctctgtcttttatattccctgtcaattggtagttagatctagagcttaatcaagttcaagtttaatatttctggcaagactacttcataagtggcttagtacatttccattgagagatacctggtagctgcttgtctcttcttttggaacattacttgccactgataatcactgcctggatttactaactcatcagaggttgccaaatgacaatattctcatcttatcattacttcttcatttattgctgcagtgcttaaataaagaccaatttctcccatcaactcttggtgacactgagatatagctcatatagggaagacaagacatatgcatcattttttcttttccaaataataaattggctccctacatcctcccaaagtgattaatgagttgagtgtgtttatttaaacatagtttatatgtttcaatcctttgcaattgcattagtctgttctcacactgctataaagaactgcccaagactgggtaatttataaaggaaagaggtttaattgactcacagttctgcagggctggggaggcctcaggaaacttacaatcatggcagaaagggaagcaaacacgtgtttcctcacatggcagcaggcaggagaagtgccgagcaaaggggaaagccccttataaaaccatcagatctcatgagaattcactcactatcatgagaacaacatgagggtaaccatccccatgattcaattacctctcactgggttcctcctatgacacatggggattatgggaattacaattcaagatgagatttgggtggggacacagccaaaccatgttaacagttattttggtgagtactctttcggatgctcaaattgctccatctttgggcacacgcagcttatccagagtctgccaccctctagtagcttctgaatacctccttgctctttagcacgatgagattttccagtctcatcttgtaagttccagtctcacacatggaatcacctatttctccaagggagctttggtttttgaaacatatgtcttcaaaccgaacctacccatctttaatctttttaattgtacagcgtaagcagaaaagcaaagtgttatcttcaagagaaggatttaacatttaagtttaaaacaccttgctaaaagcggaattcagccacattatgggccacgtcatagttttccatctcaaaggtccttatgtccccacaagcaccatcctgagcagaaaacctcttcgtgatgacgcagaaggactggccagcagggtaggcactgtacttgaactccaaccccaaaaatcacagcaggattccattataggttttgtgcaatgacaggtatgaccagtataaattactcatattttgtgattcaaatacatactgtccgtgtctatgttaggatagccttgaaactgtatgtttacacagttactttcacattaacccacatccctctctctggatacagtttccaggatgttcagacccacgactgaagttcccagcaatctctttttacttttcacagaatctctcttcagtaacttctgacttttagaagacgttatcactgtgtaaatgacctcatccttcacgacctctctaattcccacctcattctcagggtgcccattatagggtgcggagaaggttagaatggatttgaaagaatgtggttggattcaaagaagccctaggagacccaacaagtcagcatttttctcttgtgaaaagaaccacctgccaaccccagcctgttccattgctgacatcagaggatcaggtatatttaggagaagctaaggacctagcttaaatctacccaaggatgttccatattacagataagctaaattaactacaaactaagattacaccaatctccagagctttagtgagagaagggaaaatatgagaggaaaatagtatgacagatttgatccaacattgaaacaggtgcattttgagggttgtttatcctgtaaatcatctcagttacagttcacatggatgttactcatttcatgggtaggcaaactctgtctctgcctcacttgttcctaggcttccatttgccaaggtttctggctggcctctggctttgttgaagagatagctagcactagcagggagaaatacaggtgatcagagtccaggctctctgaacatacaaacagtataactgttgttcactaaatggaaaaatcccaaaatcaaaaaccaatgcaaaacagtgaagtggcttgagctcctaggaggttaggtagaaattaaagagaatcagtggatgggtagaattttaagcagtaggtagttacccaatgtagaacgaggattagcttagacacctagtctggtggttctcagcctttcagcatttgcatacgataacttttaaggcttacttaaaaaaaaaaaacaaattatgggtaagtctacaataatgatagcacaggcacagctacagcatgacagcgtctcacaatgtcttcaggagcatccacaatcatccactgaccccagagagcatgtcaaggatgagaactgctttctgccctggccacaccctctgttttgctcataatgcaaagttcttttacagtgcagtaaatgtgaaatttttctttggtattagttagacattattcctgcttgttacttgtagtgccctgtttcttgccttacacctggtcactaaccactcaattcaacttttcactctcatatgatagacaagtaatagtactgtgtgcaagtgtgaataatggatggagtaacaatgcttatctgaggatatggccattgccccaggtactgaatttgtgtgcttgtcaaaataaaaaagaatgggcaagtacttcagagtgtccacacttctggtggtagtagacttggtctattggtgagtaatggagagctgggattgtttctaagtaagcatgaactgctaatatctgtgataaattgtattaatcctctcagctattcacttcctctctgtaagaggattttacatcccattcccacccattgccagatgattgacagcagtatctctctgtaggcaatatacttccccacccagtgtcaggcttggccacctgacttgattgcgcccaaggagcagaagtggcagaatatcaattcagggcagaagctttgagaggcatcatgaattcctgacagccctcttgctccttctgtctgccacaagaacagcagatcccaaatcagtgctgttccttcagggtgggtctcagaaggagagctacagcctggagcagagctgcagcagctgacctacagctgctgtatgggacatggccaagaaaaagctgtttccccagtaaactactgctcaatgtgtctgcatgtactacagagatttgggggtatctgttcttgcagccaaatctagctaatgcaattggtcatattactagttcaactgccagagtagggttcaacacattctttggtgatgcaggggtatctgattgtggtagattaaattactgtttggagagattcacattttccctcatttccaagggatggaaatacttcctgggtctataaatgttgggcatggcaatgatattgctttggcctcatgataggtgcagtggacttcctgccctttgatgttggggttgaccatgagacctgctttggccaatggcaggttgctagatgtgatgcaatagaagcttgaaatgtgcttgtgtggttgggctttatctctgcacctcagccatcaccatgaagagaaaattcccaggcttacctatgggccccaaaggaggctgagagacattcagaatggagttgacctagcaaagcttcctcaaacctacactagcaggccccagccagcccacaaacacatgagataaatgaatgcttactattctatgctgctaagatcgatggctcttatgcagcattatttatcaatagctctgatggtaaagtgtcaacttgattggattgaaggatgcctagatagctgggaaagtattgtttctgggcgtgtctaggagggtgttgccagaggagattcacatttgagtcagtggactgggagaggaagcctcccctcaatgtgggtgagcaccatccagttggctgccagtgtggcttgaacaaagcaggcagaaaaaggtgggataggatgtcttgctgagtcttctagctttcatcattctcctgggctggatgcttcctgcccctggacatcagactccaggttcttcttgcacttacatgagtggtttgccaggggctctcgggcctttggccacatactgaaggctgcactgttggtttccctacttttgaggcttttggactcagactgagccactcctggcttcctacctcctcagcttgcagacaacctatcgtgggacttcaccttgtggccgtgtgagccaattctcctaataaactccctttcatagatatgtagatcctatcagttttgtccctctagagaaccttgaccaatataatagctgactgtgtcaacactgagtgagcagccactgtcactgtactgacctggatgatgcagctacacaggtagtaactgtggcagcagttttggtttcctttttactcccaggatcttaaaggcctttatcctaaagatccctctcctctgcccccaactgcccattcctcccgtttttgtatgggatatactcatagagaatagtaggttacagttctagctaacaagaagtctctgatactacagaactgaatatgcccatatttgttagtgatattttatcctatactggcgcaataaacccataagctttgatcgtagtttccttattttttggatacacttatgtattgtttatagtgaatctactgcttttaaaattaaacattagatgttatatcaataatgttcacacataaacatgcttgaggcagttcccatggtggtatgtttatgtgacatcaggaacggaatttgagctttccaattccaaagggattcctttaggcccatcatcaatataaatgataccattgtttgaaatatttgcaaaatagaaaaaattaaaacctcattagaaagtgtcttactgattttttttattaccttagttgtcagggagacttctagaccttaatagctaccgtcttttctataatttaatgagcagtcacacaaacaccaatattgctacagaaagggactttccagctttgggaatatctgtttctgacatttcctttcaaagggggatttttataaacataatttttcttcttccaaaagaggaaccacactatgattccaacttccatggagcccatcggggatggtggtatttcagttcaattagggtgctgtctttgttaactgttgtgtttagttgtgatctcagacttcagatttgaaatctctcttcggggaattcatttcaaaagatccagataacatagtttaggcacatggagtatctgagggcagtccagtgttggaatgagaaagattgagtctcaaaaacaaagaaagtctcaaaaacagttattagaaagaaagtcactgggaacccatccagcactttctcctgacagggctgtcttcctcctctcactttacaggatgaatggtgccaattctaccagcaaagccttcatctttaaaatgactcataaaaatggtaactgtagtatgtgtaccatcacactcactttgacttagaatatgcttgaatgcataaaaatatgtcaggacactggacatactcaacacagaatggaactttgtagccaataattaatttaataatacttcctattttaaaaaatagcctgagttctagaaatggtgtgtatttaaatggatttgtttactttccatgtctttagctttttgaaaacaagactatatcgcataaaatgagaaaactgttcatgctacagtgaaatagccataaagactgtaccagtaaacagttccatccacagcatgtcaagacccccaacttcctttctcaagcatggtgcccagcactcatttccttccccaaatggaaactcagcctggcattgctcaggtcagtgggctgtcattcaacaccgaaagatattgaggggaggatgttctcctacacctttaattgaaatggaaattcaatttaaaggtgctttaaagaatgagattgcagccttggagcaggctaggcagacttgtgggatgcctcatcagacagcagcattttgcacaaggaactatggtgagccagggcctttccccctactcacccaaactcatcttccagatttcctctgggaaaataaatcattgggccatgcctttatcaaaatgttaactatctctgctgtacaagttagggcttgaaaactattcgtcagaactgggaaaggaaagcattccacattatggtcccataaacttcctgcattcagtacttgcaatcaattctggtgtctaaggacatattactaaagagtgagtgtatttacagaacctaggacaacagagggactcaggcagtgtccagcagggtggccaactgtccttgtttgcccagggccggggagttgggagtttcagagctaacaccaaaaaaatgtctcaggcaaactgggtagagctgttccctccatgactgggtcagtcctcatctccccagttatgctcagaggaagattccaaaggtctacacttgcaggcgtcacaagtcctccctgcccatcccatgggccctgatgcctagaagaagacacgggattcccgcctggctggaggatgggtctgggatctgcttgccagccgttcccgggattgggggcaggttgctgagcctgtctagttgcctgccaggcttcccctttctttccgcaccccttcctccgcagagcctcatgtctacctggttcttcatctggtcctgagtccttgcccattctactgtggctctggccctgcttcattcctgcctgattcatcctcagaaggcaggtttctgtcctgtccctacacatccatggccttaaagcaagatgctgacgctgtcttgatggctggacctgcccttcctagagacggctgctgccaacccatttcccagacaagtacccaggccacagacagcagggccagcccacggaaaacacaaagcccttttttccctagctagcctctccatgtggaggaacaagtcctattgcttggtggagagccaaagccctttcctttctcacttactcttctctcactgctactgggatcctggcaaaccacacaggcaggtccatgtggcttatgacacaggagttccgagctttgcgacatgacattggcaactgctcagttatccgtgattagttatgaatctaaatgcgtgggcttcccccttttccttctcaaaggctgagcttccccttgccctttaggagcaaaagtcttcccctttctactccagcactccttggtaggcagagaagcatttacttcttccctgcctaccctgagccccggcagtattacaaacaaaagtaaaaagtaaaacacaaggcaaaactcatcagccacacgggcttgttttattccattaatattggttgttctcttttctgggagttgtgtaggcatctaatttgattattttagttatgtgtccctctctaatagaaaagaggagtgagtttgcctgtcctttttccgtttcaagcgtctgctggggaagggccttctgataccctctctggaatccttccactctatcagtgtgagggaagaggctcagcttcttcatgtatgttgctcctttgccatttggtccattcttgtgttttcagcaaaaccagtttccacaatccaaattactgtgcttctcgactgtggataaatgagaaaatactcctctctgtgaacgcgctccttcgtggagacagtaatgagtacaacttccagagctctgcgcagccagcaaccctggcaggagtccctgtgttattcataacccagagctgtgttgccatggcagtactgcttgctagaccttggacccaccaggaaaattatctttcttcatgaaataatcctaagaagagagatgtggcttgaggagcagatgaaattcatttaaattccacagcactagattgaacattgatgtacacagatgtgcccctttgacatagatgaagctggaggaaattgtactaaaaagtaagtaagtgagaaaacacattgtcattcaatctcttgaggtcccatccaggaagatgataaaaaaggtatcaggtgctggattttcatggaatcaatcctgctttccctagaaagaattaaaattaagatccatctcataaatgggtggatgaacacctgggttgtgttggcatagcaacacttcttcatgtgggggctgatggtgggacccacccactcatacaaacagcgtgcggcccatgcaacacagagatggctagattacttggtctggaaaaaataagatggtcctgctctgttttcttttttctctgaatttggtgctatttgagagacggaaatagaatttcatatacagaaaggcaaacagattgagacaaagtctttgtaaggtgtctgaataaaggaggtgttaattggtgatgaagtccttttatcctttgcgggagaaggatttagatgatgttagggaaaacagccagaaccaagaatatttttctgctacaggaatcttttgtgtaacagcaaaactatgtcaccttctttttttttttaagttatactttaagttctagggtacatgtgcacaacgtgcaggtttgttacatatgtatacatgtgccatgttggtgtggtgcacccagtaactcgtcatttacattaggtgtatctcctaatgctttccctgcccccttcccccaccccgcaacaggccccggtgtgtgatgttccccttcctgtgtccaagtgttctcattgttcaattcccacctatgagtgagaacatgcggtgtttggttttttgttcttgcaatagtttgctgagaatgatggtttccagcttcatgcatgtccctacaaaggacatgaactcatccttttttatggctgcatagtattccatggtgtacatgtgccacattttcttaatccagtctatcattgttggacatttgggttggttccaagtctttgctattgtgaatagtgccacaataaacatatgtgtgcatgtgcctttatagcagcatgatttataatcctctgggtatatacccagtaatgggatggctgggtcaaatggtatttctagttctagatccttgaggaatcgccacactgtcttccacaatggttgaactagtttacagtcccacaaacagtgtgaaagtgtttctatttctccacatcctctccagcacctgttgtttcctgacttatttttaatgattgccattctaactggtgtgagatggtatctcattatggttttgattttctctgatggccagtgatgatgagcattttttcatgtgtctgttggctgcataaatgtcttcttttgagaagtgtctgttcatatccttcacccactttttgatggggttgttttttcttgcaaatttgagttctttgtagattctgcatattagcccattgtcagatgagtagattgcaaaaattttctcccattctctaggttgcctgttcactctgattgtagtttcttttgctgtgcagaagctccttagtttaattagatcccatttgtcaattttggcttttgttgccattgcttttggtgttttagacatgaagtccttgcccatgcctgtgtcctgaatggtattgcctaggttttcttccagggtttttatggtttcaggtctaacatttaagtctttaatccatctcgaattaatttttgtataaggtgtaaggaagggatccagtttcagctttctacatatgtcaccttctttagtgatgaacatggagggtaagcagacagggaatcctggatgttcttgttaacacattcttgtctcctagccgctgtaacctcagccctggagttgacaaaagaatctgtcccagtgggccacagatcctagggattatggagttattgcaaagatccacaaaccattagcaaccaagccttgtctgttgaatgaattaatacatcttggattaaaaaaaatgtactttcttcagataaaattaaatacagttagaattaataaccaataataaattgaaaggtgttattaaatcatagtaaatttgtgtcattatgtggtctcaaagaacagaagtgaagagcacaattcctgtcatgtgggcaatctttaaatttttttgacattaatcagacatcctcatactcaaaaaggatgagtcactgtaagcacaaataaggtgtctctacctgttgctgagagtcttgacatacataaacttcatgacaacaacttctaggatcccttcatttctttcattcaataaaatacacttgagttcattatatttttatcttccactgaccttagggacaaattctgcatgttaagtagggtggattgagtgctgtgactcccaactgcctactgtctctcaaaggtgaataggatgggttgagtaccacggttcccagttgcctgtctctgaaagatgaatcttggccttttctggcttacatttcagagccaacacatgacaaatagtttagatcttgaggaagactgaggaaggcttatggaatactcagcacaattatttttatggcacagagtgaaatagagattgaatacttacacacgttttgattagtatgtgtgttccaccaggcttacacacttctctttgagatctgatctagacacatttttctaaagacatttcaggtattccctctgcagttcataacagtttatgcactgcattgatacaggctgctctcaagttagcaggggacaggtttttgatgtcaactaagtgaaatatagtatcaacttcatgtagggttgcttgctgccatcccctttctttccacaattctgtaaacaacttggtacattttgttgtctagaaatagccaacctctaaatttaaacaggttgagtattaaattattgcagtgaacattttttcacattaaaaactgaatcacagaattcttatttgatcaccacatgtttagaaaaactattttaatgtcactgacatgaagttgtcccagaaacgaaagggtatgggtatacacaatgtagcagtcatttatcttcatttgttcctccatttattcagcagatagttactgggtgctgtattcaaggcatctatttttaaaataacctctgtgaaatgttcatttcaaaaatgtctccccagatgcattcaagatgccttctattttctctaatcctctataattgcttttctttttccttttttctttttgagctgttttccttctctccctcccaataataggtctttatttaactatacttattttcatcatataaatatgcacaatataactaaacacgaagttgaagaacatgcatataagacatgtcttatatgcaatgtctggaaatcactacagtgtgttttaattttgattgatgtacacatagcctttcacattgaaatctaggtaaggtaagtacagctattaattatggaaactagtctatatatttgttggaatatgataaatcaagtcaccgttcaaagttgaataaatagtgaacacaatataacattaatgcagattaattaccatggcccagagaccagccatgagacataacatgcttcgctgtcaacaacattcaggattggaattccagagatggcgtgtgctactggcttatggctgatacgtaagagaatctcatctatctctaaatgttctgccttaagccgcacaaaggacgtaccagtgaacaatgagttaccatgtgcagaaaatattctgcaaatcaagaaccagaaaagtaagcaacccgcaaactccaaaactcattacatgattgtgatctttattttgaggtgcatctaaactgatcttcaaactcttcagctatacctgactttctctggaatacttctcagcattttcaaactaaaccaaaggctttctgagttacataaggaaggcaaaatttggatatttgacaaatttggaagcactgaaaatggaggaaattccgagatgacagtggttttggttcttgtggttcttgtggttttggttcttgttttttgaggggtgcagagttgggttacttaagactctgtgcatggctcaggcatcattgggctgtgcaacactcgtctcatgtcctacttgattttcttctccctttcctcctctattcccactcttccctcggcaggttcccactctaatgtgtctgatttatatgtttgctattaatgtgttcttatgaagtggagtgtgctgtgtgtattcaggtgatataaatggtattgtgccactccagatcttgtatttcttactttttcactcaactatgtgttttaagtgtctacccaacttgttcatgtgtgagtgcattgcttccaactgtcgtgtgattttcattaggccatacctgctgtgtctctggtctccatcttcctaatgatgggcacctgggttgccttcagtaattgtgatcggtgttttcatacatgtccttttctgacctgcgtaaggctctccatggagttcaaggcctagaagcggatcccttgggtcctggtgtgcaggcaagctttggaccctgaataacgccagcttgctttctgtgtctaccgcactagtgggacttgtgggtccctattgtctacatctagatttagtatgcttcagtttcctaatttttccagccccctcagtgctttaagttgcacatctctagttactggagattctttgtaccttcactggagcacttggaaaacagaagaaatcaaagggcagtcaggactctcccagcaggcagggaccaggtcagagtcaggatgaagacaaagcaccctgagcaagaagcaatggaagagatgcttgtgacccgagctgatggggaaggttgagggcaacgcagagaccccactgccgacttgccaagaaaacgtgcaggaaatgagggaaggctccattttcctgagaaaagggatggaggaacataggcagcatcaagctccagctactggctctttgagaatgtagtcgggacttgccccagtcacccttgccctgagaactcagtgactggggctaatgtcaccctggctgtccttgacctgtgcagaaagcactgccaaccctggacagtgggggaagggccttatgtgcccaggccccagcaggagacacctaccaggaatctagtgggacaaggtttcagggatcctggaatcctgcccacacccaacagctggaacagttccctcagctggcgagtggacaggtggtctgatgagctcatatgttattgatgatgatgttaaagaccatgttcccatagaaataattcccaaagcattcagaagactgagaagaagtgatgataagaattgttctgggcaggagtgtgtagttttgatttttacttgtgataaagcagaggatttgcttgaattcactcaaacttgctttttctctctcttcaaaacaactttggctgaccagtgtctcatagacactcaatttgccttttcatggaactcaaaattttgtgtatccatacatgctaccatagtttcaggcactgggcatttcaaaaatgtgatatttagaacctttctatttccgtgatgactgttggcatagatgctgctggagatttcgatgacatgttggattcagtccacgcaagttttgatgcatattcccttaatttctgagcctttagagttagagttttagaactgagagtacattctcttcttttttcctttctcctcttatgtcaacaggctcccttccccacccccccacccacacacacaaacaaaaaataaaaataaaacgctctcagctttgcttttaagaaatgaagaaatgttccaacatgtgacccacagctgcaatccgttcacctttgccaatactgaggctgccctctgggtccctcctgggactgcatagatagaggcccatttgctgttctgggcagcatgcccagtcatctaggaggtgggggactggggcactctttcaggccaactcaaactaatggaataaaatatcttctccatgcagtgtctcaaagaaagagccacctgggctgcaggtgaccagactcgctcagtgcagcacggtagctcctagcctcatgtgcagtcaaacacatgagatgtggcaagtctgaactgagatgtgctatcaagtaaactgtacaatggatttcaaagatttaataggaagcaaagagtatggaacgcctcatgaataatttcatattggttacaccttgaaatgatcatgttttggatctattggattaaatgaaaagtattataaattaatttcccctgtttctctttatgttttttaatgtggccactagaaaatgtaaatgtactcgtgtggctcacattatattcttactggacagcattggtccagaggactgggatttatttttaactactttaataaacatcaaacatttgtgataattttaatacaaatatttccccatcgtatatgctcttgcatggtaaatattttatctactcttgttttaaaggataaggaggttttcaaaaattgccgaagtactacaggtaatttggcaaattctcttttatgggtaccagatagtgctatttccacccagaagctggctacagctctgtaatgtgacatgcttactcctggctattctgggctggtcctcgtctgactaatgagctcattatccactgggcagtgggatggaaggacatgagcacctcagctgtcactgccgagcttcctcccagccccaccaggccctctggttcctgatgcttgtctgtaagaccaatgggtactttaacttttgaaggtggtttctggttcccccaatcggtgagcccaaagacctcaaataacattttattcacacagacttcttagagatgaaaagtttctagagagcagagcctttagggaactagtgatgatgcattcactgaataaatcacagtattatactcaagagcaaatacgttttctttcttattggtgtcatctttccttgtgaaatacggcaactgatgaagaagtctcctattgagaataaccagacgaaatcacaaggctagacaagcagctactaacaccatccccttccccctgcccatggtaaaccctgctggcaaatgtatttctccctcctaaacctgggaggactcacgtttctgcacccagagccccaggtggaagtaaacaatgggagtgagccgatgaggtgggattgctgttccttgactcttagtgcttctcatgctagtctcctggggatgcagttaaaatgcagattctctctctagatctggggcggggcttgttcagcaggctcccaggggagaccaacgctgctgacttgtgggtcatactttgagttgcaaggttgcgtacgtggtatttgccagtgtgcacattcatgaaaaagtatcctgccttaagcctggtagatccacagttccaaatcaacattaaatacatctggtatacttaagaaaattcaacataaaaagaaatccatttcttaaaaaaaaaaaaggcagagcaaccattctctttatctgaggacctttaaaagcatctgcctttatgtgtcacagcattagctactaattactaaaggaaaaagtgctgggtgaattatcacctgaaaataaagtaactctcaaatgttataaaacataagattaatgttgacagcctctgttactatgacatattgtgtgccaggtgtgtgtgtgttataggtgcatatatgtatatatatacacacttgtatgtatctgtgtatatgtacacatatctatgtatctatatagacagatgggttatttaaatgaattttcaaaacactaataaggtgtgatttgataaatgaataatatttaataataatattaaaataatatatttgaatattattattctcctgctactgatgagaaaactaaggctcaaacagtttaagtaacatgctagtaaaaaaatgttctgagtagaataccacatgtctggcctaaccctgactgtcttttttctgacccgagaccagtcctccacgtggggagctctgaacaccatatttataacagatggttatttataatgtctttctcttctttggcaatttaaatgaagacgtcaacttgaagaaatgtttcaccaggaagaattgcaaagagagctgaattaatcaaagaaagacgaataaagtgttcttttaatatggagtacataattgttgatggaaaacacagtcttggcagaattaaaagataatcaaagagatgaatagggcaaatagcaattcagctcattaaataaatacaaggaaactaacctctgcagtttaaccttgagcgataccttttcccatgaatagagagtgtcgatactaggcaacaagcctctgaacagatagtgttacccggaacatcacccttttctccctttgcttcaaatcaaaaccagcatcccccatttagacagcataaaaggtatgacaaaacgggacgccaagtgagacactgttgattctgtttctatgaaattctagaaaaagcaaaactgtcatgacagaaagcagatctgggtttccagagcccagagtggggggaggggactgacttcactgagacacaaggaaacttttggggatcatggaaagggtccgtaccacggttgtggtggtggttataagactatatatttgtcaaaagtcttcaaagtgtgcacttagaattgctgaagtttattgtacataaattacatgtaaatatcataaagtgcatgtaaatggtatgtgaggtaataaagcaattttttaaaaacaattttaaacctggtatagcattttgtttttttgtgtgtctgactcagaccaggcaaaatttagtaaccctttggcaacagaaaatcagcacagatatagagctagaagttattgtaaaagaataatagtggcggccgccacggcgcaggagttgccaagattttcctggatgaattccatccaagatacggttttaagtctctcaaggcctttggatgcctagaaagtgggtcatctttcagaatatctgcgtcactgtggagatgttagtaaatcaaagaccagctggatttgtccttaaatgtagcaaaatatgtacttgcttggaagtactcttaatcctttcattcactggacagggaagactggtttcacagtcctgactcccttcattcgttatcaacacctcgtattttttttttggtagatgatatatatgaggcagtaaacttgtggtctggaaaagcagagatcatattaccactctgttttaacttctaaaagtttgaaattaagacttataccaggtatttttatggagtggattggagtatagaagcaattattttaactatttgctaaaaaaaaattgcctaaaatattcagttattaaaggaaaagcattgttctggattggattcttcagataattctgcttttggcaacaagtaacagaaaaccaacttaaataggtttggacaataaggacattcactggctcgtgtgtctggaattctagcaacagcagcattcagggttggccagaccaggatctcaacaatcttattaaaccatggttttcttctcctctctccactctgttgacaggatagcttcatcttaagacttgttgtctcatcagtgtaagatgactgctaagttgtatgtgagcccacaatttcctcattcattccttgaaggaagaaaaaagatttcttcaggaacttctgacttaaaagcaatgaaggactttcccagaagtccatttctcattggcccgaattgggttgtatgcccatttctcattggttcaaattgggtcacgtgcccagtcactgacatggaggatacgatttccctaaactaatcaggcctcccgctacaactagaaggagactcagctcccacaaacatatgaattgatggtgcgggagagtgtagtgggaattctgttaggaaagagaaggaagtcaacaatcgggtacgctgtgtctaatttaagtccatgactttaaaagtgagcttttttttttgttacaaaaataatgtaggcttaattttttttttttttttgagacggagtctccccgacacccaggctggagtgcaatggcacgatctcggctcactgcaactccgcctcccaggttcaaacgattctcctgcctcagcctcccgagtagctgggattacagatgcccaccaccaagcccagatattttttgtatttttagtagagacggggtttcaccatgttggccaggctggtctcgaactcctgacctcgtgatccgcccgccttggcctcccaatgttaggcttaatttttaattttcagagattatttgtagctgaaaagaagaaagtaattaatgaaatgatacggcatacttttaaacaataaatatcatattctacatactgcagacacattaccgcataatggttaaaaatacagactttgctatcaacctcctggttcatcttcttactgtctgtgtgtcctccttgggtaaggtacatagctttcctccacctcaacttctttctctctgtaataggaatgatagtagtatctgtacaagtttctccagcactaagcctctcattcaggggtcagcaaactacagcttgcaggccagacatgcaggcaacctgtttttgtaaataaattttattggaagccatttatgtatcgtctatgactgttttcacactacgatggtaaaggtaagtagttccaacagaggtcttagagctcacaaaaccaaaaacatttaatatatagccctttgcagaaaacgttcgccaacctctaggttaatacataagatctttattttcttgtcaaaatctaacatgtcataattctgtggtcattgagtgactggagatctgccagagaaaggcaattatctacagagctaacatgaaagcaagagatgctcctttattttatcacttaaaaacagtgaagaatcatgaacaactgaaaaatctacaaatgtgtttaaatgtcaaattaggctgggcatgatggctaatgcctgtaatcccaacattttgggaggcgaagtcaggaggattgttcaagcccaggagttcaagaccagcctgggcaacacagcaagaccctgtctctactactactactactactactactactactactgctactaataagccaggtgctgtggcatgcacctgtggtaccagctactcaggaggctgaggtgggaggattgcttgagtcgggaggttgaggctgtagtgagtcatgattacaccactacactctagcctaggtgacagagagagaccctatctcaaaaaaaatttttttaaatttttttaaaaagtcaaactatctacctgaactgaccagagatcttgaaatatgtgatgtaggatttcccttcaactctgggatgttggaactggaaacttgagtgagagtgaatttgttcacactcatagctagtggatagaagtcatagctctcagcagaagtctgaagaggaaataaaagagagccagtaataaattgctgtgaatatttcttttgaaaagcaaagaaaaaagtattgccacctgtgtgtgctgaatggacagtggtattgatatgtagacaagggatcctgtcagggaatccctggagagagaagagtctgagactgacactgcccggcagccccaagtctgagttgtggaaaagccctcagtaaaaggaaaaactcatggcaacgagttggtggtgatactttcatggaggaacattgtccttgcaggatgggtggttttaatcctgaatttaaagatctttaaaatctaaattatctagtcaaatttttttcataaaacaatgttttaattaaagattcagttttaggtcaagtgcacagtgtctttctttaaaaatatttagatgtggccatcaactatcaagaatgtttctagaatgaaatatattcagcagtgctgtgtggggatcaggaacacaggctccaccaatcagttgcctggtttcaattcctggctgatttacctgtgtactattcactcctgaaagtcctaagccttgttttccttatttatagaataagattaacagaagtactaactttatagagctttgatgagacttaaaataatccacgtaaagtgcttagtctagaatctaatatcacacactccataaacactagccattttaatgtattaacacatagaaatgaattgtagaaaacattttggcaacaaactataccataatatatatttgtaatctatggagttcctatctttcacttttgatattcctgtcttgttttttgagcagcaaaggttaaggtagacacagaaaaatccctcactggggtcttcaaatttagaaggttagctcaggggtcatcttaatagacattatattagttaagatccaggtcaacttgctaatgaaacaaggccccaaaaatacagtgatgaaaaagataaaagtgtctttctctctagcacacgcacacagaggtgagcagccaagctctagtgaggcagctctactccatgagttccatttatcttgcatctccaccatcgcctacacaggtggtatggttgaagctcaccagcatcttcttccagcaagttattacctttttaaatgcagagacctagaacttatacacatgacccctacaaccacctcattgctgaggtcttaatcacagtgccactcacagttgaaaaagaggatagaaaaaatgactgtcactaggctgccatatgcccaactaaaattctacgtattctcaaaagagggaagggaagcatggatggtggaatccatcagcagtcttccacggcgattgtcatggaaactattgttttgttgactctagacactattgattttaagacacatagtcattttatgtaccactaagtaaaggaaaccatgttgtttaaactgggacacatttctttcttaccagctcagttgtaggacaccttccaattaagagatgttaaaatgtagaaaaccaatgtgtcttggtatagaagaaatacaggattgtgaagactttggaaaagaaagtgaccaaaggcattcattttgcagtctacttttgtgtgtgtgtgactttccttggagcaaagataggcataagatatcaagctttggttgatctattgaatttactgtttagaatctgtgattgacaaggtactatcagtgtgtgtatgctctaagtcaaattcctgtgattactggtagagagactttagctcaaagctttggggaaaatgtgcctagattttcattcaatctgggccaaatctggagttgtacaaatagatagcatcggtcttgtccttagtagccagcatttttgtttaataggaggagagaaacccaggacgacactgaggaacagatatgaccagggcacttgacttcttgtttcagcactcagtcattacacaaacagttgttgaacaccaggcactttgcttggtgtgacaatgcactcccgccttccaagagctccagacccacaggggagtgctgttgtacaggtgctaggatagaggtccggggagtagaggcagaatttgggagccatcagttctatctgaggctaggaaaggcttcccaggggaggtaaaatcaggactgaggaggtcaaagagccctccatgcagaggtaagagactgacaagcacaggaaactggagctgcggtcactgcagggtatcatgctctgatgcttccgcatgttagactggaggcaggatgtggggattggggggtgtcctctccagcttcgcttttttcagtgttttgtatgtagttggccaatagcagtatttgttgaattgaattgaaaagaccagtttcttggatggccagctagagatgagctgaatttttctggatcaaaaagatgttagattacaataaatgggcaaaagcaaaataatacctttggtgcttactcttaaacttaacattactctattttgtttgtgaaagtcagatttgaggatttagtttataaagaaacccttaattggttggatcccacttgcttcacccaccctgactctcatttcccccaatgtaataaatgtgcatatgtatttagaacatacagagcagtttaacataaagtactgaaattatcctaagaacagattctcagtgcagcaatcctatctttgtgaatttgatgtgttctaaatgcaagagcagaagtgttttaattgtcaggttttaagatattatgaaagcccctagtcacttcagtaagatcatttgattctggttaatgacaagtctatactatgcaggttttttttttttctgtctattgctactgaaatctgcttttcttatatagatcactgttacagatgaggaagttagtaatccatcctttttagatctggtgagaactccccaaatgaggaaatgcacacttattcttatgtttgcttggtaagtttgacttgtgatggatttaaaagcttgcgttaaatttacaatacatcccttccttcaaatcagcttgaagttatctcttacttgttctctggtgtctttcaatgttgctacaggttcacaagcgcagtggtgtatcaaggacttgtcatgcgcctgggaattatagggggcaacctctatatagactttttcatctcgggcgtggtggaactgccaggagctctcttgatcttactaaccattgagcgccttggacgacgcctcccctttgcggcaagcaatatagtggcaggggtggcatgccttgtcactgcgttcttaccagaaggtaatcttaccccacatctgtttggcagccaaaggacttgaaaacacctaaatccacaatcaagtatagggagccacttgttcttaggaaatgtgcaatggaatcacagtctttcgtgatgtcactccattttctgcttaatcagcataaaaacttcgaagtaagagattggttctgtagatgattctcttttctcaattgctaggatcatttatgaatgaaatagatattgtgcccaatctagctgatagcgatgtggtttctgttactcatctttatgtgacttacttttgtgatatctctgatgtttaacttatttcctgaacctctgccatgctgctggcaccatgctaaacgctgaggttacacatagaagaacgcagtcttgacttcgaatggttcacaatctattaagggatacagatactttaatattgtcacgattcttattgtcatgaattatttgtgagatttagaaactaattaaactatttacaagatttagaaatataaaaaaatagattggggaggagtgatcaatccagaaggggattcaagaaaagcttcccagcagtgggaactaaacatgcgtgaagactcaaatgtgccagatatcagggagagagaggacggacgtttccaaactagagaagagtttccagaagtatgaaacacctttgtgcatgcaggaactgagggttcattgctgtgactggaatccagacaaggagcctgagagatgatctgtcaccagcatgaccagccctgcaggtggtcctgtgagcttgcggaggaggaggagtgagtttggcataactgcagcagtttagagatggctatgagcagggggagatgctgagagcaggagttacaattggggcagtcagacctagatgagagagggtcatttcatttcagagatgttgcagagttaaatttttcagagctccatgcctggtagatggggtgcaggccagaaggaagcagagggtgccgtgactcctgatgtgtggtgagcagcacccagaagatccttaccctagaacacaccaggaactgggaggaggagggcttcacatcccaggggatgtgtgaagtagagcaagttcatcattcccctcttccaggaaaggcaaagtggggattacagagaaagctggattctccaggcatcctttgtgtctgcacaaaaaaggctgtctttctttgagaatcctctgaggaccagccaagaaatggacaatatagtgggagtcagaacagtcagacatttacagatgagtttattaattaaatgaaggtttatgaattcttaaaacacgaggtaggccctatttgaataaaattacaggaatgccaatattcggaacacacacacacacacatacacacacacacacacacacacacacaatgtacaagtcccctgacatgttggaatgtttgatgatgaacagcaaccacctatttacatttctcttgttttccttgagtgcattcgcttcccagggctgctgtaataagtaccacaaactcaccggcttgaaacaacagacatttatgctctcaggggtgtggaggctgggagtctagaatcaaggcatccaagggtgcttatgtccagaggttctggggaagggtctcttcatgcctctctctggcaatccttgcagctccttggtgtcactcctatctctgcctccatattcacaggacattctcttctctgcttgtgtcagtgtcaccttgactaattacatctgcaaagaccttatttccaaatgtagtagccttgaggttccaggtagacatgaattttgggagacactgttcaacccactacatggaattaacatattataagagttggaggatctgtacaatagctatctcttaatacgtatctatttagtatcgactaattgccagccactgaactaggaggggaatggagaggtgaacaaaatagacataattcctgccctcaaaattgtataatttaagaagagtgaaaaatcgcaaacattggagacgttactgttctattttactttggtactttggatataacatacaactgtagctccctgagatgaagtcttctgttctcatcgtatgtggactaggaatgaatgaaatattcattgtatatgaaattagaaatgcagcgagaaagatgcatgcttcggtgtgtcccctcatttcatgatatatatatatactttactctataagattttctctggtcctagattgattgggatgactttaatgtttgttgcattcagaatgaccccagaaagcattcaaggctgtgtgtccaaatcaagacagaagtaactagccggatagtcatataactattttgtagagaattctttcatatattggaaaatataacaccaagaacagactgttctcccttgtctaaccagttttattttatagatataatttcctgattgaagctttagaattgttctcagcaagactgaaaatgaattgctcttattctagacacattttatttttaccttctgcagacttggctgcttttatagtcatatgtcagtcatgactgactggcaagaatacatttcaaatccctaaactgagttccaaatattaacagacgatcctcccttgagtccttccaatttaaagacccattggacttgattccagccactggttaattagcacgagaaaggcttttctgctcagccattcctgacgtcagtagtaggtcgccagcccctggggagaatacagatgagcttttggagaaaccagctctaaaatagtctcacagcaaacactttaatcagctttccctccactcgacccaacttttcatggtctaaagaatggaattctggaagaagaaatagtagttctggttgtattctcatctcagactcactgcttctagtccaaatttcttaacactcattaatgtctggaaaataaagacctttctttatccatacctgtctgccatctttaatttgagtttatttaggatgaccacaaacctagcagtttggataagtcgtcttcttgaactggaaaacttgtcttccacttttatttttctttaattgaaaagtgagttcatgtatcttaatttagacatttctactagaatttctcactaaactttatttcagaactcagagtagatgatccattcagaggacatataaagttttgtttaagtcctgcatcctttcttcaggaattgaggtattattcccaatcatctctagtagcttgaccaaatccacattcagaagaaaacattttctttgttctcatgtaattccatggaagacaattttagtttcagtactcatttttaaattgccctgtggtttttgtcattgctaccaaaagatgtctatggccacctcaaaatgaatttagcttattactggaatcaagaatttctttgtgtgtatatatttattcatacttcctacagttttcttttcactctttagttaatgaaccatgcaatgccttagaaacttttctttggttacacatttcaacaaaatctctgttccagtatagtttgatactggactctactgcttggaaaagagttgggagtctttcagattaaggtcaatgttccggacagaccacatctccacaaatggggcaacttggcattttctgaatgttttactgaattgactggctatttatctgagttaaccacatatgtttatcatggtggagtgtgtgtgtgtgttttaatttggtctctggcttttgtttggatttttccatgaaacaccttgcctcggttataaacccaaattgcccttgaatttgtcggtgggactcaattttagagcaatcttgatttagtgaaaagaaacatacaatggaagaacccaacctggatagtgaagctgtttcctaggtagcaggcagccttcactgcagcccgggagtggcaggagatggtcccatcgggctgggacccagggaatctgagtctctgctcccactttccccttccagtaccacatgctcattgaaagatggagtggaatgcattacctagtctccactccccccttttcttggtcttcataaagaagcaaagcctaaataacacatgttgacaaaccttctgctccagagcagacagtggcccagcacatcagcacaaccctggcctcctcactgcctgatataaccgttaacgtgacagctacaagggttctcatgtgggaaaaacatcaagtgcaccaggatgtccttggccagtttgcagtcaaaatatgtaaacttcagactcccagaaatggtttggattcttgagccatcttttccttacacctgtgctcctgggcaattgttttatttaatgaattttagcttttttttttttttttgacttggctattctctagagagaacacacagaagtctactgggcttgattctcttctttcatcctcctcattcatttctactaaaaaattatatattcaggaaggttcaataattcccctaggaaacaggaggactatttagtgctaaatgaacatatgtgttggatataaagggaaaatgaaaggcacatgaacactggacatgcttcgttttgctttggagagcagatatggtcttgttcatgcagacatatgtcctcaataccaaatcccctaggaaaggatgacatcaattgcacaacttctaggaaagctagagcagtgactttttagagtaaaaatggttaactcctctgcccttaaaacataaaagaaagaaaatatttaatatagaatgtttggcatttcccactagttagtagggaggaaaggaaaatttcttcatacaaccagagcagtcctaaaacagttcattttaaaatggacgaaccaattagcattgatgaatgtggctccttctggatggtttattgaaggaagtagtacaatgggggatgaagaagacaatgagtgacatttgccttagtggtaacacacagaatagatgtccccttccaaagatcctggtcctcaaagcttgctccactctagaaaccaaggtcagctcacgtgaggaactccccaggtgtgctgggaacggctattccttgtgtcaccaccgcaaggtatgcacagaaaggacagctgggcaccagctgtgggaacgtggtggctcaaaggcatgactggaaactaccacctggacaaaaagccagtgtgctttggctctctaggaggtcattgcctggaacaatgttccaatgtagtggctgctcatgctcaacaaacggttcttgaattgtacttcctataatccaacctgctcagaaaggcatctagcgctctaaaatattagctatcatctagtttcattgttgttgaaatttggttacattattagaaagttctatcatttgtgcttaaaattggcaaagacttcagactggaggccactaagcaaatactttttgttgttctgtaaaatgttaatgaaatcagacccctaactcctccctttcaaactttctgtgtttgcaggaatagcatggttgaggaccacagtggctacattgggaagactagggataaccatggcctttgaaattgtttatttggtaaattcagaattgtacccaacaacattacggtaattctaacaaacgttatagtttctcctaagtaactctgtagaccagcgttttaagttgatttagttaaaaactatgaagtcgtttccttagctcagtgatatattagcctaggctgaatccagctagtaaagaagatatgctttgagttatcactgctttctgcattgctgcctgctgcattttgtgataaaagtatttctcatattaatatcttcacacactgttacggttttgaggaaaggcccttaacttgaaaccagaagacctctaggtgcccttgcttagactgtgggttaaatctgtatccatcagcatcacctgctgcaagactatggcctctctttgactgcattacatgcattgcagtggtgagcagctggttatgaaggatgaaatgaaacagtgtgcacggattagtgcaaatcaacctcctctgctgaggaagcattccctgggcacacttctccctcagatgaggaaaccaaagtagcacaaagtgacccagagcaggcagggcagtgggttgctgatggataacaccctccacccacgctggagacttcctcactagccttactgtgaaacgtgcagaatggaaatgttttgcttagagttctgagagtagtcgttttcaaagttagaaaatgcaaagtatcttcacacttcctttggttttttaatatgtctgaatatgttgattatcttgaagtcacttgttgaaatagttttcacttaaaattacttttcattcaacagaaatttcggagtttcgctctgttcaggtctgtgtgattttgggggaatcatagccccatttctgctctttcggctagcagccgtgtggctagaactacctctgatcatctttggtaagaactcatttgctattcttaagagccttcatctacattctttgtactaaaagagacctataataattgttaggttcaataatgataatgatagtcatttatcttattataataactatttcattgataattatgatattaatttcatcttattataataactatttcagttttgagagccaattcagttataatgaatggttttaatttactttatattaaagggtgctgataaataatgttcaattagcattctttgggttctgagttaaatgctgtgaaactcttatggtattggagaacataaccaataattttcaggggactaaattaaatattcaatatgtttaaaaatgagggccaggtgcagtggctcacacctgtaagaccaaaatgggaggactgcttgggcctaggagttcaagaccagcttgggcaacatagcaagactgccttgacaaaaatttaaaaattagctaggcgtggtgatccatgggtgcctgtagtccaagctactcaggatgctgaggcaggaggatcaaggctgcagtgagctatggtcacgccattgcactccagcctgggcaacagggtaagaacctatctctaaaataaataaataataaaaaataacaaaaataagcctaatgtctaattgtatttctggtgttctctctctatggatatcatatcttcattggcccgatgctaaaaatccatgtggagggagcaggtgacacaggatcccgggcacacattccatcacctcctccaggtccctcgagtgcacctctcctcctacctccagcttccataggacttgtgaaggaacagaatcatagtctaggagggaccttggggaattcagtgtcccacttctccagggctgctccaacggtcctgcctgggtttcacacacttcccctgtgccctcttctgcagtaaaggatacgggttggcccctttctcacgcaaccgtttctctatctccacttccttacttctggtagagaatgtttttcatttatggttcagcagttgaaatttgaatgaaaatactgagcccatgaggacagaccttctgcttcatgcaaaagtcctttgacagtgtgagggacaaagagtcggcttctaccacttgacaccagtgacctcaatacaggatcacctcaaattattgtatgggatttggtcatcaagctgagtgttaattctgtggtcagcatgaaaaagcagaaagtgctactgtccagcaaagacagagtagtccaggagacaagacacatgaccttcaggactgcagcccacagcccagtggcaggcaggcggggcctgagactgcgaggggagacagccgaatttctaaaaactcagcgacaacagaatgtgataactgctgagaccctgaaagcagaggctcctatgagaataggtcacagggacccttgacccctgagtggaagggagcaggccagggaggaaggaagcaatgtctaggccaagaggggctggctgtgctccaggcagtggaaagttcaaagcaagaggatgccgcatcctcaagcacctggtgctagaggatggagtgcagttgtgagctgggcctgaagagaggtggtgccagggcagggcagggactggatccggaagggcctcaaggacatgccaaggctttgctctttatacacacagcagtggaaagtgactggacagtttcaccctgaggtggacagagctggccttgcattgtagaaacacagatatgcaagtgggttggaaggaccccccagcatcgtccctctgatggtagatatgcaagtgggttggagggacgatgttgggggtgacaattcaaggatgtcatcatcaggagatcacagtggcttcttctaataaagggatggcaatgggaatggagagggtggacagattgagtaaacatcttagaagccagctgggcaagatgggatcactgatgggcacagggagaaagcatggtgacctgagaagcccaggtgcctggggagaagcaggtggcacaggcactgtagcagcaaggctgagggtctgcagggtgatggggctctgtgtccgctgtgcataatttgaggcacctggaagagttcaagtggaaatagaaaccagcaggctcaacaccgctgagctcaggagggaagagtggccttgggatgaagagctggaagccacctggcagtagagaatggttgaagctgagggagtggcccaagccccccagaaaatcaggacaaagcaagaggaaagcaggaggcctaggacagagacctgaggggtggcaaagtgacagacctaggcagaaggagaggaggtggccaaggaagtggaaaccaagtgagaagcccgaggcaggagggcacgagaccaggtccaacctaaggactgaaagtagcacatgtggatctggtggccagcaagaggcagaaggaacctgactggagcagagccagggagaatataggctgagtctggaggagggcaggcagaggcaggacctggagcatggtcctatgctggtgctggcacctcccactcaccagggccaactgctaccatattagttcgtttgccactgctataaataactgcccaagactgggtaatttataaagaaaagaggtttaattgactcacagtttcacatgactggggaagcctcaggaaacttacaatcatggtggaaggggaagcaggcacgtcctacatggtggcaggtaagagaagagcaaggaaaattaccacttataaatccatcagatctcatgggaactcactcactatcacgagaacagcatggggaaacctccccaacaatccagtcacctcccaccaggtccctccctcaacatgtggggattacaattcgagatgagatttgggtggatagacagagccaaaccatatcagctacattttcaagaattttgtaagccagtcatcaagcacagatatcatcaaaaagtaaagaatatgaaatatatgagaaatagaggcaataaatgcccaacactcatcagtccctaattcagctactacaccttcagtattacctgtgctcctgagattgtgtctgctgcatctttatggtggaagccccattgatggggctcctgcatcacttcccagtggcccaggcagggatgttgtgtcagtagcctgaagggcccacagtgggagcatttaaaccatgcacattggcagatgctgccaatcagggcttgattgcccaaagagggttgttaggcatttaccagcacaccactaggctacaaagaagtggagaaaataggggagggtggcttttcaagaagtttggctctaaagagcagaaaaagaaaggatggtaatggaaggatgaggtgagattgagagaaggctggtcaaacaggaaaggtctgaatgtgtttgaaagcttctcccaataggttgaagatactggaggaagggttgatccctacagtgaggagcccaaagagacaggcctaggagaaggctggtcccggagtagacaccaggacaagctttccactgtaaggtaacaggaggaatcggagggagaggggcaggtaagtgagagtgtcagtgagcgggcgcagaggggactcccctccagcacttctgtttccctgtgatgcaggagaggaggccttgggtgttggaccctaagaaggaactcagtcaatcactgtgagatgtgtgagtgaacacatggctgccaggtatacaggggccgtggtgctactcagggtttgaggaagggagagaacctttgaagctgtggtaagggagagctggggcattgatctgggatgcagaggttgctgtggttgagagctactccagtgagcaacatgatggcttcagagtgagcaggccccatgggagagggcccagctgtgtcttcctggagcggtaacacctttcccctattccataggtatcctggcatccatctgtggtggccttgtgatgcttttgcctgaaaccaagggtattgccttgccagagacagtggatgatgtagaaaaacttggcaggtactgtacaaaattcaatgcaccctaaataaaagcaatatttaaaaccacgggggaaagaatgacattgagaaaattgaatatccacagggaaaaaaataagaataaatcctcatctcatattgtaagcaaaaaattcaaagtagatacaaaaggttaacataaaaaaaaccctccagagctagataagagtatgcattgttttttttaatgtggttgtgaagagagaagcattttcacataaggtgctaagactcagaagtgatctaaaggcaggaactgctgatgtgggaaagtggaaattgctgggacatacagcagcaggggattaacagtgtggcttactctacagagtgaggatgcgtcaggtctgtctagatggaatggcataggcaggttttcaagactgactacaaagtgaaatgtcactgaacaacatgttatagtatttataaaaaactaaaaccaaaggtgttttctctctgcagctggataggaagacagatgacacacagatgatagataataggtagatgatagataataggtagatggatgatagatagatataatagatgataatcatagatagataagtgatagtagataaatgataaataatagatgatagatgatagatagatacagatagataaatagacaggtagatgataggtaatgtctggatagataggtggatagataaatagataatagatgatagataataaacattagtaggaagatagagagattgagttttggaggggggtgtttaaatagggtataagtaaaactaatacccagccaatatattaacatgaggagtaatgtcaagaaaaattcacattacacaaattctttctaattgtttgaaattttttcattaaaacatagagatatgaatatgagaatgtatagtacaagatgtaaaatattaatatgtgccctctgtggacacaaatgggctgggaagaggggtagggaagggaggaccatcactttcttgccagagacttccatactgtttgattttctttaaaaattagaacacgtagtcttgtgttgcttgtgtagttttaaaaaatgatgtaacagctaatttaacaactaaaaagcaatcttaataatgacttaggaattaagaacatggagctctaaatatttttaatatataaaaaatcctgaggaacagctttcttccctttgattctattccactgactgccttctgtttacacaatgagagtgatgctttcattctttatccccaaaccaatcaggatcagatttgcaaactcatcaggaaaaaatggaagaaaagggagtcctctgaaatcaagacttttctactgcttcagtaacattaaaaataaacagctaggagaggtttttttgtttttgtttttgtttgtttttggcttggggagtgtgggtggaagggggttgtctaaatggtgtgcaaggaaaatcaatacccaactaacatataaacatgaaggattataccagcaaaaatttaaggtacccagattctttctaatttttttctgtttataatttttcataatgaaaagttgggtacattaattaattatacctagtctctatacatgaaaaaaaatatagtagaagtatggtttacagtgctacaatttaagcacattaattgtgatccatggttatttactctacaaaaattacttagtgctaaattactaaaacttgctagcatttcccttttaaaaatcacactggattattttatcgtttctgctggtttttgttcatgttaacagctcatttccaaatatatgttaattcagtagaagttcataaagaacttaaatgctataatgctaacaaacccctgtatcagaggaaccagcccccaatatttcagcataggttctattttccataagtgttggccagctgagaaataaaaagagtacaaagagaggaattttacagctgggccgctgggggtgacatcacatatcggtaggactgtgatgcccacctgagccttaaagccagcaagtttttattaagggtttcaaaaggggagggggtgtaagaacagggagtaggtacaaagatcacatgcttcaaagggcaaaaaggagaacaaagatcacaaggcaaagggcaaaaacaaacatcacaagacaaagagcaaaagcagaatgactgaaaagggtctatgttcagcggtgcatgtattgtcttgataaacatcttaaacaacagaaaacagggttctagagcagagaactggtctgacctcaaatttaccagggcggggtttcccaatcctagtaagcctgagggtactgcaggaggccagggtgtatttcagtccttatctcaaccgcataaggcagactctcccagtgcgactgtttatagacctccccctaggaacgcattcctttcccagggtcttaattattaatattccttgctaggaaaagacttcagcaatatcttccctacttgcacatccatttataggctctctgcaagaagaaaaatatggctgtattctgcccgatcccacaagcagtcagaccttatggttgtcttctcttgttccctgaaaatcgctgttactctgttctatttcaaggtgcactgatttcatattgttcaaacacacatgttttataatcaatttgtacagttaacacagtagtggtcctgagggacatacatcctcagcttacaaagataacaggattaagagattaaggtaagagatgcataagaaattataaaagtattaattttgggaactgataaatgtccatattaaaatgaaatattcacaatttatgttcagagattgaagtaaagacaggcataagaaattataaaagtattaatttggggaactgataaatgtccatatcaaaatgaaatcttcacaatttatattcctctgctgtggctccagctggtccctccattcagggtccttgacttcctgcaacacccctaaggtctgaacacagacaaaaaatgatcctggagacagatattgttgttaccttagagttgggttttcatgaatgtatttgatcaaaaccagctatactattaaataataacagaacaccctcttctaactagttacataatctttcctaaagactttctcctttgttttttcagtccacattcctgtaaatgtggcaggaataagaaaaccccagtttcccgctctcacctttgaggcccccgacaaagacagaaagaaggagctatccaggagctgatcctccttgcaaagctgtgccttgcagagatgcacgtgtgcatttcagctacatcatgccgcgctgttgtaatactgtataaagacctcaatctatccagagtatttttatataatgttggatgagttaggatttgtaatgctgttgaagtttctgggaacacataatatgtagccagtttaacaaagaagctgtcaggtgcacagcccttcctgggtttttttcttgtgttccctgtggtctctgacccattaggctaaagagagacaagagaagcccccaacctgattctcatgacagctccatcaagaatgtgggatgtgccgaccaaggatttgagaaagttgtacagaaatgtgttcatcaaatctggtcaagggactaagctcctagctgaccattcattctgaagattgcatggaggatgaacatctgggaatcctgttaatgagaaggctgaatcacaggcacctgggccaaagggtgtgagcattcatgttctctgctcaccttggtttccgcacaccttcgcaatgtgaacaggtcaggagtccctcccgtccacctcctctgtaacagctggggttccaggcatggtttaggccctgttccagcaataagaaccaatctgctgtacaatctgaggacttggctctgttatttacaaaatgatgctgtggttctgagattatttgggacatttttggctctcctttagtggacacctagagccacagattcccttctttactaaacaaatcccatggattctgatttctgggtcttaggattttaaaagtgaagggatatttttcttatatttgtgagttcagttccgatggtgcccgtggtcaaaagcgaaaaacatggacaattcctattcattcttagcactttgacatgtcttggggaaaagcttacattttaatttaaaagaaagatcaattatatccatgcttaacaggatcagcaggagctttataaatgactttacagagactaataagggatttgatctttctttttttgttatcgaggcttttgaaatgtggaacttgtgtgttctgctttatatgttatattcaatatcttttcagatgcagtctatattttatgctgagttttaaaaatgaaatactttatgcaaacaggcaaaattggtaccaaagggaaacattaaccatgaggaagagcatttttctaaggagaacaggtgacaatatacacatgtgcgctaatcgtaaaatgagcatcttagtctttaaaacacatcagaattgaatacgaataatctatttgtcgatgaaataaacacaactctttgaggatttgagactacattcaccctttattcacagtcacttgcagttttgcttttctctgcatttctctgctgtaagatgactgttgcattgttgaattgtattttgagtggatatttttgtttggtaacaattaaaattttaaatcgtaaaaaatgtacatttgccctttctcttccttctaaacatgtgtctctcccacaggtcattctcctgtgacacgtgtactgcagagtgtttcaaaaccgggcaggcaattagcaatgggaaataagtggtccaccttcaatatttgcttgttgttcatttacatgctcttggcaccaatcctagactcacgtgtgccccagaataacattcagactctcagctggtcttgtgttacacatccatggaccggttcactccatcatatacagctctctgctccgtgtcccctgggctcaagtcaagcagtcggtgacagatttcattcccaataacagaatcggtttgcatgactccccatacatgttgcagctttgaaaacattcatctcagagttaggtataaagacataaaaatgtgtgtcaagccctcgttagctgatgaggtaaatgcatggacaacttcctaggacttctcggctctgccctctgtgaccttgcagcctgctcaggtctttgtgctctgttccttatcaaaaacagcacaaccccctcaatctgcccatttaaaaattggtggagtgaacaaagagcatgaaaatgttgaagaccacaaccaaaatttactcaaatcaaagttaagtacagagtttatttttaacaaatgtcatagctatgtcaccttactacagaatccagcagtcagaaaataacacaaaaaatgccgaggtttggcgtagctggtagctggggacagtgtcttcgtttgattactgccagttatttccagcatgctaaatccctacccacgttccagcctctaggtgagtcagtgcgtcactctgtctcccgtccaattaattatttctcatcactccctcaatccaagtaacaaaccttgaaacacgaacatagacaccaggcttattggggcgtgcacagccaagaccccaagaagtgactccttgtaaaatgtatttgtccttctcaaagcaaaccagaggccctccactgtcaccctaaatagaggtagggaaaaattcatgtgagggttaggctgccagccttttattgtggcatccatctattgtccatatgtatattttcccagtaattgaaattggaaaggaacttatgggcatattactggagcatctgcagtgcttaagtacttagcaaaatctattcagcgaaaccacaccacttagtttctcccaggggtaaaaaaaacccttgtgaatcctgcacacatcccttcctgtgttggtgtttcctttagactggcctagcctgggcatcactgtaaagtatgtgcagttggtctctgtcctgtaccctctacctccttccctgtcggaaaagtcaccactttagtccctggccaccagcacaccccaggagggtgagtggcctgaggtagttacggcacttaaaactcccttgctaccgatctggaactcaagccccaagacatccccttagatgatctgaatacgcattcagggacagatctaggcagtttctaaacaacacttagactggggtctaacgttgacaaatccttctagaatttgcctctttgggactgaagtctaaggggctgagaccaagaagggagagcacaagactaactttggtctcttgaccttttcttaccttgcaactgtcagtgcctccagccaaatgcctagcacagagctcagtggatttgattcttccattcagatactcatagcgattgcacactttattctcaatcacaagaagctggggcttccttgagaaggccagccccaaaggtacctgtgtttaaaagatgaaagaaatggttgctgaacccaaaacatgtttaggaggaaaaccagaagaacagagcagtggcactggacctcctgtctgtccgtgaggctgcagaacacaaggcagctcccaatgccgctggactctctgaccttggagagtgtaaacatcatatatgattctgtgttcctgagatctgggtttaacgatctgggctttgtcatggtgttttacgttcgttgttgtttgtgtactttttgcttttcttttgaaagtatgaaaatgatttttacaatgtaaagagagtctgagaaatgcaattgctattctttttatatactttttcagattttttatatgcttctttactacttacatcataatgcacaattttatatactgattttttcaatcaatgtatatcatgagtttttaaaataatgctacaatcttcataactaatattttaatggggagtgactgcttaattggcatgaggtttccttttgagatgaggaaaatgttctgaaaatatggtgatggttgcacaccattgtgaatgtactgaatgccattcagtggtaccctgcaaaataattaaaatcctaactcttatgttgtgtatattttattgcaataaaaagaatagtcgtcatttactaagaattaaccttttcaggcacaagctcttaatgtggtgtatctcacttcatcctcacaaccgtcctatgaaacaggcacttttatcattcctgttctacagctgaggaaactgaggtgtaaggaggttgtgtagttcagcttgagcctgggtctgcggagtcactgcccaaatgcttcctccccgtgcacactggccccactcttgcttccaatcttccacatgtttgttcataaagttcattctcgtttcttttctgaagtctaagttcccataattatgttgacatatttccatctcgcggtaacactcccaaattattttctaaaagtgtgtgacagtataaaatgttactggaaacaattctcctcacccttgtgagtattcaatttttaaaagattagctaacatagtaggtggaaattgtcctgattcattttctgatttacacttccttgacaattagggagaagaaatacaactagggacaattctcctagttgtagtttttcttttgtgaaaaactgctttatttcctttgccaaattgaaacagaacatctgctaaactgccagagggaatgctgtttttccagatggacacgccaaggatgtctcagtgctctcacaaataatcgttttccaattttgcaagcccagctttgattagaatttctcaaaatattttgctttgcgagcatgacacaggcaaaagctgaggcactctttctttcttcccccaaagtgttggactgcacttagatcattgtctcaggttcagaaaactgacactgaatctggagtttacatgagcagcgtagaagaggctcttcccagagcctggctccagagcccagctcagccctagccgggaaggagctgcatggaggaagctcctcctggctaccaagcatcgggcagggggtgcatgacgtggactgtgtagatgggtgagggagcacccttttcaaaatattttgctgaaaattaagttagatatcactgctctcagatctgctctcagagcaaggcaaacatcctgatcaggaggtaaacccaccactagttgccaggaaattgacgcggtctgtgagtatgtgctgccatccagtggccaatgtagagatggcacctctgagggcggggggccacgggtgaccttgaggattgaagccctccctgattgggtcctggggagagggccacaagattagcccagtgattgtggatggggctgacacggaagagccccttggcctcaacacctactctgcatttggaatatccaagtcaaggaaggtctggaaacatgaaggggagagggggagaatttcgacctccctctgagaagctgagaacacccattgatctgatgtcatcaacatatagaaaatcgataaacagatctgagttatgttttgtgactcctcctggtctagttagtgttctgagaatcattttctcagtggacatgtgggtgagaccagaggagacagggagccagtgcaggatggtggttagaaatgtgcagaagggggtgggagagaggacacagcgagtgggctctggggtcaaggcccggttacacgccggttatgtgatgtttatttatgatgatagtggtgatagtgatggtgatagtagtgataatagtgatgatggtggtggtgatgatggttgtggtaatgataatggtgatggtggtggtgatggtgatgatggtggtggtagtgataatggcaatggtggtgatggtgatggtggtgatggcggtggtagtggtagtaatgatgatgatggcggtggtgatggtgatgatggtgatggtggtggaatggtggtgatggtaatggtagtaatgatgatgacggtggtggtgatgatgttgatggggaggatgatgataagtggtggacggggatgacggcgatgattaataaccctaatcctcacaatgaccttatatgtaagtacagatgagaaagctgaggctaaaagagattaaataactgcccaacgtcacatggaatatgggagagacagaactcacacccggatccttatgattttagcttcatgctttcatcagttgaagtattgtagaggatctgtttgttttctggctgcccagcagcctttcaccctacatcccataattctggtgagggatctaccttttgctcccttctgggtatgaatggggtcctgcctccaccttcagcatggaacagctgatgcaggtcagagccaatcagtgacgggctcaggggcaggaccatgacccaatttcagtctctcagagtggacagactattgttcatgtaagggagagagtggcctttcttattctttctcaacagagccagagccaccacagcatctgcagccacgggagtgtcacctttgtgccccaaatagtgcctactccatgattgggagggtctggggtctggggcctaatgtgagcctgaagacttttcagctcactaagaatttgccttgttttgcttaatccaatttgagttggattttctgtcacttgcaagaaagagtcatgttagattcaactctattccatattattagtaggagctgccatggtggagtgcttaggatgtgctcggcacttgatgcactttcttattatttccctgtaacagccctgcagcccacatcagctaggaaacaatcagcgagagctcacgacttttctaagtcccgacagcccataggcgggagccagggttccaattcaggtttgtctgtaccaccctgcaccttctctgagccccagcttcctcaatggtaaaatggaaacaacaatacctaacgcggagaggtttctagtatttaaattttaaaatatgcaaacaccaggagcagcctggcacacccataggtgtccctcaagccaagtttccttttatttggcctcactgctcccagactaagcagtgcagaaaaagggaacaagaagtcaaggaggtgaggttttaaatataataggaaagagacagaaatagggaacccagctttgaaattatctaggccagtggtttttaacgttttttaaaaacccaaaagacatttcattaaatgaaagcttatacaaaaggctgatgtgcaaagcaccatttgaaaatgctggttttacagggattagagccaagtgatacaaagaggtaacgtgtgtagttcaggtctatctggctagtggcagagtcagaacgcttttccagggccccagactccctacgccctcacctgcacctctgagaaagcggaattgagacattaagagttttagttttcctactgccaagggggtaaggctgatgggtataataattgtcagagcatactgcaaagagaaggaatccttaaaatgtaatacaggtgctgacaccacatcctgcacaagtgttccctatcttgctctcacccatcaaccaatatcaaatgccatcctagatttggggccacggggcctgtctgcctcttccccagatgtaggtcccctgcctaacaccctcccacacacgttagacatgtgggtatttgggaagaagactagtttaactataacttttgttgttattgttttacatcatatttagaaacccagtctggtgaatttcaaatgaaaaataatctaacacccttaacttgagatctgggtcaaaaccccacttacctgggaagcaatatgaacgctgaaattatgattaagagactcagtgacctctactggctttttgcttgcttttggtgtaagggaaacatacgatcatttaaaaaaagattctgaaagcaaaaaaagagagagactcaaagaaatggccccctccccttgcccacatcccttgaagactgattatggagacatagacaggtgcccaggcaaagtcaggagagttggacaatgttcatggataggaatttgtacaaggatctctaaaatgctcattttgtaacatgcactgtaggtcaaaaacaatttgttatacgggcaatggaattgatctcaccttgggtttctccccagccagtgacataacattcaatccaggcggtgaccacgtaatttggggatggcagacaggctgggattactctgtcagtgatgatggcaggcctataagggaagtattagcagttatgtttgactgcccagctgggagctgcagtacctataactaggcatccttaccttgaggccccagagccctctgagctggcactgccttgcttcaatgacacagagcaacgtagaacacagcgtttcaccctcacatttgcctgtggttgcctatggactacacagatgtaaggtttcatttgaaaggcatgagttgcatgcaagaagctgagggtggccttcacctttgaaagtagggatgacaaaggcgcacttctcatgtatattctgtgcacgactgggatctcatgccaagtgtgtcctttcatattaattgaagagggcattgatgactgggcagaatgtctggccgagacatcatcaagagggtataaaattgcaaatgtgttgtgtgcccagagcaagttgctccatctggatcctccacttcctccttgtgaatgttactaaaccgtggttgtaagaatccagtaaaatgctagacatcaaatgaaagaggattggaaataagcaccttgcatgaacaaaatggcattgtcaaagaacatttatatatcttagagttccaggaactctcaaacattagaactctggtttttagacctgattctactttagtaagttcaaggggataaaggactggatatgagaaatgccctattctgacaaagaatcatcattgccaccaactttgaagcagacacactttctgcttctgggcaacagacatctgtggtgcagaagtcctcagtctacaggttccctggtggagcactctagggtgtgaggatgtggggtgggaactctaaactcaaaatgtatcagcctatcagagaatacctccaaaactcaaaatttccaaattgctttttcaagccatcagaacaccctagctctctgaggaccatgaggagcggagaccttgtatttgcacatttgctgggatggtggggaggagactggggagcacgagagagatcagctactgtttaggctgagccagaaaggcccttctctccttcctagagtgtcagagcatcccagaacatcacttgctttctaggactgcataggcaggtgatagctcagaatactggcgtgagccactcggagccagagtgtaatgaagagtacaggtaaaaagtgcatgttggcagcactttgataaatccatgaggacacagaccaccacgcagtttaggaaagaatgaatctgaaatatgtggaaggtgaaagctctgtaaagcaaggagtttctaaccaataaagtaaagaaaaatcaaatcaacggcagagtgaaatgcaatgtgatcaggtttgtcgatggcatttcataaacctgtgaaaagaaggctctttaggggagaacagtaattgagcacaggcaccctgatgtagaagtgagccaaggaagcaaaacatgcaggcggttgggcctgtttcctatgcgtgtttcctaagagatacccaagaaaactgaacttatgaaaggcatgttccactgaaacaatacccaaaaggtccaggttctgtgggattggactgattactcttcatttatggggatcatgacctataattttttcctgtgtttgtgaatggatgagtagcctcggcattgaattcaatgattcacaaaagtgtaaaaaaaaaatcgtgcctgtgacagactgtaggtaagtttgctcttgtcctccctcactgcaaagtgtcacatttctaaaaacttccctctcttttcatctgacagtgagacaatggatatccatagaaaccctcagagcatcttcaatattttccagcaatatcggaacttggtgcaaatcactgcgcctacagattttgatgtagctacttagatcaagaaaaagattccaattttggccattttcaaagtagagaacctaaaataatataattatttttatttttctatattttttcctccttcagaaaacatagaggcaggtactgatgcctcagctgtgtgggttctgtgtaaatgtagaaggattttgtggggcttgtgtggaaggacaatatagttggtttctgggcctgttcttaccttgttgtaagactgacttgccagggccaggaatgtgggtgggccacacaaccccctacaatgcttctgggacatttctttggctccacttgaggcttcccacaatcaaatgaagaggatgctgtggcacaaggtgggaaagaagtcgcatttgagtccaagttggcaattttgtcaattaatttaggacaaattccaagcactgagtttttgatatttcaaaggtgaaattaaacagtaaaattataaacaacttttagattttttttctaggtttcaaaagtggtagtaaacaacacaggatgctctcctttcaaggaaatataagcgctttctaattattttgttggagaagtgatcaaagaaattggtatagttcagcattagaatgagatcttttctgagaagaccttgattggcacaaatagttattcgtcattgttattccgaacaatgtttagaacatgatgtcatccttcccatcatcatcctcatcaccatccccatctaacccacataaagtttacagaaaggcatgaaggtttcttttgcatgtgagtctgtgtgtgcacgtgtgtgtgtgtgtgtatgtgtgtgtgtgattgtatgtagcaaggattactgtcctaagttgaaatgttgccttctctcagccagcttcaggacagctcctctggggagcctcccatgactaactgactgtcattggggtgacttacacacatgtgcctttatttttcttttacctcctctctgccttctctctgcagccagcctgcaagatctctggaggtaggaacagtgtcccatctttggtaccacccatcaatgcctacaacagtgcataggctacagagggtgttcaaaaatgcttctctgtctccagtccttcaaggtcttcaaaattataaacattaatatagttaataatattcacataattattaataacaactgcaagaggaatttcctccccaaccaaccagagattctacactccagtgaatttggaacctgagttttccaaataactgtccatttgccatgggctgttctgagaatctatctatataagtcttttttttttttaaagaaacattgcatggaatgaaatcatcttccctagtgctgaactcaatttctgctaatatgccttacacacagtatacatgcatcattgtactaaaagagtaactacagaaattatggtacctgctcctactatttaaaataaaactgaatgtttctatttatttttacttattatttatactgaatgtcttagtggaaatgaactagctattaaaatcattctccaccagagtctctagatttctcaagcatgagtcaaacctgaataaaccacatccttctgcaggaaaccaaacccaccaaccacatccttctgtaggaaacagttctgaggatttccaagtgtcttatgggaacccttctcacaaggcttggggttaacctcacttgtcttacatcaaggttaggtgggggacctcacagtccattgggaaaaatcttgctcccattcttccttctttctccaatctttaagaactcgattctcatacaacctttatgtgggtgaggacagcagaaatgcacctgggaagctactgcactttcctaccataggagtcaaaggtgcatgagtcccaggcacagtagtggggtctgaggcaagaaagtcagtgtgggctcaacttcaaggcagcctgtcaaggatagagagcccaggcagtgagaggcagtggcatgtaccagacggggccagaagtggagaactggaactgaccccatcgagggactttcaggtagaggagccaccaagatgcttctgcagaacaaacaaaaacacccactcaaggaaaaggtcagctatcaactcacactaaatccagagaccacaatgccacaatacaataatattggccaagtagaatctttactatcaccctaacctctcattcttccctcttgctctccccaggagtgtcccaaatgcgaagctgggcagtgtggctcccataatcttcatcactgccctgtaccgcctccccaggaaatcttggaatggtggtcttcagtggacttggaggccaagctgcttcccccagctcaaggttcttccacatggcagacccaaccacaagcaaccaaattatacaaatgaaaagtccaggacagctttctgtaactaggacagacaagttgattggtcagcagaaagtaaaataggggacaagtccaagtggctttttgttttttcagaaatacccatggatttgttatcccagttgtttaagatctaatgtcaactccacttaccctgatccaagcctcatactcccaacaagagtcttggagcaatgaagagggaacccaacaaacagtctctaagcaatgatgctaatgtgaatcgtgaggaataatcttaggagaaaggccatttggggctaaagagctgcactacccttcactgaattcctaggatggccatctgtaggtttacaatgtgcaccacatgcttagctgtgccctgctgttcgtatatttttcatacttctcagctgcagaagagaaaggttttttatccgaatgactgtaaatttctttttcatgcatcccatgctagacactgaagtgctcagtgggaccaccctggggctgattagatcacctctaagtgtgctctttgggaattttacatttattttatttatgtgtgtgtggtgggaggggcgggtgtttccctaacaaatggggccaattgtagggcaggttcaagatgcccatgacgaagggcaggctctggtggagagggcagccatctcctccccagaagcactcagctcaaaagccattttctggtttttttttttttttttttttttgcaaatccatatgaaggaagcagtttccttaccaaaacagaaggcaacttaccacagagagggatatcacagtagtcaaaaagttttcttggattcactctgtagcaccagggaccattgatgtcaccatcagggttacggcagtactgaaaacaagcaggcatgtaagctccagctcacgtggggcaagagagggagaaggtgttccaaggcacacatgtttcacatctattcttagagacataaaggagtgtttcttcttcctttctttctattattattaagatatttttcgcaaacttattttttgtatgctctaaagccatcagtcatgtgttccagttcaaaagaagctatgaggccacaccctgaccaaaggggtggtgaccctgaagggtctttggagaatgggcctcttctccagaaacccgacccagtggaggtcttagagccagagccccagagaacagtggaacatcattgatgagctttgtcccttgggggctcagggctcttctacctgataaagcctgttagctgttgagtcagaaacacttacttccatgcatcatgcaccctccacgggcgagacacacaaaggcagtgtcatccttcccttcctgtgatactgagatagaataagaaatgtatattgggtctctgccccccagttcctaacagagctcctaaatcccttggagtttcctgggtgaagggagctgcaatgaagcaactcttggtgggttcctggatagcctccggggctggttgccaagggaaccaaccatgtgatttgagtgttggaaccagtcagccccaccctccaacctccagggagggaagaggggctgaagttgagctgatcaccgatggccaatgatgtaattaactgtgcctctgtaatgaagcctccacaaaaacccaacaagactgcacttggagagcttctgggttgctgaacccttggggtacctgggggtggtgcactagagagggcacagaagctctgagctcctttccatgtgtcttgccctgtgcatctcttccgtctagctgttcaccaggatcctttgcaatatcttttacaataaatgggtaaataaaagtaacgtgttcccctgagttctgtgagccactccaggaaagtaatcaaatccacggagggtcatgtagaaaaacagaatttgtccagtacaggtcacagcctggaacttgggattggcatctgcagtggggcggtcttgtgggactgaaccttaacttgtgggatctgacactgtctccagatagagtgtcaggactgagttacattgtaggacacccagctggtatctgctggagactcacttggggtatgaggagaaatccccccacatctggtgtgagaagtgttatgttacatgggtgtgagagtgggggaacacagacacttgggtttttcctatatctcagtcttccctatacacaggccccaaagtaaaccatcccagagaatgcatgagactaggagaagtcccactgccccttgatggcccatcccaatcttttctagaaaaatctgactcaagcatcataaaggacgtctttgaccactagaacctagagcaatggtccccaacctttttggcaccagagactggtttcatacaaggcaatttttctatggacccagggtgagggggcattggtttcaggatgattcaagaacattccatttacagtgtactttatttctattaaaattacattgcaatatataccgaaataattgtgcaactcaccataatgtaaaatcagtgggaggcctgagctcattttcctgcaactagatggtcccatcttgggatgatgggagacagtgacagatcatcaggcattagattctcataaggagtgtgcaacctagattcctggcatgtgcagttcacaatagggtttgtgctatgggaatctaatgctgccactgatctgacagttggtggagctcaggtggctatgagagtgatggggagcagcagaaaatacagatgaagctttgctcacttacctgctgctcacctcctgctgtgcagcccagttcctaacagaccacagaccaggatcgggaccagtaccagtaccagtaccagtactggagtgggaacccctggcctagaggacaaggactagaaatgagaaacagcacccaacaatggaaacagtaaagaaatgtcacaaaagagaagaggcctgagaatccccacacgcgtggggctttgctggcctgtgctgcttacactttccatgctaaggccaattacgcttagcctcctgaagtctttccagtattttcctctgcccatcctctgtttgaaaagatgtgtcagcaaagggaaaaagagtccaaatcaaagtggcttacatttttttttattatactttaagtttcagggtccatgtgcacaacgtgcaggtttgttacatatgtatacatgtgccatgttggtgtgctgcacccattaactcgtcatttaacattaggtatatctcctaatgctatcctcccccctccccccaccccacaacaggccctgggtgtgatgttccccttcctgtgtccatgtgttttcgttcaattcccacctgagtgacaacatgcggtgtttggttttttgtccttgcgatagtttgctgaggatgatggtttccagcttcatccatgtccctacatttttttccagacctgcccgtggacttgtccctggagtgaacctgctgtgtctatggggctcttgggcagtccattcttggcgtggcgtcccagtaacagtggttgccttcttgccccggtatcctttcccattcccaaacatagagtctgtagaaaaaaaaaacgaaaacaaaaacaaaaagcagatgatatgcacagccacatagacccaggacaatatggaatcaatgcagtcatgtcaggtttctctaggacgacggaatcaggggccacattagtcagaagccactgctgaagactgccatgaacactattttaagaaattactggaaatgtttattggcttggaaagaatcacatagttgacagcatgttagaataggaatttgactgggacactgaaataacctattttagaacaaggtgagtggttaagttgagcaccctggaggttttgcgccagtcctaaagtcactacactcgcaagttgtgccaaaaatcacagctctggctttcccagggacggcattgaggcttccttggtcatggcagaccctcaaccaacactcgagctcccgtgcagcactaaaggcttctgcatcagtgtggtttttcatgtcttttcatcccgttgtccaagcacatagatgtctggatacagactttttacttgttcggaaggagcctctaggcttggaacctggataacagtcggaggagcgaccacagtcccttctgtctgagcatcgcgtcaggttgcagtactcccacctgacgctggggtctgtggtaaaacaccaagggcctgtatcggcatctggattcctgcagtagttcattgtcaggccactggaaattccaaagcaatacaggttacaggaggcggaagaatattcaggggcacccagcaccctctacattttgttctaacaaagccttaacaagtgactttgaaatattcccattaagggtaccatacaattactacaacaaaaaaggtcctcaatttcttttctttttttaattttttttaattttttttttttttttttttttgagacagagttttgctctggttgtccaggctggagtgcaatggcacgatctcggctcactgcaacctccgcctcctgggttcaagtgattctcctgcctcagcctcccaagtagctgggattacaggcatgcaccaccatgcctggctaattttatattttcagtagagacggggtttctccatgttggtcagcctgttctcgaactaccaacctcaggtgatcgcttgcctcggcctcccaaaatgctgggattacaggcgtgagccactgctcccggctgggtcctcaatttctaacccactgcatacccttaaaagatagattctaatttttattttaaaatatcaaaagtagtatatgcttgtattagtccgctctcacattgctatgaggaaatacccgagacagactgggtaacttacaaagaaaaatgttttaattgactcacagttccacattgctggggtggcctctggaaatttacaatcatggcagaaggcaagggaaaagcaggcaccttcttcacaggcggcaggacagagtaagtggaagcaggggaaatgccagacacttacaaaaccatcagacctcaggagactctctcactgtcatgagaacagcctgggagaaactgcccccatgatccagttacctccacctggtcctgcccttgacacatggggattatcaggattatagagattacaagtcaagatgagattttgggtggggacatagccaaaccctatcaatgctccatataaaaactgagatcatatataaatgtgcttaagaaaggaccagaataccctccttctaccttctgccaaacacatacctctggaggaaccggtatagattttgaagtgtgtcttatagattttttatgcacatacaaatacacctatgtctctctagctctctgtatctctctctataggattgtatattaatatgtgagttggaactaacatatatttctccaaggaggaaatgccatccaagatgatatcattcccaggccagcccccttactctctgtactcgttgtgtagctaaaaggctctgcatttacaatgaaaaacaaccatgaaaatagtattactgtcaataccatttcaggtgggtcaagtgggtttgagtctacaagacttataaaagctgaactggaagactagctcccaggcaggatgctgtttctctaggatgggttcctgggcaggacctcctctcttgctctcagtccatcctctgctggctgcagccactccgggactgagggaatgggggatgagatgaaggaacagtgggacccaaggcataaagggagatggctggctagactatgggatctgaagaggtcaattgactatggaggaggcagaaaaaaaattgaataagaggcacagcaggtgaggaccccagggttgggggcatcttggagaatgtgctccccttcgttcctcccaggaatgttgcttcaacctctcagaatcctcacattcatggcctgtggctgtctgagaaaacttgaaatgtattatgtaccatgggaatgcaacactatctctgtagagactctacacatgtgacccagtctcaagcccagactcccagctacaggaggctggttctccaagcaactggatatgttagcaagtgtggtattcgaaggagatttttttatctattttcaactgaggtacactttcatgtatagtcaaatgcacagatcttagatgtagtttgatgaattttgatgaacatgtacacttgtgtaactcgcaagttacgatcgtgacacagaacatttacatcaccccagaaacaacttttgttttacggcaaaatcaatctctgctaatttcttcccatggccagaggcaaatgatactggtttccttcaagagaaattatttttgcctttctggaaccttatatcaatggactcatatataatgttcatttgtgttatctgacatctttccttaatgtaatatttttgagcttcatccatgctatttgagagtatcattaatgtgctcccttttattgctgaatagtatgccattgtatgaatagatcgcaaattatttattcattttcttattgattgacactgtagttgcctacagtttggggtataaaaaataaagctgctatgaatattcttgtagatctctttgtggaattatgttttcatttctgttgggtaaatattcaggaatagaattgcagatttgtagagtagatgtgtctttattgaacttttggagaaatcatcagactgatttccaaagtggttttatcattttgcacttccaccaacatacgagagagttgcatatccttgctctacatcagtgacatcagttactgtggtgtcttcttaataatatccatgtccactgggtgtttggtgacatctcatgatggttataattctttatttttcatggctgagatattggacattttttcaagtgcttattggttttgtgtgtatccatccctgagaaatgcctattcaatctgttgccttttttgattaagttgtttgtcccctgtttagcaagtgggagaaatgtttctatactctggacacaagccatttgtcagatagatagaaagagagcatttttcttctcatgtgttatttgcttatttattttcttaaaaatgacttttggtgggcatgagttttcattttatgaaagcccacatcataatttgttttcttttatagttagtgttaatgactcctctgtcaaaaaaaattgccttctccaaggttgtgaagattttatattttctcttggaactttcatatttcagtttcagttttagctctgacacttaggtggaagagatatctcaaattaatttgagatgaaaagtaaaagtcaaggtctgtttttgttttctatcatggatatccaaatggttcagaatgatttttgtaaaagattattgtctccatttaattgctttgtcatctatatagaaaatcaattgaccatatgtatgtgggtctggtgctggaccatccctttttagttttgcatacttttttgccaataacgcatatctttttattttttgagatagggtatcattctgtcatccaggctataatgtagtggcatgatcaaggttcactcatgcctagacctcctgggtcaagtgatcctctcccctcagtgtcctgagtggctggaactacaggtgtgactcatcacacccatctaatggttcatttttttttgtggagagagggtctcgctctgttgctgaggctggtcttgaacacctgggctcaagccatcctcctgcctcagcctcccaaagtgttggggttacaagcatgagccaagggtcctggccaataccacgtcttgattcttgtacctttataagtctttatattaaggtatgtaagtcctccacattttttccaaaaaaggactgcagcaatagtgtttgggtgagagtttaatccaaagatcaatttgatgagaagtgatattgagcctttcaatctataagcatggtgtatctttatttagatcttttaattctttccttgacaacgttttctagtgcaagctgtatagtccttgtgcatagtttgttaaatttatcattaaatattttttggttttgataacattgtcagtgatattgctttaaaacatttatttttccaaatgatcatagaaagtatacagaataccaattaactgttatacacagagcatgtgtatattttttaggactgtctgcatccattaatcaggttttccttctggtttttcattctttctttttgttttcatttttgggtcttggtgcaaaggctgtgaccattgtagtgaagtggtgatgagaagtattgagagcaaacatctttcttattcccaatcttaaaaaacaataatatttcaccattaacatgatgtttgatggaggttttacacagatgccctttatgaggttaaaggagtaccattctgtttctagtttgctgagacattttattatgaaagagtgttgaattttctcatatttccttctgcatctatggatatcatgtgatttatcacttttattctgttattatgttggattacattgtttgttttgcaaatgttaaatcaaccttgcattctcaaagggaagctccagtttttttgtgatattcatcacattaatgataaaaatacattttaatattgctggatttaatatcccaatattttattaagtctttttgtgtttattcactatagacagtattctgcagtttttttttcaatatcctgtgtacttttttaactgggtttttttgttctcacaaactgagttaagaagtattcccttcatctctattttctgtaacatactgtgaaaaattggaattatttcttccataaataacaaaatttctaatgaagccatctgggcctagagatttctctgtggaagtgttttgattttttaaaatatgtattattaaggaatataaacatacgtagtcttgaataggtatcacactgtttagcaattcaacttattcagtgtctgacctgcatatttgttttcttcctaaggaatcagtccacattatctaaggtactggatttgttaacataagttgttctgagtattttcttattatctttaatacctgtagcattctgtagtgataactcctcttctcttttattcctgatggtggtggttgttctcccttctttgtttgtcatcagtttagccagaggtttatcaatcgtaaggatattttcaaggaaccaactttctgttgcacagatttactcaactgtttattatttttacattgaatttcatttttttaatcatttcctttctttcacttaccttgggtcttatttgctttttatatattgctagatccaatacctcaatattttattaagtatttttgtgtttatgtttactatagacagtattcttctctagatttttaaattgctttcttagatgaacaattcccaatatttttatttttaggatcactctgcattcttaaaaagtattggggatcccaaagagctttagtttttgtaatatatacaaatatgcaaatatattgttgcaatcgaaaccgaaacagagaattttttgaaccataatatagacaatcatacattttcctagccattagagagatgattccactacacattatgtagctgctggaaagtttcactgtacctttgtcagaaaatgaaagtgaaaagggccaataaaaaatgtttcttaattaaaaatattttatctccctagattctccgaggggatctcatgagtccccaagtttctgctttgagaattgctatctcaaatcattgatttttaaatgtttctattgtctactaagagcatttaaagttgtaaatgtataaagacattaactgtttcccacaagtttgagtatactgaattattattattattattcaggtcaaaacttttccaagttccttggagatttcttctttgactcatggaacatttagaaatgtgttgttttattaccaaatattcagcaatttcccaatgttttggttatttatatcttatttctttctgttgtgttcagaaaacatactatgtatcaatgtaattcttgaaaatttattgaaacttttgtaaggccacatatatggtataccttgatgcatgttccatgcacatttgaatgtgtattttgtagtttggcatagtattaaataggttgatgttttactttaaatcttatatattttttctgattttgtatgaatgtgttcttttacattttgtttttggaagatttataacaatagtaagagaaaaatgaagaaatttccatctctaattgtggacttatctatttctctctttagttctgtcaagaactaagttctaaagttttgcttcatatttcacaattgctgttattaggtgcaacacattgagcattgttatgtcttcgtgactggaccctttttcttatgaagtgtcctttttgtttttgtattatttcttgtcctgaaatcaactttgtctgttataaatattaccactccagtttatttatgattcaggttttcatggtatatatcatttttcatattttgctgtttgtaggtctttacactgaaagggcatttattataaaaagaacacaattaggctgcagtttttattcaatttacagtctctgccttttatttgtagtgtttaatcattttatatttaatatactcataaggtggtcataaggtggttcagtttatatctaccacaatgccatgaattttctgtgtacaccatatcatttttatcctttattcctcctttgcttccttcttttggacaagtgaactattcctcttattatccattcctctcagtttaagtttttgtggttttcctaggagttatgatatctcaccttaaactatcacattcaaccatgtattaatattatataacttcatgggtaatgcaaaaagcttataacagtatatttccatttatcttctacattatttatgccacttttttaaaataaattttacttttacatatgttacaatatattcttattaattcacatcacaaattgtcctttaaattaaaaaaaagtattttatattgacattgacatttgccatttccagggctgttcattgcttagtatggattaaagtttccatctggtattatttttcttctacctaattggctttctttaacatcttttattacagaagcctgctgtggaaacattcttttggtttttggttgtttcctctggaggtggcagaatacgtatttacatcacatttattttcaaggtttttttttttttcctggacatgttgtcctaggttgatctgattttttttctttctaatcactttaaagatgttatttcattgtttctgagaagaaggcataagtcatttgcatcactgttcctctatgtaaatgatcttttttcccctctagcttataataagattaccctggttatctttgatgttggacaatttaaccatgatatacccaagtaaatcataaatttttgcttttaagaattctatttctgattagttgagcttcttaactgtgagtatataattttcttcatatttgtaaagtcttcagccgtcattgctttcattatttttttctgtcccaatatctctttcctgttcttctgtgactgtttgtatgactttcattgcattattttctagttcactttttcttctgcaatgtataaactcagagtaagaccacccagtgaattttttattaaggacgtacttttttgtagttctttactttccatttggttgttttatatgttgcttttttcttggtattctgcacttgtttctcttatgtccatgttttctttaaatctttgaacacaattatcatatatacacagtttgaatttctttaccaattccattgtctttattatttgttggtctatttgtattgactgatttttctcccagttatggttgctttttttgcctattaaattttaaaatttatgtcactcattgtgaatgtcacattacggagtaccatgatccagttatttacctccagtgatggttgagtttccttctgtcaggcagttaatttacttggagattatttggatccttttagacttggtttttaagttttgctaaggtatttctggagtagcctttattctaagaaggcataaatcctgctcctaaagtatgactttcctggggtctctactgaatgcccgagatgttcaatgaggtgtctctccttaggctgatcagaaccccagtatcccccagtgctacgtgagctctggcgtcaccattgagctcactgtttccctgaatttgtcctttccccagtagtggttctctgtcccactttgtagaatttctccctgtgcatatgcagtttatattagacttccatgcatattccttcagctccttttcaccacaaattctttctcattattacctagcatagaaaatacacattttcttagctatcccaaactccagtctctttcctcagttcagcaagactgctgtggttggctttggctccaattccttgcccttcagtccaggaaggttctccaggaagaaagtcataatagttatggagttgcgctcattcatttccttttctaaattatctcagtcctgtactgtctcttgtccaacatgtggaaagtttttttgtatatcttttgtagtcttttatggcagaaagccagtttttgtcagaccaggttttttgtccatgtaccggcccgttttaatctgtacatctgtttacttcctgactgactctcatctacgttcttgcttttgctcttcctgtcctggcaacccatatcccttcagaaaactggagcttagaacccagtgtgttcacacaggtgtatatatataactatatataactatatatatatatacacacacacacatagttatattatgtgtgtgtgtgtgtgtgtgtgtgtcctgcagattgtatagctatatagcctgcagctgtgcagactatagggtgattggtcatcagtcagggacagacatgaaaaccttgcacatttccctagaccatatatgaatacttgcctgtatgaacacatatatgaataccaacctatatgagcacatatatatgaatactagtctgtatgaacacacctggttctatatatatgcacacacatgtgaattatttacttgtgacccatgacaaaaatggcaaacccaaccttcacttcagggaagggaggttagaatgatagaattgcatgggtgaaagactgcattgatcttttatgagccttgtagaatggcactctatttgatcgtttgttctcctgagctttatattaaaaagacctcatcagctccatgtagccttctgcttccatcagactattagaaaagatagcaagcctaaaatacctattctctcagactctttgatcaaaagcaaagttgtttggacattttgacacaccttctgggtctaagggaaatttgtcctaacaagttagcttgaagcaaggctcttctaggaggaacttaagttggctgttgctcctcttacaagtaacatcaaagacatactcatttgggtagttttctggggtcctctgatgccggtgtggtgtcatggatgaccaagattgacatgtccttcctgtgacagtggtggagaatgtgccttgataactctgtccattaccatggtagcactgccggaccacaggggtttgctcagttggtgctgaaaatagtcaaaatcaagctcagtaattactagtacgcagaaacatgtgaagcaatttatgacacaaacaggacagtagtttcagaattatgactgttctcttcagaatactcgcaagcagaaggacatgctgaagcaaaaacaatagattaatagcattctaatattttaataagttcttagaaatgttatattcaaaatctggtggggaaaggacctatcagtgttcttacaattgcaactaagactcatgtgcaagctcttttgtgtggctctctactgattgttcatgagaacaacaaggcaggaaaccagacagtgatggattaggccatgcaggctgttgggtgattggctgtcagtgggggacagacaggaaaaccttgcacatttccctagaccattgctcctgaaaagccaaagccttaagcttctggtggacggggcagttcatggtcctgcatctagtacttgcaggcaaagacacattgcttcttagagaaggatggagactaaaatcttattcccttacaggagagacagggaatcctcttgttccagcaccctgcattgcaacaaagcagaagtctactacctttcctctcaacacacaccaaagatgcacgttccacgagaagaaggcgcaagaacaccaagaaattcctgccttccaagcataggtgtgcagagcctacctaagactaagataagccagagaaataatgcattctgtggttatgtagtgtcaaatagccccaggattagatggaataaatgtcatcatattaggtttctccaggatgacaaactcagcggccacatacctcagcaaccacaatgcaatgattgcaatgaacactgcccattttacgaaaggcctggaaacactcactggtgtgaaaattggcacaaatttggccggacgttagaactggaattttgtctaggacaccaagataactcacttaacaagatgagtggtaagtcaagcgccctggagggttgcacccttcctaaagacaccattgactagcattgcaccaaaaattacacctcttgctagagcagagagggtgctgaggcttccttatgcacggcataacctcaaccaaccatcaagtttctcagtagcatggaaggcttctgcatctgtcagattttaaatcttttcatcccaatgttcaaatgtgtagatgtctggccacagacttcttacctgcttcagaatgagcctccatgcttggaactggaacaacagtgggagtctctaggacactgattctgtttctgagcattgtgtcaggttgcagtactcccacctcacacacggatcagttgtgtaacaccagggttgtttcccagaatctggattcctgcagtagttcctggtcaggccactgcaaattccagaacaatacaggttacaagagatggtaaaaaattcaggggcacccagcactgtctaaattttgttacaataaagtgttaaaaagtgacatttgaatattcccattttaggtaaaatgatattcccaaggcaaaaatggtcctcaagttctaaagaagagtgtaaatttccaagatagattataatgtatattttaaaaaatcaaaaatagtctatgctccatttaaaaactgagatcatgcataaacgtacacaagaaaaaaattagagctgggtgcgatggctcacacctgtaatcccagcactttgggaggctgaggcaggcagatcacctgaggtcgggagttcgagaccaacctgaccaatacggagaagccctgtctctagtaaaaatacaaaataagctgggagtggtggtgggtgcctgtatttccagctacttgggaggctgaggcaggagaatcccttgagcccaggaggcagagattgcagtgagccgagatcgcaccattgcactccagcctgggcaacacaagcaaaactccatctcaaaaaaaaaaaaaaaagggaaaaagatcagaatatgtttctccttctgccaactgcattcctccagaaaactgatgtggattttttaagtgtgcatcttagattttattaatgcacataaaaatatataatatattgatatctgtctacttctttatatccttctttaggactctacagtaacatgtaaattggaatcaaaatatatttcttcagggaagaaatggcatccaacatgacaccattcccaggccagcccccttactctatgtattcgcagaggagtcagaaaacaatggagtaagaggcaaaggtggtgagtaacccgcgatgggtgtatcttggagcatttgctcccccttatgaatcccaggaacattgctccaacctctcagtattctcgcattcatgacctgtggctgtctgtgaaaacgggaaatgtattatgtgccatggggatccaacactctctctcaagagactacacatgtgacctagtctcaagcacagaccctccattcaagaagcttgttctctgagcatttggatgtgctcagagccgagcagttgacttatttttcttccttggctgccttctttttgacatatggaatattcttgtcatagaatatacctcagtgttcacaattttgtgattactctagaatttgcaatatctctttgaacccaaatatttcaaacttttattatgtaccatcataggtaaggcaagaaacttacaatggtataattctttattccctacacgatttatgctacattttcaaatttttacttcaaatatgctaaaataatacatattgctgtcattttgcttcaagcagtcaaatataatttaaggcaattacgaaatttgatcaagaaaatatttttgcatttacatctacatttgccatttgctgggttcttcaatacgtgggatgcagaaagttcccaccttgtactattcttcttgtacgtaattccctttgggccacatcttttgtgacataagcctgctgattccaaattctcatagctttcggatttcttgtttggggggtgcacaaaatgcaattatacaacattgatgattttcaaggcagtgtatagctggatgtagcattctcggttgatctcttcttttccctgtttatcattttaatgatattcctttgcctgcagtttgccatgtttctgatgagaacacattagccatttgtatcaccgttcctctagcaagacacagctctttttcctacagtttctcacaagatatttcttgatactatatctttgtcttctgagagtcaaactgtgatgtaccaaagtgcttcatacagttttctttttgaaaattccctttctgatccactgagcctattgaatctgtaattacttaacgttcttcatattaggaaggttttcagccattcttgcttcaggtacttgttattgtttcagtatcaccttcctctccttctgtgccttgcttctcacaggtaggaaacctttttatattttcctttgtgtctgtgaggcttggttgatttcacttttcaggatttgtggatgtgagtgtgtttgtgtgtgtttgtaagcttgtgcatgtgtgagtgtctgtgtgtgtgtgtgtgcatgtgcgcgcgcacgcgtagctgtttctgtagtttctataaattttctttcagttagtttttcttctgaaacttctaactggatgaggaggccacccagtacttttttgatttaaggcatacctttcactagagttctaatttgatattgctcttttatactgttactttctcttgatgttacatctgctaggttccatactcatattttctgaaatcctagaactcagttaacacactgataggtccaatttttatgccattctaatcattcctgtcatttctgggcctatttgtattgactgatttttctcctggtcgtgggtcataaccaacgctttctatgtttagggagtttttaaatttaggactcacactgtcgataccgcatagctgagccacttgattttgctgtttacctctaaggagggttgtgttttcctctagcaggcagttaattgacttggaaacctttttgatgcttaagagatttgcctttaggctcagctaggttttgtctggagctgcctttattctaggaatatattaattctacccttaaagtctggcttttctggggtctctactgaatgcccaagatgttcagtgacttctctctcctctggctggttagaactctaatatccgctagtgctatgtgagcactgtcatctttactgagctcactgtttccttgtagatgctgttttctcatagttgtccccttgtagttgctcagtagatgtccttggccccactctcctgtggagcccttttctgtgcacaagcagcttcatgtttagccaatgacttgaggagacttctatgcatattcccagagctcctttcctccacacatccgctctcattcttagcaggcctagaaaaatcctagctacctgagtaatcccaaactccagtctctctcctccattcagcaagatagctacagtccacatgtgctccatctccctgctcttcagtgcaggaagggtctccaagaagacagccagggagggcacttatggagttgatcgcattgttccccctctcacaattctcttggtcctctaccgtctgttgtctaacatgcagaaaccatttttccatgtctttggtagccacttacagcagagaggctggacacagggtggtcagaccagggcttctgaccatctacccaccaggcttaattttcagcatctgcctctttgttgactctcatctgccttcttgcctttgctcttcccttgctggcaacctgtatcttgtcagagcactggtgctcaggaccaagtgtgtttctacccattggaaaagacgtagatacagaaagagagacatttgcctctccatagatatgcaaatatttttttctcttgaaccaagacagaaaaggccaacacaactttcacttcaggaattggaggttagaatgatagtattccaaggtggaaaggctgcattggccctcagtgggtggtgtggaaaggtactctaagtgtttggtcgtttgtggtcctgactttttccttaatgtgatctcctcgcctccagatatcctcctgctccataaaactaggaggaaggagagccagcctaatatttctcttctctcagactctttgctccaaggcaatgttcctgagacattttgctatgccctttgcatctgacacaagttgagtcttgagcagtcagctcgaaacatgactcttctaatagaaacttcagttggcccttccttctcttatggtaaagaacaaagacgtatgcatttgggtacttttctggggtcctactgtgctgatgtggcgtcatagatgaccaagcttggcaggttcttcctgtgacagtggtgaagtatgtgcctcgataactctgtccattaccgtggtagcactgctgctcccaaggccttggctctgttggtgctgaaattaaaagagaaaaaatcaagctgagtatttcctagaacagagaaacaggtgaagccatttatgacacaaaccaggcaggagtttctgatattacgacccttctcttttctctattcttaggcagatggacatgagaacataaacaaacaaacaccaaagcaacagatttctaccattctagaagtgtagtacattcatagaactatttcctgtaaaagctcagagaaaaacagcagatcagaatgcttctcataggaaactgtgctcacaggcaagctggtctttagggatctctactgaatgttcacgggaaccatggggcatgagagaacaaagggctgtgcatttggcggtgtgggctgtaggtggtgattggctatcaagggtggaggtggggacagacatggaaacattgcgtatttccctaaagtgttgatcacgggagccaaggccttcagcttctgggcaatcgggcagaaaacagttctgcaactaagactgcaggcaaaaacattttgcctcttagggatggttggaggccaaaatcttatccccttggaggagggatggaatcctctcgtgtcccgcattctgcatcgtaataaagcagaagtctactaatctttctctccagacccctccccactatgcacacacaggtacaagttccatcagaaggaggtgtcagaacactaagaaatcactcacttcaaagcctaggactgcagagcctgcctgggactgaggaagctcatgtactgctccatagacccagggcgatatggaattaatgccgcctgttaggtttctcgagattcacaagctagagagccacatttcctggaatctgcagtgctgaagattgcaatgaacgctgtctaccttaagaaatccctggacaagtgactcgcttcgaaagagtcacacatctgacagcatgtgacaataggaattttggctaggacactggcccaacccctttgacaacaaggtgagtggtgaagtccaccaacctggagggttcgtgcctatcctaaagacacagcccactcacgttgcaccaaaaatcactcgtctggctcccccccagagtgccctgaggattcttccaacatgacagaccatcaaccaacatgtgagcacgtgcgtagcactgaaggcttctgcaacaatgggagtttcaaaggcttttctacccagtgtccaagtgtggagatgtctggccacagacttcttacctggtttggaaggatcctctaggcttggaaccgggataacagtcagaggcacgactgcagtcccttctgtgtctgagcatcgtgtcaggttgcagtactcccatctgactttgggatccatcgtgtaacaccaagggccttccacaggatctggattcctgcagtagttcatgatcaagtcactggaaattccaaaaggatatacgtcacaagaggtgggacaatatgcaggggcacccagcaccctctccgttttgctgtaacaaggtcataaccagggcctttgaaatattcccattaaaagtaccatacaattgccacaagcatgaatggttctcaaattctcatccactctgcacatttccaataattaatggattttaactgttttatttttgttgtttgcaatgcttcttggaaacactgaaatagtacatccatgtggcctggaagggatcagaatattgtccttccacctttggccaatacatctctctggaataactggtatggattttgacgtgtgtattgtggaatcatttatgcacatacaagagtatctcgatctgtctagctgtctttatctctctctataggacttcatatagtattgtgagaggtggaactaacatagagttcctcaaagaaataatggtatccaggatgatatcaatcccaggtcagctttctaactctttgtgctcaaaatgtagcaaaaaggctctatgttattatcatggaaaaccatcatgcaaatagtgttactgtctgtcgataccgtatcaggtgggtcagaaaggtttgtttctgcaagacttattaaagcttccgtgcaaaactagcttccaggcagaatgcagtatctctaggatgggtcccagtacatgacctcctctcctgctctcagtccatcttctactggccgcagccactccgggactgagggaatgggggatgagttgaaggaacagtgggtcccaaggcataaacggagatggatgaatagactatgagatctgaagaggttaggcagctatggagaagttagtagttggtcatttgtggtcatgaccatttttttcatgtctttggtagccacttatggcagagagacttgccattggatggtgggaccccagcttctgtgcatctatctaaccccctcgcttacttttcagtctctgtctctctcctgagtctcatctgtctttttgcctttgttcgtctcttcttggaaacctggacctattcagaccactggtgctcaggacccagtgtgtttctgctgattgaaagatacatagatatttatagatagagatttacctctccatagatatgcacatatgctttcttcgagatccaagatagaaaaggccaacacaaccctcacttcaggaaatcaaggttagaatgatagaattttaaggcaaaaaggctgcattggcccttagagtgtgttgtggaaaggaactctaaacttttggtagcttgtgatcctatctttttcttaatgtgatcttctcgcctccagatatcctcctgctctataaagctataaggaaggagagtcagcttaacatttctcttctctcagaccctttgctccaaggcaatattcctgagacattttgctatgccctctgcatctgagacaagtagagtcctgagcagttatctcaaagcaggagtcttctaaaagaaacttcagttggcccttccttctcttatggtaaagaacaaagacgtacgcatttgggtacttttctgaggtcctactgtgctggtgtggtgtcatagatgaccaagcttggcaagtgcttcctgtgacggtggtgaagtatgtgcctcgataactctgtccatttccatggtagcactcctacattgaaggccttggatcaatcagtgctgaattaaaacagaaaaattcaaggtgagtatttcctaggacagggaaacagatgaagctatcaatgccacaaaccagaaatgagtcttcaagaattacaatcattctcttttttttatttgtaagcagatggacatgagaaaacaaacaaacacaaaagcaacagattcctaccattctggaaatgtagtaagttcttctaaatatttcatataaaagcttagagaaaaacaacagttcagagtccttttcatgggaaactgctctcacgtgcaagctcatctctagggatctctactgaatgttcatggagacaatggggcatgagagtacaaagtgtgatggatttggcagtgcaggctttaggtggcgataggctgttgaggtcagaggtggggacagacatggaaactttgtgtgtttccttagacccttgattaccagagcccagcccttccgcttctgggtgatcaggcagaaaacggttctgtgtctagcactgcaggcaaaaacacttcacttattaaggatgggagaaggctaaaagcttatccccttggaggagagacaaaatcctcctgtccattctcttttctttattcatagtctgatgaacattctgcattgcagtaaagcagaagtctactactccttcactccagaccccccacaggtacaaatttcatcagaagtaagtgcaagaacactgagaaatcactcccttcaaactgagggctgcagagcattcccaggactgaggaaggtctgaaagttagagcctgggagctcacgtactgctctacagacctagggtgatatgtaattaatgcagcccttctatgtttctcaagattcacaggctcgagagccccatttcttggaatctacaatgctgaagattgcattgaatgctgtctattagaggaaatccttggagaagttactggcttggaaatagtcacacatctgacagcatgttacaataggaattttggattggacactgagatagcccgttttacaacaaggtgagtggtgaagtcgagcaccctggagggtttgtgcccatcctaaagacacagcccactcatgctgcaccaaaaattactcctctggctcccccagacacggcactgaggattcctttaacatggcatatcttcagccataattcaagcactcgtgtagcatggaaggcttctgcattagcaagagtttccatggcttttctatgcagcatcgaagtgtgctgatgtctggccacagacttcttaccttgtttggaagtatcctctcgtcttggaaccgggataatagtcagaggcgcggccacagccctattcttgtctgagcatcctgtcaggttgcagtactcccacctgactttgggatccgttgtataacaccaagggcctgccgaacaatccggattcctgcagtagttggagatcaagccactgaaaatcacaaaacaatacacgttaacaagaggtgggacaatatgcaagaatatccggcaccctctccattttgctgtaacaaggtcataaccagtgcctttgaaatattcccgttatgggtaccatatgattgccacaagcatgaatggctctcaaattctcatccactctgcacatttctaataggtaatagattttaactgttttttttttaagacctgaagtagcaaattccttttagaaacactgaaataatacatacatgtggcctagaagggatgagaatatcctccttccacctttggccaatccatctctctggaataaacggtacagattttgatgtgtgtattgtggaatcacttatacacatacaagattatctcgatctgtctagctctctttctctctctctacaggacttcatatagtattgtgcaaggtggaactaacatagggttcctcaacgaaataatggcatccaggatgatatcaatcccaggccagccttctaactctatgtactcaaaatgtagtgaaaagcctctatgttacccatggaaaaccatcgtgcaaatagtattgctgtctctcgatgccgtatcaggtgagtgaaaaggtttgtttcttcaagacttataaaagctgccctagaaaacaagttcccagacatgatgcagtacctctaggatgcatccctggacaggacctcatctcctgctctcagtccatcctctactggtggcagccactctgggactgaaggaatggggaatgagttgaaagaacagtatgactcaaggcataatgggagatggctgaatagactatgagatctgaaaaggtcaggcagctacagagttgtcagcaaacaatggcgctaagaggcaaagcaactgagaatcccgtactgggtgtatcttggagcatttgccttttgttatgactcccaggaacattgctccaacctctcagaattcttacattcatgacctgtggctgtctgtgaagatgagaaatgtattatgggccatggggatccaacacctctctctagaaaatctacacatgtgagctagtctcaagcacagaatgtccattccaggaagctgattctcagagcatttggatgtgctcagagccaagcagttgaaagacttctttttcttccttggcttccctcttttggatacaaggagtcttcttgttatagaatatatttcagccttcaaaatttagggattgctctagaatttgcaatatctctctgaaaccacatacttcaacattttattaatatttttaaccatcatagataaggcaagaaacttacaatggtataattccttttatttcctatctgatttatgccatgttttcaaatttttacttcaaatatgctaaaataatatgtattgctgtcattttgcttcaagtagttaatgctcatttaagtcaattaagaaatttgatcaagaaaatattttgcatttacatctacatttgccatttgctgccttctttattatgtgggatgcagaaagttcccctcttgcactattcttcttctacctgatttcctttgggttacatcttttgttacataagcctgctgatttcaaattcttacagttttgggtttgtttcgggggggtgcacaagatgcatttatacaacatttatgatattcaaggtattttattgcttgatgtagcattctcagtatatctcttctttttcctgtgtattgtttggaccatattcctttgtatgcagcttgtcatgtttctgatgagaacgcattagtcatttgtatcactgttcctctagcagacacagctctttttcctctagtttctcatgggatttttcttgatactgtatctttgtcttctgacagacaagctgtgatgtaccaaagtgcatcatagagttttctttttgaaaattctcttgctgatccattgagctccttgaatctgtaatctttttatgcacctcatttttggaaggttttcagccatccttgcttcaagtatttgtttctgttccaatctctcattcctctatttctgtgacttctttcccacatgtaggaaatcttttatattttccttcatgtctgtgaggcttgatttcattttacagtatctgtggatgtgtgtgtgtgtgtgtgtgtctgtaacttgtgcatctgtgtgtgtgtgtgtgtgtagctgtttcttcagcttctatgggtttgttttcagttagtttttcttcttaacttttaactggatagggaggccattcaggaattttttatttaagtcatacatttcactagatttttaatttgttttgacttttttttactgttacttcctcttgatattacatttgtttggttccatatccatgtttttttaaatcctagaactcaactatcatactgatatgcccaatatttatgccattcttatcatttctgtcacctcggcctatttgtattgactgatttttctccgggtcatggatcacactaaatgattttcaatgtgtaggcagattttaattttaggactcacacagctgatatcgcacagctgagccacttgattttgttgtttacctctaaagaaggttgagtttttctctagcaggcagtaacttgacttggacaccattttgatgcttggacacctgcctttcagccttgctaggttttctctatagctgccttaattctaggaatacactaattctactcctaaagtctggcttttctggggtctctgctgaatgcccaagatgttcagtgtggtcactctcctctggctggtcagaactccaaccgcccccagtgctatctcagctctgtgagctttattgagctcactgttgccttgtagttgctgttcctcagtagatgtcctttgccctagtctcttgtggaacatttttctgtgcacaaaaaactttatgtttagtcaatgacttgaagagatttctatacgtattcctagagctcctttgccccacaaatccgttctcattcttagcttgcctagaaaatcctaggtaccggaaaatcccaaactccaatctctcctccattcagcaagatggatacagtccacatgggttccgtctccctgctcttccgtgcaggaacggtcccaagaagaaagccaaggagggcatctgtggagttgatctcattgtactccttgtcacaattctctcagtcctctaccatctgttgttttacgtgtggaaaccatttttccatgtcctttgtagccacttagaggagagagactggccatgagttggtcagacccaggcttctgtccatctgcccaccccttttatttttcagcatctgtctctttgctgtctctcatcagccttcctgcctttgcccttctctttctggcacctcatatctgagaccattgctgcttgggatcgagtttgtttctacctattggaatagacgtagaaatagaaagctttgactctccatagatatgcacatactccttcctcgtgagccaagacagaaagggccaacacaacttttacttcaggaatcagaggttagaatgatagaattccaaggtggaaaggctgcattgactcttactgtgtgttgtagaaaacactcttaagtgtttggttatttgtggtcctgacagttttcgtaatgtgatcacctcgccttcagatagcctcctgatgcataaagctatgaggaaggagagccagattaacatatcctatctctgagaccctttgcttcaaggcaatattcctgagacattttgctatgacctctacatctgaaacaagttgagtcctgagcacttagctagaagcatgactcttctaacagaaactttagtcagcccttccttctcttatggtaaagaacaaaacgtatgcatttgggtactgttccggggtcctactgtgctggtgtggtgtcatagatgaccaagcttggcaggttcttcctgtgacagtggtgaaatatgtgcctcgataactctgtccatttccatggtagcactcctgaacagaaggccttggctctgtcggtgctaaaattaaaacaggaaaaattaagctgtgtatctcctagaacagggaaacagttgaagccatttatgacacaaaccagaaagaagtcttggagaattacgaccattctcttttttttatttgtacacagatgaacatgagaaaacaaacaaacaccaaaagaacagattcctaccattctagaaatgcagtaagttcttcccaatatttcatgtaaaagcttagagaaaaacaacagttcagagtgcttcttatggaaaactgcgctcacgtgcaaactgctctctagtgatctctactgaatgttcatggggatcacggggcatgagagaataaagggctacgcatttggcagtgcagactgtagatggtgattggctgctgagggcggaggtggggacagacatggaaacctcgcacgtttccctagaccattgatcacgagagccaaggccttcagcttctggcttctggcagattgggaagaaaacggttgtgcgtctaggactgcaggcaaaaatagtttgcttcataaggatgggtggaggccaaaatcttatcccattggaagagagatggaattttctcatgccccacatcctgcattgcaataaagcagaagtctcctactctttctctccagaccccccacaagtacaacttccattagaaaaaggtgcaggaacactgagaaatcactcacttcaaagcctaggcctgcaaatcttgcccaggtctgaggaaggcctgaaagatagagccttgggagctcatgtaatgctccatgatgcaagggcaacatggaattaatgcagccctgttaagtttctcaagattcacgggcttgaaagccacacatttctgagaatctgtgatgatgaagattgcactgaatgttgtctacttgaagaaatccctggagaagcttactgcattggaaagagacacatatctgacagcatgttacaacaggaattttggactggacactgagataactcattttacaatgaagtgagtggtgatgtcgagcaccctggagggtttgtgcctatccaaagacacagcccactcatgctgcaccaaaaattactcctctggctcccccagacactgcactgaggattcctccaaaatggcagatcctcaatcaacattggagcacctgtgtagaatgaagccttctgcatcagtgggagtttccatggcttttcttcttagtgtctaagcgtggagatgtctggccacagacttcttaccttgtatgaatgaatcctctaggcttggaactgggataacagtcagaggcacgaacacagtcccttcatcgtctgagcaccgtgtcaggttgcagtactcccacctgacattgggatccgtcgtataacaccaagggcctgccgaacaatccggattcctgcagtagttcgagatcaagccactgaaaatcacaaaacaatacacgttacaagaggtgggacaatatgcaggaacatccagcaccctctccattttgctgtaacaaggtcataaccagtgcctttgaaatattcccattagaggtaccatatgattgccacaagcatgaatggctctcaaattctcatccactctgcacatttctaacaggtaatagattttaacttttttttttaagacctaaagtaggaaatacctcttagaaacactgaaataatacatacatgtggcctagaagggatgagaatatcctccttccatctttagccaatccatctctctggaatacatggtatagattttgacgtgtgtattgtggaatcacttatacacatacaagagtaccttcatctgtctagctctctttatctgtctctacaggacttcatacagtactgcgaaaggtggaactaacatagagttcctcaatgaaataatggcatccaggatgatatcaatcccaggccagccttctaactctatgtactcaaaatgtagtgaaaagcctctatgttacccatggaaaaccatcgtgcaaacagtattgctgtctctcgatgctgtatcaggtgagtgaaaaggtttgtttcttcaagacttataaaagctgccctagaaaactagttcccagacatgatgcagtacctctaggatgcatccctggacaggacctcatctcctgctctcagtccatcctctactgatggcagccactctaagggtgactgagggaatggggaatgagttgaaagaacagtatgacccaaggcataatgggagatggctgaatagactatgagatctgaaaaggtctggcagctacagagtcattggcaaacaatggcgtaagaggcaaagcaaccaagaatctcgcgctgggtgtatcttggagcattcattcgccttttgttatgactcccaggaacattgctccaacctctcagaattctcagattcacgacgtgaggctgtctgtaaagacaagaaatgtattatgggccatggggatccaactactctctctagaaaatctacacatgtgagctagtctcaagcacagaatgtccattccaggaagctgattctcagagcatttggatgtgctcagagccgagtagttgaaagacttctttttcttccttggcttccctcttggatacatggagtcttcttgttatagaatatatttcagccttcgaaatttagggattgctctagaatttgcaatgtctctttgaatgcacatatttcaaccttttattaatattttttaccatcacagataaggaaagaaacttacagtggtataattccttttattccctatctgatttatgctacattttcaaatttttacttcaaatgtgctaaaatatattgctgtcattttgcttcaagtagttaatgctcatttaagtcaattaagaaatttgatcaagaaaatattttgcatttacatctatgtgccatttgctgccttctttattatgtgggatgcagaaggttctcctctcatactattcttcttctacctaatttccattgggttacgtcttttgttgcataagcctgctgattccaaattcttatagtttttggtttccttgtttcgggagtgcacaagatgcatttacacaacattgatgatattcaaggtattttcttgcttgatgtagcattctcagtatatctcttctttttcctgtgtattgttttaagatatccctttgtacgcagcttgccatgtttctgatgagaacgcattagtcatttgcatcactgttcctctagcagacacagctctttttcctctggtttctcatgggatttttcttgatactgtatctttgtcttctgacagacaagctgtgaggtaacaaagtgcatcacagagttttctttttgcaaattctcttgctgatccattgagctccttgaatctgtaatctttttatgtacctcatttttggaaggttttcagccatccttgcttcaagtatttgtttctgttccaatctcttattccgctatttctgtgacttctttcccacatgtaggaaatctttttatattttcctccctgtctgtgaggcttggtagatttcattttacagtatttgtggatgtgtgtgtgtgtgtctgtttataagttgtgtatctgtgagagtgtgtgtgtgtagctgtttctttagcttctatggattttttttttcagttagttgttcttcttaaacttctaacaggatagggaggccatttagtaattttttatttaacacatccttttcactagatttttgatttgacttttttatactgttaattcctcttgctattacatctgtttggttccatatccatgtttttttaaatcctagaactcagttatcatactgatatgcccaatatttatgccattcttatcatttctgtcacctgggcctattcatattgactgatgtttctcctggtcatggatcacactaaatgattttcagtgtgtaggcagattttagttttaggactcacactactgatattgcatagttgagccacttggttttgttgtttacctgtaaagaaggttgagtttttctctagcaggcagtaacttgacttggacaccatttcgatgcttggacacttgcttttcagctttgctaggtttctctggagctgccttaattccaggaatatgctaattctactcctaaagtctggcttttctggggtctctactgaatgcccaagatgttcagtgtgatcactctcctctagctggtcagaactccaaccgccccagtgctatgtcagctttattgagctcactgttgtcttgtagttgctgttcctcagtagatgtcctgtgccctagtctcttgtggaacatttttctgtgcacaaggagctttatgtttagccaatgacttgacgagacttctaaatgtattcctagagctcctttgtcccacaaatccgttctcattcttagctgtcctagaaaatcctagctgcctgaataatgccaaactccagtctctcctccattcagcaagatggatacagtctgcatggattccgtctccctgctcttcagtgcaggaacggcatccaagaagaaagccagggagggcatctgtggagttgatctcattgttctccttctcgcaattctctcagtcctctaccatctgttgtttaacgtgtggaaaccgtttttccaggtcttttgtagctatttagagcagagagactggccatgagttggtcagacccggcttctgtccatgtacccaccccttttatttttcagcatctgtctctttgctgtctctcatcagccttcctgcctttgcccttttctttctggcacctcatatcttttcagaccattgatgcttgggatcgagtgtgtttctacccattggaatagatgtagaaatagaaagagattcgactctccatagatatgcacatactccttcctcgtgagccaagacagaaaaggccaacacaacttttacttcgggaatcagaggttagaatgatagaattccaaggtggaaaggctgcattgactcttactgtgtgttgtagaaaacactctaagtgtttggtcatttgtggtcctgacagttttcgtaatgtgatcacctcgccttcagatagcctcctgatgcataaagctatgaggaaggagagccagattaacatttcttatctctgagaccctttgcttcaaggcaatattcctgagacattttgctatgccctctacatctgacacaatttgattcctgagcacttagctagaagcacgtctcttctaacagaaacttcagtcagcccttccttctcaggtgggatgatatgcagggcacccagcacccagcaccctctcaattttgctgtaacaaagtcataaccagtgcctttgaaatattcccattaaaggtaccatacaattgctacaagcatgaatggctctcaaattctcatcctctctgcacatttgtaatatctaatagattttaactgttttttttttaagacctaagtagcaaatgtttcttagaagcaaagaaataatacatacatgagggctaggagggatccgaataccctcctttagcctttggccaatccatctctctggaatacctggtatggattttgatgtgtgtattgtggaattgcttatgcacatacaagagtattttcatccgtctagccctcttgatctctctctataggactttataaagtattgtgcaaagtggaactaacatagagttctccaaagaaaaaatgccatccaggacgatatcatttccaggccagccttccaactctatgtactcaaagtgtagtaaaaagcctcagtgttagccatggaaaaccatcgtgaaaatagtattactgtctgtagacactgtatcaggtgggtctagaatatttctttctgcaagacttatcaaagctgccctagaaaactggttctcaggcaggatgcagtatctctaggatgggtccctggacacgacctcgtctcctgctctcactccattctctagtggcagcagccactctgggactgagggcatgggggatgagttgaaagaacagtgggacccaaggcgtaaagggagatggttgaatgaactatgagatctgaagaggtcgggcagctacggaggagtcagcaaacaatgtagtaaaaggcaaagcaggtgagaattccgtgtttggtgtatcttggagcattcgctcccccttatgactcccagaaacattgctccaacctctcagaattctcaacattcacgacctgtggctgtttgtgaaaatgggaaatgtattatgcgccatggggatccaacactgtctctctagagactctacacatgtgacctagtctcaaccccagaccctccattccaagaacctggttctctgagcatttggaccagagcagttgaaagacttctttttcttctttggctgtcctcttttcaatacatggaatattcttgttatagaaaatactttagcattcaaaattttgtgattactctagaatttgcaatatcccatgaacccaaacaactttttattaatatttgttaccatcattgataagacaagaaacttacaatgatataattcttttattccctacatgatttatgctatgttttcaagtttttacttctacatatgctaaaataatatatattgctgacattttacttcaagttgtcaactcttatttaaggcaataaagaaatttgaacaagaaaatattttgcatttacatctacatttgccatttgctggcttcttcattacctgggatgcagaaagttcccctcttgtactattcttctacctattttcctatcggtcacactttttgttacgtaagtgtcctgattccaaatttttatagtttttggttttcttgtttggggaattgcaaaatacgcatttatacaacattgatgatattgaaggcattttatggctggatgtagcattctcagttgatctctttttttttttcctgtttatcattttgatgatattcctttgtatgtggcttgccatatttctgatgagaacacattagccatttgtatcactgttcctctagaaagacacagcactttttcctctggtttctctgagatttttatgatactatgtcgttgtcttgatagtcgaactgtaatgtatcaaagtgcatcatagaattttctttttgaaaattctctttctgacatattgagcttcttgtacctgtaatttttaaatgttcattatatttggaaggttttaagccacttttgcttcaggtatttatttctgttccagtttctcattcctctacttctgtgacttgcttcccacatgaagaaaatctttgcatgttttccttcatgtctgtgaggcttggttgattcaattttaccgcatttgtggatgtgtgtgtgtgtttgtaaggttgtgcatgtgtgagtgtgtgtgtgtgtgtgtgtgtgtgtgcatgtagctgcttctttagcttctatggatttttttttcagttaattttttttctgaaacttctaactggatgaggaggcaatccagtgatttttttttttatttaagacataccttttactagatttttaatttaatttggctcttttatactgttacttcctcttgatattacatctgatagttccatatccatgttttcttaaatcctagaactcagttatcatactgatatgtccaatttttatgccacttttatcatttctgtcacctgggcctatttgtattgactgatgtttctcctggtcatggatcacactaagtgattttctatgtgtaggtagattttaaattttggactcacactgcggatattgcatagttgagccacttgattttgttgtttacctctaaagaaggttgagttttcctctggcaggcagttaattgacttggacaccattttgatgcttggaaacttgcttttcagctttgctagggtttgtctggagctgcctttattataggaatatactaattttatgtctaaagtctggcttttctggggtctctactgaatgcccaagttgttcagtgaggtctctctcctttggctggtcagaactccagtctccagcagtgctatgtgagctctttcatctttattgagctcactgttcccttgtagttgctgttcctcaggagatatcctttgccctactctcttgtgaaacctttttctgtgcacaagcagctttatgtttagccaatgacttgaggagattctatacgtattcctacagctcctttcctccacaaatccattctcattcttagctggcctagatcagcctacctgagtaatcccaaactccaatccctctccttcattaaggaacatggctacagtccatttgggctccatctctccactcttagggaagggtctccaagaagaaagccaagaagggcatctacggagttgatctcattgttccccctctcgcaactctctcggtcctctaccgtctgttgtccaacatgtggaaaccatttttctatgtctttggtagccacttatggcagagaaactggcaatggggtggtcagacctgggcttctgtccatctatctgccccttttacttttcagcatctgtctgtttgctgactctcatctgccttcctgcctttgctcttttcttcctggcaaaccgtatcttttagatcactggtgctcaggactgagtgtgcttctacccattggaatagatgtagatacagaaagagagagctttgcctctccacagaattgcacatatgctatccttgtgagccaagatagaaaagaacaacgcaaccttcacttcaagaatcggaggtcagaatgctagagttcaaaggtggaaaggctgcagtgacccttagtgtgtgttgtggaagggtacgctaagtgtttggtcatttgtggtcctgatatttttcttaatgtgatctcctcacctccagatatgctcctgctctatcaagctaggaggaaggagaggcagcttaacatttctcttctctcagaccctttgctccaaggcaatgttcctgagacattttgctacgccctctgcatctgacacaagttgagtcctgagcagtcagcttgaagcatgactcttctaacagaaacttcagttggcccttccttctcttatggtaaagaacaaatacatactcatttgggtacttttctggggttctactgtgctggtgtggcgtcatagatgaccaagcttggcaggttcttcctgtgacagtggtgaagtatgtgcctcgataactctgtccattactgtggtagcactcctgcacagaaggccctgtctcagtcggtgctgaaatcaaaacagaaaaaattaagctgagtatttcttagaacagtgaaacaggtaaagccatttataacagaaaacagaaaggagtctctgaaaattatgaccgttctcatttctttatttgtaggcaaatggaaatgagaaacaaacaccaaaacaacagattcctaccattctagaagtgtagtaagttcttagaaatatttcacctaaaagtttagagaaaaacaacagatcagagtgattctcatgggaaactgcactcacatgcaaactggtctttagggatctctactgaatgttcatggcgaccatggggcatgagagaacaaagggcaatgtatttggtggtgcaggctgtaggtggtgactgggtgtcgagggcagaggtggggacagacatggaaaccttgcatgtttccctagaccattgatcacaagagccaaggccttcagcttcttggcgattgggcagaaaatggttctgcatctactactgcaggcaaaaacactatgcttcttagtggtgggtggaggccaaaagcttatcccattggaggagagatgtaactttcttgtgccccacatcctgcattgcaataaagcagaagtctcctactctttctctccagacaccccacaggtaaaagtttcatcagaacgaggtgcaagaaccctgagaaatcactgtcttcaaagcctaggggtgcagaacctgcctaggactgaggaaggcctgaaatatagagcattgggagctcatgtaccgctccatagacacagggcattatggaattattccaaccttgttagctttctcgagatttataggcttgagagtcatgtttctcagaatctgcaatgctaaagattgcactggatgctctctactttatgatatcttggagaagcttactggcttggaaaaagtcatccatctgacagcatgtttcattaggaattttgggctccaaacagcatccctatctgccactaacatgtcaagcaccactggatctgctgggtcatatggcccaagtggcagagcagcttgcacagcagcctgattctgttgcagagccttctcttgttctcgacttcactcaaaactggcagcctttcaggtcactaaataaatgggccagagtaacagacccaaatgaggaaggtgttgcctccaaaatccaagtaggcccactaggcattgtacctctttcttggttgtaggagggttcaaattcagcaacttatgcttcaccttagaaggaatagctccataggccttacacctctgaaccgctagaaattttactgaggtagaaggtccctaaattttagtcagatttatttcctatcctctggcatcaaatgtctcaccagtaagtacaatgtgtttactacttcttgctcactggatccaatcagcataatatcatcaatataatggaccagtgtgatatcttgcagaagtgaaaagtgattaaggtctctcgaataagattatgacacaaagctggagagttgttatacccttgaggtaggacagtaaaggtatattattggccttgtcagctgaaggtcaattgcttctgatgggccttatggacaggaatggagaaaaaggcatttgccaagtcaatggccgcatattaggtacaagaacatgtgttaatttgctcaagcaatgaaagcacatctggtagagcagctgcaattggagtcaccactaggttaagcttacaataacccactgtcattctccaagatccatctgtcctctgcacaggccaaatgggggagttgaacagggatgtggtagaatcaccaccccctcatctttcaagttcttgacggtgatgtcaatctctgcaatccctccaaggatataatattgtttttgatttactatttttctaggtagaggcaactctaatggcttcaatttggcctttcccatccataacagccctcaccctaccagtaagggagccaatgtggggtttctgccagctgctaagtatgtctatgccaattaggcagtttggcactggggaaatgaccacaggataagtccaggtactaactggacccagtgtaagtcagacatgaactaaaatgccattaattacctgacctccatatgcccctactttaactggaggaccacaatgatgttttgtgttttctgggatcaacatcagctcagagccagttttcagtagtttccaaaacgtctgattatttccctgtccccagtgcacagttaccctggtaaaaggccagaggtgtccttgggaaggatgggagaaagataaacaccataaattgttggtagcttagtagggtgcttccttaaggggacccagcctcccctttattcaagggcttctgggtctgtaaactggctcgagtctggaaattaattgaggggccatgattctgtgtcttaataattcagattagtcttttgtccattctacctggtagttttctgtttatataaattaagtaggaatgcagtaggctttctatcaatttcacttctaggaacatcgtgattaattagccagtgccagagctctacacgagtcagactattctgattgttgctttgcctctgctgtgcattatggtagctatacccagcattcctctgatgggtcagtgttgccacttggtccctggccctcaggatccaattattcccattgtatttaaattttgtagttgagtgactgtggttcccactgttagatctgacatacagagaagagcaattacagggctcttgaaaaatgcaggtgctaccctcacaaaactatttcacaaagcattggtcaagggtatatattctggaccctcccagctgggatgagtaggtctaaagtgactaatccaacaccaccatcctaatctccctaagcctttggaccccttcctctacattaaactaagggagatcaggcatttccagctcactcacagcaggccatcttttaatctatatttcgggtaatcaagcaaataaactattagaaccttttttaagtccccaagctgcaacattaaatgcagaatccctacttagtaggcccacattaataaattcagcctgatccaactctatgttacttccaccattatcccagacccttaatatccattcccatgcctgttctccatatttctgcttatataaattagaaaactcaagcaattcttttagagtgtaatgcatctcctcatgggtcacactctgaacctcacctcttgggcccaacaggactttagtctagttgtaggtctagaagcaaacaagggcattggagatgcctcctaaggagaatcaacattatcttgcctggcaactgcctcacgggaggccgtcactgttgcctcaagcagcgcagggttcacatcctcagacaaaggtggaaaggctaatggcagcatgggtcagggaggggatgttgccagtactgaggatggggaagctgtttcttttcgcaaaaaaaggttaatcaaagttcataaattcagtgtcctcagtttcatcagggtcctctcacatgtccccattccaagttacagggttccattcttttctattcaatgccctcactttaacagtagacacctggcagggctgggcatgcacctttcattgcaggttagctactcgcataataagagcttgcatctgttttactacaatttcaactctttctctacaggagttaagactctcactcagggcaatcttagcagatgtgtggctcagtatctgcttctgaagccaggagacagaatccctgagtttatcattttctttcatcactttctccagtgaacttaggaccaaccaacttcattatgctccttggttctccacatatagtcaaaggtattatgcatagagtcactaaactccttccctcatgagtggtgaatcaggagtgtcaaatgcatttattttgcctaactctttaaacagttcatgccaaggactatcagtgttctccatactattagaagtagagtgctttgcatttttgggtctaatcatattaagcagtcatatccagaaatctcaaaaccaatgaaagaactccatccttaatattctgttcctccagaaccactcctggttccaaaatctatattactcagggttctctagagggataaaactaataggatacacacacacacacacacacacacacacacacacacacacacacacacacaaaggggaccttattaagtagtatcaactcacaagattacatggtcccataataggccatctgcaacctgagaagcaaggaagccattctgagtcccaaagctgaagaacttggagtccgatgttcaagggtagaaagcatccagcacaagagaaagacgtaggctgggaagctaggccagtctagcctttttacatttttctgtctgctttgtattctggccacgctgccaactgattagatggtgcccacccagattaagggtgggtctgccttcccagcccacttactaaatgttaatctcctttggcaacaacctcacagacacacccaggatcaatattttgcatccttcaatccaatcaagttgacaatccgtattaatcatcacataaacaatagtaatatactcaaatatgtatcttaaggatggttgttctttatactacctcattgaggttctctgaatttcctgagtttgtgtgtcgacctctctagttagactgggtaaattttcatggacaatatcctcaaatatgttttcctcattgcttgctctctctccctttcagggacaccaatgagtcatagatttggtctctttacataatcccatatttctcagaggctttgttcattttttaaaattctttttctttatttttgtctgattatgttaatttgaagaaacagtattcaaggtctgagattctttccttaactacttttattatactaatatttctgattgtgctatgacattcaatagtgaggttttttttagctctacaagagagggttggttctttcttcaaatggcttgtctttcatctcttgaatcattttacttcattccttagattacttgaaagaggtttcaattttctcctgaatctcaatgctctttgttgccacccagattctgaattccatgtctgtcatttcagtcatttccttctggttaggagccattgctggagagctagtgtggtcatttggagataaggagacagtttggcttgcagagttgccagaattcttgcgctgcttctttctcatttgtgtgacctgatgttcctttaactatgatataagttgacttcatttctggatgttttcagagggccaaggtggtgtacaaggtctttatttgtggttgaattcttgtccatggttttacggggtggtatattagcaaagtattttggattttgaagattttgattttgcaaactaacaggtaggctcttcttcagccacatggctcctctgtatttcctcacaattgcttctctcccacttgagttccagttgcagatctcagcttagcattctttggccgtgtaccgcagccctggggcaagctcagctttgttttggctccatagcttgggagaagcaggggcaggggcgtcagggtgcttgcagggaagacacactaaactttttttccatagggtggctggggcctgctggtgtgtgaataaagcactcaggatctttgtttcttccccagtccacagacagcaagggcagtaccactgcagtggcagtggcagatgggctttcagtcgcctctgggagctccatcccacagaaacacagagagctgccaatggaaatgttcagctgagtatgaggtgcctgtactgtgtgcccaagccaggaaacctgcctggtgaagagtaacaggtgggagctcacagggaagagagactggaattctttccatataacccctgctgtgtgctggaggcatcagggaagtgaccaggtcactttgttccttctgcagcccaagggcagtaagggcggtattgctgcagctgaaatggcagaggggctgtgggttgcctctgggatttcctccccagagaagcacagtgccttcctgtgctcccctgcctgaacacactgactaaagtgttcaggcagggccagggtggttgtgctctgcccattgaggagtagccggggcaggcaccagtgcagaaaacagtgtgcccacttttccgtaaggcagctatactgtgctggaggtccgtgagagtcctgaagctcctcgctccctcctgagcctgcactttaatctcaagagtttgagatacacagtggtaaggaagaaagtaacaatgagtacagctaatgaaacccaggaagacaagtcaattctctttgaggggtgaggtgggggccacaggcagatggagtggattaaagaatgactagtagagtggcagcagaagacaagcctggctctgaaggcagcaagtcctccagggaatatggggtcaggggaggttctgaaagatgacacactgcagagagcgtgtgtttgtatgctgatgacaagtatcagtagaaaggcagatgaacggattctcattctcagaaagacctgtcctgacctcctgagagtacacagagcatgagggcaggacagggaggaggacgcactctcattgattcatgcaaagtcctcggccaaagtttacctgctctccacctctgtgactttgtcctttctcaatttaagatgctccaagctggaaaaatggattattgaccagtagttggctgatgcaattctaaagctaatacaccaaaaagcacttgagagatgtatcagaagatcatagagaaatagatactttaagctaatatccatgtcttcaccctgcactgtcttcttatttgaactcccttttccgggaagagttgtgcttgggtaaattctatagctcacagtgtgacttcaaaacacataataaattcataaactgtttcctacagtgagttatgttaaactttctgaagagtgtttttgaaatgtaggagagcagactacaattgatattcaaaaagaggaaacttcctggccgggcgcggtggctcacgcctgtaatcccagcactttgggaggccgaggcgggcggatcacgaggtcaggagatcgagaccatcctggctaacacggtgaaaccccgtctctattaaaaatacaaaaaattagccgggcgtggtagcgggcgcctgtagtcccagctactcgggaggctgaggcaggagaatggcgtgaacccgggaggcggagcttgcagtgagccgagatggcgccactgccctccagcctgggcgacagagcaagactccgtctcaaaaaaaaaaaaaaaaaaaaaagaggaaacttccatttattttcccctttaaccaagttaacccataactgtgtcgtgttattatgcttcatagcgcatcactgctgagatcccctctccagctcatgtcgctaggaaatatcccaaaacagaatcaattttttgtctttttaatttgataatttttccccaaggcttttgtttctttgattcacctcatatggcaaggggttttaatagggtttatttgctggtgaagaaacattaaacacatcttttaatagaaaaaatacttttataggcaaatgtagttgtatattgctagtgaagtaactggattttattaatggtgtgagaatttcttcctcactgtggtatgtgaatgtgtatgcatatttgcatatgtaaaagaaatagtaatttgtgaaatggaagatgattctccacttctcactgagtatggctgtaaaataaacttgctctcctcgggcactgaagtacactggaaagatgacacatctacagaaagcattcctctccaaaagagcttataagagaatgtgccccccaggttggttaaaacgtatgttaatcctatggcagagaagtttgatttagcctttgggtgtagcccactgagtaagttcatagtgatctatgtcccagttttcatgggctgaaatataatactcatccccctgcccccatctacacacacacacacacacacacacacacgtagacaccaatttccccccaaaacctctttgcttaatcttctcctgctttgtattttctttgatactttgttgaatggcttccactaagttcaaatgtattttactctaaagtcaatttgtaaaattttatctggtaatttatatattaaattgaaagcatctacacattgcttatatgaagaactaccatgaaaacgtatggttccatttatatgaagtgtccaaaataggcaaatctataaaagcagaaaaatacatttgtggttgccagaagctgcagacgcagggcaacagctaaggagagtggagtccctttttggtgtatgaaaatattttaacatttattgtgatgatggctgcacaactctgaatatatttacatccattggcttgcatgccgtaaatggatgaattagacctcaataaagctgtttaagaaaaacctattagtgagaccaaggacccccatttttctaagagatggtttaattttttgtgtgttttttgtctccccagctccccactttatacttggccctttagaattgcaaatatggctttttacctccccttcatctgaatctccctacagggcaagtcatctaactatgtgcttcaagatggaactcctgagttcacagttgatttataaaccaaagaatgtccactacaaaactctcaccctccagagacttgtctcaagcgacaacatcctgttcacaagggcaccagcaggcaccagctggtggataaggtgccaaagctagcatggacccctgaaccattgcttgcttcctcccctgccttttaaaagtaccactttctgctccaaaagtgaagtggtaaaacaggactcttgtatttcttcccctaagctagcgttggaagtacatcactttctttacaccagacttctctcttcttaattagattctgcaagtggcaaataactaacctgctcttccgttacactgggatgtgtttagattagtttttcaaaggttagaacaaagttgaaaaagtgagaagagccctagattttttttcttggaaatctggcttaaatcctggttcctttatttccttgatttgggaatttggtctctgggcaaataacatcattttcctaagtctagatttccttgtttataaaattgggatgaatatgctatgctatctacaccatgagctcaataagctactgtctgtaaagaaatcacactcacactgacatgtacacacagatatgtatgtacgtatacaaatgtgaggacctaacccaagcactgagtgagtatttgctgagtcactatctgcatacagcatctggcaaatggatgtgtgtaatttctccagggataagaagcaaacgtatacctatggaaagccttattcaggccataacaaggagagaaatgctgagtcccactccatgaatgggtcaaggagcagaatatattcaaagagatatttcattgtttcaacttgaaagtgaggggagcatggcacctgaacagagatgatataaattaatgaaactgagaaccaggtgaagagcttgaccatcctttgcgtagttagctacattgcctatttttttctttcaactagactggccatgatgtaggcacactcttttagattagaaacaccacccattatgcccattcattccagcaccctgacagtcttcatgtggaaattaaacttagttttttaagaattcctcaatcttatacaagattttgaactggcgattttatgaatcaaaaattaaattaaatgaattgcacataaagatatggcatatgtatttttactacattgtgggagaaaaaataattattacacaaaaaaaactatgtcttacctgatttcagaaataacagaagtagaagaaccacttccttatgttccattttgggactggccagcagtgcccagaaagtgtgtcccaatcccaggatgttgttgacttacatgagagtaaacgcatccacaaaccagatagtcaaattaagttaatgattctctcagagacccatgccactggctcagcagagttcaaacattacctgaatttttagaagcccttttctgaatccaaccatccctcagtcccctaagagtggccagagaagaaggactccactgagctgacattccagttcagtaagttgtagggacagttttttaaaagaatctgtaggtaggggagcaccaacccataacatactgtaagcctgcagggttctccagcccagtcccttctagaacccttccctgtccacaaccagtgacggattattccaaacgttgttcagtttcatttgacctagtttgaaaaccaagttccaataatgaataactatgggagcttgggttgctgtattcggtctctgagtagcttttccacctacccaggccacaatcaataataatcaccattgaacatccttgaagcaattcttttgaaattactttgcatcaaagattgcacaagaactgctttaagtagaaggactgatgtcagcaatcaagggtcccaccgagggctgcagatagttcttgaccttcacatgggaaggcgtcattgagttctgacactctggcaatcctctaggactcaactttctgataagctaataaatggaaaggtgaaattagataaagacattattacctcatgggacaactaagcattagaggttagtctaaacatgctcttttagaatctttactacactactagctaccatttattgtaggtttgtctatctgtgccagatacaaacaacactatgaattacccataccatgctataatcaccagatttttctctgttattcccattaccattttatagatcaggacattgaaatttgcttgcccatagttatatcaaaggaaattgggaaagccaggttccaatgcaaatatctcaaatgattacttttttttttttgagtcagtctcactctgtctcccactgtggaatatagtggcacaatctcagctcactgcaaccttcatctatgggtttcaagcgattctcccgcttcggctttccaagtaggtgggattacaggtgtggaccaccacgcctagctatttttttgtatttttagtagagatggggtttcaccatcttggccaagctggtctcaaactcctgatttcaagcaatccccccacctcagcctcccaaactgtggggattataggcatgagccaccatgcctggcctcaagtgattactttcaaccactgcactacaatttactactgggaggaaagaataataataatagtaatggctataatttattgattaatggctatatgcttggcattgtactcacttttgaagttttaatttataattgacacatagtaattgtacgtatttatggggtacagtgtgatgtttcaatgcatggatacactgtataaggatcaaatcagggtgattagcatactcctcacttcaaacattttttctttgtggtgagaagattcaaaattctctcttctagttatcttgaaatacacaacacataatcatgaactatagtcaccctagtgtataatagaacaccagaacttattctttctatctaactagaaatttgtacacattgaccaaccccttttcattctcctctccctgactaccctgctcaccctctggtaaccactattctactgtctaattctgtgaaatcaagattttactaagttttattgataaataataattgcatatgttcatggagtacacagatatatttcaatacaaatgatgtacagagatcaaagaggggtagttagcatatcaatcaatcacctcaaacatttatcactcatttgttttggaaacactcaatatcctctttctagttgcttgaaactatacaatataatattgttagctagactcatcctacagcggcataaaacactagaacatatttctgatacctaggtgtaattttgtagcctttaacaaatctctccctattccccctttccccactcttccagacctggaacatcctccatattactttttacttctttgatataagcttttttagctgccacatattgttactggcagcaaatccatatgggtctgcagcaacctcaattgttgcttccttggacgaaagattttaagaggcaggaggcaaaaggacagcaggagtgacagtttatttaaaagctttagagcaggaatgacaggaagaaaagcacactcggaagagggccaggcaggtgacgaaaggcaagtggggtggctggcaaaatggccgaataggaacagctccattctgcagctcccagcgagatcaacacagaaggcagatgatttcttcatttccaactgaggtacccggcttatctcactgtgactagttagacagtgggtgcacctcatggagggttagcagaagcaggctgggacatcgcctcacctgggaagtgcaaggggtcagagaactccctcccctagccaagggaatccatgagggtctgtgccgtgaggaatggtgcactctggcacagatactatgcatttcccatggtcttcacaacccacagaccaggagattcccttgggtgcctatgccaccagggccctgggtttcaagcacaaaactggatagccatttgggtagacactgagctagctgcaggagctttttttccataccccagtggtgcctgggatgccagtgagacagaactgttcactcccctggaaaggggactgaagccagggagccgaggggtccagctcagtggatcccacccccatggagccaagcaccctaagatctactggcttggaattc

SEQ ID NO: 31 - DNA, 3105 bp, Homo sapiens
gactgtggaagcacgagcttacataagtggttaggcaagctggcgtgtggacttgtgtgggatgggtgcaggtctgcatataaatcatgttcctgggtttctgggtgtgaagcgcacaccgacggggacgagagcgcataggatggcgcttttagatacttccagtcttccgatgttgtgatcgcagtgtgagtgaagacactcaggatggctcaaggatccggggatcaaagagcagtgggggttgctgacccagaggagagttctccaaacatgatcgtttactgcaaaattgaagacatcattacaaagatgcaagatgacaagacagggggtgtgcccatcagaacagtcaagagctttctctccaaaatccccagtgtcgtcacaggtactgacattgtgcagtggcttatgaagaacctttccattgaggacccagttgaagcaatacacttggggagccttatcgctgcccagggctacatctttccaatctcagaccatgttctcaccatgaaggatgatggcaccttttatcgtttccaggctccgtacttctggccttcgaactgctgggaacctgaaaacactgactatgccatctatctctgtaagaggacaatgcaaaataaagcaaggctggaactggcagattatgaagcagaaaacttagcaagactccagagggcctttgcgaggaagtgggaattcatctttatgcaagcagaagcacaagtaaagattgaccggaaaaaagacaagacagaaaggaaaattttggatagtcaagaacgagccttttgggatgtccacaggcctgtgccaggctgtgtgaacacaacagaaatggatatccgaaaatgtcgacgtttgaagaatccacaaaaggttaaaaagtccgtgtatggcgtgactgaagagtcccaggcacagagcccggtgcatgtactcagccaaccaatcaggaaaacaacaaaagaggacatccggaaacagataacatttttgaacgcacagatcgacagacattgtttgaaaatgtccaaagtggctgaaagtttaattgcctacacggaacaatatgtggaatatgaccctttgataacaccagctgagccatccaacccttggatcagcgatgacgttgctttgtgggacatagagatgagcaaagagcccagccaacagcgagtaaaaagatggggcttctctttcgatgagatattgaaggaccaggtggggcgggaccagtttctacgattcctggagtccgaattcagttcagaaaacctcaggttctggctggctgtccaagatcttaagaaacaacccctacaggatgtggccaagagggtagaagaaatctggcaagagtttctggctccaggggctccaagtgcaatcaacctggattctcacagctatgagataaccagtcaaaatgtcaaagatggagggagatatacatttgaagacgcccaggagcacatctacaagctgatgaagagtgacagctatgcccgcttcctccggtcaaatgcttaccaggatttgctgctggccaagaagaagggaaagtcgctggcgggcaagcgcctcacgggcctgatgcagtcctcctgaccgttcctaccgcaggtccagggcctgggcccgcggaccccacaggcaggcggcggcgctccacatctgcggacagagtttccttacgaggagacttggtcactgtgaaggagaaagagtgagggcaatgaagggcgatggtggggagactcggtgggtgaatggggagaccagaaagaaagagtccacagagcctgggctgggcccggtggaggctcctgtttacagccctctcttctttgtacagttgtattccaacactccactcgctaagaggccctgatcccagctcattcaggggagaacacgtcgtggggttcctgtcacgactatcacttgagattatattattatcctcactccctcgctgtctggagacggtcacaccttctggcaaattcaagaggcatgagtggaccgagagcacatccagcatttgcatcacctccctggcacctcccatagttacagccactacatccccaccgccgcctgtcatccctcacgtctctgtggccacccgggtgtcactgccagcaccaggcactgctttcacgtaaaatgtccaaatcacatcttcagcaagaaagcaacctcacggattgccccgcctcaaggtctttccaccctctttgagatcagcgttcggagaggtccccgccagcccggtggctgccctcaggtcccgtcacatgtctcaagggtccagcgttcgaggaagcactgttgtagatgacagagctttttgtttcacagctgcggggattctgctgaccatcttttagaaaattatttcataatttattctggctttgcccccttgctccttggtttattccctcatgttttactcaaaataaacatggccatgaactcaggggccattgggattctcccctctggcagccccaggtgggagctgggagggtccccagcccaggaggtggctggattgcagggctggcagcccccacgtggtggcatcctgacaggcacagtgagacccttgctgggtcaggtctcctttgtgtgtgaagctggggcagagtatgctacgaagggaccaggccaggccacagggctctcctcccagtctttatctgaaggcccagccaaagcacccctctaaaccggtcacctaggggaaatgcaaaaacctgcaaaagatccacctgtattccctgtgtgtgtgtgtcaaaatgaccatttgccaacctggctgtgaggtaatgtatgtaaagcaccgagtcaggtgcctgatacgcagtaggtgcttagcaaacggtagctaatactatcatcattgtcattgttattgtgacacccatgagcccacagacagagctgtattaagtaacctgttttgaatccagacattcactcccccacttgctatgactaccctcctttgatctatttttggtcataggtaaaaaaaaaaatctctcttgggagcatataaacaaaaaacatagaagc